SEQUENCE LISTING (139 sequences)

SEQ ID NO: 1 - protein, 21 aa, synthetic construct
MALPVTALLLPLALLLHAARP

SEQ ID NO: 2 - protein, 119 aa, synthetic construct
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 3 - protein, 5 aa, synthetic construct
GGGGS

SEQ ID NO: 4 - protein, 118 aa, synthetic construct
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 5 - protein, 45 aa, synthetic construct
TTTPAPRPPTPAPTIASQPLSLRPEACRPAAGGAVHTRGLDFACD

SEQ ID NO: 6 - protein, 24 aa, synthetic construct
IYIWAPLAGTCGVLLLSLVITLYC

SEQ ID NO: 7 - protein, 42 aa, synthetic construct
KRGRKKLLYIFKQPFMRPVQTTQEEDGCSCRFPEEEEGGCEL

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 63 aa, synthetic construct
ATGGCTCTGCCCGTCACCGCTCTGCTGCTGCCTCTGGCTCTGCTGCTGCACGCTGCTCGCCCT

SEQ ID NO: 10 - protein, 357 aa, synthetic construct
CAGGTCAAACTGGAAGAATCTGGCGGAGGCCTGGTGCAGGCAGGACGGAGCCTGCGCCTGAGCTGCGCAGCATCCGAGCACACCTTCAGCTCCCACGTGATGGGCTGGTTTCGGCAGGCCCCAGGCAAGGAGAGAGAGAGCGTGGCCGTGATCGGCTGGAGGGACATCTCCACATCTTACGCCGATTCCGTGAAGGGCCGGTTCACCATCAGCCGGGACAACGCCAAGAAGACACTGTATCTGCAGATGAACAGCCTGAAGCCCGAGGACACCGCCGTGTACTATTGCGCAGCAAGGAGAATCGACGCAGCAGACTTTGATTCCTGGGGCCAGGGCACCCAGGTGACAGTGTCTAGC

SEQ ID NO: 11 - protein, 15 aa, synthetic construct
GGAGGAGGAGGATCT

SEQ ID NO: 12 - protein, 354 aa, synthetic construct
GAGGTGCAGCTGGTGGAGAGCGGAGGCGGCCTGGTGCAGGCCGGAGGCTCTCTGAGGCTGAGCTGTGCAGCATCCGGAAGAACCTTCACAATGGGCTGGTTTAGGCAGGCACCAGGAAAGGAGAGGGAGTTCGTGGCAGCAATCAGCCTGTCCCCTACCCTGGCCTACTATGCCGAGAGCGTGAAGGGCAGGTTTACCATCTCCCGCGATAACGCCAAGAATACAGTGGTGCTGCAGATGAACTCCCTGAAACCTGAGGACACAGCCCTGTACTATTGTGCCGCCGATCGGAAGAGCGTGATGAGCATTAGACCAGACTATTGGGGGCAGGGAACACAGGTGACCGTGAGCAGC

SEQ ID NO: 13 - protein, 135 aa, synthetic construct
ACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGAT

SEQ ID NO: 14 - protein, 72 aa, synthetic construct
ATCTACATCTGGGCGCCCTTGGCCGGGACTTGTGGGGTCCTTCTCCTGTCACTGGTTATCACCCTTTACTGC

SEQ ID NO: 15 - protein, 126 aa, synthetic construct
AAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTG

SEQ ID NO: 16 - protein, 339 aa, synthetic construct
AGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAA

SEQ ID NO: 17 - protein, 488 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 5 aa, synthetic construct
SHVMG

SEQ ID NO: 19 - protein, 17 aa, synthetic construct
VIGWRDISTSYADSVKG

SEQ ID NO: 20 - protein, 11 aa, synthetic construct
ARRIDAADFDS

SEQ ID NO: 21 - protein, 5 aa, synthetic construct
TFTMG

SEQ ID NO: 22 - protein, 17 aa, synthetic construct
AISLSPTLAYYAESVKG

SEQ ID NO: 23 - protein, 13 aa, synthetic construct
ADRKSVMSIRPDY

SEQ ID NO: 24-100 - skipped (77 entries)

SEQ ID NO: 101 - protein, 5 aa, synthetic construct
GYTMN

SEQ ID NO: 102 - protein, 17 aa, synthetic construct
LINPYNSDTNYAQKLQG

SEQ ID NO: 103 - protein, 9 aa, synthetic construct
VALRVALDY

SEQ ID NO: 104 - protein, 11 aa, synthetic construct
KASQNVATHVG

SEQ ID NO: 105 - protein, 7 aa, synthetic construct
SASYRYS

SEQ ID NO: 106 - protein, 9 aa, synthetic construct
QQYNRYPYT

SEQ ID NO: 107 - protein, 5 aa, synthetic construct
TYAMN

SEQ ID NO: 108 - protein, 19 aa, synthetic construct
RIRSKYNNYATYYAASVKG

SEQ ID NO: 109 - protein, 14 aa, synthetic construct
HGNFGNSYVSWFAY

SEQ ID NO: 110 - protein, 14 aa, synthetic construct
RSSTGAVTTSNYAN

SEQ ID NO: 111 - protein, 7 aa, synthetic construct
GTNKRAP

SEQ ID NO: 112 - protein, 9 aa, synthetic construct
ALWYSNLWV

SEQ ID NO: 113 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 114 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 115 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 116 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 117 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 120 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 121 - protein, 345 aa, synthetic construct
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 122 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 123 - protein, 104 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 124 - protein, 7 aa, synthetic construct
GYSFTGY

SEQ ID NO: 125 - protein, 6 aa, synthetic construct
NPYNSD

SEQ ID NO: 126 - protein, 7 aa, synthetic construct
GFTFNTY

SEQ ID NO: 127 - protein, 8 aa, synthetic construct
RSKYNNYA

SEQ ID NO: 128 - protein, 10 aa, synthetic construct
GYSFTGYTMN

SEQ ID NO: 129 - protein, 10 aa, synthetic construct
LINPYNSDTN

SEQ ID NO: 130 - protein, 10 aa, synthetic construct
GFTFNTYAMN

SEQ ID NO: 131 - protein, 12 aa, synthetic construct
RIRSKYNNYATY

SEQ ID NO: 132 - protein, 8 aa, synthetic construct
GYSFTGYT

SEQ ID NO: 133 - protein, 8 aa, synthetic construct
INPYNSDT

SEQ ID NO: 134 - protein, 11 aa, synthetic construct
ARVALRVALDY

SEQ ID NO: 135 - protein, 6 aa, synthetic construct
QNVATH

SEQ ID NO: 136 - protein, 8 aa, synthetic construct
GFTFNTYA

SEQ ID NO: 137 - protein, 10 aa, synthetic construct
IRSKYNNYAT

SEQ ID NO: 138 - protein, 16 aa, synthetic construct
ARHGNFGNSYVSWFAY

SEQ ID NO: 139 - protein, 9 aa, synthetic construct
TGAVTTSNY

(19) United States
(12) Patent Application Publication
(10) Pub. No.: US 2026/0041768 A1
(43) Pub. Date: Feb. 12, 2026

(54) METHODS FOR TREATING MULTIPLE MYELOMA WITH CAR-T CELLS AND BISPECIFIC ANTIBODIES

(71) Applicant: Janssen Biotech, Inc., Horsham, PA (US)

(72) Inventors: Christoph Johann Heuck, Princeton, NJ (US); Thomas Renaud, Princeton, NJ (US); Gary E. Mason, Doylestown, PA (US); Colleen Marie Kane, Flourtown, PA (US); Jiangxiu Zhou, Willow Grove, PA (US)

(21) Appl. No.: 19/197,644

(22) Filed: May 2, 2025

Related U.S. Application Data
(60) Provisional application No. 63/642,013, filed on May 3, 2024.

(51) Int. Cl. A61K 40/11; A61K 39/00; A61K 39/395; A61K 40/31; A61K 40/42; A61P 35/00

(52) U.S. Cl. CPC A61K 40/11; A61K 39/39558; A61K 40/31; A61K 40/4202; A61P 35/00; A61K 2039/505; A61K 2039/545; A61K 2239/38; A61K 2239/48

(57) ABSTRACT

Provided herein are methods of treating cancer in a subject in need thereof by administering an anti-BCMA CAR-T cell and a GPRC5D×CD3 bispecific antibody. In some embodiments, the subject has relapsed and/or refractory multiple myeloma. In some embodiments, the subject has received at least one prior line of therapy. In some embodiments, the subject has newly diagnosed multiple myeloma and is transplant ineligible.

### CROSS-REFERENCE TO RELATED APPLICATIONS

This application claims priority to Unites States Provisional Application Ser. No. 63/642,013, filed May 3, 2024, the entire contents of which are incorporated herein by reference in their entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “258199091601 (JBI6856USNP1) Sequence Listing.xml”, was created on Apr. 24, 2025, and is 74,680 bytes in size.

### BACKGROUND

Multiple myeloma is a neoplasm of plasma cells that is aggressive. Multiple myeloma is considered to be a B-cell neoplasm that proliferates uncontrollably in the bone marrow. Symptoms include one or more of hypercalcemia, renal insufficiency, anemia, bony lesions, bacterial infections, hyperviscosity and amyloidosis. Multiple myeloma is still considered to be an almost incurable disease, despite availability of new therapies that include proteasome inhibitors, immunomodulatory drugs, and monoclonal antibodies that have significantly improved patient outcomes. Because most patients will either relapse or become refractory to treatment, there is an ongoing need for new therapies for multiple myeloma.

### SUMMARY OF THE DISCLOSURE

Disclosed herein are methods of treating multiple myeloma in a subject comprising administering anti-BCMA CAR-T cells and GPRC5D×CD3 bispecific antibodies.

Disclosed herein is a method of treating multiple myeloma in a subject in need thereof, the method comprising administering ciltacabtagene autoleucel to the subject, and administering a GPRC5D×CD3 bispecific antibody to the subject, wherein the administration of the GPRC5D×CD3 bispecific antibody occurs after the administration of ciltacabtagene autoleucel, and wherein the subject has relapsed and/or refractory multiple myeloma, and received at least three prior lines of therapies, including a proteasomal inhibitor (PI), an immunomodulatory drug (IMiD), and an anti-CD38 antibody.

In some embodiments, ciltacabtagene autoleucel is administered at a dosage of 0.5-1.0×10⁶ CAR-positive viable T cells/kg of the subject.

In some embodiments, the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104, the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109, the LCDR1 of SEQ ID NO: 110, the LCDR2 of SEQ ID NO: 111 and the LCDR3 of SEQ ID NO: 112. In some embodiments, the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116. In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index. In some embodiments, the GPRC5D×CD3 bispecific antibody further comprises proline at position 228, alanine at position 234 and alanine at position 235 in both the HC1 and the HC2. In some embodiments, the GPRC5D×CD3 bispecific antibody comprises a first heavy chain (HC1) having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120. In some embodiments, the GPRC5D×CD3 bispecific antibody is talquetamab.

In some embodiments, the administration of the GPRC5D×CD3 bispecific antibody is once every 2 weeks (Q2W) at a dosage of 0.8 mg/kg. In some embodiments, the administration of the GPRC5D×CD3 bispecific antibody is weekly at a dosage of 0.4 mg/kg.

In some embodiments, the method further comprises administering a conditioning regimen to the subject prior to administering ciltacabtagene autoleucel, wherein the conditioning regimen comprises one or more of cyclophosphamide and/or fludarabine. In some embodiments, the conditioning regimen comprises cyclophosphamide at a dosage of 300 mg/m². In some embodiments, the conditioning regimen comprises fludarabine at a dosage of 30 mg/m². In some embodiments, the conditioning regimen comprises cyclophosphamide at a dosage of 300 mg/m² and fludarabine at a dosage of 30 mg/m². In some embodiments, the conditioning regimen is administered to the subject daily, for up to 3 days.

In some embodiments, the ciltacabtagene autoleucel is administered to the subject 5 to 7 days after the start of the administration of the conditioning regimen. In some embodiments, the ciltacabtagene autoleucel is administered to the subject at a dose of 0.75×10⁶ CAR-positive viable T cells/kg.

In some embodiments, the GPRC5D×CD3 bispecific antibody is administered subcutaneously. In some embodiments, the subject is administered up to 12 cycles of the GPRC5D×CD3 bispecific antibody. In some embodiments, the subject receives three consecutive step-up doses of the GPRC5D×CD3 bispecific antibody of 0.01 mg/kg, 0.06 mg/kg, and 0.4 mg/kg, prior to receiving the 0.8 mg/kg dose once every 2 weeks. In some embodiments, the three consecutive step-up doses are administered 2, 3, 4, or 5 days apart.

In some embodiments, the first cycle of the up to 12 cycles of the GPRC5D×CD3 bispecific antibody comprises the administration of a first step-up dose of 0.01 mg/kg of the GPRC5D×CD3 bispecific antibody, the administration of a second step-up dose of 0.06 mg/kg of the GPRC5D×CD3 bispecific antibody 2, 3, 4, or 5 days after the administration of the first step-up dose, the administration of a third step-up dose of 0.4 mg/kg of the GPRC5D×CD3 bispecific antibody 2, 3, or 4 days after the administration of the second step-up dose, and the administration of 0.8 mg/kg of the GPRC5D×CD3 bispecific antibody 2, 3, 4, 5, 6, 7, 8, 9, or 10 days after the administration of the third step-up dose.

In some embodiments, the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W), and wherein each remaining cycle past the fourth comprises the administration of 0.8 mg/kg of the GPRC5D×CD3 bispecific antibody once every 4 weeks (Q4W).

In some embodiments, the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W), and wherein each remaining cycle past the fifth comprises the administration of 0.8 mg/kg of the GPRC5D×CD3 bispecific antibody once every 4 weeks (Q4W).

In some embodiments, the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W), and wherein each remaining cycle past the sixth comprises the administration of 0.8 mg/kg of the GPRC5D×CD3 bispecific antibody once every 4 weeks (Q4W).

In some embodiments, the administration of the GPRC5D×CD3 bispecific antibody occurs no earlier than 56 days from the administration of ciltacabtagene autoleucel.

In some embodiments, the method achieves a partial response (PR), very good partial response (VGPR), complete response (CR) or stringent complete response (sCR) in the subject, according to IMWG criteria. In some embodiments, the method achieves a very good partial response (VGPR), complete response (CR) or stringent complete response (sCR) in the subject, according to IMWG criteria. In some embodiments, the method achieves a complete response (CR) or stringent complete response (sCR) in the subject, according to IMWG criteria. In some embodiments, the method achieves a stringent complete response (sCR) in the subject, according to IMWG criteria.

In some embodiments, the method achieves MRD-negativity at a threshold of 10⁻⁵ before disease progression or start of a subsequent anti-myeloma therapy. In some embodiments, the method achieves sustained MRD-negative status, as determined by NGS with sensitivity of 10⁻⁵, for at least 6 months without examination showing MRD-positive or progressive disease (PD) in between.

Also disclosed herein are methods of treating multiple myeloma in a subject in need thereof, the method comprising administering ciltacabtagene autoleucel to the subject, and administering a GPRC5D×CD3 bispecific antibody to the subject, wherein the administration of the GPRC5D×CD3 bispecific antibody occurs after the administration of ciltacabtagene autoleucel, and wherein the subject has newly diagnosed multiple myeloma and is transplant ineligible.

In some embodiments, ciltacabtagene autoleucel is administered at a dosage of 0.5-1.0×10⁶ CAR-positive viable T cells/kg of the subject.

In some embodiments, the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104, the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109, the LCDR1 of SEQ ID NO: 110, the LCDR2 of SEQ ID NO: 111 and the LCDR3 of SEQ ID NO: 112. In some embodiments, the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116. In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index. In some embodiments, the GPRC5D×CD3 bispecific antibody further comprises proline at position 228, alanine at position 234 and alanine at position 235 in both the HC1 and the HC2. In some embodiments, the GPRC5D×CD3 bispecific antibody comprises a first heavy chain (HC1) having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120. In some embodiments, the GPRC5D×CD3 bispecific antibody is talquetamab.

In some embodiments, the administration of the GPRC5D×CD3 bispecific antibody is once every 2 weeks (Q2W) at a dosage of 0.8 mg/kg. In some embodiments, the administration of the GPRC5D×CD3 bispecific antibody is weekly at a dosage of 0.4 mg/kg.

In some embodiments, the method further comprises administering a conditioning regimen to the subject prior to administering ciltacabtagene autoleucel, wherein the conditioning regimen comprises one or more of cyclophosphamide and/or fludarabine. In some embodiments, the conditioning regimen comprises cyclophosphamide at a dosage of 300 mg/m². In some embodiments, the conditioning regimen comprises fludarabine at a dosage of 30 mg/m². In some embodiments, the conditioning regimen comprises cyclophosphamide at a dosage of 300 mg/m² and fludarabine at a dosage of 30 mg/m². In some embodiments, the conditioning regimen is administered to the subject daily, for up to 3 days.

In some embodiments, the ciltacabtagene autoleucel is administered to the subject 5 to 7 days after the start of the administration of the conditioning regimen. In some embodiments, the ciltacabtagene autoleucel is administered to the subject at a dose of 0.75×10⁶ CAR-positive viable T cells/kg.

In some embodiments, the method comprises administering 4 cycles of daratumumab, lenalidomide, and dexamethasone (DRd) to the subject prior to the administration of the conditioning regimen, wherein daratumumab is administered at a dosage of 1,800 mg subcutaneously weekly for cycles 1 and 2, followed by once every 2 weeks (Q2W) for cycles 3 and 4, wherein lenalidomide is administered at a dosage of 25 mg orally for 21 days for all 4 cycles, and wherein dexamethasone is administered weekly for all 4 cycles.

In some embodiments, the GPRC5D×CD3 bispecific antibody is administered subcutaneously. In some embodiments, the subject is administered up to 12 cycles of the GPRC5D×CD3 bispecific antibody. In some embodiments, the subject receives three consecutive step-up doses of the GPRC5D×CD3 bispecific antibody of 0.01 mg/kg, 0.06 mg/kg, and 0.4 mg/kg, prior to receiving the 0.8 mg/kg once every 2 weeks (Q2W) dose. In some embodiments, the three consecutive step-up doses are administered 2, 3, 4, or 5 days apart.

In some embodiments, the first cycle of the up to 12 cycles of the GPRC5D×CD3 bispecific antibody comprises the administration of a first step-up dose of 0.01 mg/kg of the GPRC5D×CD3 bispecific antibody, the administration of a second step-up dose of 0.06 mg/kg of the GPRC5D×CD3 bispecific antibody 2, 3, 4, or 5 days after the administration of the first step-up dose, the administration of a third step-up dose of 0.4 mg/kg of the GPRC5D×CD3 bispecific antibody 2, 3, or 4 days after the administration of the second step-up dose, and the administration of 0.8 mg/kg of the GPRC5D×CD3 bispecific antibody 2, 3, 4, 5, 6, 7, 8, 9, or 10 days after the administration of the third step-up dose.

In some embodiments, the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W), and wherein each remaining cycle past the fourth comprises the administration of 0.8 mg/kg of the GPRC5D×CD3 bispecific antibody once every 4 weeks (Q4W).

In some embodiments, the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W), and wherein each remaining cycle past the fifth comprises the administration of 0.8 mg/kg of the GPRC5D×CD3 bispecific antibody once every 4 weeks (Q4W).

In some embodiments, the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W), and wherein each remaining cycle past the sixth comprises the administration of 0.8 mg/kg of the GPRC5D×CD3 bispecific antibody once every 4 weeks (Q4W).

In some embodiments, the administration of the GPRC5D×CD3 bispecific antibody occurs no earlier than 56 days from the administration of ciltacabtagene autoleucel.

In some embodiments, the method achieves a partial response (PR), very good partial response (VGPR), complete response (CR) or stringent complete response (sCR) in the subject, according to IMWG criteria. In some embodiments, the method achieves a very good partial response (VGPR), complete response (CR) or stringent complete response (sCR) in the subject, according to IMWG criteria. In some embodiments, the method achieves a complete response (CR) or stringent complete response (sCR) in the subject, according to IMWG criteria. In some embodiments, the method achieves a stringent complete response (sCR) in the subject, according to IMWG criteria.

In some embodiments, the method achieves MRD-negativity at a threshold of 10⁻⁵ before disease progression or start of a subsequent anti-myeloma therapy. In some embodiments, the method achieves sustained MRD-negative status, as determined by NGS with sensitivity of 10⁻⁵, for at least 6 months without examination showing MRD-positive or PD in between.

Also disclosed herein is a method of treating multiple myeloma in a subject in need thereof, the method comprising administering a GPRC5D×CD3 bispecific antibody to the subject, and administering ciltacabtagene autoleucel to the subject, wherein the administration of ciltacabtagene autoleucel occurs after the administration of the GPRC5D×CD3 bispecific antibody, and wherein the subject has relapsed and/or refractory multiple myeloma, and received at least three prior lines of therapies, including a proteasomal inhibitor (PI), an immunomodulatory drug (IMiD), and an anti-CD38 antibody.

In some embodiments, ciltacabtagene autoleucel is administered at a dosage of 0.5-1.0×10⁶ CAR-positive viable T cells/kg of the subject.

In some embodiments, the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104, the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109, the LCDR1 of SEQ ID NO: 110, the LCDR2 of SEQ ID NO: 111 and the LCDR3 of SEQ ID NO: 112. In some embodiments, the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116. In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index. In some embodiments, the GPRC5D×CD3 bispecific antibody further comprises proline at position 228, alanine at position 234 and alanine at position 235 in both the HC1 and the HC2. In some embodiments, the GPRC5D×CD3 bispecific antibody comprises a first heavy chain (HC1) having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120. In some embodiments, the GPRC5D×CD3 bispecific antibody is talquetamab.

In some embodiments, the administration of the GPRC5D×CD3 bispecific antibody is once every 2 weeks (Q2W) at a dosage of 0.8 mg/kg. In some embodiments, the administration of the GPRC5D×CD3 bispecific antibody is weekly at a dosage of 0.4 mg/kg.

In some embodiments, the method further comprises administering a conditioning regimen to the subject prior to administering ciltacabtagene autoleucel, wherein the conditioning regimen comprises one or more of cyclophosphamide and/or fludarabine. In some embodiments, the conditioning regimen comprises cyclophosphamide at a dosage of 300 mg/m². In some embodiments, the conditioning regimen comprises fludarabine at a dosage of 30 mg/m². In some embodiments, the conditioning regimen comprises cyclophosphamide at a dosage of 300 mg/m² and fludarabine at a dosage of 30 mg/m². In some embodiments, the conditioning regimen is administered to the subject daily, for up to 3 days.

In some embodiments, the ciltacabtagene autoleucel is administered to the subject 5 to 7 days after the start of the administration of the conditioning regimen. In some embodiments, the method further comprises collecting apheresis material from the subject for the production of ciltacabtagene autoleucel. In some embodiments, the collection of apheresis material occurs before the administration of the GPRC5D×CD3 bispecific antibody. In some embodiments, the collection of apheresis material occurs after the administration of the GPRC5D×CD3 bispecific antibody. In some embodiments, the ciltacabtagene autoleucel is administered to the subject at a dose of 0.75×10⁶ CAR-positive viable T cells/kg.

In some embodiments, the GPRC5D×CD3 bispecific antibody is administered subcutaneously. In some embodiments, the subject is administered 2, 3, or 4 cycles of the GPRC5D×CD3 bispecific antibody. In some embodiments, the subject receives three consecutive step-up doses of the GPRC5D×CD3 bispecific antibody of 0.01 mg/kg, 0.06 mg/kg, and 0.4 mg/kg, prior to receiving the 0.8 mg/kg dose. In some embodiments, the three consecutive step-up doses are administered 2, 3, 4, or 5 days apart.

In some embodiments, the first cycle of the 2, 3, or 4 cycles of the GPRC5D×CD3 bispecific antibody comprises the administration of a first step-up dose of 0.01 mg/kg of the GPRC5D×CD3 bispecific antibody, the administration of a second step-up dose of 0.06 mg/kg of the GPRC5D×CD3 bispecific antibody 2, 3, 4, or 5 days after the administration of the first step-up dose, the administration of a third step-up dose of 0.4 mg/kg of the GPRC5D×CD3 bispecific antibody 2, 3, or 4 days after the administration of the second step-up dose, and the administration of 0.8 mg/kg of the GPRC5D×CD3 bispecific antibody 2, 3, 4, 5, 6, 7, 8, 9, or 10 days after the administration of the third step-up dose.

In some embodiments, every cycle of the GPRC5D×CD3 bispecific antibody beyond the first comprises the administration of 0.8 mg/kg of the GPRC5D×CD3 bispecific antibody once every 2 weeks (Q2W).

In some embodiments, the method achieves a partial response (PR), very good partial response (VGPR), complete response (CR) or stringent complete response (sCR) in the subject, according to IMWG criteria. In some embodiments, the method achieves a very good partial response (VGPR), complete response (CR) or stringent complete response (sCR) in the subject, according to IMWG criteria. In some embodiments, the method achieves a complete response (CR) or stringent complete response (sCR) in the subject, according to IMWG criteria. In some embodiments, the method achieves a stringent complete response (sCR) in the subject, according to IMWG criteria.

In some embodiments, the method achieves MRD-negativity at a threshold of 10⁻⁵ before disease progression or start of a subsequent anti-myeloma therapy. In some embodiments, the method achieves sustained MRD-negative status, as determined by NGS with sensitivity of 10⁻⁵, for at least 6 months without examination showing MRD-positive or PD in between.

### BRIEF DESCRIPTION OF THE DRAWINGS

FIGS. 1A and 1B show the treatment pathways across three cohorts in the clinical trial, starting with apheresis and varying cycles of ciltacabtagene autoleucel and talquetamab, leading to primary endpoints assessing safety and efficacy, and secondary endpoints, followed by a long-term follow-up.

FIG. 2 shows the expression of BCMA antigen on the surface of GC, memory and plasmablast cells in the lymph node, long-lived plasma cells in the bone marrow LN and MALT, and on multiple myeloma cells. BAFF-R antigen is not expressed on plasmablast cells, long-lived plasma cells, or multiple myeloma cells. TACI is expressed on memory and plasmablast cells, long-lived plasma cells, and multiple myeloma cells. CD138 is expressed only on long-lived plasma cells and multiple myeloma cells.

FIG. 3 shows the design of the ciltacabtagene autoleucel CAR. Ciltacabtagene autoleucel comprises two VHH domains, as opposed to a single VL domain and a single VH domain found on various other CARs. Ciltacabtagene autoleucel comprises intracellular CD137 and human CD3 zeta domains.

FIG. 4 shows a schematic for preparing virus encoding ciltacabtagene autoleucel CAR, transduction of the virus into a T cell from the patient, and then preparation of CAR T cells expressing ciltacabtagene autoleucel.

FIG. 5 shows bar graphs comparing the expression levels of CD28 and the distribution of T cell memory subtypes in CD3+ T cells from multiple myeloma patients, following in vitro exposure to bispecific T cell engagers talquetamab and teclistamab. The upper graphs show the percentage of CD28+ cells across different days, while the lower graphs detail the percentage of T cell subtypes, including naïve, central memory (CM), effector memory (EM), and terminally differentiated effector memory (TEMRA) cells.

FIG. 6 shows scatter plots demonstrating the fold expansion and the percentage of CAR-positive T cells after ciltacabtagene autoleucel manufacturing from T cells pre-exposed to bispecific T cell engagers. Data from three donors show that previous exposure to bispecifics appears to negatively affect both the expansion capacity of CAR-T cells and the proportion of CAR-positive cells in the resulting drug product, suggesting compromised CAR-T cell manufacturing efficiency.

FIGS. 7A-7B show the functional analysis of CAR-T cells derived from donor T cells pre-exposed to bispecific T cell engagers. FIG. 7A indicates that CAR-T cells pre-exposed to talquetamab and teclistamab have a lower killing efficacy compared to unexposed controls. FIG. 7B displays cytokine production profiles upon stimulation, with the pre-exposed CAR-T cells demonstrating diminished secretion of key cytokines like IFNγ, TNFα, IL-2, and IL-13.

FIG. 8 shows box-and-whisker plots displaying the distribution of lymphocyte counts, monocyte, and neutrophil percentages within the white blood cell (WBC) population in patients who had received CAR-T therapy, those exposed to antibody-drug conjugates (ADC), and those treated with bispecific antibodies (BsAbs).

FIG. 9 shows box-and-whisker plots comparing the percentage of T cells and natural killer (NK) cells within the lymphocyte population in peripheral blood in patients who had received CAR-T therapy, those exposed to antibody-drug conjugates (ADC), and those treated with bispecific antibodies (BsAbs).

FIG. 10 shows the expression of the CD38 marker on CD4+ and CD8+ T cells in patients who had received CAR-T therapy, those exposed to antibody-drug conjugates (ADC), and those treated with bispecific antibodies (BsAbs). The left plot illustrates the percentage of CD4+ T cells expressing CD38, while the right plot shows the percentage of CD8+ T cells with CD38 expression.

FIG. 11 shows two box-and-whisker plots assessing baseline soluble B-cell maturation antigen (sBCMA) levels and the effector-to-target (E:T) ratio in patients who had received CAR-T therapy, those exposed to antibody-drug conjugates (ADC), and those treated with bispecific antibodies (BsAbs). The left plot shows baseline sBCMA concentrations, while the right plot illustrates the E:T ratio, which is calculated as the maximum concentration of sBCMA achieved post-treatment divided by the baseline sBCMA level.

FIG. 12 shows the overall response rate to ciltacabtagene autoleucel therapy in patients previously treated with BCMA targeted therapies, along with the median duration of response (DOR) and progression-free survival (PFS). On the left, a stacked bar graph indicates the percentages of patients achieving partial response (PR), very good partial response (VGPR), complete response (CR), and stringent complete response (sCR). The full cohort shows a 60% PR and 5% sCR, while the ADC exposed group shows a 62% VGPR, and the BsAb exposed group has a 57% VGPR and 14% sCR. On the right, a table compares the median DOR and PFS between the full cohort, ADC exposed, and BsAb exposed groups.

FIGS. 13A-13C show the results of a cytotoxicity assay using spheroids formed from Incucyte® NucLight Green H929 cells to evaluate the effects of bispecific antibodies in combination with CAR-T cells versus MOCK cells. FIG. 13A shows the relative size of spheroids over time after the addition of MOCK cells at various concentrations of talquetamab. FIG. 13B illustrates the same for CAR-T cells, showing that the spheroids treated with CAR-T cells exhibit a change in size indicative of cytotoxicity across a gradient of bispecific antibody concentrations. FIG. 13C shows an Area Under the Curve (AUC) analysis comparing the potency of bispecific antibody engagement between MOCK and CAR-T cells across a logarithmic concentration range.

FIGS. 14A-14B show flow cytometry results corresponding to CAR-positive T cell populations and their enrichment. FIG. 14A illustrates flow cytometry plots demonstrating the distinction between MOCK cells, double-positive T cells expressing both CD4 and CD8, and CAR-T cells enriched using biotinylated BCMA and anti-biotin magnetic beads. FIG. 14B provides a tabulated summary of the percentage of CAR-positive cells along with the total T cell count and the absolute number of CAR-positive T cells at varying levels of dilution.

FIG. 15 shows a dose-response curve, presenting the Area Under the Curve (AUC) analysis of the effect of varying log concentrations of talquetamab on spheroid size over time. Each curve represents a different dilution of CAR-positive T cells.

FIGS. 16A-16B show the efficacy of talquetamab engagement with varying levels of CAR-T cell concentrations. FIG. 16A presents a bar graph of the half maximal effective concentration (EC50) values for different dilutions of CAR-positive T cells, indicating the concentration of talquetamab required to achieve half of the maximum effect. Lower EC50 values suggest higher potency. FIG. 16B shows the fold reduction in EC50 relative to the MOCK cells, illustrating the increased sensitivity of talquetamab in the presence of CAR-T cells.

FIGS. 17A-17C show the impact of talquetamab concentration on T cell activation and expansion in the context of CAR-T cell therapy. FIG. 17A and FIG. 17B depict the mean fluorescence intensity (MFI) of CD25 on CD4 and CD8 T cells respectively after 48 hours, with the MFI serving as an indicator of T cell activation. FIG. 17C shows the fold expansion of T cells over five days in relation to the log concentration of talquetamab.

FIGS. 18A-18B show the effect of talquetamab on the expression of interferon-gamma (IFN-γ) by T cells at different time points post-treatment. FIG. 18A, corresponding to the 48-hour mark, displays a dose-response curve where the IFN-γ concentration is plotted against the log concentration of talquetamab. FIG. 18B shows the 120-hour time point.

FIG. 19 shows the expression levels of the Fas ligand and CD54 molecules (ICAM-1) in H929 cells following the addition of CAR-T cells, compared to MOCK cells (T cells without CAR construct) and no T cell controls at both 24 and 48 hours after T cell addition.

### DETAILED DESCRIPTION

The present disclosure provides methods of treating patients with multiple myeloma with CAR-T cells and anti-GPRC5D antibodies. In some embodiments, the methods relate to treating patients with multiple myeloma who have relapsed and/or have refractory multiple myeloma and/or have received prior lines of therapies. In some embodiments, the methods relate to treating patients with multiple myeloma who have newly diagnosed multiple myeloma and are transplant ineligible.

Several aspects and embodiments of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure.

Any references in the description or in the claims to methods of treatment refer to the compounds, compositions, pharmaceutical compositions and medicaments for use in a method of treatment of the human (or animal) body by therapy (or for diagnosis).

Any references in the description or in the claims to methods of treatment refer to the use of the compounds, compositions, pharmaceutical compositions for the manufacture of a medicament for the treatment of the human (or animal) body by therapy (or for diagnosis).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### Definitions

The terminology used herein is for the purpose of describing particular aspects or embodiments only and is not intended to be limiting.

As used herein, the indefinite articles “a”, “an” and “the” should be understood to include plural reference unless the context clearly indicates otherwise.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein. Antibodies contemplated herein include single-domain antibodies, such as heavy chain only antibodies. The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotype (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Antibody light chains of any vertebrate species may be assigned to one of two clearly distinct types, namely kappa (κ) and lambda (λ), based on the amino acid sequences of their constant domains.

“Antigen binding fragment” or “antigen binding domain” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

The antibodies, polypeptides, and proteins of embodiments of the disclosure (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The antibodies, polypeptides, and/or proteins of embodiments of the disclosure (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the antibodies, polypeptides, and proteins of the disclosure (including functional portions and functional variants thereof) can be isolated and/or purified from a source, such as a plant, a bacterium, an insect, a mammal, etc. Methods of isolation and purification are known in the art. Alternatively, the antibodies, polypeptides, and/or proteins described herein (including functional portions and functional variants thereof) can be commercially synthesized. In this respect, the antibodies, polypeptides, and proteins can be synthetic, recombinant, isolated, and/or purified.

The terms “B-cell maturation antigen” and “BCMA” as used herein include human B cell maturation antigen, also known as BCMA, CD269, and TNFRSF17 (UniProt Q02223), which is a member of the tumor necrosis receptor superfamily that is preferentially expressed in differentiated plasma cells. The extracellular domain of human BCMA consists, according to UniProt of amino acids 1-54 (or 5-51).

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 122. SEQ ID NO: 123 shows the extracellular domain of CD3 epsilon.

“CD38” refers to the human CD38 protein (UniProt accession no. P28907) (synonyms: ADP-ribosyl cyclase 1, cADPr hydrolase 1, cyclic ADP-ribose hydrolase 1). CD38 is a single pass type II transmembrane protein with amino acid residues 1-21 representing the cytosolic domain, amino acid residues 22-42 representing the transmembrane domain, and residues 43-300 representing the extracellular domain.

“CH3 region” or “CH3 domain” refers to the CH3 region of an immunoglobulin. The CH3 region of human IgG1 antibody corresponds to amino acid residues 341-446. However, the CH3 region may also be any of the other antibody isotypes as described herein. The substitutions in the CH3 region are expressed as modified position(s) in the first CH3 domain of the first heavy chain/modified position(s) in the second CH3 domain of the second heavy chain. For example, F405L/K409R refers to a F405L mutation in the first CH3 region and K09R mutation in the second CH3 region. L351Y_F405A_Y407V/T394W refers to L351Y, F40FA and Y407V mutations in the first CH3 region and T394W mutation in the second CH3 region. D399FHKRQ/K409AGRH refers to mutation in which D399 may be replaced by F, H, K R or Q, and K409 may be replaced by A, G, R or H.

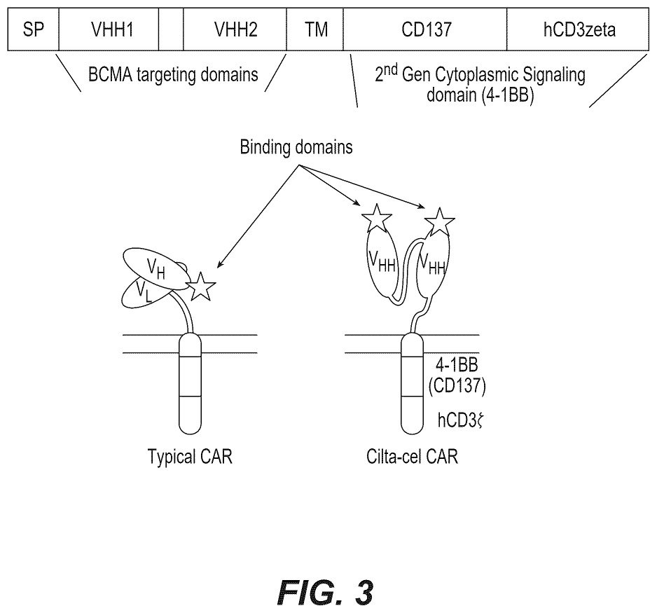

“Ciltacabtagene autoleucel” (“cilta-cel”) is a chimeric antigen receptor T cell (CAR-T) therapy comprising two B-cell maturation antigen (BCMA)-targeting VHH domains designed to confer avidity for BCMA. Ciltacabtagene autoleucel can comprise T lymphocytes transduced with the ciltacabtagene autoleucel CAR, a CAR encoded by a lentiviral vector. The CAR targets the human B cell maturation antigen (anti-BCMA CAR). A diagram of the lentiviral vector encoding ciltacabtagene autoleucel CAR is provided in FIG. 3. The amino acid sequence of the ciltacabtagene autoleucel CAR is the amino acid sequence of SEQ ID NO: 17.

A “chimeric antigen receptor” or “CAR” is an artificially constructed hybrid protein or polypeptide containing the antigen binding domains of at least one antibody (or antibody fragment) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, advantageously, CARs do not dimerize with endogenous T cell receptor (TCR) α- and β-chains. T cells expressing a CAR are referred to herein as CAR T cells, CAR-T cells or CAR modified T cells, and these terms are used interchangeably herein. The cell can be genetically modified to stably express at least one antigen-binding domain on its surface, conferring novel antigen specificity that is MHC independent. “BCMA CAR” refers to a CAR having an extracellular binding domain specific for BCMA. “Bi-epitope CAR” refers to a CAR having an extracellular binding domain specific for two different epitopes of an antigen, such as BCMA.

“Combination” means that two or more therapeutics are administered to a subject together in a mixture, concurrently as single agents or sequentially as single agents in any order.

“Complementarity determining regions” (CDR) are antibody regions that bind an antigen. CDRs may be defined using various delineations such as Kabat (Wu et al. J Exp Med 132:211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196:901-17, 1987), IMGT (Lefranc et al. Dev Comp Immunol 27:55-77, 2003) and AbM (Martin and Thornton J Bmol Biol 263:800-15, 1996). The correspondence between the various delineations and variable region numbering are described (see e.g., Lefranc et al. Dev Comp Immunol 27:55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat; Chothia; Martin; Lefranc et al.).

TABLE 1
Kabat, IMGT, AbM, and Chothia numbering systems.

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Cycle” as used herein, refers to a recurring period of 28 days, demarcated for the purpose of administering the prescribed interventions. Within this framework, the term “cycle” encapsulates the entire sequence of events, activities, and drug administration protocols designated for this 28-day interval.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In some embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in-between and above 1, e.g., 1.5, 1.6, 1.7. 1.8, etc.) the response (reference response) produced by vehicle, a control composition, or the response in a particular cell lineage.

The terms “daratumumab” and “daratumumab SC” refers to the anti-CD38 antibody DARZALEX FASPRO® PI 2020.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In some embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30, or more times (e.g., 500, 1000 times) (including all integers and decimal points in-between and above 1, e.g., 1.5, 1.6, 1.7. 1.8, etc.) the response produced by vehicle or a control composition. “Enhance” or “enhanced” also refers to enhancement in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g., ADCC, CDC and/or ADCP) or binding to an Fcγ receptor (FcγR) or FcRn. “Enhanced” may be an enhancement of about 10%, 20%, 30%, 40%, 50%, 60%, 70%, 80%, 90%, 100% or more, or a statistically significant enhancement.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

“Fc gamma receptor” (FcγR) refers to well-known FcγRI, FcγRIIa, FcγRIIb or FcγRIII. Activating FcγR includes FcγRI, FcγRIIa and FcγRIII.

The terms “fragment of an antibody”, “antibody fragment”, “functional fragment of an antibody”, and “antigen-binding portion” are used interchangeably herein to mean one or more fragments or portions of an antibody that retain the ability to specifically bind to an antigen (see, generally, Holliger et al., Nat. Biotech., 23(9): 1 126-1129 (2005)). The antigen recognition moiety of the CAR encoded by the nucleic acid sequence disclosed herein can contain any BCMA-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242:423-426 (1988); Huston et al., Proc. Natl. Acad. Sci. USA, 85:5879-5883 (1988); and Osbourn et al., Nat. Biotechnol, 16:778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)2, Fd and Fv fragments, domain antibodies (dAb) consisting of one VH domain or one VL domain, shark variable IgNAR domains, VH domains modified to function without a corresponding VL domain, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

A “full length antibody” is comprised of two heavy chains (HC) and two light chains (LC) inter-connected by disulfide bonds as well as multimers thereof (e.g., IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FW). Each VH and VL is composed of three CDRs and four FW segments, arranged from amino-to-carboxy-terminus in the following order: FW1, CDR1, FW2, CDR2, FW3, CDR3 and FW4.

“GPRC5D” refers to human G-protein coupled receptor family C group 5 member D having the amino acid sequence shown in SEQ ID NO: 121.

“GPRC5D×CD3 bispecific antibody” refers to a molecule containing two or more binding regions, wherein one of the binding regions specifically binds the cell surface antigen G Protein-Coupled Receptor Class C Group 5 Member D antigen (GPRC5D) on a target cell or tissue and wherein a second binding region of the molecule specifically binds a T cell antigen CD3. This dual/multi-target binding ability recruit T cells to the target cell or tissue leading to the eradication of the target cell or tissue.

The phrase “hazard ratio” refers to a measure of the relative rate of progression to an endpoint as compared to a control group. In outcome-based clinical trials, a reduction in the hazard ratio for a test arm as compared to the control indicates the treatment used in the test arm reduces the risk of the endpoint, in the case of the studies described herein, disease progression or death. Preferably hazard ratio is calculated per a stratified constant piecewise weighted log-rank test.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “line of therapy,” as used in connection with methods of treatment herein, refers to one or more cycles of a planned treatment program, which may have consisted of one or more planned cycles of single-agent therapy or combination therapy, as well as a sequence of treatments administered in a planned manner. For example, a planned treatment approach of induction therapy followed by autologous stem cell transplantation followed by maintenance is one line of therapy. A new line of therapy is considered to have started when a planned course of therapy has been modified to include other treatment agents or medicaments (alone or in combination) as a result of disease progression, relapse, or toxicity. A new line of therapy is also considered to have started when a planned period of observation off therapy had been interrupted by a need for additional treatment for the disease.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Multispecific” refers to an antibody that specifically binds at least two distinct antigens or at least two distinct epitopes within the same antigen. Multispecific antibody may bind for example two, three, four or five distinct antigens or distinct epitopes within the same antigen.

“Mutation” refers to an engineered or naturally occurring alteration in a polypeptide or polynucleotide sequence when compared to a reference sequence. The alteration may be a substitution, insertion or deletion of one or more amino acids or polynucleotides.

“Newly diagnosed multiple myeloma” (NDMM) refers to the initial identification of multiple myeloma in a patient, which has not been previously diagnosed or treated. Diagnostic criteria for myeloma must be met when the participant was diagnosed. In some embodiments, multiple myeloma is defined as clonal bone marrow plasma cells ≥10% or biopsy-proven bony or extramedullary plasmacytoma and any one or more of the following myeloma-defining events:
Evidence of end organ damage that can be attributed to the underlying plasma cell proliferative disorder, specifically:
   C: Hypercalcemia: serum calcium >0.25 mmol/L (>1 mg/dL) higher than the ULN or >2.75 mmol/L (>11 mg/dL)
   R: Renal insufficiency: CrCl<40 mL/min (measured or estimated by validated equations) or serum creatinine >177 μmol/L (>2 mg/dL)
   A: Anemia: hemoglobin value >20 g/L below the lower limit of normal, or hemoglobin value <100 g/L
   B: Bone lesions: ≥1 osteolytic lesions on skeletal radiography, CT, or PET/CT (If bone marrow has less than 10% clonal plasma cells, more than one bone lesion is required to distinguish from solitary plasmacytoma with minimal marrow involvement; PET/CT=¹⁸F-fluorodeoxyglucose PET with CT)
Any one or more of the following biomarkers of malignancy:
   Clonal bone marrow plasma cell percentage ≥60% (these values are based on the serum Freelite assay [The Binding Site Group, Birmingham, United Kingdom]; the involved FLC must be ≥100 mg/L)
   Involved: uninvolved serum FLC ratio ≥100 (each focal lesion must be ≥5 mm in size)
   >1 focal lesion on MRI studies (Rajkumar 2014)

Clonality should be established by showing κ/λ-light chain restriction on flow cytometry, immunofluorescence, or IHC. Bone marrow plasma cell percentage should preferably be estimated from a biopsy specimen; in case of a disparity between the aspirate and biopsy, the highest value should be used.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis material” refers to material obtained using apheresis.

“Non-fixed combination” refers to separate pharmaceutical compositions of the T cell redirecting therapeutic and the anti-CD38 antibody administered as separate entities either simultaneously, concurrently or sequentially with no specific intervening time limits, wherein such administration provides effective levels of the two compounds in the body of the subject.

The phrase “nonresponsive disease” refers to either failure to achieve minimal response or to development of progressive disease while on therapy.

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

“Pomalidomide” also termed “POMALYST®” refers to an analog of thalidomide, which is a third generation IMiD (immunomodulatory drug) with antineoplastic activity. IMiDs, such as lenalidomide and pomalidomide, form the backbone of several current multiple myeloma treatment regimens. Their exact mechanism of action is not fully understood, but IMiDs have an immunomodulatory effect on the multiple myeloma tumor microenvironment and may affect expression of tumor suppressor genes, promote apoptosis of myeloma cells, and enhance NK mediated myeloma cell lysis. The combination of daratumumab with IMiDs has been evaluated in multiple studies and demonstrated significant improvement in efficacy.

“Pharmaceutical composition” refers to composition that comprises an active ingredient and a pharmaceutically acceptable carrier.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

“Pharmaceutically acceptable carrier” or “excipient” refers to an ingredient in a pharmaceutical composition, other than the active ingredient, which is nontoxic to a subject.

“Philadelphia chromosome” or “Ph” refers to a well-known chromosomal translocation between chromosomes 9 and 22, resulting in the oncogenic BCR-ABL gene fusion with constitutively active tyrosine kinase activity. The translocation results in a portion of the BCR gene from chromosome 22q11 becoming fused with a portion of the ABL gene from chromosome 9q34, and is designated as t (9;22) (q34;q11) under the International System for Human Cytogenetic Nomenclature (ISCN). Depending on the precise location of the fusion, the molecular weight of the resulting fusion protein can range from 185 to 210 kDa. “Philadelphia chromosome” refers to all BCR-ABL fusion proteins formed due the (9;22) (q34;q11) translocation.

The term “protein” or “polypeptide” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

“Recombinant” refers to DNA, antibodies and other proteins that are prepared, expressed, created or isolated by recombinant means when segments from different sources are joined to produce recombinant DNA, antibodies or proteins.

“Reduce” or “reduced” refers to a reduction in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g., ADCC, CDC and/or ADCP) or binding to an Fcγ receptor (FcγR) or FcRn. “Reduced” may be a reduction of about 10%, 20%, 30%, 40%, 50%, 60%, 70%, 80%, 90%, 100% or more, or a statistically significant enhancement.

The term “refractory,” as used in connection to treatment with a particular treatment agent or medicament or line of therapy herein, refers to diseases or disease subjects that fail to respond to said treatment agent or medicament or line of therapy. The phrase “refractory myeloma” refers to multiple myeloma that is nonresponsive while on primary or salvage therapy or that has progressed within 60 days of last therapy.

“Relapsed” refers to a cancer that responded to treatment but then returns.

The term “single-domain antibody” or “sdAb” refers to a single antigen-binding polypeptide having three complementarity determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs”. Some VHHs may also be known as “Nanobodies”. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabch et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

As used herein, the terms “specifically binds”, “specifically recognizes”, or “specific for” refer to measurable and reproducible interactions such as binding between a target and an antigen binding protein (such as a CAR or a VHH), which is determinative of the presence of the target in the presence of a heterogeneous population of molecules including biological molecules.

The term “specificity” refers to selective recognition of an antigen binding protein (such as a CAR or a VHH) for a particular epitope of an antigen. Natural antibodies, for example, are monospecific. The term “multispecific” denotes that an antigen binding protein (such as a CAR or antibody) has two or more antigen-binding sites of which at least two bind different antigen-binding specificities. “Bispecific” as used herein denotes that an antigen binding protein (such as a CAR or antibody) has two different antigen-binding specificities.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovines (cows) and swines (pigs) or of the order Perssodactyla, including equines (horses). The mammals may be of the order Primates, Ceboids, or Simoids (monkeys) or of the order Anthropoids (humans and apes). In some embodiments, the mammal is a human.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs are typically transcription factor Foxp3-positive CD4+T cells and can also include transcription factor Foxp3-negative regulatory T cells that are IL-10-producing CD4+T cells.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Transplant-ineligible” means patients who, due to older age, high comorbid burden, or poor performance status, are at an increased risk of treatment-related toxicities. Grant, Shakira J et al., Journal of Geriatric Oncology, vol. 12, 4 (2021).

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilization (i.e., a cessation in the worsening) of the state of disease, delay or slowing of disease progression, amelioration or palliation of the disease state, and/or remission (whether partial or total and whether detectable or undetectable). “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The term “variable” refers to the fact that certain segments of the variable domains differ extensively in sequence among antibodies. The V domain (i.e., variable domain) mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a β-sheet configuration, connected by three HVRs, which form loops connecting, and in some cases forming part of, the β-sheet structure. The HVRs in each chain are held together in close proximity by the FR regions and contribute to the formation of the antigen binding site of antibodies (with the HVRs from the other chain, if the antibody is not a sdAb or HCAb) (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH” domain. VHH is thus a special type of variable region.

The dosing frequencies provided for herein are understood to be synonymous with standard terms in the art. For example, “weekly” dosing is understood to be synonymous with “QW”. For example, “biweekly” dosing is understood to be synonymous with “Q2W”. For example, “once every four weeks” is understood to be synonymous with “Q4W”. Unless explicitly stated to the contrary, “once every four weeks” and “monthly” are used interchangeably in the context of dosing frequencies. Accordingly, “monthly” or “once a month” is also understood to be synonymous with “Q4W” unless explicitly stated otherwise.

When referring to a dosage amount, “μg/kg” or “mg/kg” refers to the amount of an active agent, such as a bispecific antibody or antibody, in microgram (μg) or milligram (mg) administered to a subject per kilogram (kg) body weight of the subject.

Additionally, throughout this disclosure, various aspects and embodiments of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 2.

TABLE 2
Amino acid abbreviations.

| | Three-letter | One-letter |
|---|---|---|
| Amino acid | code | code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Chimeric Antigen Receptors and Immune Effector Cell Compositions

International Patent Publication No. WO 2018/028647 is incorporated by reference herein in its entirety. US Patent Publication No. 2018/0230225 is incorporated by reference herein in its entirety. International Patent Application No. PCT/CN2020/133598 is incorporated by reference herein in its entirety.

The disclosure provides for methods of treating a subject with cells expressing a chimeric antigen receptor (CAR). The CAR comprises an extracellular antigen binding domain comprising one or more single-domain antibodies. In various embodiments, there is provided a CAR targeting BCMA (also referred herein as “BCMA CAR”) comprising a polypeptide comprising: (a) an extracellular antigen binding domain comprising an anti-BCMA binding moiety; (b) a transmembrane domain; and (c) an intracellular signaling domain. In some embodiments, the anti-BCMA binding moiety is camelid, chimeric, human, or humanized. In some embodiments, the intracellular signaling domain comprises a primary intracellular signaling domain of an immune effector cell (such as T cell). In some embodiments, the primary intracellular signaling domain is derived from CD4. In some embodiments, the primary intracellular signaling domain is derived from CD3-zeta. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In some embodiments, the co-stimulatory signaling domain is derived from CD137.

In some embodiments, the BCMA CAR further comprises a hinge domain (such as a CD8-alpha hinge domain) located between the C-terminus of the extracellular antigen binding domain and the N-terminus of the transmembrane domain. In some embodiments, the BCMA CAR further comprises a signal peptide (such as a CD8-alpha signal peptide) located at the N-terminus of the polypeptide. In some embodiments, the polypeptide comprises from the N-terminus to the C-terminus: a CD8-alpha signal peptide, the extracellular antigen-binding domain, a CD8-alpha hinge domain, a CD28 transmembrane domain, a first co-stimulatory signaling domain derived from CD28, a second co-stimulatory signaling domain derived from CD137, and a primary intracellular signaling domain derived from CD4. In some embodiments, the polypeptide comprises from the N-terminus to the C-terminus: a CD8-alpha signal peptide, the extracellular antigen-binding domain, a CD8-alpha hinge domain, a CD8-alpha transmembrane domain, a second co-stimulatory signaling domain derived from CD137, and a primary intracellular signaling domain derived from CD3-zeta. In some embodiments, the BCMA CAR is monospecific. In some embodiments, the BCMA CAR is monovalent.

The present application also provides CARs that have two binding moieties that specifically bind to an antigen, such as BCMA. In some embodiments, one or both binding moieties are antigen binding fragments. In some embodiments, one or both binding moieties comprise single-domain antibodies. In some embodiments, one or both binding moieties comprise a VHH.

In some embodiments, the CAR is a bivalent CAR comprising a polypeptide comprising: (a) an extracellular antigen binding domain comprising two binding moieties specifically binding to an antigen (such as a tumor antigen); (b) a transmembrane domain; and (c) an intracellular signaling domain.

In some embodiments, the binding moieties, such as VHHs (including the plurality of VHHs, or the first VHH and/or the second VHH) are camelid, chimeric, human, or humanized. In some embodiments, the binding moieties or VHHs are connected to each other via peptide bonds or peptide linkers. In some embodiments, each peptide linker is no more than about 50 (such as no more than about any one of 35, 25, 20, 15, 10, or 5) amino acids long.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH.

In some embodiments, the first anti-BCMA binding moiety comprises a first complementarity determining region (CDR1) comprising the amino acid sequence of SEQ ID NO: 18. In some embodiments, the first anti-BCMA binding moiety comprises a second complementarity determining region (CDR2) comprising the amino acid sequence of SEQ ID NO: 19. In some embodiments, the first anti-BCMA binding moiety comprises a third complementarity determining region (CDR3) comprising the amino acid sequence of SEQ ID NO: 20.

In some embodiments, the first anti-BCMA binding moiety comprises a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20.

In some embodiments, the first BCMA binding moiety comprises the amino acid sequence of SEQ ID NO: 2. In some embodiments, the first BCMA binding moiety comprises a polypeptide encoded by the nucleic acid sequence of SEQ ID NO: 10. In some embodiments, the first anti-BCMA binding moiety comprises one or more of, or all of, SEQ ID NO: 18, SEQ ID NO: 19, SEQ ID NO: 20 and SEQ ID NO: 2. These sequences correspond to the sequences present in ciltacabtagene autoleucel.

In some embodiments, the second BCMA binding moiety comprises a first complementarity determining region (CDR1) comprising the amino acid sequence of SEQ ID NO: 21. In some embodiments, the second BCMA binding moiety comprises a second complementarity determining region (CDR2) comprising the amino acid sequence of SEQ ID NO: 22. In some embodiments, the second BCMA binding moiety comprises a third complementarity determining region (CDR3) comprising the amino acid sequence of SEQ ID NO: 23.

In some embodiments, the second BCMA binding moiety comprises a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23.

In some embodiments, the second BCMA binding moiety comprises the amino acid sequence of SEQ ID NO: 4. In some embodiments, the second BCMA binding moiety comprises a polypeptide encoded by the nucleic acid sequence of SEQ ID NO: 12. In some embodiments, the second anti-BCMA binding moiety comprises one or more of, or all of, SEQ ID NO: 21, SEQ ID NO: 22, SEQ ID NO: 23 and SEQ ID NO: 4. These sequences correspond to the sequences present in ciltacabtagene autoleucel.

In some embodiments, the first BCMA binding moiety and the second BCMA binding moiety are connected to each other via a peptide linker. In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR further comprises a hinge domain (such as a CD8-alpha hinge domain) located between the C-terminus of the extracellular antigen binding domain and the N-terminus of the transmembrane domain. In some embodiments, the CAR further comprises a signal peptide (such as a CD8-alpha signal peptide) located at the N-terminus of the polypeptide.

Without wishing to be bound by theory, the CARs that are multivalent, or those CARs comprising an extracellular antigen binding domain comprising a first BCMA binding moiety and a second BCMA binding moiety, may be suitable for targeting multimeric antigens via synergistic binding by the different antigen binding sites, or for enhancing binding affinity or avidity to the antigen. Improved avidity may allow for a substantial reduction in the dose of CAR-T cells needed to achieve a therapeutic effect, such as a dose ranging from 4.0×10⁴ to 1.0×10⁶ CAR-T cells per kilogram of the mass of the subject, or 3.0×10⁶ to 1.0×10⁸ total CAR-T expressing cells. Monovalent CARs, such as idecabtagene vicleucel, may need to be dosed at 5 to 10 times these amounts to achieve a comparable effect. In various embodiments, reduced dosage ranges may provide for substantial reduction in cytokine release syndrome (CRS) and other potentially dangerous side-effects of CAR-T therapy.

The various binding moieties (e.g., an extracellular antigen binding domain comprising a first BCMA binding moiety and a second BCMA binding moiety) in the CARs described herein may be connected to each other via peptide linkers. The peptide linkers connecting different binding moieties (such as VHHs) may be the same or different. Different domains of the CARs may also be connected to each other via peptide linkers. In some embodiments, the binding moieties (such as VHHs) are directly connected to each other without any peptide linkers.

The peptide linker in the CARs described herein can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The CARs of the present application comprise a transmembrane domain that can be directly or indirectly connected to the extracellular antigen binding domain.

The CAR may comprise a T-cell activation moiety. The T-cell activation moiety can be any suitable moiety derived or obtained from any suitable molecule. In one embodiment, for example, the T-cell activation moiety comprises a transmembrane domain. The transmembrane domain can be any transmembrane domain derived or obtained from any molecule known in the art. For example, the transmembrane domain can be obtained or derived from a CD8α molecule or a CD28 molecule. Without wishing to be bound by theory, CD8 is a transmembrane glycoprotein that serves as a co-receptor for the T-cell receptor (TCR) and is expressed primarily on the surface of cytotoxic T-cells. The most common form of CD8 exists as a dimer composed of a CD8 alpha (CD8α) and CD8 beta (CD8β) chain. CD28 is expressed on T-cells and provides co-stimulatory signals required for T-cell activation. CD28 is the receptor for CD80 (B7.1) and CD86 (B7.2). In a preferred embodiment, the CD8α and CD28 are human.

In addition to the transmembrane domain, the T-cell activation moiety may further comprise an intracellular (i.e., cytoplasmic) T-cell signaling domain. The intercellular T-cell signaling domain can be obtained or derived from a CD28 molecule, a CD3 zeta (¿) molecule or modified versions thereof, a human Fc receptor gamma (FcRy) chain, a CD27 molecule, an OX40 molecule, a 4-1BB molecule, or other intracellular signaling molecules known in the art. Without wishing to be bound by theory: (1) CD28 is a T-cell marker important in T-cell co-stimulation; (2) CD33 associates with TCRs to produce a signal and contains immunoreceptor tyrosine-based activation motifs (ITAMs); and (3) 4-1BB, also known as CD137, transmits a potent costimulatory signal to T-cells, promoting differentiation and enhancing long-term survival of T lymphocytes. In a preferred embodiment, the CD28, CD3 zeta, 4-IBB, OX40, and CD27 are human.

The T-cell activation domain of the CAR encoded by the nucleic acid sequence disclosed herein can comprise any one of aforementioned transmembrane domains and any one or more of the aforementioned intercellular T-cell signaling domains in any combination. For example, the nucleic acid sequence disclosed herein can encode a CAR comprising a CD28 transmembrane domain and intracellular T-cell signaling domains of CD28 and CD3 zeta. Alternatively, for example, the nucleic acid sequence disclosed herein can encode a CAR comprising a CD8α transmembrane domain and intracellular T-cell signaling domains of CD28, CD3 zeta, the Fc receptor gamma (FcRy) chain, and/or 4-1 BB.

In some embodiments, the CAR polypeptide further comprises a signal peptide located at the N-terminus of the polypeptide. In some embodiments, the signal peptide is derived from CD8-alpha (CD8α SP). In some embodiments, the signal peptide comprises the amino acid sequence of SEQ ID NO: 1. In some embodiments, the signal peptide comprises a polypeptide encoded by the nucleic acid sequence of SEQ ID NO: 9.

In some embodiments, the transmembrane domain comprises the amino acid sequence of SEQ ID NO: 6. In some embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3ζ. In some embodiments, the intracellular signaling domain comprises at least one co-stimulatory signaling domains. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises an amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the CAR comprises a first and a second anti-BCMA binding moiety, wherein the first anti-BCMA binding moiety comprises a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; wherein the second BCMA binding moiety comprises a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23; wherein the CAR further comprises: a transmembrane domain derived from CD8α, wherein optionally the transmembrane domain comprises the amino acid sequence of SEQ ID NO: 6; a primary intracellular signaling domain derived from CD3ζ, wherein optionally the primary intracellular signaling domain comprises the amino acid sequence of SEQ ID NO: 8; a co-stimulatory signaling domain comprising a cytoplasmic domain of CD137, wherein optionally the co-stimulatory signaling domain comprises the amino acid sequence of SEQ ID NO: 7; and a hinge domain located between the C-terminus of the extracellular antigen binding domain and the N-terminus of the transmembrane domain, wherein the hinge domain is derived from CD8α, wherein optionally the hinge domain comprises the amino acid sequence of SEQ ID NO: 5. In certain such embodiments, the first VHH domain comprises the amino acid sequence of SEQ ID NO: 2 and the second VHH domain comprises the amino acid sequence of SEQ ID NO: 4.

In some embodiments, the CAR comprises one or more of, or all of, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 18, SEQ ID NO: 19, SEQ ID NO: 20, SEQ ID NO: 21, SEQ ID NO: 22 and SEQ ID NO: 23. In some embodiments, the CAR comprises SEQ ID NO: 17. In some embodiments, the CAR comprises a polypeptide encoded by the nucleic acid sequence of one or more of, or all of, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

In preferred embodiments, the CAR comprises a first VHH domain comprising a CDR1, a CDR2 and a CDR3 of the VHH domain comprising the amino acid sequence of SEQ ID NO: 2, and a second VHH domain comprising a CDR1, a CDR2 and a CDR3 of the VHH domain comprising the amino acid sequence of SEQ ID NO: 4. In preferred embodiments, the first VHH domain is linked to the second VHH domain via a linker comprising the amino acid sequence of SEQ ID NO: 3. In particularly preferred embodiments, the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23. In further preferred embodiments, the CAR comprises a first VHH domain comprising the amino acid sequence of SEQ ID NO: 2, and a second VHH domain comprising the amino acid sequence of SEQ ID NO: 4.

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils. In some embodiments, the immune effector cells are T cells. In some embodiments, the T cells are autologous T cells. In some embodiments, the T cells are allogeneic T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells, such as CD20+ or CD19+ tumor cells. In some embodiments, the CD8+ T cells lyse antigen-specific target cells upon expressing the CAR and binding to the target cells.

Biological methods for introducing the vector into an immune effector cell include the use of DNA and RNA vectors. Viral vectors have become the most widely used method for inserting genes into mammalian, e.g., human cells.

Provided herein are dosage forms comprising 3.0×10⁷ to 1.0×10⁸ CAR-T cells comprising a CAR comprising a polypeptide provided herein. Provided herein are dosage forms comprising 3.0×10⁷ to 1.0×10⁸ CAR-T cells comprising a CAR comprising a polypeptide comprising: (a) an extracellular antigen binding domain comprising a first BCMA binding moiety specifically binding to a first epitope of BCMA, and a second BCMA binding moiety specifically binding to a second epitope of BCMA; (b) a transmembrane domain; and (c) an intracellular signaling domain, wherein the first epitope and the second epitope are different. In some embodiments, there are provided dosage forms comprising 3.0×10⁷ to 1.0×10⁸ engineered immune effector cells (such as T-cells) comprising a CAR comprising a polypeptide comprising: (a) an extracellular antigen binding domain comprising a first anti-BCMA VHH specifically binding to a first epitope of BCMA, and a second anti-BCMA VHH specifically binding to a second epitope of BCMA; (b) a transmembrane domain; and (c) an intracellular signaling domain, wherein the first epitope and the second epitope are different.

In some embodiments, the dosage form comprises 3.0×10⁷ to 4.0×10⁷ of the CAR-T cells. In some embodiments, the dosage form comprises 3.5×10⁷ to 4.5×10⁷ of the CAR-T cells. In some embodiments, the dosage form comprises 4.0×10⁷ to 5.0×10⁷ of the CAR-T cells. In some embodiments, the dosage form comprises 4.5×10⁷ to 5.5×10⁷ of the CAR-T cells. In some embodiments, the dosage form comprises 5.0×10⁷ to 6.0×10⁷ of the CAR-T cells. In some embodiments, the dosage form comprises 5.5×10⁷ to 6.5×10⁷ of the CAR-T cells. In some embodiments, the dosage form comprises 6.0×10⁷ to 7.0×10⁷ of the CAR-T cells. In some embodiments, the dosage form comprises 6.5×10⁷ to 7.5×10⁷ of the CAR-T cells. In some embodiments, the dosage form comprises 7.0×10⁷ to 8.0×10⁷ of the CAR-T cells. In some embodiments, the dosage form comprises 7.5×10⁷ to 8.5×10⁷ of the CAR-T cells. In some embodiments, the dosage form comprises 8.0×10⁷ to 9.0×10⁷ of the CAR-T cells. In some embodiments, the dosage form comprises 8.5×10⁷ to 9.5×10⁷ of the CAR-T cells. In some embodiments, the dosage form comprises 9.0×10⁷ to 1.0×10⁸ of the CAR-T cells.

In some embodiments, the cell population of the CAR-T dosage forms described herein comprise a T cell or population of T cells, e.g., at various stages of differentiation. Stages of T cell differentiation include naïve T cells, stem central memory T cells, central memory T cells, effector memory T cells, and terminal effector T cells, from least to most differentiated. After antigen exposure, naïve T cells proliferate and differentiate into memory T cells, e.g., stem central memory T cells and central memory T cells, which then differentiate into effector memory T cells. Upon receiving appropriate T cell receptor, costimulatory, and inflammatory signals, memory T cells further differentiate into terminal effector T cells. Sec, e.g., Restifo. Blood. 124.4 (2014):476-77; and Joshi et al. J. Immunol. 180.3 (2008):1309-15.

Naïve T cells can have the following expression pattern of cell surface markers: CCR7+, CD62L+, CD45RO−, CD95−. Stem central memory T cells (Tscm) can have the following expression pattern of cell surface markers: CCR7+, CD62L+, CD45RO−, CD95+. Central memory T cells (Tcm) can have the following expression pattern of cell surface markers: CCR7+, CD62L+, CD45RO+, CD95+. Effector memory T cells (Tem) can have the following expression pattern of cell surface markers: CCR7−, CD62L−, CD45RO+, CD95+. Terminal effector T cells (Teff) can have the following expression pattern of cell surface markers: CCR7−, CD62L−, CD45RO−, CD95+. See, e.g., Gattinoni et al. Nat. Med. 17 (2011):1290-7; and Flynn et al. Clin. Translat. Immunol. 3 (2014):e20.

Further provided by the present application are pharmaceutical compositions comprising any one of the engineered immune effector cells comprising any one of the CARs (such as BCMA CARs) as described herein, and a pharmaceutically acceptable carrier. Pharmaceutical compositions can be prepared by mixing any of the immune effector cells described herein, having the desired degree of purity, with optional pharmaceutically acceptable carriers, excipients or stabilizers (Remington's Pharmaceutical Sciences 16th edition, Osol, A. Ed. (1980)), in the form of lyophilized formulations or aqueous solutions. In some embodiments, a pharmaceutical composition of CAR-T cells further comprises an excipient selected from dimethylsulfoxide or dextran-40.

The compositions described herein may be administered as part of a pharmaceutical composition comprising one or more carriers. The choice of carrier will be determined in part by the particular nucleic acid sequence, vector, or host cells expressing the CAR disclosed herein, as well as by the particular method used to administer the nucleic acid sequence, vector, or host cells expressing the CAR disclosed herein. Accordingly, there are a variety of suitable formulations of the pharmaceutical composition of the disclosure.

For example, the pharmaceutical composition can contain preservatives. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

In addition, buffering agents may be used in the composition. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The composition comprising the nucleic acid sequence encoding the CAR disclosed herein, or host cells expressing the CAR disclosed herein, can be formulated as an inclusion complex, such as cyclodextrin inclusion complex, or as a liposome. Liposomes can serve to target the host cells (e.g., T-cells or NK cells) or the nucleic acid sequence disclosed herein to a particular tissue. Liposomes also can be used to increase the half-life of the nucleic acid sequence disclosed herein. Many methods are available for preparing liposomes, such as those described in, for example, Szoka et al., Ann. Rev. Biophys. Bioeng., 9: 467 (1980), and U.S. Pat. Nos. 4,235,871; 4,501,728; 4,837,028; and 5,019,369. The composition can employ time-released, delayed release, and sustained release delivery systems such that the delivery of the composition disclosed herein occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition embodiments of the disclosure.

In some embodiments, the CAR-T cells are formulated at a dose of about 0.5×10⁶ cells/kg to about 1.0×10⁶ cells/kg, of about 0.55×10⁶ cells/kg to about 0.95×10⁶ cells/kg, of about 0.6×10⁶ cells/kg to about 0.90×10⁶ cells/kg, of about 0.65×10⁶ cells/kg to about 0.85×10⁶ cells/kg, and of about 0.7×10⁶ cells/kg to about 0.80×10⁶ cells/kg. In some embodiments, the dosage form comprises 0.75×10⁶ cells/kg. In a preferred embodiment, the dose is formulated at approximately 0.75×10⁶ cells/kg. In some embodiments, the CAR-T cells are formulated at a dose of less than 1.0×10⁸ CAR-T cells per subject.

### GPRC5D×CD3 Bispecific Antibodies

G Protein-Coupled Receptor Class C Group 5 Member D (GPRC5D) is a 7 transmembrane receptor protein that is classified as a type C G protein-coupled receptor based on the sequence homology score and is an orphan receptor whose ligand and signaling mechanisms are yet to be identified. GPRC5D messenger ribonucleic acid (mRNA) is predominantly expressed in cells with a plasma cell phenotype and also expressed in all malignant plasma cells from patients with multiple myeloma. The expression of GPRC5D on the plasma cell lineage makes it a target for T cell mediated therapy to treat plasma cell disorders like multiple myeloma. A GPRC5D×CD3 bispecific antibody targets the CD3 receptor complex on T cells and GPRC5D on plasma cells. The dual binding sites allow the GPRC5D×CD3 bispecific antibody to draw CD3+ T cells in close proximity to myeloma cells, without regard to T cell receptor specificity or reliance on MHC Class 1 molecules on the surface of antigen presenting cells for activation, leading to cell death of the GPRC5D-positive cells.

Any suitable GPRC5D×CD3 bispecific antibody can be used in a method of the application. Exemplary multispecific and/or bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F (ab) 2 (Medarex/AMGEN), Dual-Action or Bis-Fab (Genentech), Dock-and-Lock (DNL) (ImmunoMedics), Bivalent Bispecific (Biotecnoly and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12:276 and in Nuncz-Prado et al., (2015) Drug Discovery Today 20(5):588-594.

In some embodiments, the GPRC5D×CD3 bispecific antibody is an antigen binding fragment. Exemplary antigen binding fragments are Fab, F(ab′)2, Fd and Fv fragments.

In some embodiments, the GPRC5D×CD3 bispecific antibody is chimeric, humanized or human.

In some embodiments, the GPRC5D×CD3 bispecific antibody comprises a GPRC5D binding domain comprising a VH having the HCDR1 of SEQ ID NO: 101, the HCDR2 of SEQ ID NO: 102, the HCDR3 of SEQ ID NO: 103, and a VL having the LCDR1 of SEQ ID NO: 104, the LCDR2 of SEQ ID NO: 105 and the LCDR3 of SEQ ID NO: 106, and a CD3 binding domain comprising a VH having the HCDR1 of SEQ ID NO: 107, the HCDR2 of SEQ ID NO: 108, the HCDR3 of SEQ ID NO: 109, and a VL having the LCDR1 of SEQ ID NO: 110, the LCDR2 of SEQ ID NO: 111 and the LCDR3 of SEQ ID NO: 112. The HCDRs and LCDRs of the GPRC5D×CD3 bispecific antibody are recited in Table 3 below:

TABLE 3
Exemplary CDRs of GPRC5D x CD3
bispecific antibody

| Binding | | | SEQ |
|---|---|---|---|
| Arm | Region | Sequence | ID NO: |
| GPRC5D | HCDR1 | GYTMN | 101 |
| | HCDR2 | LINPYNSDTNYAQKLQG | 102 |
| | HCDR3 | VALRVALDY | 103 |
| | LCDR1 | KASQNVATHVG | 104 |
| | LCDR2 | SASYRYS | 105 |
| | LCDR3 | QQYNRYPYT | 106 |
| CD3 | HCDR1 | TYAMN | 107 |
| | HCDR2 | RIRSKYNNYATYYAASVKG | 108 |
| | HCDR3 | HGNFGNSYVSWFAY | 109 |
| | LCDR1 | RSSTGAVTTSNYAN | 110 |
| | LCDR2 | GTNKRAP | 111 |
| | LCDR3 | ALWYSNLWV | 112 |

The CDRs recited in the table above are of the Kabat numbering system. However, as provided for herein, the CDRs of the present disclosure may be provided by any appropriate numbering system, such as any of the Kabat, Chothia, IMGT, or AbM numbering systems. Tables 4-6 below provide exemplary CDRs utilizing the Chothia, AbM, and IMGT numbering systems:

TABLE 4
Exemplary CDRs of GPRC5D x CD3 bispecific
antibody-Chothia numbering system:

| Binding Arm | Region | Sequence | SEQ ID NO: |
|---|---|---|---|
| GPRC5D | HCDR1 | GYSFTGY | 124 |
| | HCDR2 | NPYNSD | 125 |
| | HCDR3 | VALRVALDY | 103 |
| | LCDR1 | KASQNVATHVG | 104 |
| | LCDR2 | SASYRYS | 105 |
| | LCDR3 | QQYNRYPYT | 106 |
| CD3 | HCDR1 | GFTFNTY | 126 |
| | HCDR2 | RSKYNNYA | 127 |
| | HCDR3 | HGNFGNSYVSWFAY | 109 |
| | LCDR1 | RSSTGAVTTSNYAN | 110 |
| | LCDR2 | GTNKRAP | 111 |
| | LCDR3 | ALWYSNLWV | 112 |

TABLE 5
Exemplary CDRs of GPRC5D x CD3 bispecific
antibody-AbM numbering system:

| Binding Arm | Region | Sequence | SEQ ID NO: |
|---|---|---|---|
| GPRC5D | HCDR1 | GYSFTGYTMN | 128 |
| | HCDR2 | LINPYNSDTN | 129 |
| | HCDR3 | VALRVALDY | 103 |
| | LCDR1 | KASQNVATHVG | 104 |
| | LCDR2 | SASYRYS | 105 |
| | LCDR3 | QQYNRYPYT | 106 |
| CD3 | HCDR1 | GFTFNTYAMN | 130 |
| | HCDR2 | RIRSKYNNYATY | 131 |
| | HCDR3 | HGNFGNSYVSWFAY | 109 |
| | LCDR1 | RSSTGAVTTSNYAN | 110 |
| | LCDR2 | GTNKRAP | 111 |
| | LCDR3 | ALWYSNLWV | 112 |

TABLE 6
Exemplary CDRs of GPRC5D x CD3 bispecific
antibody-IMGT numbering system:

| Binding Arm | Region | Sequence | SEQ ID NO: |
|---|---|---|---|
| GPRC5D | HCDR1 | GYSFTGYT | 132 |
| | HCDR2 | INPYNSDT | 133 |
| | HCDR3 | ARVALRVALDY | 134 |
| | LCDR1 | QNVATH | 135 |
| | LCDR2 | SAS | NA |
| | LCDR3 | QQYNRYPYT | 106 |
| CD3 | HCDR1 | GFTFNTYA | 136 |
| | HCDR2 | IRSKYNNYAT | 137 |
| | HCDR3 | ARHGNFGNSYVSWFAY | 138 |
| | LCDR1 | TGAVTTSNY | 139 |
| | LCDR2 | GTN | NA |
| | LCDR3 | ALWYSNLWV | 112 |

In some embodiments, the GPRC5D×CD3 bispecific antibody comprises a GPRC5D binding domain comprising a VH having the HCDR1 of SEQ ID NO: 101, the HCDR2 of SEQ ID NO: 102, the HCDR3 of SEQ ID NO: 103, and a VL having the LCDR1 of SEQ ID NO: 104, the LCDR2 of SEQ ID NO: 105 and the LCDR3 of SEQ ID NO: 106, and a CD3 binding domain comprising a VH having the HCDR1 of SEQ ID NO: 107, the HCDR2 of SEQ ID NO: 108, the HCDR3 of SEQ ID NO: 109, and a VL having the LCDR1 of SEQ ID NO: 110, the LCDR2 of SEQ ID NO: 111 and the LCDR3 of SEQ ID NO: 112.

In some embodiments, the GPRC5D×CD3 bispecific antibody comprises a GPRC5D binding domain comprising a VH having the HCDR1 of SEQ ID NO: 124, the HCDR2 of SEQ ID NO: 125, the HCDR3 of SEQ ID NO: 103, and a VL having the LCDR1 of SEQ ID NO: 104, the LCDR2 of SEQ ID NO: 105 and the LCDR3 of SEQ ID NO: 106, and a CD3 binding domain comprising a VH having the HCDR1 of SEQ ID NO: 126, the HCDR2 of SEQ ID NO: 127, the HCDR3 of SEQ ID NO: 109, and a VL having the LCDR1 of SEQ ID NO: 110, the LCDR2 of SEQ ID NO: 111 and the LCDR3 of SEQ ID NO: 112.

In some embodiments, the GPRC5D×CD3 bispecific antibody comprises a GPRC5D binding domain comprising a VH having the HCDR1 of SEQ ID NO: 128, the HCDR2 of SEQ ID NO: 129, the HCDR3 of SEQ ID NO: 103, and a VL having the LCDR1 of SEQ ID NO: 104, the LCDR2 of SEQ ID NO: 105 and the LCDR3 of SEQ ID NO: 106, and a CD3 binding domain comprising a VH having the HCDR1 of SEQ ID NO: 130, the HCDR2 of SEQ ID NO: 131, the HCDR3 of SEQ ID NO: 109, and a VL having the LCDR1 of SEQ ID NO: 110, the LCDR2 of SEQ ID NO: 111 and the LCDR3 of SEQ ID NO: 112.

In some embodiments, the GPRC5D×CD3 bispecific antibody comprises a GPRC5D binding domain comprising a VH having the HCDR1 of SEQ ID NO: 132, the HCDR2 of SEQ ID NO: 133, the HCDR3 of SEQ ID NO: 134, and a VL having the LCDR1 of SEQ ID NO: 135, a LCDR2 having the amino acid sequence SAS, and the LCDR3 of SEQ ID NO: 106, and a CD3 binding domain comprising a VH having the HCDR1 of SEQ ID NO: 136, the HCDR2 of SEQ ID NO: 137 the HCDR3 of SEQ ID NO: 138, and a VL having the LCDR1 of SEQ ID NO: 139, a LCDR2 having the amino acid sequence GTN, and the LCDR3 of SEQ ID NO: 112.

In some embodiments, the GPRC5D×CD3 bispecific antibody comprises a GPRC5D binding domain comprising the VH of SEQ ID NO: 113 and the VL of SEQ ID NO: 114, and a CD3 binding domain comprising the VH of SEQ ID NO: 115 and the VL of SEQ ID NO: 116.

In some embodiments, the GPRC5D×CD3 bispecific antibody that binds GPRC5D comprises a first heavy chain (HC1) of SEQ ID NO: 117, a first light chain (LC1) of SEQ ID NO: 118, a second heavy chain (HC2) of SEQ ID NO: 119, and a second light chain (LC2) of SEQ ID NO: 120.

In some embodiments, the CD3 binding arm of the GPRC5D×CD3 bispecific antibody and the GPRC5D binding arm of the GPRC5D×CD3 bispecific antibody comprise the amino acid sequences as provided for in Tables 7A-7B.

TABLE 7A
Sequences of GPRC5D binding arm of a
GPRC5D x CD3 bispecific antibody.

| | | SEQ |
|---|---|---|
| Region | Sequence | ID NO: |
| HCDR1 | GYTMN | 101 |
| HCDR2 | LINPYNSDTNYAQKLQG | 102 |
| HCDR3 | VALRVALDY | 103 |
| LCDR1 | KASQNVATHVG | 104 |
| LCDR2 | SASYRYS | 105 |
| LCDR3 | QQYNRYPYT | 106 |
| VH | QVQLVQSGAEVKKPGASVKVSCKASGYSFT | 113 |
| | GYTMNWVRQAPGQGLEWMGLINPYNSDTN | |
| | YAQKLQGRVTMTTDTSTSTAYMELRSLRSD | |
| | DTAVYYCARVALRVALDYWGQGTLVTVSS | |
| VL | DIQMTQSPSSLSASVGDRVTITCKASQNVAT | 114 |
| | HVGWYQQKPGKAPKRLIYSASYRYSGVPSR | |
| | FSGSGSGTEFTLTISNLQPEDFATYYCQQYNR | |
| | YPYTFGQGTKLEIK | |
| HC | QVQLVQSGAEVKKPGASVKVSCKASGYSFT | 117 |
| | GYTMNWVRQAPGQGLEWMGLINPYNSDTN | |
| | YAQKLQGRVTMTTDTSTSTAYMELRSLRSD | |
| | DTAVYYCARVALRVALDYWGQGTLVTVSS | |
| | ASTKGPSVFPLAPCSRSTSESTAALGCLVKD | |
| | YFPEPVTVSWNSGALTSGVHTFPAVLQSSGL | |
| | YSLSSVVTVPSSSLGTKTYTCNVDHKPSNTK | |
| | VDKRVESKYGPPCPPCPAPEAAGGPSVFLFPP | |
| | KPKDTLMISRTPEVTCVVVDVSQEDPEVQFN | |
| | WYVDGVEVHNAKTKPREEQFNSTYRVVSVL | |
| | TVLHQDWLNGKEYKCKVSNKGLPSSIEKTIS | |
| | KAKGQPREPQVYTLPPSQEEMTKNQVSLTCL | |
| | VKGFYPSDIAVEWESNGQPENNYKTTPPVLD | |
| | SDGSFFLYSRLTVDKSRWQEGNVFSCSVMH | |
| | EALHNHYTQKSLSLSLGK | |
| LC | DIQMTQSPSSLSASVGDRVTITCKASQNVAT | 118 |
| | HVGWYQQKPGKAPKRLIYSASYRYSGVPSR | |
| | FSGSGSGTEFTLTISNLQPEDFATYYCQQYNR | |
| | YPYTFGQGTKLEIKKAAPSVTLFPPSSEELQA | |
| | NKATLVCLISDFYPGAVTVAWKGDSSPVKA | |
| | GVETTTPSKQSNNKYAASSYLSLTPEQWKSH | |
| | RSYSCQVTHEGSTVEKTVAPTECS | |

TABLE 7B
Sequences of CD3 binding arm of a
GPRC5D x CD3 bispecific antibody.

| | | SEQ |
|---|---|---|
| Region | Sequence | ID NO: |
| HCDR1 | TYAMN | 107 |
| HCDR2 | RIRSKYNNYATYYAASVKG | 108 |
| HCDR3 | HGNFGNSYVSWFAY | 109 |
| LCDR1 | RSSTGAVTTSNYAN | 110 |
| LCDR2 | GTNKRAP | 111 |
| LCDR3 | ALWYSNLWV | 112 |
| VH | EVOLVESGGGLVQPGGSLRLSCAASGFTFNT | 115 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSS | |
| VL | QTVVTQEPSLTVSPGGTVTLTCRSSTGAVTT | 116 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQP | |
| HC | EVOLVESGGGLVQPGGSLRLSCAASGFTFNT | 119 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSSASTKGPSVFPLAPCSRSTSESTAALGC | |
| | LVKDYFPEPVTVSWNSGALTSGVHTFPAVL | |
| | QSSGLYSLSSVVTVPSSSLGTKTYTCNVDHK | |
| | PSNTKVDKRVESKYGPPCPPCPAPEAAGGPS | |
| | VFLFPPKPKDTLMISRTPEVTCVVVDVSQED | |
| | PEVQFNWYVDGVEVHNAKTKPREEQFNSTY | |
| | RVVSVLTVLHQDWLNGKEYKCKVSNKGLPS | |
| | SIEKTISKAKGQPREPQVYTLPPSQEEMTKNQ | |
| | VSLTCLVKGFYPSDIAVEWESNGQPENNYKT | |
| | TPPVLDSDGSFLLYSKLTVDKSRWQEGNVFS | |
| | CSVMHEALHNHYTQKSLSLSLGK | |
| LC | QTVVTQEPSLTVSPGGTVTLTCRSSTGAVTT | 120 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQPKAAPSVTLFP | |
| | PSSEELQANKATLVCLISDFYPGAVTVAWKA | |
| | DSSPVKAGVETTTPSKQSNNKYAASSYLSLT | |
| | PEQWKSHRSYSCQVTHEGSTVEKTVAPTECS | |

In some embodiments, the GPRC5D×CD3 bispecific antibody can be, but is not limited to, talquetamab, a GPRC5D×CD3 bispecific antibody described in Kodama et al. Mol Cancer Ther. 2019. 18(9): 1555-1564, the entire content of which is incorporated herein by reference, or a bispecific antibody that uses a human GPRC5D binding domain described in U.S. Pat. No. 10,590,196, the entire content of which is incorporated herein by reference, or a GPRC5D binding domain that competes with talquetamab or the human GPRC5D binding domain described in U.S. Pat. No. 10,590,196 for binding to human GPRC5D.

In some embodiments, talquetamab comprises a first heavy chain (HC1), a first light chain (LC1), a second heavy chain (HC2), and a second light chain (LC2), wherein the HC1 is associated with LC1 and the HC2 is associated with LC2, wherein HC1 and LC1 form a first antigen-binding site that immunospecifically binds to GPRC5D and wherein HC2 and LC2 form a second antigen-binding site that immunospecifically binds to CD3. In some embodiments, talquetamab comprises a HC1 of SEQ ID NO: 117, a LC1 of SEQ ID NO: 118, a HC2 of SEQ ID NO: 119, and a LC2 of SEQ ID NO: 120. In some embodiments, the CD3 arm and the GPRC5D arm of talquetamab form a functional bispecific antibody through an interaction between their respective Fc domains.

In some embodiments, the GPRC5D×CD3 bispecific antibody comprises any one of GPRC5D binding domains described in U.S. Pat. No. 10,906,956 or WO2020/092854 the entire content of which is incorporated herein by reference, or a GPRC5D binding domain that competes with such GPRC5D binding domain for binding to human GPRC5D.

In some embodiments, the GPRC5D×CD3 bispecific antibody is an IgG1, an IgG2, an IgG3 or an IgG4 isotype.

In some embodiments, the GPRC5D×CD3 bispecific antibody is an IgG1 isotype.

In some embodiments, the GPRC5D×CD3 bispecific antibody is an IgG2 isotype.

In some embodiments, the GPRC5D×CD3 bispecific antibody is an IgG3 isotype.

In some embodiments, the GPRC5D×CD3 bispecific antibody is an IgG4 isotype.

The GPRC5D×CD3 bispecific antibody can be of any allotype. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host may be determined in part by the allotype of the antibody (Stickler et al., (2011) Genes and Immunity 12:213-21). Antibody allotype is related to amino acid sequence variations at specific locations in the constant region sequences of the antibody. Table 8 shows select IgG1, IgG2 and IgG4 allotypes.

TABLE 8
IgG1, IgG2 and IgG4 allotypes.

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n-) | P | V | | | | | | |
| G2m(n)/(n- | T | V | | | | | | |
| nG4m(a) | | | L | R | | | | |
| G1m(17) | | | | | K | E | M | A |
| G1m(17, 1) | | | | | K | D | L | A |

In some embodiments, the one or more Fc substitutions is selected from the group consisting of F234A/L235A on IgG4, L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4 and S228P/F234A/L235A/G236-deleted/G237A/P238S on IgG4, wherein residue numbering is according to the EU index.

In some embodiments, the one or more Fc substitutions is F234A/L235A on IgG4.

In some embodiments, the one or more Fc substitutions is L234A/L235A on IgG1.

In some embodiments, the one or more Fc substitutions is V234A/G237A/P238S/H268A/V309L/A330S/P331S on IgG2.

In some embodiments, the one or more Fc substitutions is F234A/L235A on IgG4.

In some embodiments, the one or more Fc substitutions is S228P/F234A/L235A on IgG4.

In some embodiments, the one or more Fc substitutions is N297A on all Ig isotypes.

In some embodiments, the one or more Fc substitutions is V234A/G237A on IgG2.

In some embodiments, the one or more Fc substitutions is K214T/E233P/L234V/L235A/G236-deleted/A327G/P331A/D365E/L358M on IgG1.

In some embodiments, the one or more Fc substitutions is H268Q/V309L/A330S/P331S on IgG2.

In some embodiments, the one or more Fc substitutions is S267E/L328F on IgG1. In some embodiments, the one or more Fc substitutions is L234F/L235E/D265A on IgG1.

In some embodiments, the one or more Fc substitutions is L234A/L235A/G237A/P238S/H268A/A330S/P331S on IgG1.

In some embodiments, the one or more Fc substitutions is S228P/F234A/L235A/G237A/P238S on IgG4 and S228P/F234A/L235A/G236-deleted/G237A/P238S on IgG4.

In some embodiments, the multispecific antibody further comprises a S228P substitution.

In some embodiments, the multispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F450L/K409R, wild-type/F409L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In some embodiments, the one or more asymmetric substitutions is F450L/K409R.

In some embodiments, the one or more asymmetric substitutions is wild-type/F409L_R409K.

In some embodiments, the one or more asymmetric substitutions is T366Y/F405A.

In some embodiments, the one or more asymmetric substitutions is T366W/F405W.

In some embodiments, the one or more asymmetric substitutions is F405W/Y407A.

In some embodiments, the one or more asymmetric substitutions is T394W/Y407T.

In some embodiments, the one or more asymmetric substitutions is T394S/Y407A.

In some embodiments, the one or more asymmetric substitutions is T366W/T394S.

In some embodiments, the one or more asymmetric substitutions is F405W/T394S.

In some embodiments, the one or more asymmetric substitutions is T366W/T366S_L368A_Y407V.

In some embodiments, the one or more asymmetric substitutions is L351Y_F405A_Y407V/T394W.

In some embodiments, the one or more asymmetric substitutions is T366I_K392M_T394W/F405A_Y407V.

In some embodiments, the one or more asymmetric substitutions is T366L_K392M_T394W/F405A_Y407V.

In some embodiments, the one or more asymmetric substitutions is L351Y_Y407A/T366A_K409F.

In some embodiments, the one or more asymmetric substitutions is L351Y_Y407A/T366V_K409F.

In some embodiments, the one or more asymmetric substitutions is Y407A/T366A_K409F.

In some embodiments, the one or more asymmetric substitutions is T350V_L351Y_F405A_Y407V/T350V_T366L_K392L_T394W.

In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the GPRC5D×CD3 bispecific antibody further comprises proline at position 228, alanine at position 234 and alanine at position 235 in both the HC1 and the HC2, wherein residue numbering is according to the EU Index.

The antibodies used in the methods of the disclosure binding specific antigens may be selected de novo from, for example, a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-14, 1996; Sheets et al., PITAS (USA) 95:6157-62, 1998; Hoogenboom and Winter, J Mol Biol 227:381, 1991; Marks et al., J Mol Biol 222:581, 1991). Phage display libraries expressing antibody heavy and light chain variable regions as fusion proteins with bacteriophage pIX coat protein as described in Shi et al (2010) J. Mol. Biol. 397:385-96 and Int'l Pat. Pub. No. WO2009/085462. The antibody libraries may be screened for binding to the desired antigen, such as GPRC5D and the obtained positive clones may be further characterized and the Fabs isolated from the clone lysates, and subsequently cloned as full-length antibodies. Such phage display methods for isolating human antibodies are established in the art. See for example: U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915; and 6,593,081.

T cell redirecting bispecific antibodies may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and/or F405L.

Additional CH3 mutations that may be used include technologies such as Duobody® mutations (Genmab), Knob-in-Hole mutations (Genentech), electrostatically-matched mutations (Chugai, Amgen, NovoNordisk, Oncomed), the Strand Exchange Engineered Domain body (SEEDbody) (EMD Serono), and other asymmetric mutations (e.g., Zymeworks).

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

SEEDbody mutations involve substituting select IgG residues with IgA residues to promote heavy chai heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO2011/143545, WO2013/157954, WO2013/096291 and US2018/0118849.

Additional bispecific or multispecific structures that can be used as GPRC5D×CD3 bispecific antibodies include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv) 2-Fab (National Research Center for Antibody Medicine—China), Dual-Action or Bis-Fab (Genentech), Dock-and-Lock (DNL) (ImmunoMedics), Bivalent Bispecific (Biotecnol and Fab-Fv (UCB-Celltech). ScFv-, diabody-based, and domain antibodies, include but are not limited to, Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

The Fc region of the GPRC5D×CD3 bispecific antibodies such as bispecific or multispecific antibodies or the anti-CD38 antibodies may comprise at least one substitution in the Fc region that reduces binding of the GPRC5D×CD3 bispecific antibodies to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4.

Fc substitutions that may be used to reduce CDC is a K322A substitution.

Well-known S228P substitution may further be made in IgG4 antibodies to enhance IgG4 stability.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells. For example, NK cells express FcγRIIIa, whereas monocytes express FcγRI, FcγRII and FcγRIIIa. ADCC activity of the antibodies may be assessed using an in vitro assay using cells expressing the protein the antibody binds to as target cells and NK cells as effector cells. Cytolysis may be detected by the release of label (e.g., radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis is measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

“Antibody-dependent cellular phagocytosis” (“ADCP”) refers to a mechanism of elimination of antibody-coated target cells by internalization by phagocytic cells, such as macrophages or dendritic cells. ADCP may be evaluated by using monocyte-derived macrophages as effector cells and cells that express the protein the antibody binds to as target cells also engineered to express GFP or another labeled molecule. In an exemplary assay, effector: target cell ratio may be for example 4:1. Effector cells may be incubated with target cells for 4 hours with or without the antibody of the disclosure. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods.

“Complement-dependent cytotoxicity”, or “CDC”, refers to a mechanism for inducing cell death in which the Fc effector domain of a target-bound antibody binds and activates complement component C1q which in turn activates the complement cascade leading to target cell death. Activation of complement may also result in deposition of complement components on the target cell surface that facilitate CDC by binding complement receptors (e.g., CR3) on leukocytes. CDC of cells may be measured for example by plating Daudi cells at 1×10⁵ cells/well (50 μL/well) in RPMI-B (RPMI supplemented with 1% BSA), adding 50 μL of test antibodies to the wells at a final concentration between 0-100 μg/mL, incubating the reaction for 15 min at room temperature, adding 11 μL of pooled human serum to the wells, and incubating the reaction for 45 min at 37° C. Percentage (%) lysed cells may be detected as % propidium iodide stained cells in FACS assay using standard methods.

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hours at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel, respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

### Methods

The present application further relates to methods and compositions for use in cell immunotherapy. In particular, described herein are methods of treating multiple myeloma in a subject in need thereof, the methods comprising: administering anti-BCMA CAR T cells to the subject, and administering a GPRC5D×CD3 bispecific antibody to the subject. In some embodiments, the administration of the GPRC5D×CD3 bispecific antibody occurs after the administration of the anti-BCMA CAR T cells. In some embodiments, the administration of the GPRC5D×CD3 bispecific antibody occurs before the administration of the anti-BCMA CAR T cells.

In some embodiments, the cell immunotherapy is for treating cancer in a subject, including but not limited to hematological malignancies and solid tumors. In some embodiments, the cell immunotherapy is for treating multiple myeloma in a subject. In some embodiments, the subject is human. In some embodiments, the methods are suitable for treatment of adults and pediatric population, including all subsets of age, and can be used as any line of treatment, including first line or subsequent lines.

The methods described herein may be used for treating various cancers, including both solid cancer and liquid cancer. In some embodiments, the methods are used to treat multiple myeloma. The methods described herein may be used as a combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In some embodiments, the cancer is stage I, stage II or stage III, and/or stage A or stage B multiple myeloma based on the Durie-Salmon staging system. In some embodiments, the cancer is stage I, stage II or stage III multiple myeloma based on the International staging system published by the International Myeloma Working Group (IMWG). In some embodiments, the multiple myeloma is progressive.

In some embodiments, the subject received prior treatment with at least an initial therapy. In some embodiments, the initial therapy comprises treatment with a medicament that is a proteasomal inhibitor (PI). Non-limiting examples of a PI include bortezomib, carfilzomib and ixazomib. In some embodiments, the initial therapy comprises treatment with a medicament that is an immunomodulatory drug (IMiD). Non-limiting examples of an IMiD include lenalidomide, pomalidomide, thalidomide, and cereblon E3 ligase modulatory drugs (CELMOD), such as iberdomine or mezigdomide. In some embodiments, the patient is lenalidomide-refractory. In some embodiments the IMiD therapy comprises a combination of lenalidomide and pomalidomide. In some embodiments, the initial therapy comprises treatment with a medicament that is a corticosteroid. Non-limiting examples of a corticosteroid include dexamethasone and prednisone. In some embodiments, the initial therapy comprises treatment with a medicament that is an alkylating agent. In some embodiments, the initial therapy comprises treatment with a medicament that is an anthracycline. In some embodiments, the initial therapy comprises treatment with a medicament that is an anti-CD38 antibody. Non-limiting examples of an anti-CD38 antibody include daratumumab, isatuximab and the investigational antibody TAK-079. In some embodiments, the initial therapy comprises treatment with a medicament that is elotuzumab. In some embodiments, the initial therapy comprises treatment with a medicament that is panobinostat. In some embodiments, the subject has received prior treatment with an anti-CD38 antibody as part of one or more of the 1 to 3 prior lines of therapy. In some embodiments, the anti-CD38 antibody is daratumumab and/or isatuximab. In some embodiments, the prior treatment comprises an IMiD (e.g., lenalidomide), a proteasome inhibitor and an anti-CD38 antibody (e.g., as 3 prior lines of therapy). In some embodiments, the subject has received 1 prior line of therapy including lenalidomide and is lenalidomide-refractory, and optionally has received one or two further lines of therapy. In some embodiments, the subject has received at least one prior lines of therapy including lenalidomide and a proteasome inhibitor, and optionally has received one or two further lines of therapy. In some embodiments, the cancer is refractory to one or more of, or all of, bortezomib, carfilzomib, ixazomib, lenalidomide, pomalidomide, thalidomide, dexamethasone, prednisone, alkylating agents, daratumumab, isatuximab, TAK-079, elotuzumab, Panobinostat, or a cereblon E3 ligase modulatory drug (CELMOD), such as iberdomine or mezigdomide. In some embodiments, the initial therapy includes surgery, radiotherapy, or autologous or allogeneic transplant, including tandem transplants, or any combination of such treatments.

In some embodiments, the multiple myeloma is refractory to at least one medicament. In some embodiments, the multiple myeloma is refractory to at least two medicaments. In some embodiments, the multiple myeloma is refractory to at least three medicaments. In some embodiments, the multiple myeloma is refractory to at least four medicaments. In some embodiments, the multiple myeloma is refractory to at least five medicaments.

In some embodiments, the subject has relapsed and/or refractory multiple myeloma, and received at least three prior lines of therapies, including a proteasomal inhibitor (PI), an immunomodulatory drug (IMiD), and an anti-CD38 antibody.

In some embodiments, the subject has newly diagnosed multiple myeloma and is transplant-ineligible.

Any of the anti-BCMA VHHs, CARs, and engineered immune effector cells (such as CAR-T cells) described herein may be used in the method of treating cancer. In some embodiments, the immune effector cells are autologous. In some embodiments, the immune effector cells are allogeneic. In a preferred embodiment, ciltacabtagene autoleucel (“cilta-cel”) CAR-T cells are administered to the subject.

In some embodiments, apheresis material is collected from the subject for the production of CAR-T cells. In some embodiments, apheresis material is collected from the subject for the production of ciltacabtagene autoleucel.

In some embodiments, the CAR-T cells are administered at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg, 1.0×10⁶ to 2.0×10⁶ cells/kg, 1.5×10⁶ to 2.5×10⁶ cells/kg, 2.0×10⁶ to 3.0×10⁶ cells/kg, 2.5×10⁶ to 3.5×10⁶ cells/kg, 3.0×10⁶ to 4.0×10⁶ cells/kg, 3.5×10⁶ to 4.5×10⁶ cells/kg, 4.0×10⁶ to 5.0×10⁶ cells/kg, 4.5×10⁶ to 5.5×10⁶ cells/kg, or 5.0×10⁶ to 6.0×10⁶ cells/kg. In some embodiments, the dose comprises approximately 0.75×10⁶ cells/kg. In some embodiments, the dose comprises approximately 0.68×10⁶ cells/kg. In some embodiments, the CAR-T cells are administered at a dose of about 1.0×10⁸ cells per subject.

In some embodiments, the CAR-T cells are administered at a dose of less than 1.0×10⁸ cells per subject. In some embodiments, the CAR-T cells are administered at a dose of about 3.0 to 4.0×10⁷ cells. In some embodiments, the CAR-T cells are administered at a dose of about 3.5 to 4.5×10⁷ cells. In some embodiments, the CAR-T cells are administered at a dose of about 4.0 to 5.0×10⁷ cells. In some embodiments, the CAR-T cells are administered at a dose of about 4.5 to 5.5×10⁷ cells. In some embodiments, the CAR-T cells are administered at a dose of about 5.0 to 6.0×10⁷ cells. In some embodiments, the CAR-T cells are administered at a dose of about 5.5 to 6.5×10⁷ cells. In some embodiments, the CAR-T cells are administered at a dose of about 6.0 to 7.0×10⁷ cells. In some embodiments, the CAR-T cells are administered at a dose of about 6.5 to 7.5×10⁷ cells. In some embodiments, the CAR-T cells are administered at a dose of about 7.0 to 8.0×10⁷ cells. In some embodiments, the CAR-T cells are administered at a dose of about 7.5 to 8.5×10⁷ cells. In some embodiments, the CAR-T cells are administered at a dose of about 8.0 to 9.0×10⁷ cells. In some embodiments, the CAR-T cells are administered at a dose of about 8.5 to 9.5×10⁷ cells. In some embodiments, the CAR-T cells are administered at a dose of about 9.0×10⁷ to 1.0×10⁸ cells.

In some embodiments, the CAR-T cells are administered at a dose of about 0.693×10⁶ CAR-positive viable T-cells/kg. In some embodiments, the CAR-T cells are administered at a dose of about 0.52×10⁶ CAR-positive viable T-cells/kg. In some embodiments, the CAR-T cells are administered at a dose of about 0.94×10⁶ CAR-positive viable T-cells/kg. In some embodiments, the CAR-T cells are administered at a dose of about 0.709×10⁶ CAR-positive viable T-cells/kg. In some embodiments, the CAR-T cells are administered at a dose of about 0.51×10⁶ CAR-positive viable T-cells/kg. In some embodiments, the CAR-T cells are administered at a dose of about 0.95×10⁶ CAR-positive viable T-cells/kg. In some embodiments, the CAR-T cells are administered in an outpatient setting.

In some embodiments, the CAR-T cells (e.g., at any of the foregoing doses) are administered in one or more intravenous infusions. In some embodiments, said administration of said CAR-T cells is via a single intravenous infusion. In some embodiments, said single intravenous infusion is administered using a single bag of said CAR-T cells. In some embodiments, said administration of said single bag of said CAR-T cells is completed between the time at which said single bag of CAR-T cells is thawed and three hours after said single bag of CAR-T cells is thawed. In some embodiments, single intravenous administration is administered using two bags of said CAR-T cells. In some embodiments, said administration of each of said two bags of said CAR-T cells is completed between the time at which a first bag of said two bags of CAR-T cells is thawed and three hours after said first bag of CAR-T cells is thawed.

The composition comprising the host cells expressing the CAR-encoding nucleic acid sequence disclosed herein, or a vector comprising the CAR-encoding nucleic acid sequence disclosed herein, can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral”, as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. Most preferably, the composition is administered by intravenous infusion.

In some embodiments, prior to the administration of CAR-T cells, subjects may be administered a conditioning regimen.

Patients undergoing a CAR-T cell therapy may be prepared with a so-called conditioning regimen that can suppress the patient's immune system and improve the efficacy of CAR-T cell therapy (Blood (2019) 133 (17): 1799-1800).

The intensity of conventional conditioning regimens can vary significantly. Description of the regimens can refer to genotoxic or non-genotoxic regimens, which may overlap with reference to myeloablative or non-myeloablative regimens. See, for example, Bacigalupo et al. (2009) Biol Blood Marrow Transplant. 15(12):1628-1633, herein specifically incorporated by reference.

Myeloablative conditioning regimens are combination of agents expected to produce profound pancytopenia and myeloablation within 1-3 weeks from administration; pancytopenia is long lasting, usually irreversible and in most instances fatal, unless hematopoiesis is restored by hemopoietic stem cell infusion. Examples include total body irradiation and/or administration of high doses of alkylating agents; busulfan, melphalan, cyclophosphamide; etc.

Non-myeloablative conditioning regiments typically cause minimal cytopenia, and little early toxicity, but are immunosuppressive to the extent that, when followed by administration of an effective dose of HSPC, will result in engraftment of donor lympho-hemopoietic stem cells.

In certain embodiments the conditioning regimens provided herein are non-myeloablative.

In some embodiments, the conditioning regimen comprises one or more of cyclophosphamide and/or fludarabine.

In some embodiments, the conditioning regimen comprises cyclophosphamide administered at a dosage of 300 mg/m². In some embodiments, the conditioning regimen comprises fludarabine administered at a dosage of 30 mg/m². In some embodiments, the conditioning regimen comprises cyclophosphamide administered at a dosage of 300 mg/m² and fludarabine at a dosage of 30 mg/m².

In some embodiments, the conditioning regimen is administered to the subject daily, for up to 3 days. In some embodiments, the CAR-T therapy is administered to the subject 5 to 7 days after the start of the administration of the conditioning regimen.

In some embodiments, subjects are administered daratumumab, lenalidomide, and dexamethasone (DRd) prior to the administration of the conditioning regimen. In some embodiments, 4 cycles of DRd are administered to the subject prior to the administration of the conditioning regimen.

In some embodiments, daratumumab is administered weekly. In some embodiments, daratumumab is administered once every 2 weeks (Q2W). In a preferred embodiment, daratumumab is administered weekly for cycles 1 and 2, followed by once every 2 weeks (Q2W) for cycles 3 and 4. In some embodiments, the daratumumab is administered subcutaneously at a dose of 1800 mg.

In some embodiments, lenalidomide is administered for 21 days for all 4 cycles. In some embodiments, lenalidomide is administered at a dosage of 25 mg orally.

In some embodiments, dexamethasone is administered weekly in cycles 1 and 2 and once every 2 weeks (Q2W) in cycles 3 and 4. In some embodiments, dexamethasone is administered at a dose of 40 mg.

The composition comprising the host cells expressing the CAR-encoding nucleic acid sequence disclosed herein, or a vector comprising the CAR-encoding nucleic acid sequence disclosed herein, can be administered with one or more additional therapeutic agents, which can be coadministered to the subject. By “coadministering” is meant administering one or more additional therapeutic agents and the composition comprising the host cells disclosed herein or the vector disclosed herein sufficiently close in time such that the CAR disclosed herein can enhance the effect of one or more additional therapeutic agents, or vice versa. In this regard, the composition comprising the host cells disclosed herein or the vector disclosed herein can be administered first, and the one or more additional therapeutic agents can be administered second, or vice versa.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In some embodiments, the at least one additional therapeutic is a GPRC5D×CD3 bispecific antibody. In some embodiments, the administration of the GPRC5D×CD3 bispecific antibody occurs after the administration of the CAR-expressing cell, e.g., ciltacabtagene autoleucel. In some embodiments, the administration of the CAR-expressing cell, e.g., ciltacabtagene autoleucel occurs after the administration of the GPRC5D×CD3 bispecific antibody.

In some embodiments, the collection of apheresis material collected for the production of CAR-T cells, including ciltacabtagene autoleucel, occurs before the administration of the GPRC5D×CD3 bispecific antibody. In some embodiments, the collection of apheresis material collected for the production of CAR-T cells, including ciltacabtagene autoleucel, occurs after the administration of the GPRC5D×CD3 bispecific antibody.

In some embodiments, the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104 the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109, the LCDR1 of SEQ ID NO: 110, the LCDR2 of SEQ ID NO: 111 and the LCDR3 of SEQ ID NO: 112. In some embodiments, the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116. In some embodiments, the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index. In some embodiments, the GPRC5D×CD3 bispecific antibody further comprises proline at position 228, alanine at position 234 and alanine at position 235 in both the HC1 and the HC2. In some embodiments, the GPRC5D×CD3 bispecific antibody comprises the HC1 having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120. In some embodiments, the GPRC5D×CD3 bispecific antibody is talquetamab.

In some embodiments, the GPRC5D×CD3 bispecific antibody is administered weekly or once every 2 weeks (Q2W).

In some embodiments, the GPRC5D×CD3 bispecific antibody is administered in an amount sufficient to alleviate or at least partially arrest the disease being treated (“therapeutically effective amount”).

In some embodiments, the GPRC5D×CD3 bispecific antibody is subcutaneously administered.

In some embodiments, the GPRC5D×CD3 bispecific antibody is administered at a dose of 0.4 mg/kg to 0.8 mg/kg weekly or once every 2 weeks (Q2W), or any frequency in-between.

In some embodiments, the subject is administered the GPRC5D×CD3 bispecific antibody for up to 12 cycles, inclusive of cycle 12, wherein the length of each cycle is 28 days.

In some embodiments, the subject is administered three consecutive step-up doses of the GPRC5D×CD3 bispecific in the first cycle. In some embodiments, the step-up doses of the GPRC5D×CD3 bispecific antibody are 0.01 mg/kg, 0.06 mg/kg, and 0.4 mg/kg. In some embodiments, the step-up doses are administered prior to the administration of the 0.8 mg/kg dose. In some embodiments, the step-up doses may be administered between 2-5 days apart, inclusive. For example, in cycle 1, a 0.01 mg/kg dose may be administered on day 1, a 0.06 mg/kg dose may be administered on day 6 (5 days from day 1), a 0.4 mg/kg dose may be administered on day 10 (4 days from day 6), and a 0.8 mg/kg Q2W dose may be started on day 15 (5 days from day 10). For another example, in cycle 1, a 0.01 mg/kg dose may be administered on day 1, a 0.06 mg/kg dose may be administered on day 3 (2 days from day 1), a 0.4 mg/kg dose may be administered on day 5 (2 days from day 3), and a 0.8 mg/kg Q2W dose may be started on day 7 (2 days from day 5). For another example, in cycle 1, a 0.01 mg/kg dose may be administered on day 1, a 0.06 mg/kg dose may be administered on day 4 (3 days from day 1), a 0.4 mg/kg dose may be administered on day 8 (4 days from day 4), and a 0.8 mg/kg biweekly dose may be started on day 15 (7 days from day 8).

In some embodiments, the first cycle of the up to 12 cycles of the GPRC5D×CD3 bispecific antibody comprises the administration of a first step-up dose of 0.01 mg/kg of the GPRC5D×CD3 bispecific antibody, the administration of a second step-up dose of 0.06 mg/kg of the GPRC5D×CD3 bispecific antibody 2, 3, 4, or 5 days after the administration of the first step-up dose, the administration of a third step-up dose of 0.4 mg/kg of the GPRC5D×CD3 bispecific antibody 2, 3, or 4 days after the administration of the second step-up dose, and the administration of 0.8 mg/kg of the GPRC5D×CD3 bispecific antibody 2, 3, 4, 5, 6, 7, 8, 9, or 10 days after the administration of the third step-up dose.

In some embodiments, the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W). In some embodiments, each remaining cycle past the fourth comprises the administration of 0.8 mg/kg of the GPRC5D×CD3 bispecific antibody once every 4 weeks (Q4W).

In some embodiments, the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W). In some embodiments, each remaining cycle past the fifth comprises the administration of 0.8 mg/kg of the GPRC5D×CD3 bispecific antibody once every 4 weeks (Q4W).

In some embodiments, the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W). In some embodiments, each remaining cycle past the sixth comprises the administration of 0.8 mg/kg of the GPRC5D×CD3 bispecific antibody once every 4 weeks (Q4W).

In some embodiments, the administration of the GPRC5D×CD3 bispecific antibody occurs no earlier than 56 days from the administration of CAR-T cells.

In some embodiments, every cycle of the GPRC5D×CD3 bispecific antibody beyond the first comprises the administration of 0.8 mg/kg of the GPRC5D×CD3 bispecific antibody once every 2 weeks (Q2W).

In some embodiments, a subject's response to the method of treatment is assessed using the International Myeloma Working Group (IMWG)-based response criteria, which are summarized in Table 9. In some embodiments, the response may be classified as a stringent complete response (sCR). In some embodiments, the response may be classified as a complete response (CR), which is worse than a stringent complete response (sCR). In some embodiments, the response may be classified as a very good partial response (VGPR), which is worse than a complete response (CR). In some embodiments, the response may be classified as a partial response (PR), which is worse than a very good partial response (VGPR). In some embodiments, the response may be classified as a minimal response (MR), which is worse than a partial response (PR). In some embodiments, the response may be classified as a stable disease (SD), which is worse than a minimal response (MR). In some embodiments, the response may be classified as a progressive disease (PD), which is worse than a stable disease (SD).

In some embodiments, the method achieves an overall response rate of about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of treated subjects. The overall response rate may be deciphered by calculating the proportion of patients who achieve a partial response, a very good partial response, a complete response, or a stringent complete response.

In some embodiments, the tests used to assess International Myeloma Working Group (IMWG)-based response criteria are Myeloma protein (M-protein) measurements in serum and urine, serum calcium corrected for albumin, bone marrow examination, skeletal survey and documentation of extramedullary plasmacytomas.

Non-limiting examples of tests for M-protein measurement in blood and urine are known to one of ordinary skill in the art and comprise serum quantitative Ig, serum protein electrophoresis (SPEP), serum immunofixation electrophoresis, serum FLC assay, 24-hour urine M-protein quantitation by electrophoresis (UPEP), urine immunofixation electrophoresis, and serum β2-microglobulin.

Calculating serum calcium corrected for albumin in blood samples for detection of hypercalcemia is known to one of ordinary skill in the art. Without wishing to be bound by theory, calcium binds to albumin and only the unbound (free) calcium is biologically active; therefore, the serum calcium level must be adjusted for abnormal albumin levels (“corrected serum calcium”).

In some embodiments, a skeletal survey of any one of, or all of, the skull, the entire vertebral column, the pelvis, the chest, the humeri, the femora, and any other bones, may be performed and evaluated by either roentgenography (“Xrays”) or low-dose computed tomography (CT) diagnostic quality scans without the use of IV contrast, both of which are known to one of ordinary skill in the art. In some embodiments, following T cell administration and before disease progression is confirmed, X-rays or CT scans may be performed locally, whenever clinically indicated based on symptoms, to document response or progression. In some embodiments, magnetic resonance imaging (MRI) may be used for evaluating bone disease but does not replace a skeletal survey. MRI is known to one of ordinary skill in the art. In some embodiments, if a radionuclide bone scan is used at screening, in addition to the complete skeletal survey, both methods may be used to document disease status. Radionuclide bone scans are known to one of ordinary skill in the art. In some embodiments, the radionuclide bone scan and complete skeletal survey may be performed at the same time. In some embodiments, a radionuclide bone scan may not replace a complete skeletal survey. In some embodiments, if a subject presents with disease progression manifested by symptoms of pain due to bone changes, then disease progression may be documented by skeletal survey or other radiographs, depending on the symptoms that the subject experiences.

In some embodiments, extramedullary plasmacytomas may be documented by clinical examination or MRI. In some embodiments, if there was no contraindication to the use of IV contrast, extramedullary plasmacytomas may be documented by CT scan. In some embodiments, extramedullary plasmacytomas may be documented by a fusion of positron emission tomography (PET) and CT scans if the CT component is of sufficient diagnostic quality. In some embodiments, assessment of measurable sites of extramedullary disease may be performed, measured, or evaluated locally every 4 weeks for subjects until development of confirmed CR or confirmed disease progression. In some embodiments, evaluation of extramedullary plasmacytomas may be done every 12 weeks.

In some embodiments, to qualify for VGPR or PR or MR, the sum of products of the perpendicular diameters of the existing extramedullary plasmacytomas may have decreased by over 90% or at least 50%, respectively. In some embodiments, to qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. In some embodiments, to qualify for disease progression when not all existing extramedullary plasmacytomas are reported, the sum of products of the perpendicular diameters of the reported plasmacytomas had increased by at least 50%. In some embodiments, if the study treatment interferes with the immunofixation assay, CR may be defined as the disappearance of the original M-protein associated with multiple myeloma on immunofixation.

In some embodiments, a subject's response to the method of treatment is assessed in terms of change in disease burden or tumor burden. Disease burden or tumor burden represents the type of measurable disease in the subject. In some embodiments, the change in tumor burden may be assessed in terms of paraprotein level changes upon treatment. In some embodiments, the paraprotein is an M-protein in the serum. In some embodiments, the change in tumor burden is assessed in terms of the difference between involved and uninvolved free light chain (dFLC). In some embodiments, the change in tumor burden is assessed in terms of the maximum paraprotein reduction from baseline, i.e., from prior to the administration of the CAR-T cells. In some embodiments, the change in tumor burden is assessed at a median follow-up time of greater than or equal to 28 days following the administration of CAR-T cells. In some embodiments, the change in tumor burden is assessed at a median follow-up time of greater than or equal to 1 month following the administration of CAR-T cells. In some embodiments, the change in tumor burden is assessed at a median follow-up time of greater than or equal to 3 months following the administration of CAR-T cells. In some embodiments, the change in tumor burden is assessed at a median follow-up time of greater than or equal to 6 months following the administration of CAR-T cells. In some embodiments, the change in tumor burden is assessed at a median follow-up time of greater than or equal to 9 months following the administration of CAR-T cells. In some embodiments, the change in tumor burden is assessed at a median follow-up time of greater than or equal to 12 months following the administration of CAR-T cells.

In some embodiments, bone marrow aspirate or biopsy may be performed for clinical assessments or bone marrow aspirate may be performed for biomarker evaluations. In some embodiments, clinical staging (morphology, cytogenetics, and immunohistochemistry or immunofluorescence or flow cytometry) may be done. In some embodiments, a portion of the bone marrow aspirate may be immunophenotyped and monitored for BCMA, checkpoint ligand expression in CD138-positive multiple myeloma cells, and checkpoint expression on T cells. In some embodiments, minimal residual disease (MRD) may be monitored in subjects using next generation sequencing (NGS) of bone marrow aspirate DNA. The NGS of bone marrow aspirate DNA is known to one of ordinary skill in the art. In some embodiments, the NGS is performed via clonoSEQ. In some embodiments, baseline bone marrow aspirates may be used to define the myeloma clones, and post-treatment samples may be used to evaluate MRD negativity. In some embodiments, the MRD negativity status may be based on samples that are evaluable. In some embodiments, evaluable samples are those that passed one or more of, or all of, calibration, quality control, and sufficiency of cells evaluable at a particular sensitivity level. In some embodiments, the sensitivity level is 10⁻⁶. In some embodiments, the sensitivity level is 10⁻⁵. In some embodiments, the sensitivity level is 10⁻⁴. In some embodiments, the sensitivity level is 10⁻³.

In some embodiments, the method achieves MRD-negativity at a threshold of 10⁻⁵ before disease progression or start of a subsequent antimyeloma therapy. In some embodiments, the method achieves sustained MRD-negative status, as determined by NGS with sensitivity of 10⁻⁵, for at least 6 months without examination showing MRD-positive or PD in between. In some embodiments, the method achieves sustained MRD-negative status, as determined by NGS with sensitivity of 10⁻⁵, for at least 12 months without examination showing MRD-positive or PD in between. In some embodiments, the method achieves sustained MRD-negative status, as determined by NGS with sensitivity of 10⁻⁵, for at least 18 months without examination showing MRD-positive or PD in between. In some embodiments, the method achieves sustained MRD-negative status, as determined by NGS with sensitivity of 10⁻⁵, for at least 24 months without examination showing MRD-positive or PD in between. In some embodiments, the method achieves sustained MRD-negative status, as determined by NGS with sensitivity of 10⁻⁵, for at least 30 months without examination showing MRD-positive or PD in between. In some embodiments, the method achieves sustained MRD-negative status, as determined by NGS with sensitivity of 10⁻⁵, for at least 36 months without examination showing MRD-positive or PD in between. In some embodiments, the method achieves sustained MRD-negative status, as determined by NGS with sensitivity of 10⁻⁵, for at least 42 months without examination showing MRD-positive or PD in between. In some embodiments, the method achieves sustained MRD-negative status, as determined by NGS with sensitivity of 10⁻⁵, for at least 48 months without examination showing MRD-positive or PD in between. In some embodiments, the method achieves sustained MRD-negative status, as determined by NGS with sensitivity of 10⁻⁵, for at least 54 months without examination showing MRD-positive or PD in between. In some embodiments, the method achieves sustained MRD-negative status, as determined by NGS with sensitivity of 10⁻⁵, for at least 60 months without examination showing MRD-positive or PD in between.

In some embodiments, the method achieves MRD-negativity at a threshold of 10⁻⁶ before disease progression or start of a subsequent antimyeloma therapy. In some embodiments, the method achieves sustained MRD-negative status, as determined by NGS with sensitivity of 10⁻⁶, for at least 6 months without examination showing MRD-positive or PD in between. In some embodiments, the method achieves sustained MRD-negative status, as determined by NGS with sensitivity of 10⁻⁶, for at least 12 months without examination showing MRD-positive or PD in between. In some embodiments, the method achieves sustained MRD-negative status, as determined by NGS with sensitivity of 10⁻⁶, for at least 18 months without examination showing MRD-positive or PD in between. In some embodiments, the method achieves sustained MRD-negative status, as determined by NGS with sensitivity of 10⁻⁶, for at least 24 months without examination showing MRD-positive or PD in between. In some embodiments, the method achieves sustained MRD-negative status, as determined by NGS with sensitivity of 10⁻⁶, for at least 30 months without examination showing MRD-positive or PD in between. In some embodiments, the method achieves sustained MRD-negative status, as determined by NGS with sensitivity of 10⁻⁶, for at least 36 months without examination showing MRD-positive or PD in between. In some embodiments, the method achieves sustained MRD-negative status, as determined by NGS with sensitivity of 10⁻⁶, for at least 42 months without examination showing MRD-positive or PD in between. In some embodiments, the method achieves sustained MRD-negative status, as determined by NGS with sensitivity of 10⁻⁶, for at least 48 months without examination showing MRD-positive or PD in between. In some embodiments, the method achieves sustained MRD-negative status, as determined by NGS with sensitivity of 10⁻⁶, for at least 54 months without examination showing MRD-positive or PD in between. In some embodiments, the method achieves sustained MRD-negative status, as determined by NGS with sensitivity of 10⁻⁶, for at least 60 months without examination showing MRD-positive or PD in between.

Also provided in further embodiments is the medical use of the therapies (including CARs, CAR T cells, and antibodies) provided herein for use in the methods of treatment disclosed herein. Also provided in further embodiments is the use of the therapies (including CARs, and CAR T cells), and antibodies provided herein for use in the manufacture of a medicament for the use in the methods of treatment disclosed herein.

### ENUMERATED EMBODIMENTS

The following examples are illustrative, but not limiting, of the compounds, compositions and methods described herein. Other suitable modifications and adaptations known to those skilled in the art are within the scope of the following embodiments.

1. A method of treating multiple myeloma in a subject in need thereof, the method comprising:
administering ciltacabtagene autoleucel to the subject at a dosage of 0.5-1.0×10⁶ CAR-positive viable T cells/kg, and
administering a GPRC5D×CD3 bispecific antibody to the subject;
wherein the administration of the GPRC5D×CD3 bispecific antibody occurs after the administration of ciltacabtagene autoleucel; and
wherein the subject has relapsed and/or refractory multiple myeloma, and received at least three prior lines of therapies, including a proteasomal inhibitor (PI), an immunomodulatory drug (IMiD), and an anti-CD38 antibody.

2. The method of embodiment 1, wherein the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104, the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109, the LCDR1 of SEQ ID NO: 110, the LCDR2 of SEQ ID NO: 111 and the LCDR3 of SEQ ID NO: 112.

3. The method of embodiment 1 or 2, wherein the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116.

4. The method of any one of embodiments 1 to 3, wherein the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

5. The method of embodiment 4, wherein the GPRC5D×CD3 bispecific antibody further comprises proline at position 228, alanine at position 234 and alanine at position 235 in both the HC1 and the HC2.

6. The method of embodiments 1 to 5, wherein the GPRC5D×CD3 bispecific antibody comprises a first heavy chain (HC1) having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120.

7. The method of any one of embodiments 1 to 6, wherein the GPRC5D×CD3 bispecific antibody is talquetamab.

8. The method of any one of embodiments 1 to 7, wherein the administration of the GPRC5D×CD3 bispecific antibody is once every 2 weeks (Q2W) at a dosage of 0.8 mg/kg.

9. The method of any one of embodiments 1 to 8, wherein the administration of the GPRC5D×CD3 bispecific antibody is weekly at a dosage of 0.4 mg/kg.

10. The method of any one of embodiments 1 to 9, further comprising administering a conditioning regimen to the subject prior to administering ciltacabtagene autoleucel, wherein the conditioning regimen comprises one or more of cyclophosphamide and/or fludarabine.

11. The method of embodiment 10, wherein the conditioning regimen comprises cyclophosphamide at a dosage of 300 mg/m².

12. The method of embodiment 10, wherein the conditioning regimen comprises fludarabine at a dosage of 30 mg/m².

13. The method of embodiment 10, wherein the conditioning regimen comprises cyclophosphamide at a dosage of 300 mg/m² and fludarabine at a dosage of 30 mg/m².

14. The method of any one of embodiments 10 to 13, wherein the conditioning regimen is administered to the subject daily, for up to 3 days.

15. The method of any one of embodiments 10 to 14, wherein the ciltacabtagene autoleucel is administered to the subject 5 to 7 days after the start of the administration of the conditioning regimen.

16. The method of any one of embodiments 1 to 15, wherein the ciltacabtagene autoleucel is administered to the subject at a dose of 0.75×10⁶ CAR-positive viable T cells/kg.

17. The method of any one of embodiments 1 to 16, wherein the GPRC5D×CD3 bispecific antibody is administered subcutaneously.

18. The method of any one of embodiments 1 to 17, wherein the subject is administered up to 12 cycles of the GPRC5D×CD3 bispecific antibody.

19. The method of any one of embodiments 1 to 18, wherein the subject is administered up to 12 cycles of the GPRC5D×CD3 bispecific antibody, and wherein the first cycle of the up to 12 cycles comprises three consecutive step-up doses of the GPRC5D×CD3 bispecific antibody of 0.01 mg/kg, 0.06 mg/kg, and 0.4 mg/kg, prior to receiving the 0.8 mg/kg dose once every 2 weeks.

20. The method of embodiment 19, wherein the three consecutive step-up doses are administered 2, 3, 4, or 5 days apart.

21. The method of embodiments 19 or 20, wherein the subject is administered up to 12 cycles of the GPRC5D×CD3 bispecific antibody, and wherein the first cycle of the up to 12 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and the administration of 0.8 mg/kg of the GPRC5D×CD3 bispecific antibody 2, 3, 4, 5, 6, 7, 8, 9, or 10 days after the administration of the third step-up dose.

22. The method of embodiment 21, wherein the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the fourth comprises the administration of 0.8 mg/kg of the GPRC5D×CD3 bispecific antibody once every 4 weeks (Q4W).

23. The method of embodiment 21, wherein the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the fifth comprises the administration of 0.8 mg/kg of the GPRC5D×CD3 bispecific antibody once every 4 weeks (Q4W).

24. The method of embodiment 21, wherein the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the sixth comprises the administration of 0.8 mg/kg of the GPRC5D×CD3 bispecific antibody once every 4 weeks (Q4W).

25. The method of any one of embodiments 1 to 24, wherein the administration of the GPRC5D×CD3 bispecific antibody occurs no earlier than 56 days from the administration of ciltacabtagene autoleucel.

26. The method of any of embodiments 1 to 25, wherein the method achieves a partial response (PR), very good partial response (VGPR), complete response (CR) or stringent complete response (sCR) in the subject, according to IMWG criteria.

27. The method of any of embodiments 1 to 25, wherein the method achieves a very good partial response (VGPR), complete response (CR) or stringent complete response (sCR) in the subject, according to IMWG criteria.

28. The method of any of embodiments 1 to 25, wherein the method achieves a complete response (CR) or stringent complete response (sCR) in the subject, according to IMWG criteria.

29. The method of any of embodiments 1 to 25, wherein the method achieves a stringent complete response (sCR) in the subject, according to IMWG criteria.

30. The method of any one of embodiments 1 to 29, wherein the method achieves MRD-negativity at a threshold of 10⁻⁵ before disease progression or start of a subsequent antimyeloma therapy.

31. The method of any one of embodiments 1 to 30, wherein the method achieves sustained MRD-negative status, as determined by NGS with sensitivity of 10⁻⁵, for at least 6 months without examination showing MRD-positive or PD in between.

32. A method of treating multiple myeloma in a subject in need thereof, the method comprising:
administering ciltacabtagene autoleucel to the subject at a dosage of 0.5-1.0×10⁶ CAR-positive viable T cells/kg, and
administering a GPRC5D×CD3 bispecific antibody to the subject;
wherein the administration of the GPRC5D×CD3 bispecific antibody occurs after the administration of ciltacabtagene autoleucel, and
wherein the subject has newly diagnosed multiple myeloma and is transplant ineligible.

33. The method of embodiment 32, wherein the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104, the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109, the LCDR1 of SEQ ID NO: 110, the LCDR2 of SEQ ID NO: 111 and the LCDR3 of SEQ ID NO: 112.

34. The method of embodiments 32 or 33, wherein the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116.

35. The method of any one of embodiments 32 to 34, wherein the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

36. The method of embodiment 35, wherein the GPRC5D×CD3 bispecific antibody further comprises proline at position 228, alanine at position 234 and alanine at position 235 in both the HC1 and the HC2.

37. The method of embodiment 35 or 36, wherein the GPRC5D×CD3 bispecific antibody comprises the HC1 having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120.

38. The method of any one of embodiments 32 to 37, wherein the GPRC5D×CD3 bispecific antibody is talquetamab.

39. The method of any one of embodiments 32 to 38, wherein the administration of the GPRC5D×CD3 bispecific antibody is once every 2 weeks (Q2W) at a dosage of 0.8 mg/kg.

40. The method of any one of embodiments 32 to 39, wherein the administration of the GPRC5D×CD3 bispecific antibody is weekly at a dosage of 0.4 mg/kg.

41. The method of any one of embodiments 32 to 40, further comprising administering a conditioning regimen to the subject prior to administering ciltacabtagene autoleucel, wherein the conditioning regimen comprises one or more of cyclophosphamide and/or fludarabine.

42. The method of embodiment 41, wherein the conditioning regimen comprises cyclophosphamide at a dosage of 300 mg/m².

43. The method of embodiment 41, wherein the conditioning regimen comprises fludarabine at a dosage of 30 mg/m².

44. The method of embodiment 41, wherein the conditioning regimen comprises cyclophosphamide at a dosage of 300 mg/m² and fludarabine at a dosage of 30 mg/m².

45. The method of any one of embodiments 41 to 44, wherein the conditioning regimen is administered to the subject daily, for up to 3 days.

46. The method of any one of embodiments 41 to 45, wherein the ciltacabtagene autoleucel is administered to the subject 5 to 7 days after the start of the administration of the conditioning regimen.

47. The method of any one of embodiments 41 to 46, comprising administering 4 cycles of daratumumab, lenalidomide, and dexamethasone (DRd) to the subject prior to the administration of the conditioning regimen,
wherein each cycle is about 28 days,
wherein daratumumab is administered at a dosage of 1,800 mg subcutaneously weekly for cycles 1 and 2, followed by once every 2 weeks (Q2W) for cycles 3 and 4,
wherein lenalidomide is administered at a dosage of 25 mg orally for 21 days for all 4 cycles, and
wherein dexamethasone is administered at a dosage of 40 mg weekly in cycles 1 and 2 and once every 2 weeks (Q2W) in cycles 3 and 4.

48. The method of any one of embodiments 32 to 47, wherein the ciltacabtagene autoleucel is administered to the subject at a dose of 0.75×10⁶ CAR-positive viable T cells/kg.

49. The method of any one of embodiments 32 to 48, wherein the GPRC5D×CD3 bispecific antibody is administered subcutaneously.

50. The method of any one of embodiments 32 to 49, wherein the subject is administered up to 12 cycles of the GPRC5D×CD3 bispecific antibody.

51. The method of embodiment 50, wherein the subject receives three consecutive step-up doses of the GPRC5D×CD3 bispecific antibody of 0.01 mg/kg, 0.06 mg/kg, and 0.4 mg/kg, prior to receiving the 0.8 mg/kg once every 2 weeks (Q2W) dose.

52. The method of embodiment 51, wherein the three consecutive step-up doses are administered 2, 3, 4, or 5 days apart.

53. The method of embodiments 51 or 52, wherein the subject is administered up to 12 cycles of the GPRC5D×CD3 bispecific antibody, and wherein the first cycle of the up to 12 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose.

54. The method of embodiment 53, wherein the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the fourth comprises the administration of 0.8 mg/kg of the GPRC5D×CD3 bispecific antibody once every 4 weeks (Q4W).

55. The method of embodiment 53, wherein the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the fifth comprises the administration of 0.8 mg/kg of the GPRC5D×CD3 bispecific antibody once every 4 weeks (Q4W).

56. The method of embodiment 53, wherein the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the sixth comprises the administration of 0.8 mg/kg of the GPRC5D×CD3 bispecific antibody once every 4 weeks (Q4W).

57. The method of any one of embodiments 32 to 56, wherein the administration of the GPRC5D×CD3 bispecific antibody occurs no earlier than 56 days from the administration of ciltacabtagene autoleucel.

58. The method of any of embodiments 32 to 57, wherein the method achieves a partial response (PR), very good partial response (VGPR), complete response (CR) or stringent complete response (sCR) in the subject, according to IMWG criteria.

59. The method of any of embodiments 32 to 57, wherein the method achieves a very good partial response (VGPR), complete response (CR) or stringent complete response (sCR) in the subject, according to IMWG criteria.

60. The method of any of embodiments 32 to 57, wherein the method achieves a complete response (CR) or stringent complete response (sCR) in the subject, according to IMWG criteria.

61. The method of any of embodiments 32 to 57, wherein the method achieves a stringent complete response (sCR) in the subject, according to IMWG criteria.

62. The method of any one of embodiments 32 to 57, wherein the method achieves MRD-negativity at a threshold of 10⁻⁵ before disease progression or start of a subsequent anti-myeloma therapy.

63. The method of any one of embodiments 32 to 57, wherein the method achieves sustained MRD-negative status, as determined by NGS with sensitivity of 10⁻⁵, for at least 6 months without examination showing MRD-positive or PD in between.

64. A method of treating multiple myeloma in a subject in need thereof, the method comprising:
administering a GPRC5D×CD3 bispecific antibody to the subject, and
administering ciltacabtagene autoleucel to the subject at a dosage of 0.5-1.0×10⁶ CAR-positive viable T cells/kg;
wherein the administration of ciltacabtagene autoleucel occurs after the administration of the GPRC5D×CD3 bispecific antibody, and
wherein the subject has relapsed and/or refractory multiple myeloma, and received at least three prior lines of therapies, including a proteasomal inhibitor (PI), an immunomodulatory drug (IMiD), and an anti-CD38 antibody.

65. The method of embodiment 64, wherein the GPRC5D×CD3 bispecific antibody comprises a GPRC5D binding domain comprising the HCDR1 of SEQ ID NO: 101, the HCDR2 of SEQ ID NO: 102, the HCDR3 of SEQ ID NO: 103, the LCDR1 of SEQ ID NO: 104, the LCDR2 of SEQ ID NO: 105 and the LCDR3 of SEQ ID NO: 106, and a CD3 binding domain comprising the HCDR1 of SEQ ID NO: 107, the HCDR2 of SEQ ID NO: 108, the HCDR3 of SEQ ID NO: 109 the LCDR1 of SEQ ID NO: 110, the LCDR2 of SEQ ID NO: 111 and the LCDR3 of SEQ ID NO: 112.

66. The method of embodiments 64 or 65, wherein the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116.

67. The method of any one of embodiments 64 to 66, wherein the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

68. The method of embodiment 67, wherein the GPRC5D×CD3 bispecific antibody further comprises proline at position 228, alanine at position 234 and alanine at position 235 in both the HC1 and the HC2.

69. The method of embodiment 67 or 68, wherein the GPRC5D×CD3 bispecific antibody comprises the HC1 having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120.

70. The method of any one of embodiments 64 to 69, wherein the GPRC5D×CD3 bispecific antibody is talquetamab.

71. The method of any one of embodiments 64 to 70, wherein the administration of the GPRC5D×CD3 bispecific antibody is once every 2 weeks (Q2W) at a dosage of 0.8 mg/kg.

72. The method of any one of embodiments 64 to 71, wherein the administration of the GPRC5D×CD3 bispecific antibody is weekly at a dosage of 0.4 mg/kg.

73. The method of any one of embodiments 64 to 72, further comprising administering a conditioning regimen to the subject prior to administering ciltacabtagene autoleucel, wherein the conditioning regimen comprises one or more of cyclophosphamide and/or fludarabine.

74. The method of embodiment 73, wherein the conditioning regimen comprises cyclophosphamide at a dosage of 300 mg/m².

75. The method of embodiment 73, wherein the conditioning regimen comprises fludarabine at a dosage of 30 mg/m².

76. The method of embodiment 73, wherein the conditioning regimen comprises cyclophosphamide at a dosage of 300 mg/m² and fludarabine at a dosage of 30 mg/m².

77. The method of any one of embodiments 73 to 76, wherein the conditioning regimen is administered to the subject daily, for up to 3 days.

78. The method of any one of embodiments 64 to 77, wherein the ciltacabtagene autoleucel is administered to the subject 5 to 7 days after the start of the administration of the conditioning regimen.

79. The method of any one of embodiments 64 to 78, wherein the method further comprises collecting apheresis material from the subject for the production of ciltacabtagene autoleucel.

80. The method of embodiment 79, wherein the collection of apheresis material occurs before the administration of the GPRC5D×CD3 bispecific antibody.

81. The method of embodiment 79, wherein the collection of apheresis material occurs after the administration of the GPRC5D×CD3 bispecific antibody.

82. The method of any one of embodiments 64 to 81, wherein the ciltacabtagene autoleucel is administered to the subject at a dose of 0.75×10⁶ CAR-positive viable T cells/kg.

83. The method of any one of embodiments 64 to 82, wherein the GPRC5D×CD3 bispecific antibody is administered subcutaneously.

84. The method of any one of embodiments 64 to 83, wherein the subject is administered 2, 3, or 4 cycles of the GPRC5D×CD3 bispecific antibody.

85. The method of embodiment 84, wherein the subject receives three consecutive step-up doses of the GPRC5D×CD3 bispecific antibody of 0.01 mg/kg, 0.06 mg/kg, and 0.4 mg/kg, prior to receiving the 0.8 mg/kg dose once every 2 weeks.

86. The method of embodiment 85, wherein the three consecutive step-up doses are administered 2, 3, 4, or 5 days apart.

87. The method of embodiments 85 or 86, wherein the first cycle of the 2, 3, or 4 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose.

88. The method of embodiment 87, wherein every cycle of the GPRC5D×CD3 bispecific antibody beyond the first comprises the administration of 0.8 mg/kg of the GPRC5D×CD3 bispecific antibody once every 2 weeks (Q2W).

89. The method of any of embodiments 64 to 88, wherein the method achieves a partial response (PR), very good partial response (VGPR), complete response (CR) or stringent complete response (sCR) in the subject, according to IMWG criteria.

90. The method of any of embodiments 64 to 88, wherein the method achieves a very good partial response (VGPR), complete response (CR) or stringent complete response (sCR) in the subject, according to IMWG criteria.

91. The method of any of embodiments 64 to 88, wherein the method achieves a complete response (CR) or stringent complete response (sCR) in the subject, according to IMWG criteria.

92. The method of any of embodiments 64 to 88, wherein the method achieves a stringent complete response (sCR) in the subject, according to IMWG criteria.

93. The method of any one of embodiments 64 to 88, wherein the method achieves MRD-negativity at a threshold of 10⁻⁵ before disease progression or start of a subsequent anti-myeloma therapy.

94. The method of any one of embodiments 64 to 88, wherein the method achieves sustained MRD-negative status, as determined by NGS with sensitivity of 10⁻⁵, for at least 6 months without examination showing MRD-positive or PD in between.

95. A method of treating multiple myeloma in a subject in need thereof, the method comprising:
administering ciltacabtagene autoleucel to the subject at a dosage of 0.75×10⁶ CAR-positive viable T cells/kg, and
administering a GPRC5D×CD3 bispecific antibody comprising a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116 to the subject;
wherein the subject is administered up to 12 cycles of the GPRC5D×CD3 bispecific antibody,
wherein the first cycle of the up to 12 cycles of the GPRC5D×CD3 bispecific antibody comprises three consecutive step-up doses of the GPRC5D×CD3 bispecific antibody of 0.01 mg/kg, 0.06 mg/kg, and 0.4 mg/kg,
wherein the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W),
wherein each remaining cycle past the fourth comprises the administration of 0.8 mg/kg of the GPRC5D×CD3 bispecific antibody once every 4 weeks (Q4W), and
wherein the administration of the GPRC5D×CD3 bispecific antibody occurs after the administration of ciltacabtagene autoleucel.

96. A method of treating multiple myeloma in a subject in need thereof, the method comprising:
administering ciltacabtagene autoleucel to the subject at a dosage of 0.75×10⁶ CAR-positive viable T cells/kg, and
administering a GPRC5D×CD3 bispecific antibody comprising a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116 to the subject;
wherein the subject is administered up to 12 cycles of the GPRC5D×CD3 bispecific antibody,
wherein the first cycle of the up to 12 cycles of the GPRC5D×CD3 bispecific antibody comprises three consecutive step-up doses of the GPRC5D×CD3 bispecific antibody of 0.01 mg/kg, 0.06 mg/kg, and 0.4 mg/kg,
wherein the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W),
wherein each remaining cycle past the fifth comprises the administration of 0.8 mg/kg of the GPRC5D×CD3 bispecific antibody once every 4 weeks (Q4W), and
wherein the administration of the GPRC5D×CD3 bispecific antibody occurs after the administration of ciltacabtagene autoleucel.

97. A method of treating multiple myeloma in a subject in need thereof, the method comprising:
administering ciltacabtagene autoleucel to the subject at a dosage of 0.75×10⁶ CAR-positive viable T cells/kg, and
administering a GPRC5D×CD3 bispecific antibody comprising a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116 to the subject;
wherein the subject is administered up to 12 cycles of the GPRC5D×CD3 bispecific antibody,
wherein the first cycle of the up to 12 cycles of the GPRC5D×CD3 bispecific antibody comprises three consecutive step-up doses of the GPRC5D×CD3 bispecific antibody of 0.01 mg/kg, 0.06 mg/kg, and 0.4 mg/kg,
wherein the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W),
wherein each remaining cycle past the sixth comprises the administration of 0.8 mg/kg of the GPRC5D×CD3 bispecific antibody once every 4 weeks (Q4W), and
wherein the administration of the GPRC5D×CD3 bispecific antibody occurs after the administration of ciltacabtagene autoleucel.

98. A method according to the preceding embodiments, wherein the subject has relapsed and/or refractory multiple myeloma, and received at least three prior lines of therapies, including a (PI), an immunomodulatory drug (IMiD), and an anti-CD38 antibody.

99. A method of treating multiple myeloma in a subject in need thereof, the method comprising:
administering 4 cycles of daratumumab, lenalidomide, and dexamethasone (DRd) to the subject prior to the administration a conditioning regimen,
wherein daratumumab is administered at a dosage of 1,800 mg subcutaneously weekly for cycles 1 and 2, followed by once every 2 weeks (Q2W) for cycles 3 and 4,
wherein lenalidomide is administered at a dosage of 25 mg orally for 21 days for all 4 cycles, and
wherein dexamethasone is administered at a dosage of 40 mg weekly in cycles 1 and 2 and once every 2 weeks (Q2W) in cycles 3 and 4,
administering ciltacabtagene autoleucel to the subject at a dosage of 0.75×10⁶ CAR-positive viable T cells/kg, and
administering a GPRC5D×CD3 bispecific antibody to the subject;
wherein the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116,
wherein the administration of the GPRC5D×CD3 bispecific antibody occurs after the administration of ciltacabtagene autoleucel,
wherein the subject is administered up to 12 cycles of the GPRC5D×CD3 bispecific antibody,
wherein the first cycle of the up to 12 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose, wherein the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W),
wherein each remaining cycle past the fourth comprises the administration of 0.8 mg/kg of the GPRC5D×CD3 bispecific antibody once every 4 weeks (Q4W), and
wherein the subject has newly diagnosed multiple myeloma and is transplant ineligible.

100. A method of treating multiple myeloma in a subject in need thereof, the method comprising:
administering 4 cycles of daratumumab, lenalidomide, and dexamethasone (DRd) to the subject prior to the administration a conditioning regimen,
wherein daratumumab is administered at a dosage of 1,800 mg subcutaneously weekly for cycles 1 and 2, followed by once every 2 weeks (Q2W) for cycles 3 and 4,
wherein lenalidomide is administered at a dosage of 25 mg orally for 21 days for all 4 cycles, and
wherein dexamethasone is administered weekly in cycles 1 and 2 and once every 2 weeks (Q2W) in cycles 3 and 4,
administering ciltacabtagene autoleucel to the subject at a dosage of 0.75×10⁶ CAR-positive viable T cells/kg, and
administering a GPRC5D×CD3 bispecific antibody to the subject;
wherein the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116,
wherein the administration of the GPRC5D×CD3 bispecific antibody occurs after the administration of ciltacabtagene autoleucel,
wherein the subject is administered up to 12 cycles of the GPRC5D×CD3 bispecific antibody,
wherein the first cycle of the up to 12 cycles of the GPRC5D×CD3 bispecific antibody comprises:
   the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose,
   wherein the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W),
wherein each remaining cycle past the fifth comprises the administration of 0.8 mg/kg of the GPRC5D×CD3 bispecific antibody once every 4 weeks (Q4W), and
wherein the subject has newly diagnosed multiple myeloma and is transplant ineligible.

101. A method of treating multiple myeloma in a subject in need thereof, the method comprising:
administering 4 cycles of daratumumab, lenalidomide, and dexamethasone (DRd) to the subject prior to the administration a conditioning regimen,
wherein daratumumab is administered at a dosage of 1,800 mg subcutaneously weekly for cycles 1 and 2, followed by once every 2 weeks (Q2W) for cycles 3 and 4,
wherein lenalidomide is administered at a dosage of 25 mg orally for 21 days for all 4 cycles, and
wherein dexamethasone is administered weekly in cycles 1 and 2 and once every 2 weeks (Q2W) in cycles 3 and 4,
administering ciltacabtagene autoleucel to the subject at a dosage of 0.75×10⁶ CAR-positive viable T cells/kg, and
administering a GPRC5D×CD3 bispecific antibody to the subject;
wherein the GPRC5D binding domain comprises a heavy chain variable region (VH) having the amino acid sequence of SEQ ID NO: 113 and a light chain variable region (VL) having the amino acid sequence of SEQ ID NO: 114, and the CD3 binding domain comprises a VH having the amino acid sequence of SEQ ID NO: 115 and a VL having the amino acid sequence of SEQ ID NO: 116,
wherein the administration of the GPRC5D×CD3 bispecific antibody occurs after the administration of ciltacabtagene autoleucel,
wherein the subject is administered up to 12 cycles of the GPRC5D×CD3 bispecific antibody,
   wherein the first cycle of the up to 12 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose,
wherein the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W),
wherein each remaining cycle past the sixth comprises the administration of 0.8 mg/kg of the GPRC5D×CD3 bispecific antibody once every 4 weeks (Q4W), and
wherein the subject has newly diagnosed multiple myeloma and is transplant ineligible.

102. A method of treating multiple myeloma in a subject in need thereof, the method comprising:
administering a GPRC5D×CD3 bispecific antibody to the subject,
wherein the GPRC5D×CD3 bispecific antibody comprises the HC1 having the amino acid sequence of SEQ ID NO: 117, a first light chain (LC1) having the amino acid sequence of SEQ ID NO: 118, the HC2 having the amino acid sequence of SEQ ID NO: 119 and a second light chain (LC2) having the amino acid sequence of SEQ ID NO: 120,
wherein the subject is administered 2, 3, or 4 cycles of the GPRC5D×CD3 bispecific antibody,
   wherein the first cycle of the 2, 3, or 4 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose,
wherein every cycle of the GPRC5D×CD3 bispecific antibody beyond the first comprises the administration of 0.8 mg/kg of the GPRC5D×CD3 bispecific antibody once every 2 weeks (Q2W), and
administering ciltacabtagene autoleucel to the subject at a dosage of 0.75×10⁶ CAR-positive viable T cells/kg;
wherein the administration of ciltacabtagene autoleucel occurs after the administration of the GPRC5D×CD3 bispecific antibody.

103. The method of embodiment 102, wherein the subject has relapsed and/or refractory multiple myeloma, and received at least three prior lines of therapies, including a proteasomal inhibitor (PI), an immunomodulatory drug (IMiD), and an anti-CD38 antibody.

104. A method of treating multiple myeloma in a subject in need thereof, the method comprising: administering anti-BCMA CAR T cells to the subject, and administering a GPRC5D×CD3 bispecific antibody to the subject. In certain embodiments, the administration of the GPRC5D×CD3 bispecific antibody occurs after the administration of the anti-BCMA CAR T cells. In certain embodiments, the administration of the GPRC5D×CD3 bispecific antibody occurs before the administration of the anti-BCMA CAR T cells.

105. The method of embodiment 104, wherein the method comprises one or more of the features of any one of embodiments 1 to 103.

### EXAMPLES

The following examples are provided to further describe some of the aspects and embodiments disclosed herein. The examples are intended to illustrate, not to limit, the disclosed aspects or embodiments.

### Example 1: Ciltacabtagene Autoleucel and Talquetamab

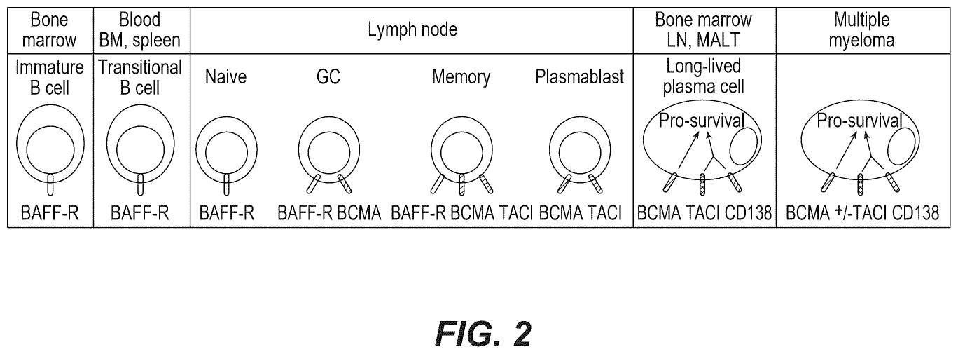

B-cell maturation antigen (BCMA, also known as CD269 and TNFRSF17) is a 20 kilodalton, type III membrane protein that is part of the tumor necrosis receptor superfamily. BCMA is a cell surface antigen that is predominantly expressed in B-lineage cells at high levels. FIG. 2 shows the expression of BCMA on various immune-derived cells. Comparative studies have shown a lack of BCMA in most normal tissues and absence of expression on CD34-positive hematopoietic stem cells. BCMA binds 2 ligands that induce B cell proliferation and plays a critical role in B cell maturation and subsequent differentiation into plasma cells. The selective expression and the biological importance for the proliferation and survival of myeloma cells makes BCMA a promising target for CAR-T based immunotherapy.

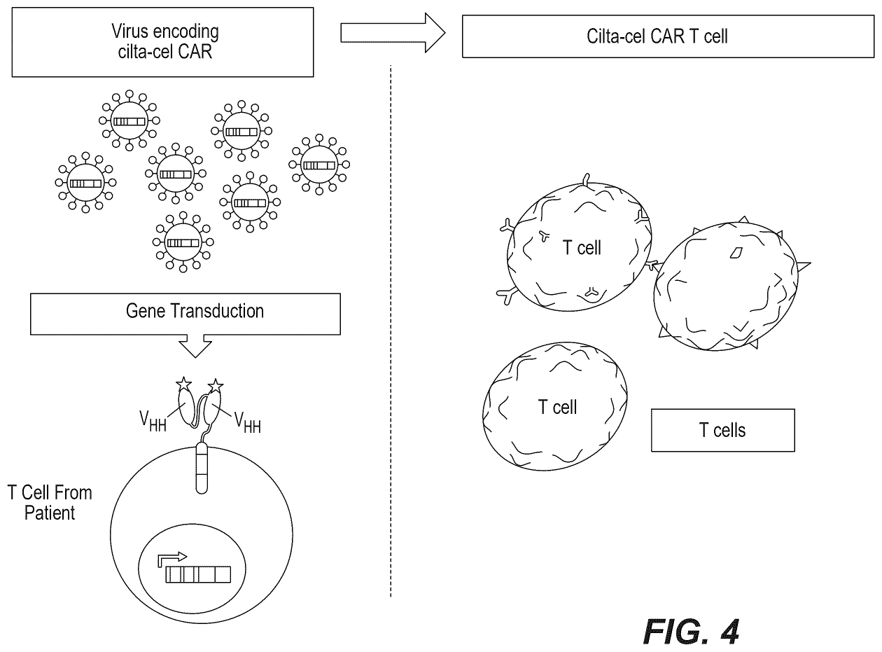

Ciltacabtagene autoleucel (also known as cilta-cel) is an autologous chimeric antigen receptor T-cell (CAR-T) therapy that targets BCMA. The ciltacabtagene autoleucel chimeric antigen receptor (CAR) comprises two BCMA-targeting VHH domains designed to confer avidity. A map of the construct is depicted in FIG. 3 and a schematic of the CAR-T cell production is shown in FIG. 4. Ciltacabtagene autoleucel includes a VHH domain comprising the amino acid sequence set forth in SEQ ID NO: 102 and a VHH domain comprising the amino acid sequence set forth in SEQ ID NO: 104.

Talquetamab is a humanized IgG4 bispecific antibody designed to target the CD3 receptor complex on T-cells and GPRC5D-expressing plasma cells (PCs). Talquetamab comprises a CD3 binding arm CD3B219 and a GPRC5d binding arm GC5B596, the amino acid sequences of which are shown in Table 7A and Table 7B, respectively.

### Example 2: Considerations for Optimal Sequencing and/or Combination of CAR-T and CD3 Bispecifics

Recent advancements in multiple myeloma therapy have shown significant clinical responses in their respective trials. Carvykti® (cilta-cel) was approved in February 2022 with a 97.9% response rate as observed in CARTITUDE-1. Tecvayli® (teclistamab) was approved in October 2022, with a 63% overall response rate (ORR) registered in the MajesTEC-1 trial and a median response duration of approximately 22 months. Talvey™ (talquetamab) was approved in August 2022 and saw a response rate of 73% in the MonumenTAL-1 trial with the median response duration for the 0.4 mg/kg group measuring at 9.5 months.

These drugs show a similarity in their mode of action (MOA), functioning through direct cytotoxicity (examples include perforin, granzyme, Fas/FasL) and cytokine production (which include agents like IFN-γ, TNF-α, IL-2, etc.). This group of drugs, as a franchise, addresses various multiple myeloma therapy lines and patient segments.

To optimize patient outcomes with ciltacabtagene autoleucel, it is hypothesized that T cell fitness and CAR-T outcomes are influenced by previous exposure to bispecific T cell redirectors. This informs an intervention strategy that may involve therapy sequencing (for instance, administering ciltacabtagene autoleucel earlier and/or performing apheresis prior to Tec/Tal and suchlike), screening patients for T cell fitness and addressing this prior to aphaeresis, and potentially aiming for synergy through adjuvant/combination therapies as opposed to rudimentary sequential therapies. Still, the sequencing and/or combination of CAR-T and CD3 bispecifics, specifically ciltacabtagene autoleucel, teclistamab, and talquetamab, calls for careful considerations. One of the most crucial aspects to consider is whether treating with one or more of these drugs affects the potential for subsequent treatment with another. To decide an optimal course of action, considerations of safety, efficacy, and timing need to be factored into whether these drugs are administered together or in close succession. This involves seeking ways to enhance efficacy by combining or sequencing these drugs without compromising safety.

Clinical data, based on the CARTITUDE-2 study (NCT04133636), indicates that ciltacabtagene autoleucel patients previously treated with BCMA targeted bispecifics show responses far below expectations. T-cell analyses from these patients exhibit a decreased CD4:CD8 ratio and increase in CD38 expression, potentially indicating a lower level of “fitness”. Preclinical data, too, suggests that exposure to bispecifics may reduce T cell “fitness.” However, preclinical data also suggests that CAR-T can enhance bispecific responses. Thus, it is vital to take a closer look at how prior exposure to bispecific antibodies influences CAR-T cell manufacturing and function. The following section offers an examination of in vitro assessments regarding this interplay, to elucidate the potential effects and thus, enhance therapeutic strategies in multiple myeloma treatment.

### Example 3: Effect of Prior Exposure of Bispecific Antibodies to CAR-T Cell Manufacturing and Function

Target cell-mediated T cell bispecific activation influences T cell fitness. As an explanation of this effect, it was hypothesized that extended activation could potentially lead to alterations in T cell fitness within the apheresis material, resulting in sub-optimum CAR-T manufacturing and compromised efficacy. To study this, the effects of prior exposure to T cell redirectors, or bispecific antibodies (BsAbs) on CAR-T manufacturing and functionality were examined through in vitro assessments.

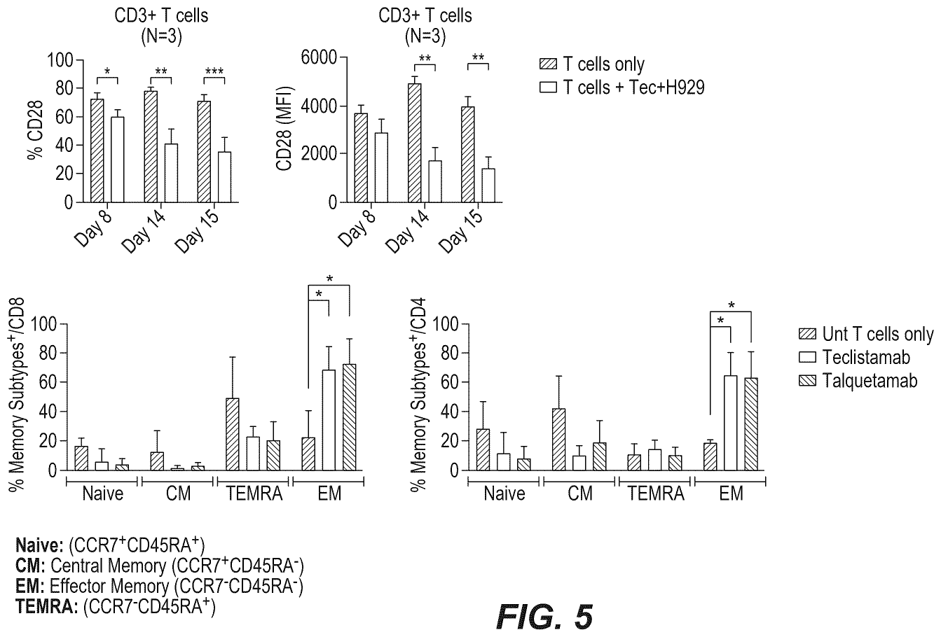

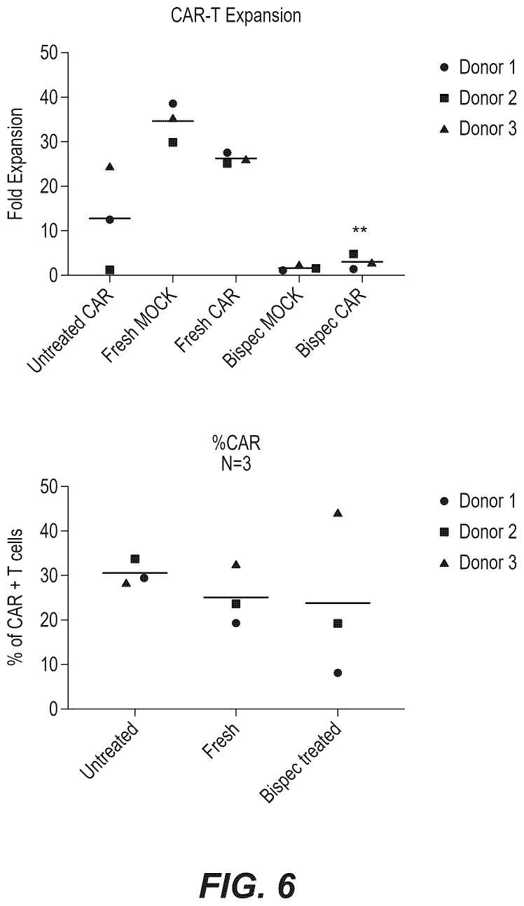

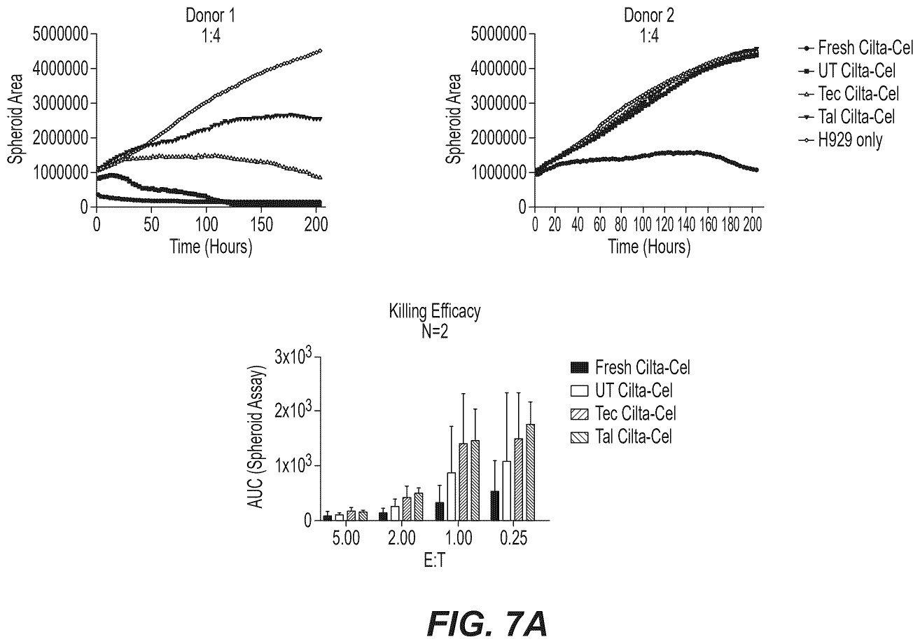

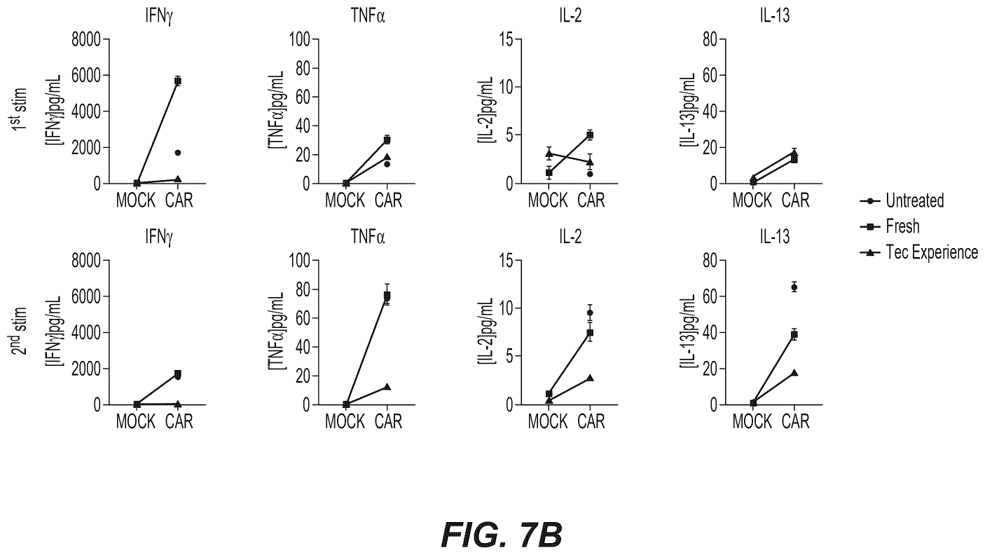

An experiment was performed to assess the impact of prior exposure to T cell redirectors on the manufacturing and functionality of CAR-T cells. T cells from multiple myeloma patients were exposed to bispecific T cell engagers, either talquetamab or teclistamab, for 12 days in vitro. Results depicted in FIG. 5 indicate that T cells pre-exposed to bispecifics saw a decrease in CD28 cells, while also leading to an increase in the proportion of effector memory population. These T cells were subsequently processed through ciltacabtagene autoleucell manufacturing. Results depicted in FIG. 6 indicate that such pre-exposure also negatively impacted CAR-T production, reducing the amount of CAR-T drug product that could be manufactured, subsequently decreasing the level of CAR positivity within said drug product. Finally, the resulting CAR-T cells were subjected to functional analysis to evaluate their effectiveness. FIGS. 7A-7B depict that CAR-T cells derived from these pre-exposed cells yielded reduced cytotoxicity and cytokine production when compared to control groups.

Collectively, these results indicate that T cells with prior exposure to teclistamab/talquetamab bispecifics show decreased CD28 expression as well as an enriched fraction of Effector memory cells. This means that teclistamab or talquetamab pretreatment decreases CAR-T manufacturing efficacy. Further, the noted reduction in cytokine production and killing efficacy, as well as the reduced CAR+ expansion indicates that CAR-T made from teclistamab and/or talquetamab pre-treated T cells exhibit decreased functionality. Overall, treatment with bispecifics before ciltacabtagene autoleucel may hinder T cell fitness, reduce CAR-T production, and limit CAR-T functionality. Thus, these results indicate that it may be best to perform apheresis before administering bispecific treatment to achieve optimal CAR-T production and functionality.

### Example 4—CARTITUDE-2 Cohort C: T Cell Fitness in Patients with Prior Exposure to BCMA Targeted Therapy

The CARTITUDE-2 study (MMY2003; NCT04133636), specifically with respect to Cohort C, focused on T cell fitness in patients who had previous exposure to BCMA targeted therapy. Patients were monitored for a median duration of 18 months, ranging from 0.6 to 22.7 months. Among these, 13 patients had previously undergone BCMA antibody-drug conjugate (ADC) therapy and seven had undergone bispecific antibody (BsAb) therapy. One patient from the ADC group had prior exposure to BsAb therapy. Approximately 3% of the patients (comprising six individuals) had anti-BCMA therapy as their last line of therapy (LOT), which included four patients from the ADC group and two from the BsAb group.

Baseline patient characteristics were first assessed: the median age was 62.5 years (range 44-81 years); 15% had high-risk cytogenetics (all del17p); 25% had extramedullary disease; patients had previously received a median of 8 LOTs (ranging between 4 and 13); and 90% were diagnosed as anti-BCMA refractory. The median time from the last anti-BCMA treatment to ciltacabtagene autoleucel infusion was 180 days (range, 62-749 days) for ADC-exposed patients and 227 days (range, 84-329 days) for BsAb-exposed patients. The median ciltacabtagene autoleucel dose that was administered was 0.61×10\hat{ }6 CAR viable T cells/kg (range between 0.21×10\hat{ }6-0.83×10\hat{ }6). Notably, one patient received a dose below the target level. Patients treated with BCMA ADC and BsAbs demonstrated comparable baseline demographics and disease characteristics.

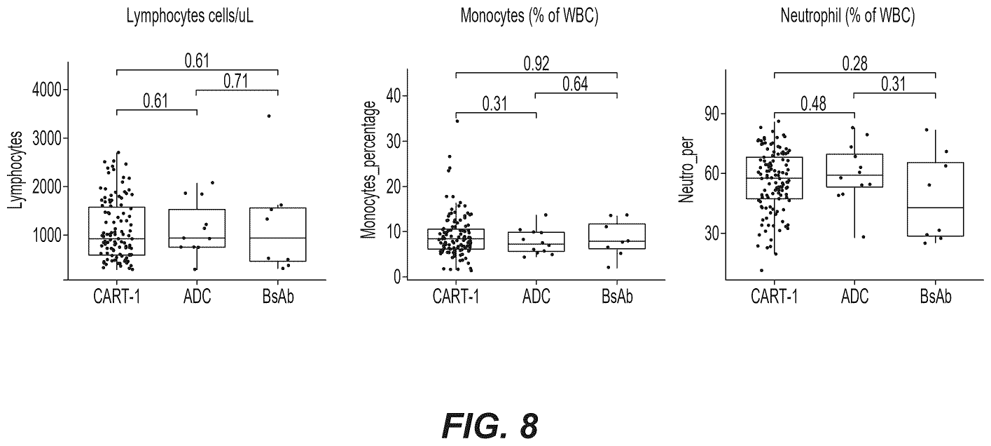

The data from MMY2003 (cohort C) was compared against data from patients enrolled in the CARTITUDE-1 (MMY2001; NCT03548207) study. The levels of lymphocytes, monocytes, and neutrophils in peripheral blood were found to be comparable between the ADC and BsAb groups, as indicated in the MMY2001 and MMY2003 (Cohort C) studies (FIG. 8). Patients with prior BsAb exposure displayed a lower CD4:CD8 ratio at apheresis. These patients also exhibited lower CD4+ T cell count in the peripheral blood, while CD8+ T cell counts remained similar to patients without BsAb exposure. Patients from the MMY2001 study showed similar CD4 and CD8 counts as compared to the ADC group. The observed decrease in CD4+ cells and the CD4:CD8 ratio points toward potentially suboptimal T cell fitness. No significant difference was observed in CD25+ or CD28+ T cells levels or T cell memory subsets between ADC and BsAb groups.

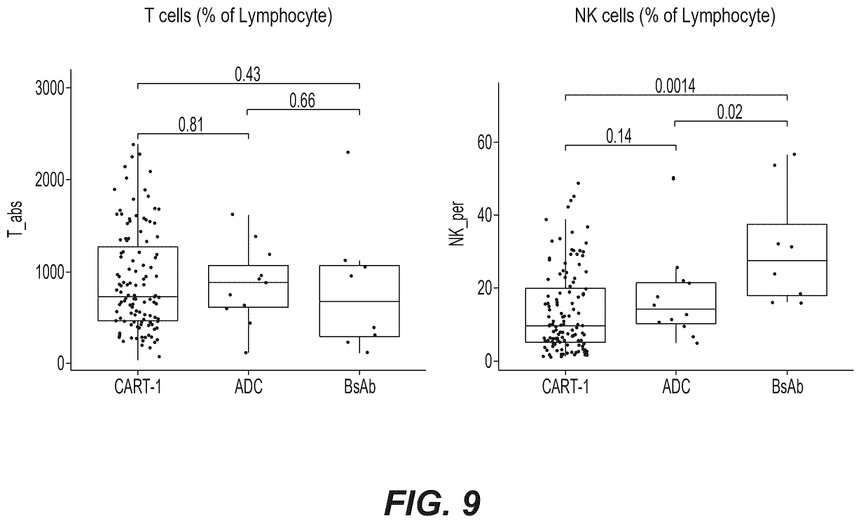

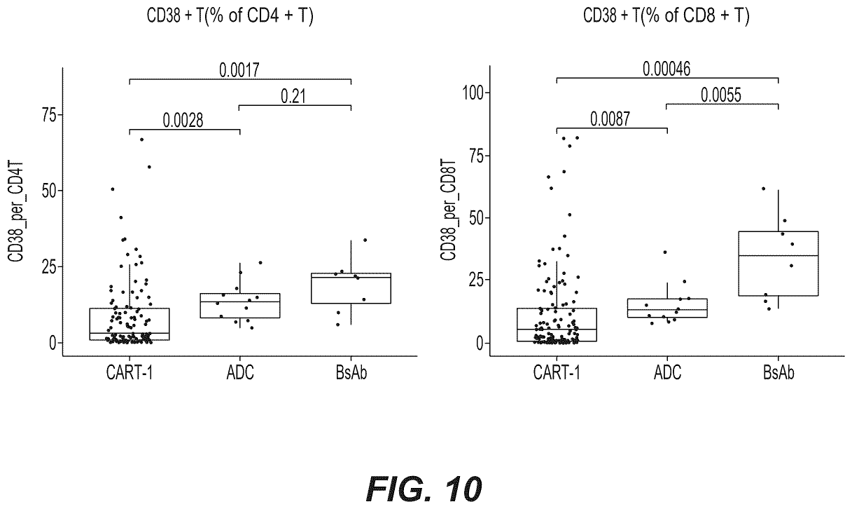

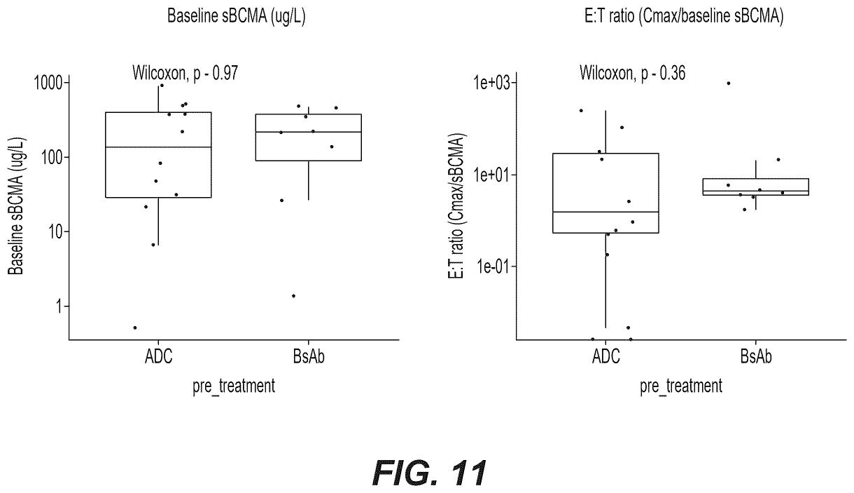

Patients with prior BsAb exposure had higher levels of circulating natural killer (NK) compared to the ADC and MMY2001 groups, while showing a comparable T cell count (FIG. 9). The expression of CD38 in CD4+/CD8+ T cells was shown to be associated with a decrease in CD4+ T cells in cellular immunity; this expression was found to negatively correlate with treatment-free survival in male chronic lymphocytic leukemia (CLL) patients (FIG. 10). These results align with the observed increase in CD38+ T cells following teclistamab treatment (MajesTEC-1 study). Deeksha et al., ASH 2003. While the ADC group also showed elevated CD38+ T cells, it remains unclear whether this effect is a consequence of BCMA targeting therapies due to a lack of specific reports on the issue. Finally, there was no substantial difference in baseline tumor burden and effector-to-target (E:T) ratio between the ADC and BsAb groups, according to (FIG. 11).

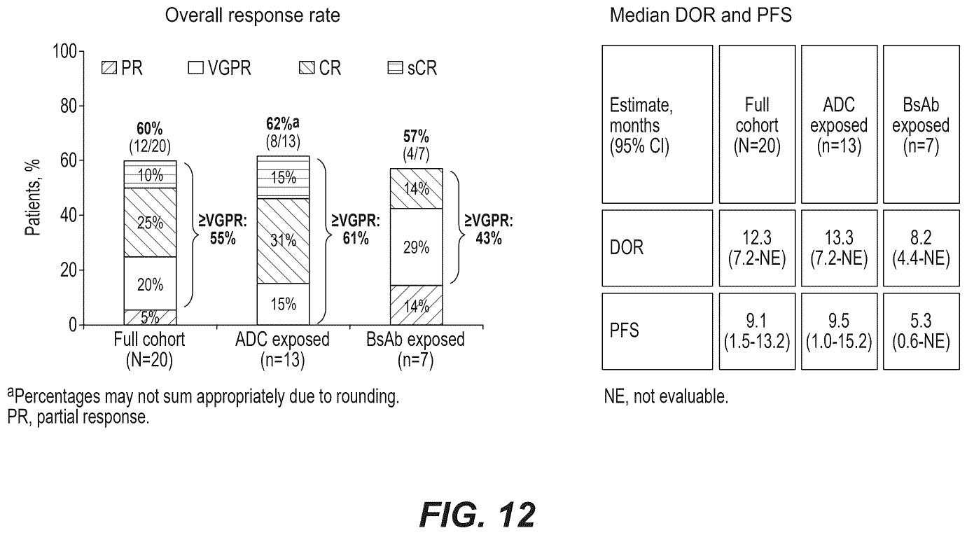

To summarize, patients previously exposed to BsAb exhibited higher levels of NK cells, lower levels of CD4+ T cells, and a lower CD4:CD8 ratio, potentially pointing towards suboptimal T cell fitness. No difference was found in lymphocytes, monocytes, and neutrophils in peripheral blood between patients with prior exposure to targeted ADC vs. BsAb or none of the two before ciltacabtagene autoleucel administration. Furthermore, patients previously exposed to BsAb showed similar levels of CD25+, CD28+ T cells, and T cell memory subsets but higher levels of CD38+ T cells compared to those in the ADC group. Comparing ADC and BsAb groups, no significant difference was found in baseline tumor burden (sBCMA) or E:T ratio (Cmax/sBCMA), and no substantial difference was detected in baseline pro-inflammatory cytokines. Generally, the results, as depicted in FIG. 12, demonstrated that the efficacy of ciltacabtagene autoleucel was compromised in patients previously treated with BCMA bispecific antibodies (BsAb), compared to those exposed to BCMA antibody-drug conjugates (ADC) therapies.

Overall, efficacy of ciltacabtagene autoleucel in patients with prior BCMA BsAb is compromised compared to prior BCMA ADC therapies or no BCMA directed therapy. These results support the preclinical data presented in Example 4 and further suggest that clinical efficacy may be compromised in ciltacabtagene autoleucel patients previously treated with bispecifics.

### Example 5: Preclinical Data Supporting Combinations of Ciltacabtagene Autoleucel with Talquetamab

Preclinical research was conducted to study the effects of administering teclistamab or talquetamab simultaneously with or after the administration of ciltacabtagene autoleucel, assessing whether their application in this order augments or diminishes the therapeutic efficacy of the individual treatments. Methodologically, the study involved conducting cytotoxicity assays and cytokine profiling on ciltacabtagene autoleucel, both in isolation and in conjunction with teclistamab and talquetamab. The primary aim of these assays was to evaluate the relative efficacy of the different treatment combinations and to ascertain the survival rates of T cells under varying conditions. These approaches aimed to help elucidate the therapeutic viability of the combination, specifically focused on addressing treatment scenarios envisioned in the MonumenTAL-8 study design, as presented in Example 6, below.

### Suboptimal Quantity of CAR-T can Medicate Enhanced Killing Via Bispecific Engagement

First, an assay was designed to utilize Incucyte-based spheroid formation to provide real-time kinetic data on the cytotoxic effects of CAR-T cell therapy. This assay began on Day 0 with the seeding of Incucyte® NucLight Green H929 cells into uncoated round-bottom wells. During the initial three days, these cells proliferated and coalesced to form spheroid structures. On Day 3, bispecific antibodies were introduced into the wells. This was followed by the addition of T cells, which were genetically modified to express CARs, enabling the targeted destruction of the cancer cell spheroids. MOCK cells were used as a comparator and as effectors in the bispecific killing assay; these cells were activated and expanded according to the manufacturing protocol with the exception that lentiviral transduction was not performed.

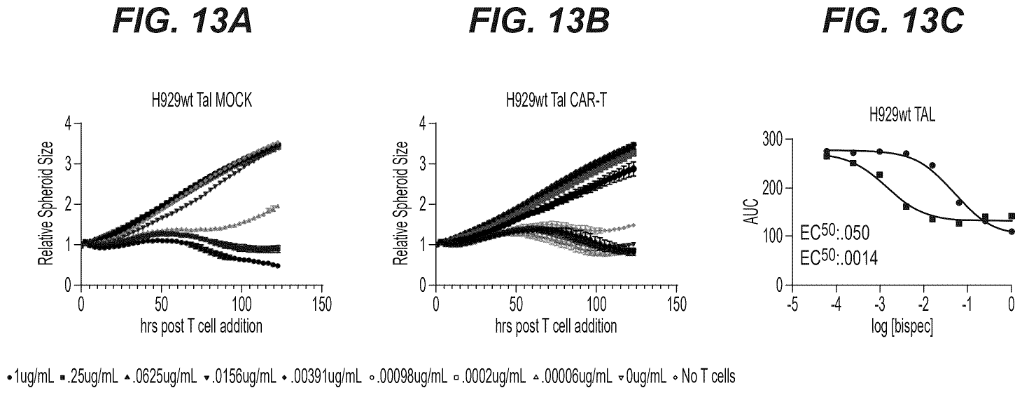

Comparisons were then made between untreated spheroids and those treated with the combination of CAR-T cells and bispecific antibodies. FIGS. 13A-13B depict the change in relative spheroid size over time following T cell addition, at varying concentrations of talquetamab, with FIG. 13A comparing the responses of MOCK cells and FIG. 13B comparing the responses of CAR-T cells. FIG. 13C depicts an Area Under the Curve (AUC) analysis, depicting the responses of MOCK cells against CAR-T cells over a log-transformed range of bispecific antibody concentrations. The findings indicate a marked difference in the potency of bispecific engagement in the presence of CAR-T cells (EC50=0.0014) as compared to MOCK cells (EC50=0.050), underscoring an enhanced effect of the bispecific antibodies when used in tandem with CAR-T cells.

Overall, these results show that even relatively low quantities of CAR-T cells can significantly bolster the killing of cancer cells when combined with bispecific antibodies. Further, coculturing ciltacabtagene autoleucel with teclistamab or talquetamab results in greater in vitro killing capacity, such that maximal killing can be achieved via a combination of doses that provide no monotherapeutic efficacy. This observation implies a synergistic interaction between these therapeutic agents, potentially enabling a greater efficacy of CAR-T cell therapy. Similar results were observed, though not depicted in the figures, with teclistamab.

Next, experiments were performed to determine the minimum threshold of CAR positivity required for achieving the desired therapeutic effect in CAR-T cell treatments. Flow cytometry was performed to differentiate between various T cell populations: MOCK cells, double positive (DP) T cells expressing both CD4 and CD8, and T cells enriched used biotinylated BCMA and anti-biotin magnetic beads.

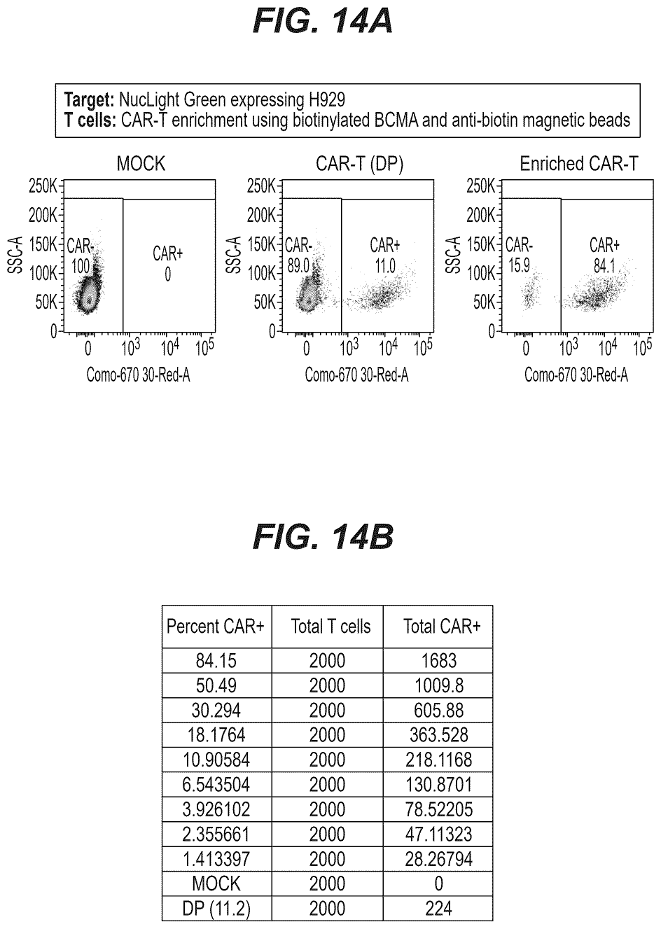

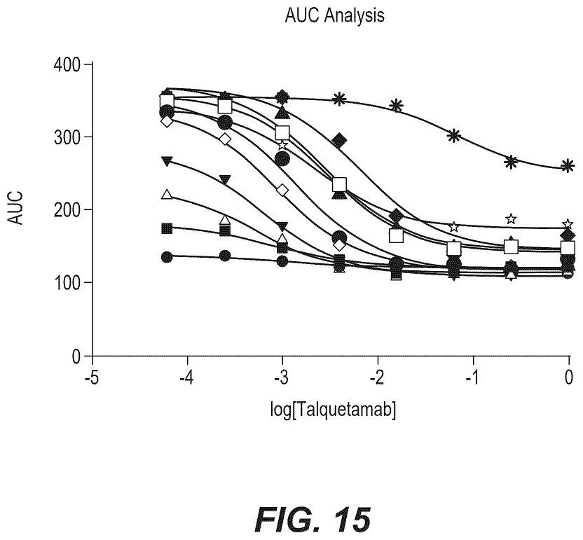

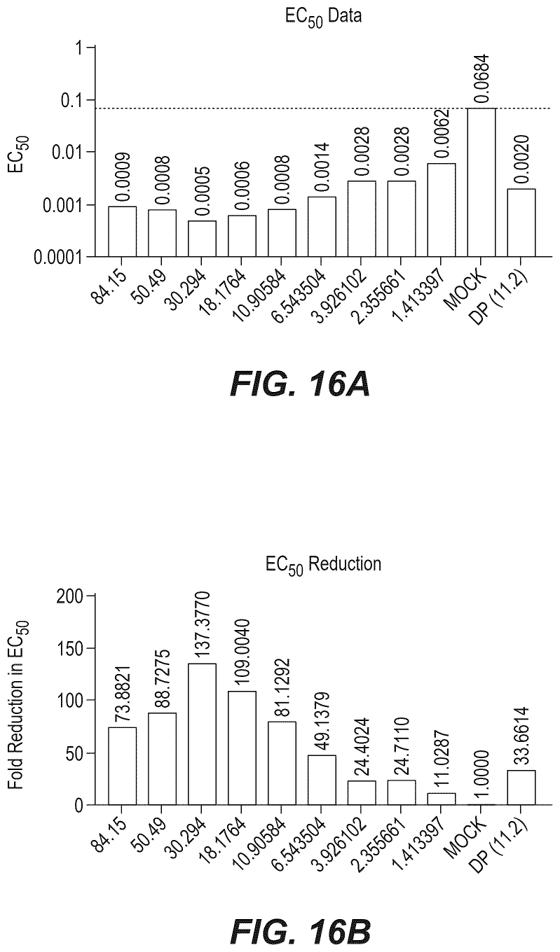

FIG. 14A depicts flow cytometry plots, illustrating the proportion of CAR-positive cells in relation to the total T cell population. FIG. 14B presents the percentage of CAR-positive cells and the absolute numbers of both T cells and CAR-positive T cells at different dilution levels. FIG. 15 depicts the relationship between the log concentration of talquetamab and the AUC, which is indicative of the overall effect of the drug over time. Each curve on the graph corresponds to a different dilution of CAR positive T cells, showing that as the proportion of CAR-positive T cells decreases, the AUC generally decreases, indicating a lesser effect. FIG. 16A compares the half maximal effective concentration (EC50) values for each dilution level of CAR-positive T cells while FIG. 16B compares the fold reduction in EC50 for each dilution level. The results indicate that although the magnitude of the cytotoxic effect decreases with lower levels of CAR-positive T cells, even as few as 1% of remaining CAR-positive cells can significantly enhance the killing effect when engaged by talquetamab, further suggesting that talquetamab retain substantial therapeutic efficacy across a range of CAR-T cell concentrations. Overall, the results indicate that even a diluted presence of CAR-positive T cells within a population of non-modified cells can amplify the cytotoxic effect when engaged by bispecific antibodies. This supports the emerging understanding that bispecific antibodies can lower the threshold of CAR-T cells needed to observe a therapeutic effect.

### Activation of CAR− Cells in the Tumor Microenvironment

CAR-T cell therapy has been shown to exert a wider therapeutic impact beyond their direct cytolytic activity by recruiting and activating a diverse array of immune effector cells. Evidence from post-infusion biopsies of diffuse large B cell lymphoma (DLBCL) patients treated with axicabtagene ciloleucel (YESCARTA®), a CAR-T cell therapy, supports the hypothesis that CAR-T cells, upon engagement with the target antigen on DLBCL cells, can trigger an activation cascade. JCI Insight, June 18, 5(12), 3134612. Latent T cells recognize the CD19 antigen on DLBCL cells, leading to their transformation into CAR-T cells. Once activated, these CAR-T cell release interferon gamma (IFN-γ). This release activates macrophages and induces activation of non-CAR T cells, which subsequently exhibit markers such as PD1, Ki67, and Granzyme B (GsmB), signaling their readiness to participate in the immune response. In sum, this upregulation implies that CAR-cell cell infusion not only directly targets cancer cells but also broadly primes the immune system, potentially enhancing the overall antitumor response.

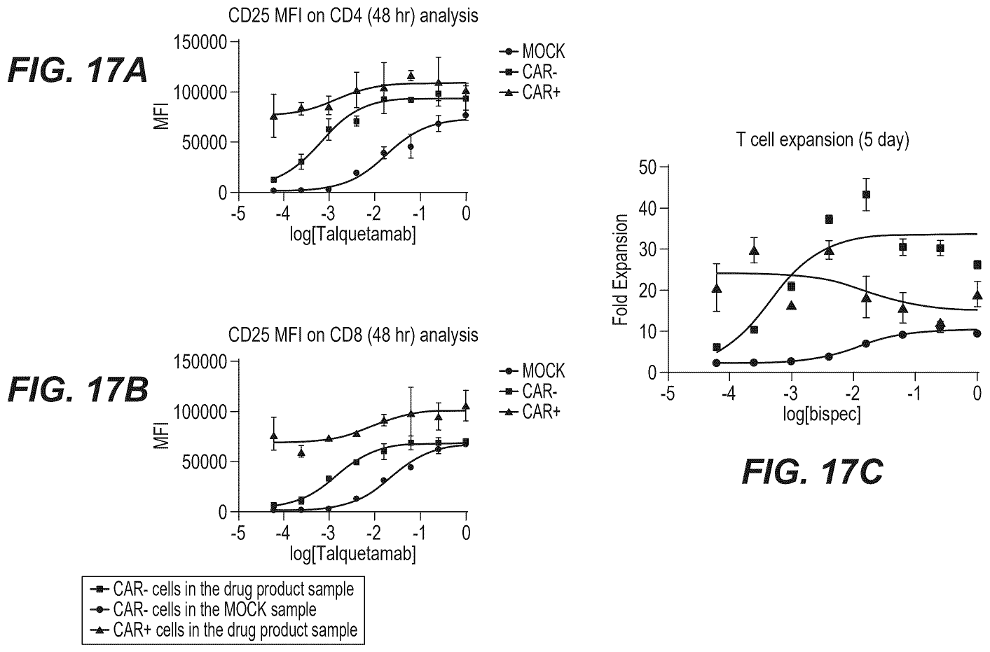

Experiments were conducted to evaluate the activation and proliferation of CAR-negative (CAR−) T cells in the presence of CAR-positive (CAR+) T cells and the impact of talquetamab on the process. FIGS. 17A-17B provide an analysis of CD25 Mean Fluorescence Intensity (MFI) on both CD4 and CD8 T cells after 48 hours, in relation to the log concentration of talquetamab. CD25 is a subunit of the IL-2 receptor, and its upregulation is a marker of T cell activation. The graphs depict three curves each, representing MOCK cells (T cells without CAR construct), CAR− cells (T cells without the CAR construct in the drug product sample), and CAR+ cells (T cells with the CAR construct in the drug product sample). The MFI levels of CD25 increase with the concentration of talquetamab, indicating heightened T cell activation. FIG. 17C depicts T cell expansion over five days, again in relation to the log concentration of talquetamab. These results indicate that the presence of talquetamab facilitates the early activation and expansion of CAR− T cells. This finding is significant as it indicates a bispecific antibody-mediated cross-talk between CAR+ and CAR− T cells, leading to an amplified immune response against the tumor cells. Moreover, while both CAR− and CAR+ cells expand, the presence of the bispecific antibody appears necessary for the expansion of CAR− cells in a dose-dependent manner, suggesting that talquetamab may play a crucial role in the broader activation of the immune system, beyond the direct effects of CAR-T cell therapy.

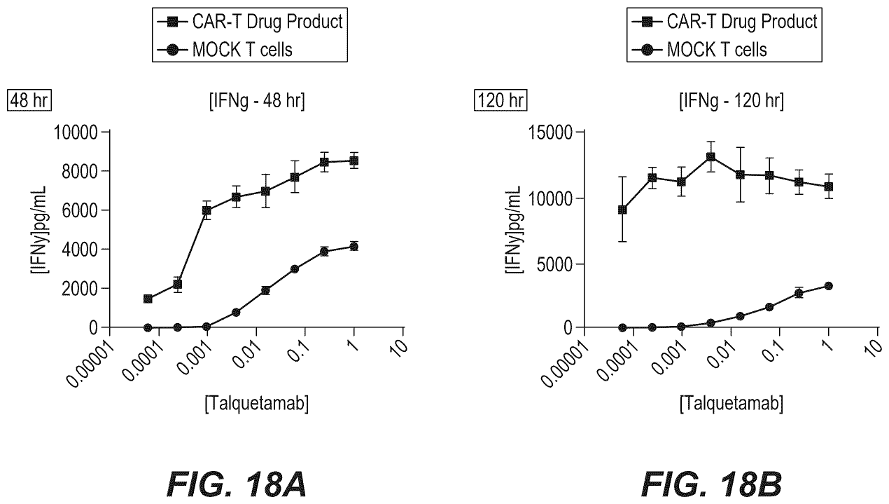

Experiments were conducted to determine the effect of bispecific antibodies on the expression of inflammatory cytokines. FIGS. 18A-18B depict the concentration of interferon-gamma (IFN-γ), a mediator in the immune response against tumors, against the logarithmic concentration of talquetamab. FIG. 18A, representing the 48-hour time point, demonstrates a clear dose-response relationship, with increasing talquetamab levels correlating to a higher expression of IFN-γ, notably more pronounced in the sample containing the CAR-T drug product. At the 120-hour mark, as depicted in FIG. 18B, the IFN-γ levels remained elevated in the presence of talquetamab, with the CAR-T drug product continuing to exhibit a higher cytokine expression compared to the MOCK T cells. These results indicate the potential of bispecific antibodies to enhance cytokine responses beyond the levels typically observed with single-agent therapy, suggesting that bispecific antibodies like talquetamab not only engage tumor cells directly but also amplify the release of cytokines, further stimulating the immune system. Additional results not depicted indicate that similar trends were observed with other cytokines, such as IL-2, TNF-α, and IL-13, pointing to a more extensive influence of bispecific antibody treatment on the cytokine environment within the tumor microenvironment.

There exist two primary pathways by which BCMA CAR-T cells mediate the destruction of cancer cells: 1) directing targeting, or redirected lysis of Tumor-Associated Antigen (TAA) positive target cells and 2) indirect effects, or bystander lysis of TAA-negative cells. Sec pLoS One. 2017; 12(8): e1083390. Concerning the direct targeting pathway, CAR-T cells, engineered to target specific Tumor-Associated Antigens (TAA) on the surface of cancer cells, bind to these antigens via their CARs. Upon engagement, the CAR-T cells become activated and release cytotoxic molecules, such as IFN-γ and TNF-α. These molecules induce apoptosis in the target cells. Furthermore, the interaction between the CAR-T cell and the cancer cell upregulates certain molecules on the T cell surface, specifically the Fas ligand, which is involved in the strengthening the cell-to-cell interaction and enhancing the cytotoxic response. CAR-T cells can also indirectly cause the death of neighboring cancer cells that do not express the targeted antigen (TAA-negative). This occurs through the interaction of adhesion molecules, specifically ICAM-1 on the cancer cells and LFA-1 on the T cells, and the Fas/Fas Ligand pathway, which can trigger pseudo-synapse, referring to the formation of an immune synapse-like structure that facilitates cell killing without the TAA being present on the bystander cell.

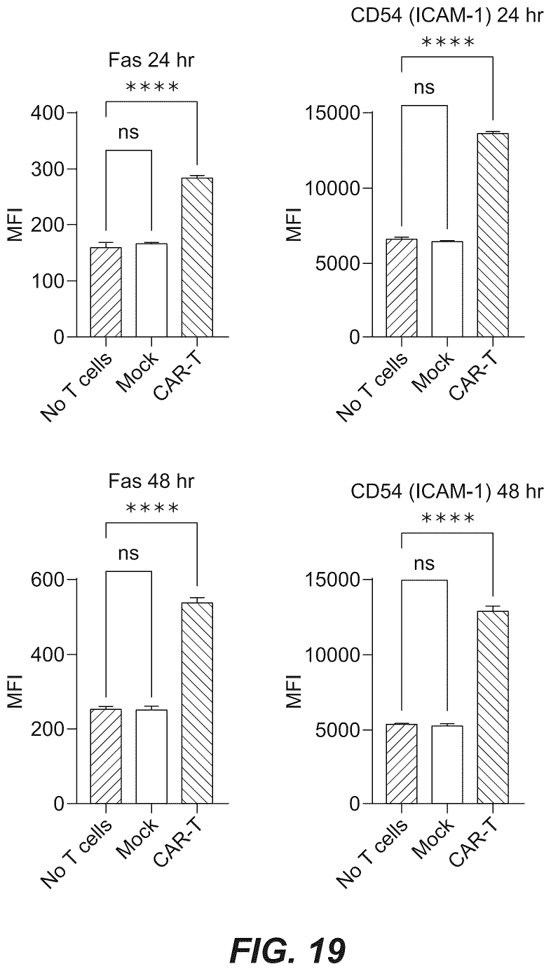

In vitro experiments were conducted to study the impact of a suboptimal dose of BCMA CAR-T cells on the expression of cell adhesion molecules and cell death receptors in H929 cells. Incucyte® NucLight Green H929 cells were plated in an uncoated round-bottom well until they formed into a spheroid structure on Day 3, at which time T cells were added to the culture. FIG. 19 depicts expression levels of two specific markers at 24- and 48-hours post T cell addition: the Fas ligand and the CD54 molecule (also known as ICAM-1), an intercellular adhesion molecule. The graphs indicate that both Fas and CD54 expression levels were significantly higher in the presence of CAR-T cells compared to the MOCK and no treatment controls. These results indicate that the presence of CAR-T cells, even at suboptimal doses, leads to the upregulation of markers associated with cell susceptibility to killing and bystander T cell killing. This suggests that suboptimal doses of CAR-T cells can alter the tumor microenvironment in a manner that increases the susceptibility of cancer cells to immune-mediated killing, thereby potentially enhancing the overall efficacy of the therapy.

### SUMMARY

To summarize, these results indicate that both teclistamab and talquetamab have demonstrated an ability to enhance the cytotoxicity of the CAR-T cell drug product. The level of CAR-positive T cell necessary for effective monotherapy was found to be significantly lower than the threshold required to see notable increase in cytokine levels. This implies that even low concentrations of CAR+ cells can exert a substantial therapeutic impact. No notable adverse or beneficial effects were observed on the number or activation status of CAR-positive cells when combined with the bispecific antibodies. Additionally, the presence of CAR-positive cells in the culture environment was shown to increase the vulnerability of tumor cells to killing by CAR-negative cells, suggesting a synergistic interaction. Finally, an increase in the activation of CAR-T cells and the upregulation of cell adhesion molecules like ICAM-1 and death receptors, such as Fas, on target cells were observed. Overall, bispecific antibodies demonstrate a potential to amplify the cytokine response beyond that typically seen with single-agent therapy, indicating that a combination of these agents with CAR-T cells could offer a more dynamic and potent therapeutic approach against multiple myeloma. Overall, these results indicate that bispecifics may synergistically enhance tumor cell killing, outpacing either monotherapy. Combining these modalities in patients with high disease burden and suboptimal drug product attributes could further enhance curative potential.

### Example 6: Combination Approach Targeting Two Highly Expressed Surface Proteins in Multiple Myeloma

High-risk MM represents a group of patients with a significant unmet medical need both in front line and at the relapsed/refractory setting. There is no designated systemic therapy for these patients, and resistance to therapies is not uncommon. Further, participants who have been treated with multiple prior lines of therapy likely have limited effective treatment options. Therefore, new therapeutic options directed at alternative mechanisms of action that can better control the disease and provide deeper, more sustained responses are urgently needed.

Talquetamab monotherapy has previously demonstrated significant efficacy in patients with heavily pretreated relapsed/refractory multiple myeloma (RRMM) in Study 64407564MMY1001 (hereafter MonumenTAL-1).

Ciltacabtagene autoleucel is also highly efficacious in heavily pretreated RRMM. The CARTITUDE-1 study, a single arm Phase 1b/2 study of ciltacabtagene autoleucel in participants with RRMM who had received a median of 6 prior lines of therapy, demonstrated significant antimyeloma activity and a safety profile consistent with the known mechanism of action of CAR-T therapies (Berdeja 2001). The phase 2 CARTITUDE-2 study showed efficacy of ciltacabtagene autoleucel in small cohorts at earlier disease stages, with response rates of 95%-100%, and median duration of response (DOR) and median progression free survival (PFS) not reached after ˜1.5-year follow-up (van de Donk et al., Blood 2022; 140:7536-7; Einsele et al., American Society of Clinical Oncology Annual Meeting; 2022 Jun. 3-7; Chicago, IL).

Additionally, preliminary data from Study 68284628MMY2003 supported the use of ciltacabtagene autoleucel in patients with newly developed multiple myeloma (NDMM). Overall, the safety profile of ciltacabtagene autoleucel in this study was similar to that of ciltacabtagene autoleucel in prior and ongoing studies, without new safety signals, and thus ciltacabtagene autoleucel administration presents a promising benefit-risk profile in a NDMM population.

Nevertheless, the occurrence of relapse due to heterogenous antigen expression on malignant PCs remains a challenge when only a single antigen is targeted. Additionally, outcomes for patients with high-risk disease are inferior compared with those with standard risk.

Notably, the data from MonumenTAL-1 show that prior exposure to CAR-T cell therapy did not limit the efficacy of talquetamab and that the safety profile was comparable in participants with or without prior T-cell redirection therapy. Further, Study 64007957MMY1003 (hereafter RedirecTT-1), a Phase 1b study of talquetamab in combination with an anti-BCMA bispecific antibody teclistamab demonstrated unprecedented efficacy in a subset of patients with extramedullary soft-tissue plasmacytoma (STP).

Taken together, these data support the hypothesis that targeting two distinct antigens on PCs may overcome some of the common mechanisms of resistance to monotherapy by reducing the risk of loss of activity due to single target antigen loss and enhancing antigen-antibody interaction. Further, as the efficacy of both CAR-T therapy and bispecific therapy are dependent on a patient's immune response, the combination of ciltacabtagene autoleucel and talquetamab also has the potential to be more efficacious and produce more durable responses in the frontline MM setting when administered to patients with NDMM who have limited exposure to immunomodulatory and cytotoxic therapy.

### Study Overview

MonumenTAL-8 is a phase 2, open-label, multicenter, multi-cohort study of ciltacabtagene autoleucel (cilta-cel) and talquetamab in adult participants with high-risk multiple myeloma (MM). More specifically, MonumenTAL-8 is a phase 2, open-label, multicenter, multi-cohort study of ciltacabtagene autoleucel and talquetamab in adult patients with relapsed and/or refractory MM whose disease has progressed after at least 3 prior lines of therapy and in adult participants with high-risk newly developed MM.

A primary objective of the study is to evaluate the safety of the sequential use of talquetamab and ciltacabtagene autoleucel in this population due to targeting multiple tumor surface antigens (GPRC5D and BCMA) compared with either compound alone. Secondary objectives of the study are to evaluate the preliminary efficacy of ciltacabtagene autoleucel and talquetamab and to characterize minimal residual disease (MRD) negativity.

### Participants

Screening for eligible participants will be performed within 28 days of apheresis according to the Schedule of Activities (SoA), as outlined in Tables 10-17. The inclusion and exclusion criteria for enrolling participants in this study are described in Table 18 and Table 19, respectively. A participant is considered eligible if the last observation before administration of the study treatment satisfied the inclusion and exclusion criteria. If a participant's clinical status changed (including any available laboratory results or receipt of additional medical records) after screening but before apheresis such that the participant no longer met all eligibility criteria, then the participant will be excluded from participation in the study.

Participants were assigned to cohorts in a non-randomized manner (Cohort 1 [RRMM] and Cohort 2 [NDMM], Cohort 3 [RRMM]). At least 10 participants will be enrolled into each cohort. Participants participated in one of the following cohorts based on eligibility criteria defined in Table 18 and Table 19. Cohort 1 and Cohort 2 will be enrolled concurrently, and the enrollment of Cohort 3 commenced only after the completion of Cohort 1 enrollment.

### Treatment: Overview

To acquire peripheral blood mononuclear cells (PBMCs), participants across all cohorts will undergo apheresis 7 to 14 days after enrollment and prior to receiving any study treatment. If apheresis is not feasible at this point in time, the sponsor will be consulted to determine a new timing for the apheresis. If apheresis could not be performed 7 to 14 days after enrollment, a shorter timeframe can be acceptable. Ciltacabtagene autoleucel will then be generated from the participant's T-cells selected from the apheresis product. Participants for whom apheresis or manufacturing failed will be allowed a second attempt at apheresis.

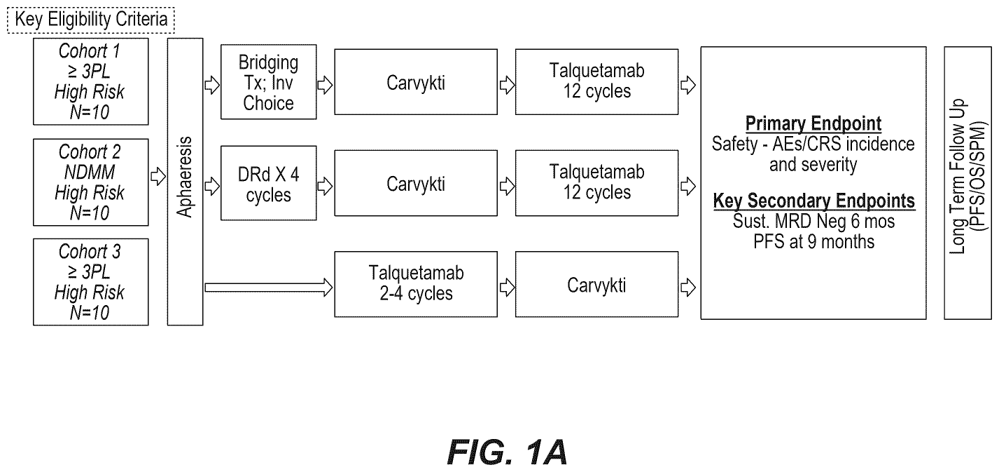

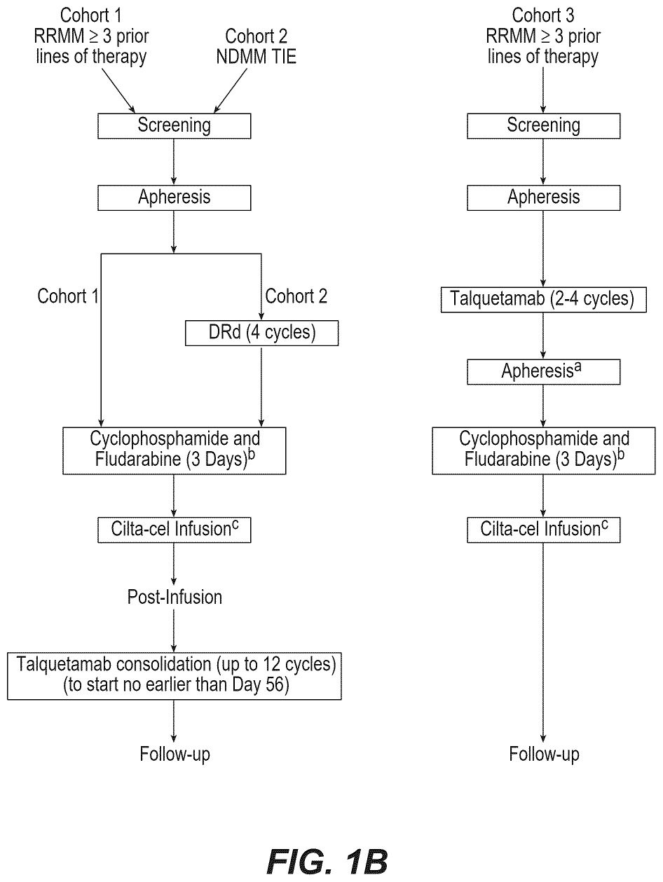

Treatment will be administered in 28-day cycles and according to the dosing administration schedule as summarized below and as detailed in FIGS. 1A-1B and Table 20A-20B, and according to dose modifications permitted for toxicity.

All participants will receive the currently approved talquetamab subcutaneous (SC) monotherapy dose schedule consisting of 3 step-up doses, 0.01 mg/kg, 0.06 mg/kg, and 0.4 mg/kg. All cohorts then receive 0.8 mg/kg Talquetamab SC every 2 weeks (Q2W).

Prior to ciltacabtagene autoleucel infusion, all participants will receive a conditioning regimen of IV cyclophosphamide 300 mg/m² and fludarabine 30 mg/m² daily for 3 days; sponsor approval will be obtained to modify the conditioning regimen schedule or dose. The conditioning regimen is intended to lead to lymphodepletion and to help promote CAR-T cell expansion in the participants. Participants with severe renal impairment (i.e., CrCl 10 to 24 mL/min), will be monitored closely for signs and symptoms of cyclophosphamide toxicity, including hemorrhagic cystitis, pyelitis, urethritis, and hematuria. The dose of fludarabine will be reduced to 24 mg/m² for participants with an eGFR of 30 to 70 mL/min/1.73 m². Additionally, prior to ciltacabtagene autoleucel infusion, participants will receive premedication as noted in Table 21. Corticosteroids will not be used as pre-infusion medication.

Ciltacabtagene autoleucel will be administered to all participants as a single infusion 5 to 7 days after the start of the conditioning regimen (the first day of conditioning is Day-7 to Day-5, and the day of ciltacabtagene autoleucel infusion is Day 1). Ciltacabtagene autoleucel will be administered as described in the Cell Therapy Product Procedures Manual (CTPPM) and Investigational Product Preparation Instructions (IPPI).

Ciltacabtagene autoleucel will be administered at a targeted infused dose of 0.75×10⁶ CAR-positive viable T cells/kg (range: 0.5-1.0×10⁶ CAR-positive viable T cells/kg, maximum dose: 1.0×10⁸ CAR-positive viable T-cells). This dose was aligned with the label and was established from data from the CARTITUDE-1 study.

### Treatment: Cohorts 1 and 2 (Ciltacabtagene Autoleucel+Tal Consolidation Post CAR-T Therapy)

Following apheresis, Cohort 1 participants will receive a conditioning regimen of cyclophosphamide and fludarabine daily for 3 days. Ciltacabtagene autoleucel will be administered 5 to 7 days after the start of the conditioning regiment.

Participants in Cohort 1 (RRMM) with highly aggressive MM will also be allow to receive up to 4 cycles of investigator-selected bridging therapy between apheresis and the administration of ciltacabtagene autoleucel when clinically indicated to maintain disease stability while waiting for manufacturing of ciltacabtagene autoleucel. This bridging therapy is required to be a short-term treatment that previously generated at least stable disease for the participant. Participants who progressed during bridging therapy or prior to ciltacabtagene autoleucel infusion will be allowed to receive treatment with ciltacabtagene autoleucel but will be considered non-evaluable for response.

Participants in Cohort 2 (NDMM) first receive 4 cycles of daratumumab, lenalidomide and dexamethasone (DRd) induction therapy after apheresis. The chemotherapy induction regimen (daratumumab 1,800 mg SC weekly for 2 cycles followed by Q2W for the next 2 cycles, lenalidomide 25 mg oral for 21 days each cycle and dexamethasone weekly in Cycles 1 and 2 and Q2W in Cycles 3 and 4) used in this study is considered standard in this setting in previously untreated patients with MM who are ineligible for hematopoietic stem cell transplant. Daratumumab, lenalidomide and dexamethasone were administered as summarized in Tables 22A-22B and Table 23.

Prior to ciltacabtagene autoleucel infusion, Cohort 2 participants will receive a conditioning regimen of cyclophosphamide and fludarabine daily for 3 days. Ciltacabtagene autoleucel will be administered 5 to 7 days after the start of the conditioning regiment.

Participants in Cohorts 1 and 2 who had confirmed progressive disease (PD) prior to ciltacabtagene autoleucel administration will be permitted to receive ciltacabtagene autoleucel if requested by the investigator and agreed with the sponsor but will be considered non-evaluable for response.

For Cohorts 1 and 2, participants will receive up to 12 cycles of talquetamab consolidation treatment ≥56 days after ciltacabtagene autoleucel infusion. Participants in Cohorts 1 and 2 who experienced symptoms of worsening disease but who do not meet PD criteria after 12 cycles could be re-treated with talquetamab upon consultation with the sponsor. Dosing for talquetamab will proceed in every 4 weeks (Q4W) intervals from Cycle 7 onward (and as early as Cycle 5 for participants in confirmed very good partial response [VGPR] or better) in order to decrease exposure while maintaining efficacy and improving convenience for participants.

### Treatment: Cohort 3 (Tal Bridging Therapy Pre-CAR-T Therapy+Ciltacabtagene Autoleucel)

Cohort 3 will begin enrollment after the tenth participant in Cohort 1 receive their ciltacabtagene autoleucel infusion. Following apheresis, participants in Cohort 3 (RRMM) will receive up to 4 cycles of talquetamab bridging therapy to maintain disease stability while waiting for manufacturing of ciltacabtagene autoleucel; of these, the first 5 participants enrolled will also undergo a second apheresis after bridging to determine the viability of T cells collected after talquetamab treatment. Participants who progress during talquetamab bridging therapy or prior to ciltacabtagene autoleucel infusion will be allowed to receive treatment with ciltacabtagene autoleucel but are considered non-evaluable for response and did not receive a second apheresis (if applicable); additionally, they will be replaced to complete the cohort of evaluable participants.

### Treatment: Dose Modification

Toxicities will be attributed, whenever possible, to a specific component of study treatment so that dose delays/interruption, dose skipping, or dose reduction can be made accurately.

If a participant experiences an AE such that talquetamab could not be given on Day 1 of a cycle, the cycle will be delayed. If this occurs, the full cycle duration will remain 28 days, and Day 1 of subsequent cycles will be adjusted accordingly to maintain the cycle duration. Day 1 of a cycle is not skipped.

For talquetamab, dosing interruption is the primary method of management of AEs. Dose reductions during step-up dosing and Cycle 1 are not permitted. Changes in dosing frequency and/or dose reduction(s) of talquetamab by 50% are considered starting in Cycle 2 for participants with talquetamab-related AEs after consultation with an approval by the sponsor. A re-escalation or return to protocol-specified dose schedule will be permitted in the absence of a recurrence of the AE that led to a reduction and if there was a clinical benefit and acceptable risk profile in the investigator's judgment. If a dose interruption was >35 days, treatment continuation will be required to be discussed with and approved by the sponsor. Repeat of step-up dose(s) and pretreatment medication may be required.

Guidance for talquetamab skipped doses is provided in Table 24 and guidance for restarting doses is provided in Table 25. If a dose of talquetamab is delayed, therapy will be restarted based on the recommendations in Table 25. The treatment schedule will be adjusted to maintain the appropriate intervals as specified in the SoA, as outlined in Tables 10-17. Disease evaluations as specified in the relevant SoA will be performed by the central laboratory for all cohorts, even if treatment had been delayed for any reason, regardless of any changes to the dosing regimen.

As noted in Table 25, dose delays beyond a specific interval from the planned dose will be discussed and approved by the sponsor before restarting treatment therapy. Pretreatment medications, including dexamethasone, will be administered prior to all repeat step-up doses, and the first treatment dose of talquetamab.

Once dose reduction has been implemented for a participant, a return to the protocol-specific dosing level will not occur for that participant unless in the investigator's judgment there is clinical benefit and a reasonable and acceptable risk profile.

Doses of talquetamab will be required to be interrupted due to hematologic toxicity per Table 26. Doses of talquetamab will be required to be interrupted for non-hematologic AEs per Table 27.

Following a dose interruption of talquetamab due to one or more of the non-hematologic AEs listed below, any clinically significant non-hematologic AEs other than weight loss, CRS, or ICANS will be required to resolve to Grade ≤1 or to baseline (as documented in the medical history) before proceeding to the next dose. Participants with Grade 3 weight loss with improvement to Grade 2 could resume dosing with sponsor approval. CRS (fever, hypoxia, and hypotension) and ICANS (in the opinion of the investigator) will be required to fully resolve (i.e., end date reported in the eCRF) before the next administration of talquetamab and until the following criteria are met: minimum of 12 hours since the resolution of fever without use of antipyretics; minimum of 24 hours since the last administration of tocilizumab or equivalent. Additionally, following dosing interruption of talquetamab there must have been no evidence of an active bacterial, viral, or fungal infection before proceeding to the next talquetamab dose.

Ciltacabtagene autoleucel will not be administered to participants with active infection. For participants requiring systemic antimicrobial treatment, or with temperature ≥38.0° C./100.4° F. within 48 hours before ciltacabtagene autoleucel infusion, investigator will be required to consult with the sponsor prior to dosing. Investigator is required consult with the sponsor prior to dosing if participant scored Grade ≥3 on non-hematologic toxicities of cyclophosphamide and fludarabine conditioning (except for Grade 3 nausea, vomiting, diarrhea, or constipation).

If resolution of these events to Grade ≤1 takes more than 14 days, the conditioning regimen will be readministered (cyclophosphamide 300 mg/m² and fludarabine 30 mg/m² daily for 3 days) after a minimum of 21 days following the first dose of the first conditioning regimen (cyclophosphamide and fludarabine).

For daratumumab SC, dosing interruption is the primary method of management of AEs. Dose reduction is not permitted for daratumumab SC. For lenalidomide and dexamethasone, dose reduction and dosing interruptions will be implemented as indicated.

If a participant experienced an AE such that any component of the DRd regimen could not be given on Day 1 of a cycle, the cycle will be delayed. If this occurs, all components of DRd will be held. If Day 1 of a cycle is delayed, the full cycle duration will remain at 28 days, and Day 1 of subsequent cycles will be adjusted accordingly to maintain the cycle duration. Day 1 of a cycle cannot be skipped.

If a component of the DRd regimen cannot be given on a dosing day after Day 1 for reasons other than CRS or ICANS (see Table 28), the other study drugs will be administered as scheduled.

If the parameters indicated in Table 29 are not met, the start of the next cycle will be held for a minimum of 1 week and a maximum of 28 days until recovery to the specified levels. Supportive care medications, including transfusions, can be administered at the investigator's discretion.

If there is a delay in the start of a new cycle (i.e., none of the study treatments are given during this period) due to insufficient recovery from toxicity, re-initiation, continuation, or discontinuation of any study treatment(s) need to be approved by the sponsor.

Dose reduction is not permitted for daratumumab SC. Dose reduction guidelines are provided in Table 30 for lenalidomide, and Table 31 for dexamethasone. Once dose reduction is implemented for a participant, a return to the protocol-specified dosing level will not occur for that participant unless in the investigator's judgment there is clinical benefit and a reasonable and acceptable risk profile.

Guidance for daratumumab skipped doses is provided in Table 32 and guidance for restarting doses is provided in Table 33 and Table 28.

Lenalidomide should be taken at the same time each day. If the expected dose time of lenalidomide was missed and <12 hours have elapsed from the time of the missed regular dose administration, the participant should take the missed dose as soon as possible and continued with the next dose at the normal time. If ≥12 hours elapsed, the missed dose should be skipped and not made up. The participant should not take 2 doses of lenalidomide at the same time. If dosing of lenalidomide was interrupted, the skipped doses should not be made up.

If a weekly dexamethasone dose was missed, it may be taken if <4 days have elapsed since the time when the dose should have been taken. If the next dose was scheduled to be taken within 3 days, the missed dose of dexamethasone should be skipped.

Guidance for restarting daratumumab doses after interruption due to hematologic toxicity is provided in Table 33. Guidance for restarting daratumumab after non-hematologic AEs is provided in Table 28.

Once dose reduction of DRd due to AEs is implemented for a participant, a return to the protocol-specific dosing level should not occur for that participant unless in the investigator's judgment there is a clinical benefit and a reasonable and acceptable risk profile.

Lenalidomide is primarily excreted unchanged by the kidney. Therefore, adjustments to the dose of lenalidomide are recommended to provide appropriate drug exposure in participants with moderate or severe renal impairment. Lenalidomide dose adjustment is instituted for participants with a CrCl≤60 mL/minute. To be enrolled in the study, participants are required to have CrCl≥30 mL/min. If during treatment a participant's renal status changes, the dose will be adjusted. The recommended doses for participants with MM and renal impairment are shown in Table 34.

Doses of daratumumab will be interrupted due to hematologic toxicity per Table 33 and will be interrupted due to non-hematologic toxicity per Table 38.

### Concomitant Therapy

Throughout the study, investigators will prescribe any concomitant medications or treatments deemed necessary to provide adequate supportive care. All medications (including prescriptions and over-the-counter products, and transfusions of blood products) different from the study drugs are recorded in the appropriate section of the eCRF throughout the study beginning with the signing of the informed consent form (ICF) until 30 days after the last dose of daratumumab, lenalidomide or dexamethasone, or until 30 days after the last dose of talquetamab, or until Day 112 after infusion of ciltacabtagene autoleucel, whichever is later, or until subsequent antimyeloma therapy is started.

Any subsequent antimyeloma therapy can only be administered following disease progression as confirmed by the sponsor. All such exceptions, deviations, and treatments have to be documented and approved by the sponsor in advance.

### Discontinuation of Study Treatment and Participant Discontinuation/Withdrawal

A participant's study treatment will be discontinued for the following reasons.
The participant has confirmed disease progression per IMWG response criteria after receiving ciltacabtagene autoleucel therapy.
The participant receives concurrent (non-protocol) systemic anticancer treatment.
The participant experiences a secondary primary malignancy (SPM) that could not be treated by surgery alone; participants who require radiation therapy for treatment of SPM must have study treatment discontinued unless, upon consultation with the sponsor and review of data, continuation is agreed upon.
The participant has an intercurrent illness that prevents further administration of treatment.
The participant withdraws consent to receive study treatment.
The investigator believes that for safety reasons or tolerability reasons (e.g., AE) it is in the best interest of the participant to discontinue study treatment.
The participant becomes pregnant.
   Participants who discontinue all study treatment continue assessments in the Follow-up Phase.

Talquetamab will be discontinued for any of the following reasons.
If a first event of Grade 3 CRS is not resolved in ≤48 hours.
If a second event of Grade 3 CRS or any event Grade 4 CRS occurs.
If a second event of Grade 3 ICANS or any event Grade 4 ICANS occurs.
If a second event of Grade 3 sARR or any event Grade 4 sARR associated with administration of talquetamab SC occurs.
If a grade ≥3 injection-site reaction associated with administration of talquetamab occurs.

Ciltacabtagene autoleucel will be discontinued for the following reasons.
If a grade ≥3 non-hematologic toxicity is occurs and precludes retreatment with cyclophosphamide and fludarabine prior to ciltacabtagene autoleucel infusion.

Participants could be withdrawn from the study due to loss of follow-up, withdrawal of consent, or other reasons documented in the Case Report Form (CRF). If a participant withdraws consent for the usage of samples for research, these will be destroyed once no longer needed for the study. If a participant is found to be deceased, it will be documented, and they would no longer be considered lost to follow-up.

### Follow-Up Phase

The Follow-up Phase begins once a participant discontinues study treatment. During the Follow-Up Phase, participants who discontinue for reasons other than PD, death, or withdrawal of consent continue to have disease evaluations according to the SoA (Tables 10A-10B [Cohort 1], Tables 11A-11B [Cohort 2], Table 12 [talquetamab consolidation treatment, Cohorts 1 and 2], Tables 13A-13B [Cohort 3], and Tables 14A-14B [(talquetamab bridging treatment, Cohort 3]). The Follow-up Phase continues until death, lost to follow-up, consent withdrawal, or study end, whichever occurs first.

### End of Study

The end of study is considered as 2 years after the last participant received their dose of ciltacabtagene autoleucel. The final data from the study site will be provided to the sponsor (or designee) after completion of the final participant visit at that study site.

A participant is considered to have completed the study if the participant has completed the last scheduled contact per the relevant SoA or died, whichever comes first. Participants who prematurely discontinue study treatment for any reason will continue to be followed for protocol-specified efficacy assessment and follow-up procedures. However, if a participant develops disease progression, they will be discontinued from the efficacy follow-up portion of the study but will continue to be followed for survival status and subsequent antimyeloma therapy (SST).

### Endpoints

The primary objective of the study is to evaluate the safety of the sequential use of talquetamab and ciltacabtagene autoleucel in this population due to targeting multiple tumor surface antigens (GPRC5D and BCMA) compared with either compound alone. Endpoints for this objective include measurement of the incidence and severity of adverse events (AEs).

Exploratory objectives of the study are to further characterize MRD-negativity. To characterize the pharmacokinetics and pharmacodynamics of ciltacabtagene autoleucel and talquetamab, to assess the immunogenicity of ciltacabtagene autoleucel and talquetamab, and to explore patient-reported oral toxicities associated with talquetamab. MRD-negativity endpoints include measurement of MRD-negative CR/sCR at 12 months, time to MRD-negativity, duration of MRD-negativity, correlation between MRD-negativity, PFS, and OS, and imaging plus MRD-negative rate. Pharmacokinetics and pharmacodynamic endpoints include measurement of pharmacokinetics parameters and pharmacodynamic markers including, but not limited to, systemic cytokine concentrations, markers of T-cell activation and CAR-T expansion and persistence, and CAR transgene levels. Immunogenicity endpoints include measurement of the presence of anti-drug antibody(ies) (ADAs) to ciltacabtagene autoleucel or talquetamab. Oral toxicity endpoints include measurement of the proportion of participants who reported oral toxicities over time, measurement of the proportion of participants with overall side effect burden over time, and measurement of the proportion of participants with global impression of disease severity and impact.

### Trial Oversight

This study will be conducted in accordance with the Declaration of Helsinki and International Council for Harmonisation guidelines for Good Clinical Practice. All patients will provide written informed consent. An independent ethics committee or institutional review board at each site will approve the study protocol. A data review committee will be established to monitor safety data collected in the clinical program and to evaluate the interim safety and efficacy data. This committee will consist of at least 1 medical expert in the relevant therapeutic area and at least 1 statistician.

### COVID-19 Safety Measures

Measures to prevent and mitigate risk of COVID-19 infection will be introduced during the study, including education on the importance of re-vaccination after ciltacabtagene autoleucel, and other preventative measures, and investigators will be asked to consider the use of prophylaxis and antiviral therapies such as Evusheld, (tixagevimab/cilgavimab), and early use of Paxlovid (nirmatrelvir/ritonavir) where available. Sites will be asked to follow recommendations from the American Society of Hematology and American Society for Transplantation and Cellular Therapy (ASH-ASTCT COVID-19 Vaccination for Hematopoietic Cell Transplant and CAR-T Cell Recipients: Frequently Asked Questions) and the European Society for Blood and Marrow Transplantation (EMBT) (Coronavirus Disease COVID-19: EBMT Recommendations).

### Example 7: Evaluation of Efficacy of Method of Treatment with Ciltacabtagene Autoleucel and Talquetamab

Secondary objectives of the study are to evaluate the preliminary efficacy of ciltacabtagene autoleucel and talquetamab and to characterize minimal residual disease (MRD) negativity. Endpoints for these objectives include measurement of overall response (proportion of participants with partial response or better), very good partial response (VGPR) or better, complete response or stringent complete response (CR/sCR), duration of response (DOR) and time to response (TTR), progression-free survival (PFS), overall survival (OS), MRD-negativity, and sustained MRD-negativity (≥6 months).

Randomization to cohort assignment will not be implemented because each cohort was analyzed separately without between-cohort comparisons.

Talquetamab and ciltacabtagene autoleucel pharmacokinetic and immunogenicity samples will be collected to characterize pharmacokinetics and immunogenicity of both drugs given sequentially. Additionally, exploratory analysis of pharmacokinetic/immunogenicity data will be conducted to evaluate its impact on safety and efficacy endpoints.

Biomarker samples will be collected to evaluate the mechanism of action of talquetamab and ciltacabtagene autoleucel, evaluate pharmacodynamics, possibly investigate any inter-participant variability in clinical outcomes, and identify any subgroups of participants that responded differently to study treatment.

Patient reported outcomes (PROs) will be collected to explore treatment-related symptoms including oral toxicities associated with talquetamab. PROs were also collected to evaluate health-related quality of life.

The tests performed to assess IMWG-based response criteria are as follows:

### M-Protein Measurements in Serum and Urine

Blood and 24-hour urine samples for M-protein measurements will be sent to and analyzed by a laboratory, as specified in the SoA.

Only one serum and one 24-hour urine sample per timepoint are required to perform the following tests: Serum quantitative Ig; serum M-protein quantitation by electrophoresis (SPEP); 24-hour urine M-protein quantitation by electrophoresis (UPEP); serum free light chain (FLC) assay; and serum immunofixation electrophoresis (SIFE)/urine immunofixation electrophoresis (UIFE).

Blood and 24-hour urine samples will be collected as specified in the relevant SoA until the development of confirmed disease progression, death, or start of a new antimyeloma treatment. Disease progression based on one of the laboratory tests alone will be confirmed by ≥1 repeat investigation. Disease evaluations continue beyond relapse from CR until disease progression is confirmed.

The investigational sites are required to notify the sponsor if a participant was diagnosed with disease progression (which were confirmed with a consecutive assessment ≥1 calendar day apart if based on M-protein/serum FLC levels) and provide documentation of disease progression (e.g., by completing a Disease Progression form or by contacting the IWRS). The sponsor will review the data provided to confirm that the IMWG response criteria for PD had been met. If disease progression is confirmed by the sponsor, then the participant discontinues study treatment, completes the end of treatment (EOT) Visit, and enters the Follow-up Phase. If disease progression has not occurred at the time of the EOT Visit, disease evaluations continue until disease progression was confirmed. SST was not started until after disease progression is confirmed by the sponsor.

Daratumumab may be detected on SPEP and SIFE assays used for monitoring M-protein, which can lead to false SPEP and SIFE results for participants with IgG kappa M-protein and affect assessments of responses based on IMWG response criteria. Therefore, a daratumumab-specific immunofixation electrophoresis (DSIFE) will be performed to confirm VGPR or better in participants with IgG kappa myeloma when daratumumab interference was suspected based on SPEP and SIFE results. This reflex assay relies on the use of daratumumab-specific murine anti-idiotype antibody that binds and shifts daratumumab's migration pattern during electrophoresis, thus distinguishing daratumumab from endogenous M-protein (McCudden 2016).

### Bone Marrow Examination

Bone marrow aspirate (or core biopsy if aspirate was not possible) will be collected for clinical assessments and biomarker evaluations, including cytogenetics (see SoA and Table 35). Clinical staging (morphology, cytogenetics, and immunohistochemistry or immunofluorescence or flow cytometry) will be done. A portion of the bone marrow aspirate will be immunophenotyped and monitor for BCMA, checkpoint ligand expression in CD138-positive multiple myeloma cells, and checkpoint expression on T cells. If feasible, bone marrow aspirate will also be performed to confirm CR and sCR and at disease progression. Additionally, since minimal residual disease (MRD) negativity is being evaluated as a potential surrogate for PFS and OS in multiple myeloma treatment, MRD will be monitored in subjects using next generation sequencing (NGS) on bone marrow aspirate DNA. Baseline bone marrow aspirates will be used to define the myeloma clones, and post-treatment samples will be used to evaluate MRD negativity. A fresh bone marrow aspirate will be collected prior to the first dose of conditioning regimen (≤7 days).

### Minimal Residual Disease Evaluations

Bone marrow aspirates will be collected to identify the MRD clones at screening and to monitor MRD status throughout the study. MRD will be evaluated using next-generation sequencing (NGS) on bone marrow aspirate DNA by a central laboratory.

If the myeloma calibration clone is not identified successfully from the fresh bone marrow aspirate taken at screening, another bone marrow aspirate or archival non-decalcified diagnostic tissue (e.g., bone marrow aspirate or clot slides) or formalin-fixed, paraffin-embedded block (clot section only, no bone marrow biopsy) will be requested. Alternatively, if MRD calibration has previously been determined locally using the NGS clonoSEQ® assay (Adaptive Biotechnologies, Seattle, WA), this deidentified data could be provided for the screening MRD assessment.

If, after all efforts, a calibration clone for MRD assessment could be identified based on central or local material, the sponsor notified the investigator, and the participant did not need to continue with bone marrow aspirate collections specifically aimed at MRD determination. Also, if a bone marrow aspirate collected during treatment to evaluate MRD-negativity is deemed of low quality and/or yields no results, another fresh bone marrow aspirate could be requested.

### Skeletal Survey

Imaging for the assessment of lytic bone lesions will be performed at screening and thereafter as clinically indicated. Imaging was interpreted locally. Any of the following modalities are acceptable but must have included imaging of skull, entire vertebral column, pelvis, chest, humeri, femora, and any other bones for which the investigator suspects involvement by disease:
A skeletal survey (including skull, entire vertebral column, pelvis, chest, humeri, femora, and any other bones for which the investigator suspects involvement by disease).
Whole-body MRI.
Low-dose whole-body CT.
PET/CT with diagnostic CT component. If a CT scan is used, it must have been of diagnostic quality per the IMWG diagnostic criteria.

The modality used for screening will be maintained for any subsequent imaging procedures and, if indicated, to evaluate for possible disease progression.

Following the start of study treatment, imaging for the assessment of lytic bone lesions will be performed locally, whenever clinically indicated based on symptoms, to document disease progression. If a participant presented with disease progression manifested by symptoms of pain due to bone changes, then disease progression will be documented by skeletal survey or other radiographs, depending on the symptoms that the participant experiences. If the diagnosis of disease progression is obvious by radiographic investigations, then no repeat confirmatory imaging will be necessary. If changes were equivocal, then a repeat imaging is needed in 1 to 3 weeks.

### Documentations of Soft-Tissue Plasmacytoma

There are 2 types of soft-tissue plasmacytomas (STPs) in MM: 1) extramedullary plasmacytomas involving only soft-tissues and 2) paraskeletal plasmacytomas with tumor masses arising from skeletal lesions (Rosinol 2021). Instructions in this section applied to both extramedullary and paraskeletal STPs.

STPs must be assessed and documented by physical examination or radiologic imaging for all participants during Screening. Physical examination, MRI, or CT with IV contrast (only if there was no contraindication to the use of IV contrast) maybe used to document the presence and size of STPs. PET scan or ultrasound are not acceptable methods. However, PET/CT scans may be used to document the presence and size of STPs if the CT component of the PET/CT scan was of sufficient diagnostic quality.

STPs should be evaluated until plasmacytoma(s) met CR criteria or confirmed disease progression or start of subsequent antimyeloma therapy. The methodology used for evaluation of each plasmacytoma should be consistent across all visits. The frequency of assessment depended on the method being used: if measurable by physical examination, evaluate Q4W (±7 days); if measurable by radiologic imaging, evaluate on Day 56 and then Q12W (±7 days).

If a STP can be assessed by both radiology and physical examination, one method should be selected and used for response determination, i.e., imaging and physical examination results for the same lesion should not be reported in the eCRF.

Irradiated or excised lesions will be considered “not measurable” and will be monitored only for disease progression.

### Patient-Reported Outcomes

PRO data will be collected to assess disease- and treatment-related symptoms and impacts using the following PRO instruments: PRO-CTCAE; EORTC-Q168; and PGI-S.

The timing of PRO assessments will be per the SoA (Tables 10A-10B [Cohort 1], Tables 11A-11B [Cohort 2], Table 12 [talquetamab consolidation treatment, Cohorts 1 and 2], Tables 11A-11B [Cohort 3], and Tables 14A-14B [(talquetamab bridging treatment, Cohort 3]).

PRO instruments should be completed by the participant before any clinical tests, procedures, or other consultations that would influence his or her perceptions of their current health state. PRO and AE data was not reconciled with one another.

### Treatment Response

Overall response rate (ORR) is defined as the proportion of subjects who achieved a PR or better according to the IMWG criteria (sCR+CR+VGPR+PR).

VGPR or better response rate is defined as the proportion of subjects who achieve a VGPR or better response according to the IMWG criteria (sCR+CR+VGPR).

Duration of response (DOR) will be calculated among responders (with a PR or better response) from the date of initial documentation of a response (PR or better) to the date of first documented evidence of progressive disease, as defined in the IMWG criteria. Relapse from CR by positive immunofixation or trace amount of M-protein will not be considered as disease progression. Disease evaluations will continue beyond relapse from CR until disease progression is confirmed.

Time to response (TTR) is defined as the time between date of the initial infusion of ciltacabtagene autoleucel and the first efficacy evaluation at which the subject had met all criteria for PR or better.

Progression-free survival (PFS) is defined as the time from the date of the first study treatment to the date of first documented disease progression, as defined in the IMWG criteria, or death due to any cause, whichever occurs first.

Overall survival (OS) will be measured from the date of the initial infusion of ciltacabtagene autoleucel to the date of the subject's death.

### Example 8: Evaluation of Safety of Method of Treatment with Ciltacabtagene Autoleucel and Talquetamab

Disease status will be evaluated according to the International Myeloma Working Group (IMWD) response criteria for MM. Responses or progression will be evaluated by investigators, and use of a validated computerized algorithm. PROs will also be assessed. An internal Data Review Committee (DRV) will be established to monitor data on an ongoing basis and to ensure the continuing safety of the participants enrolled in the study.

Safety assessments will include physical examinations, neurologic examinations, Easter Cooperative Oncology Group (ECOG) performance status score, clinically laboratory tests, vital signs, and adverse event (AE) monitoring. The severity of AEs will be assessed using National Cancer Institute Common Terminology Criteria for Adverse Events (NCI-CTCAE) Version 5.0, except for grading cytokine release syndrome (CRS) and immune effector cell-associated neurotoxicity syndrome (ICANS), which should be assessed based on American Society for Transplantation and Cellular Therapy (ASTCT) guidelines. Concomitant medication use will be recorded.

Adverse events will be reported and followed by the investigator. Any clinically relevant changes occurring during the study must be recorded on the Adverse Event section of the CRF. Any clinically significant abnormalities persisting at the end of the study/early withdrawal will be followed by the investigator until resolution or until a clinically stable condition was reached. The study included the following evaluations of safety and tolerability according to the time points provided in the SoA.

Medical history, including but not limited to all ongoing relevant and clinically significant comorbidities according to NCI-CTCAE Version 5.0, including severity grade, will be recorded during screening. Clinically significant post-baseline abnormalities should be recorded as AEs.

A complete physical examination will be conducted at screening. Symptom-directed physical examinations will be conducted thereafter.

Body weight will be measured at screening and per the SoA timings. For participants on DRd treatment, body weight will be measured, on Day 1 of each treatment cycle, and as clinically indicated.

Temperature, pulse/heart rate, respiratory rate, blood pressure, and oxygen saturation will be assessed. Blood pressure and pulse/heart rate measurements were assessed sitting with a completely automated device. Manual techniques will be used only if an automated device is not available. Blood pressure and pulse/heart rate measurements were preceded by at least 5 minutes of rest in a quiet setting without distractions (e.g., television, cell phones).

Any additional vital signs assessments supporting the start and end dates of an AE (e.g., fever, hypertension) should be reported in the eCRF.

During the collection of 12-lead ECGs, participants should be in a quiet setting without distractions (e.g., television, cell phones). Participants should rest in a supine position for at least 5 minutes before ECG collection and refrain from talking or moving arms or legs.

In addition to screening, assessment of cardiac function using either transthoracic echocardiogram or MUGA scan is required as clinically indicated if the participant developed signs/symptoms of heart failure. At a minimum, this includes assessment of left ventricular ejection fraction reported as a percentage. This value should be recorded in the eCRF.

Blood samples for serum chemistry and hematology will be collected. The investigator must review the laboratory results, document this review, and record any clinically relevant changes occurring during the study in the AE section of the CRF. For example, laboratory abnormalities leading to an action regarding study treatment (dose change, dose interruption, cycle delay, or treatment discontinuation) or the start of concomitant therapy should be reported. For each laboratory abnormality reported as an AE, the following laboratory values should be reported in the laboratory section of the eCRF: the value indicative of the onset of each toxicity grade, the most abnormal value observed during the AE, and the value supporting recovery to Grade ≤1 or to baseline values. The laboratory reports must be filed with the source documents.

Any additional chemistry and hematologic laboratory assessment supporting the start and end dates of an AE should be reported in the eCRF.

A serum pregnancy test is required at screening, and then further serum or urine pregnancy tests are required per timings in the relevant SoA. Additional serum or urine pregnancy tests may be performed, as determined necessary by the investigator or required by local regulation, to establish the absence of pregnancy at any time during the participation in the study.

HBV serology is not required at screening if this was performed as part of standard of care within 3 months prior to the start of administration of study treatment. The HBV screening guide in Table 36 will be used to determine participant eligibility for the study.

HCV antibody testing is not required at screening if this was performed as part of standard of care within 3 months prior to the start of administration of study treatment. Participants with a history of HCV antibody positivity must undergo HCV-RNA testing. If a participant with history of chronic HCV infection (defined as both HCV antibody and HCV-RNA positive) completed antiviral therapy and had undetectable HCV-RNA for ≥24 weeks following the completion of therapy, the participant is eligible for the study.

During and following study treatment, participants who are at risk of HBV reactivation (e.g., anti-hepatitis B core-positive irrespective of anti-hepatitis B surface status, anti-hepatitis B surface-positive and unknown HBV vaccination history, or known history of prior HBV infection irrespective of hepatitis B serology findings) will be closely monitored for clinical and laboratory signs (including DNA PCR) of reactivation of HBV, by PCR testing, Q12W (±7 days) from C1D1 for up to 6 months after the last dose of study treatment and as clinically indicated.

Participants with history of HCV antibody positivity will be monitored for HCV-RNA testing Q12W (±7 days) from C1D1 for up to 6 months after the last dose of study treatment and as clinically indicated.

A neurologic examination including the ICE Tool evaluation will be performed prior to Step-up Dose 1 of talquetamab (to establish baseline neurologic status) and during the Treatment Phase if ICANS was suspected. Repeat ICE evaluation at least once per day, and with any change in clinical status until fully resolved.

Qualitative changes in handwriting from baseline are being explored by the sponsor as a potential early clinical predictive marker for neurotoxicity. Currently, no standardized toxicity gradings are available in NCI-CTCAE Version 5.0. for these type of changes in handwriting. Therefore, the sponsor has developed a handwriting assessment criterion to assess participants for occurrence of the following types of changes in handwriting: micrographia, dysgraphia, or agraphia, as potential early indicators for neurotoxicity.

The Eastern Cooperative Oncology Group (ECOG) performance status scale will be used to grade changes in the participant's activities of daily living (ADL). When scheduled, ECOG performance status assessment should be obtained prior to any other study procedures planned for the same day whenever possible.

In exceptional circumstances, home health care and telemedicine visits may be implemented by or with approval from the sponsor and per the clinical judgment of the investigator, where feasible and permissible by local policy.

Participants for whom there was no safety concern may have had home health care and telemedicine (conducted via phone or video conference) visits.

Study procedures such as ECOG assessment, AE and concomitant medication reporting, review of body systems, and collection of information on the participant's current health status may be performed with home health care and telemedicine visits. Protocol-specified laboratory assessments for efficacy and safety may be collected during home health care visits.

Telemedicine visits (conducted via phone or video conference) may be implemented by or with approval from the sponsor and per investigator's clinical judgment for certain circumstances when warranted where feasible and permissible by local policy, regulations (as applicable) and for participants for whom there was no safety concern.

Source documentation and, if applicable, the appropriate eCRFs should be completed and detail how each assessment was collected (e.g., remote versus on-site, central versus local laboratory, vital signs taken at home by delegated in-home nursing).

### Adverse Events, Serious Adverse Events, and Other Safety Reporting

Adverse events (AEs) were reported by the participant (or, when appropriate, by a caregiver, surrogate, or the participant's legally acceptable representative) for the duration of the study.

All AEs except CRS and ICANS will be graded per NCI-CTCAE Version 5.0. CRS and ICANS will be graded based on the American Society for Transplantation and Cellular Therapy (ASTCT) guidelines (Lee 2019; see Table 37 and Table 38, respectively). Symptoms of CRS and ICANS will be graded according to NCI-CTCAE Version 5.0 and reported in the eCRF. See also supplementary information for grading of systemic administration-related reactions (sARRs) in Table 39. Neurotoxicity that is not temporarily associated with CRS, or any other neurologic AEs that do not qualify as ICANS, will be graded by CTCAE criteria. Any adverse event or serious adverse event not listed in the NCI CTCAE Version 5.0 will be graded according to investigator clinical judgment by using the standard grades as follows:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

Changes in handwriting (i.e., micrographia, dysgraphia, or agraphia) should be graded using the criteria outlined in Table 40 and reported as an AE in the eCRF. If a participant experienced a serious CAR-T associated neurotoxicity (either ICANS or other neurotoxicity), then a copy of the handwriting assessment log should be submitted with the serious AE report.

All AEs (with the exception of progression of MM, delayed AEs, HBV reactivation, and COVID-19 infection, and events of neurotoxicity or exacerbation of existing neurologic AEs [reported until the end of study]) and special reporting situations, whether serious or non-serious, will be reported from the time a signed and dated ICF was obtained until 30 days after the last dose of study treatment or until the start of SST, if earlier, or until Day 112 post ciltacabtagene autoleucel infusion (whichever is later) and regardless if PD occurs prior to Day 112 or subsequent antimyeloma therapy was started prior to Day 112.

Beyond this AE reporting period, only SAEs regardless of causality and non-serious AEs that are considered related to a study drug need to be reported until the end of the study except as defined for delayed AEs below. In addition, events of HBV reactivation and COVID-19 infection will be reported during the first-year post-infusion of ciltacabtagene autoleucel, regardless of severity or causality. All AEs and special reporting situations, whether serious or non-serious, will be collected for participants who are unable to be apheresed, or receive conditioning regimen or ciltacabtagene autoleucel infusion until PD or until the start of antimyeloma therapy, whichever is earlier.

All AEs with an onset date after the signing of the ICF and up to 30 days after talquetamab discontinuation or 112 days after ciltacabtagene autoleucel infusion, whichever was the latest (up to EOS for participants who enter a continued access program on the same day as the EOS visit) must be recorded on specific AE pages of the eCRF.

All serious AEs (SAEs) occurring during the study must be reported to the appropriate sponsor contact person by study site personnel immediately, but no later than 24 hours of their knowledge of the event.

SAEs (regardless of causality) must be reported until study completion and subsequently will be collected at least once per year in the long-term follow-up study, CARTINUE, for up to 15 years post-infusion of ciltacabtagene autoleucel. The sponsor will evaluate any safety information that is spontaneously reported by an investigator beyond the time frame specified in the protocol.

Events that required an escalation of care when the participant was already hospitalized should be recorded as an SAE. Examples of such events include movement from routine care in the hospital to the intensive care unit or if that event resulted in a prolongation of the existing planned hospitalization.

All SAEs occurring after signature of the ICF and up to 30 days after talquetamab discontinuation and 112 days after ciltacabtagene autoleucel infusion (up to EOS for participants who enter a continued access program on the same day as the EOS visit) must be recorded on AE pages in the eCRF and on an SAE form, regardless of the investigator-attributed causal relationship with study treatment or study mandated procedures.

Information regarding SAEs will be transmitted to the sponsor using the Serious Adverse Event Form and Safety Report Form of the eCRF, which must be completed and reviewed by a physician from the study site, and transmitted to the sponsor immediately but no later than within 24 hours of their knowledge of the event.

Care will betaken not to introduce bias when detecting AEs or SAEs. Open-ended and nonleading verbal questioning of the participant is the preferred method to inquire about AE occurrence. Solicited AEs are predefined local and systemic events for which the participant is specifically questioned. Unsolicited AEs are all AEs for which the participant is not specifically questioned.

The investigator is obligated to perform or arrange for the conduct of supplemental measurements and evaluations as medically indicated to elucidate the nature and causality of the AE or SAE as fully as possible. This included additional laboratory tests or investigations, histopathological examinations, or consultation with other health care professionals.

All initial reports of pregnancy in female participants or partners of male participants must be reported to the sponsor by the study site personnel within 24 hours of their knowledge of the event using the appropriate pregnancy notification form. Abnormal pregnancy outcomes (e.g., spontaneous abortion, fetal death, stillbirth, congenital anomalies, ectopic pregnancy) are considered SAEs and must be reported using an SAE reporting form. Any participant who becomes pregnant during the study must discontinue from further study treatment. Because the effect of the study treatment on sperm is unknown, pregnancies in partners of male participants included in the study will be reported as noted above.

Follow-up information regarding the outcome of the pregnancy for female participants who become pregnant, or where the pregnancy was the result of male participant and his partner, and any postnatal sequelae in the infant will be required.

All events that meet the definition of an SAE will be reported as SAEs, regardless of whether they are protocol-specific assessments. Expected progression of disease should not considered or reported as an AE (or SAE). However, if determined by the investigator to be more likely related to the study treatment than being expected from the underlying disease, the treatment-invoked progression (i.e., the treatment-invoked signs/symptoms of such progression) should be reported.

Progression of disease and death due to PD should be documented in the appropriate eCRFs (e.g., the Disease Progression form and the Death Information form). Disease progression should not be recorded as an AE or SAE. However, signs and symptoms of PD that are of clinical significance, e.g., spinal cord compression, vena cava superior syndrome, major vessel rupture, efflux obstruction or organ failure, should be reported.

As part of standard safety monitoring activities by the sponsor, any occurrence of an adverse event of special interest (AESI) must be followed until resolution. All AESIs will be reported to the sponsor within 24 hours of awareness of the event irrespective or seriousness (i.e., serious and non-serious AEs). Serious AESIs will be reported via standard SAE reporting while non-serious AESIs will be reported solely via the eCRF. Grade ≥2 CRS and Grade ≥2 ICANS are considered AESIs for talquetamab.

SPMs are AESIs and will be followed as part of standard safety monitoring activities by the sponsor, regardless of severity or causality, from the time of signing of the ICF until study completion.

CRS and neurotoxicity (including CAR-T cell-related neurotoxicity, i.e., ICANS and other neurotoxicities resulting from ciltacabtagene autoleucel) are also AESIs and will be followed as part of standard safety monitoring activities by the sponsor, regardless of severity. These events will require enhanced data collection in the eCRF, will be reported to the sponsor in a timely manner irrespective of seriousness, and will be followed until resolution or stabilization of the event.

In addition, from the Day 1 ciltacabtagene autoleucel infusion and for the duration of the study, the following must be reported to the sponsor using the Serious Adverse Event Form within 24 hours of awareness of the event, irrespective of seriousness (e.g., serious or non-serious AEs) or causality:
≥Grade 3 CRS
≥Grade 3 neurotoxicity
Any grade movement or neurocognitive toxicity (i.e., parkinsonism)·
Any grade SPM (including recurrence of pre-existing malignancies)

The following delayed AEs will be collected, regardless of causality, from the time of ciltacabtagene autoleucel infusion (except for SPMs, which are collected from the time of signing of the ICF) until end of study or in a separate long-term follow-up study (CARTINUE):
New primary malignancies or recurrence of pre-existing malignancy (all grades), except for MM (which should be reported as PD), must be reported to the sponsor within 24 hours of awareness of the event for the duration of the study, irrespective of seriousness or causality. A tumor sample must be collected, and DNA, RNA, or protein analysis will be performed to investigate the presence of LV elements. Additional samples (including but not limited to blood, tissue, tumor) may be requested as clinically indicated.
New incidence or exacerbation of a pre-existing neurologic disorder (all grades) and exacerbation of new neurologic disorders. Grade ≥3 neurotoxicity and any grade movement and neurocognitive toxicity (e.g., parkinsonism; see Appendix 21) must be reported to the sponsor within 24 hours of awareness of the event for the duration of the study, irrespective of seriousness or causality.
New incidence or exacerbation of a pre-existing rheumatologic or other autoimmune disorder (all grades).
New incidence of ≥Grade 3 hematologic disorder (including hypogammaglobulinemia).
New incidence of ≥Grade 3 infection.

### Example 9: Assessment of Pharmacokinetics, Pharmacodynamics, Immunogenicity, and Oral Toxicities of Ciltacabtagene Autoleucel and Talquetamab Treatment

### Pharmacokinetic Assessments: Analytical Procedures

Serum from venous blood samples will be collected to evaluate the pharmacokinetics of talquetamab and ADAs according to the SoA (see Tables 41A-41B, Tables 42A-42B, and Table 43) using specific and sensitive assays that are validated by or under the supervision of the sponsor.

Post ciltacabtagene autoleucel infusion blood samples will be analyzed to determine CAR+ T cell concentration and transgene levels of ciltacabtagene autoleucel based on the SoA using specific and sensitive assays that are validated by or under the supervision of the sponsor.

Scrum collected for the analysis of talquetamab pharmacokinetics/immunogenicity and ciltacabtagene autoleucel pharmacokinetics/immunogenicity may additionally be used to evaluate safety or efficacy aspects that address concerns arising during or after the study period. Genomic analyses will not be performed on these serum samples. Participant confidentiality will be maintained.

Flow cytometry based and transgene level pharmacokinetic analyses will be performed on the PK-evaluable Analysis Set. All concentrations below the lowest quantifiable concentration or missing data will be labeled as such in the concentration database. Concentrations below the lowest quantifiable concentration will be treated as zero in the summary statistics. Descriptive statistics will be used to summarize CAR+ T cell concentration and transgene level in the blood at each sampling timepoint. If sufficient data is available, population-PK analysis will be performed and may be combined with data from other studies. If the population-PK analysis was conducted, details will be given in a population-PK analysis plan and the results of the analysis will be presented in a separate report. Exposure-response analyses may also be performed and may use data from other studies; if performed, details will be provided in a separate analysis plan and report.

PK analyses will be performed on the talquetamab PK-evaluable Analysis Set. Descriptive statistics will be used to summarize talquetamab serum concentrations at each sampling time for each cohort. All serum concentrations below the lowest quantifiable concentration or missing data will be labeled as such in the concentration data presentation. Concentrations below the lowest quantifiable concentration will be treated as zero in the summary statistics. All participants and samples excluded from the analysis will be clearly documented in the study report.

### Pharmacokinetic Assessments: Parameters and Evaluations

Sparse blood samples will be collected from all participants to measure serum concentrations of talquetamab and ciltacabtagene autoleucel CAR+ T cell transgene level and may be analyzed using a population pharmacokinetic approach if feasible, which may have included data from other studies, and the results may have been reported separately. Descriptive summaries for talquetamab serum concentrations, CAR+ T cell concentration, and transgene level at each timepoint will be presented.

### Pharmacokinetic Assessments: Biomarkers

Biomarker studies were designed to identify markers predictive of response (or resistance) to talquetamab and ciltacabtagene autoleucel. Analyses will be stratified by clinical covariates or molecular subgroups using the appropriate statistical methods (e.g., parametric or non-parametric, univariate, or multivariate, analysis of variance, or survival analysis, depending on the endpoint). Correlation of baseline expression levels or changes in expression levels with response to time-to-event endpoints may identify responsive (or resistant) subgroups in addition to genes and pathways attenuated following treatment with talquetamab and ciltacabtagene autoleucel.

Any pharmacodynamic measures were listed, tabulated, and where appropriate, plotted. Participants were grouped by cohort, dose schedule, or clinical response. As this was an open-label study with no control arm, statistical analyses were done to aid in the understanding of the results.

For biomarker evaluations, whole blood and bone marrow aspirate samples will be collected at baseline and during treatment per the SoA (see Tables 41A-41B, Tables 42A-42B, and Table 43).

Biomarker assessments focused on several objectives:
To evaluate depth and duration of response for the different regimens, bone marrow MRD evaluations will be performed using NGS method clonoSEQ® assay.
To evaluate apheresis (including but not limited to scRNAseq) and infused CAR-positive T cell subsets and activation and exhaustion markers, including but not limited to, CD4+, CD8+, CD25+, PD-1+, central memory, effector memory and/or other T cell subsets.
Immunophenotyping of immune cells, e.g., CD4+ and CD8+ T cells, regulatory T cells, B cells, and natural killer cells, following ciltacabtagene autoleucel and talquetamab administration.
Assessment of biomarkers of response/resistance on myeloma cells and/or the tumor microenvironment (e.g., BCMA and GPRC5D).
Serum or plasma proteomic profiling, including but not limited to, cytokines (e.g., IL-6, IL-10, and IL-15) and/or other immune-related proteins.

Additional biomarker samples may be collected to help understand an unexplained AE including but not limited to serum or PBMCs from whole blood. Additional sample(s) for cytokines will be collected as clinically indicated.

The potential presence of replication competent lentivirus (RCL) will be evaluated from whole blood samples of participants treated with ciltacabtagene autoleucel in combination with talquetamab. RCL will be evaluated using a qPCR assay against the lentiviral vesicular stomatitis virus-G gene up to 1 year from the start of study. Additional samples may be collected if triggered by events that may be relevant, including but not limited to RCL per clinical assessment.

Biomarker analyses are dependent upon the availability of appropriate biomarker assays and clinical response rates.

### Pharmacokinetic Assessments: Pharmacodynamic/Predictive Markers

The baseline immune subsets and dynamic changes/persistence and activation of CAR-positive viable T cells as well as talquetamab-mediated T cell activation may be associated with the depth and durability of response. An evaluation of these cell populations may be performed by flow cytometry and/or cytometry by time of flight (CyTOF) and correlated with response. Additional immunophenotyping may be performed on bone marrow aspirate (BMA) and whole blood samples to evaluate expression of biomarkers on myeloma cells (including but not limited to BCMA and GPRC5D antigen expression) and on immune cell populations (including on CD4+ and CD8+ T cells) by flow cytometry or CyTOF.

Circulating serum biomarkers present at baseline and following infusion of CAR-T cells and talquetamab administration may be associated with response to some CAR-T cell-based therapies. Evaluation of cytokines (such as IL-6 and IFN-γ) and other circulating proteins (granzymes, or perforin) will be analyzed to identify potential pharmacodynamic and predictive biomarkers of response or resistance.

### Pharmacokinetic Assessments: Immunogenicity

Antibodies to talquetamab and ciltacabtagene autoleucel will be evaluated in serum samples collected from all participants according to the SoA. Additionally, serum samples should also be collected at the final visit from participants who discontinued study treatment or were withdrawn from the study. These samples will be tested by the sponsor or sponsor's designee. Serum samples will be screened for antibodies binding to talquetamab and the titer of confirmed positive samples will be reported. Positive samples for binding antibodies will be tested for neutralizing antibodies to talquetamab. Immunogenicity bioanalysis may be conducted on pharmacokinetic samples collected at other timepoints, if deemed necessary. Samples collected for immunogenicity analyses may additionally be used to evaluate safety or efficacy aspects that address concerns arising during or after the study period. Genomic analyses will not be performed on these serum samples. Participant confidentiality will be maintained.

Immunogenicity analyses will be performed on the ciltacabtagene autoleucel Immunogenicity-evaluable Analysis Set. The incidence of antibodies to ciltacabtagene autoleucel will be summarized separately for all participants who receive a ciltacabtagene autoleucel infusion and had at least 1 post-dose immunogenicity sample. A listing of any participants who were positive for ADAs to ciltacabtagene autoleucel will be presented.

Immunogenicity analyses will be performed on the talquetamab Immunogenicity-evaluable Analysis Set. The incidence of antibodies to talquetamab will be summarized separately for all participants who received at least 1 dose of talquetamab and had at least 1 post-dose immunogenicity sample. A listing of any participants who were positive for anti-drug antibody(ies) ADAs to talquetamab will be presented.

### Patient-Reported Outcome Assessments

PRO data analyses will be performed on the PRO-evaluable Analysis Set. Each item from these PRO instruments will be descriptively summarized at each time point.

### Statistical Considerations

No formal statistical hypothesis testing will be performed. For each cohort, at least 10 participants will be enrolled with the possibility of expansion based on emerging data. The sample size of each cohort has been selected to allow collection of the required preliminary efficacy and safety data. If participants had progressive disease, withdraw consent, or die prior to ciltacabtagene autoleucel infusion, additional participants may be enrolled to complete the cohorts.

For purposes of analysis, the following analysis sets are defined according to Table 44.

Continuous variables will be summarized using the number of observations, mean, standard deviation, coefficient of variation, median, and range as appropriate. Categorical variables will be summarized using number of observation and percentage as appropriate. For time-to-event variables, the Kaplan-Meier method will be used for descriptive summaries.

The primary endpoint is incidence and severity of AEs. Secondary efficacy endpoints include overall response, VGPR or better, CR/sCR, overall MRD-negativity, sustained MRD-negativity (≥6 months), DOR, TTR, PFS, and OS. Summary statistics are summarized below in Table 45.

Analysis of overall response, VGPR or better, CR/sCR, and sustained MRD-negativity (≥6 months), was summarized with rate and corresponding 2-sided 95% Clopper-Pearson exact confidence interval. TTR was summarized descriptively. For time-to-event endpoints, including DOR, PFS, and OS, the Kaplan-Meier method was used.

Safety analyses will be made on the All-Treated Analysis Set. The safety parameters evaluated are the incidence, severity, and type of AEs, clinically significant changes in the participant's physical examination findings, vital signs measurements, and clinical laboratory results. Exposure to investigational product and reasons for discontinuation of study drug will be tabulated. Adverse events will be summarized by system organ class, preferred term, worst grade experienced by the participant.

The verbatim terms used in the eCRF by investigators to identify AEs will be coded using the MedDRA. Any new or worsening AE occurring at or after the signing of the ICF through the day of last dose plus 112 (if ciltacabtagene autoleucel)) or 30 (if not ciltacabtagene autoleucel study treatment) days or prior to the start of subsequent anticancer therapy, whichever is earlier, or any AE that is considered treatment-related regardless of the start date of the event, is considered to be treatment-emergent. All reported treatment-emergent adverse events will be included in the analysis. For each AE, the percentage of participants who experience at least 1 occurrence of the given event will be summarized by dose group. Summaries, listings, datasets, or participant narratives may be provided, as appropriate, for those participants who died, experienced an AE of special interest, discontinued treatment due to an AE, or who experienced a severe or a serious AE. AEs that occurred after administration of the conditioning regimen and before ciltacabtagene autoleucel infusion will be summarized and listed separately.

Laboratory data will be summarized by type of laboratory test. Reference ranges and markedly abnormal results (per specifications in the SAP) will be used in the summary of laboratory data. Descriptive statistics will be calculated for each laboratory analyte at baseline and for observed values and changes from baseline at each scheduled time point. Worst toxicity grade during treatment will be presented according to NCI-CTCAE Version 5.0. Change from baseline to the worst toxicity grade experienced by the participant during the study will be provided as shift tables. A listing of participants with any laboratory results outside the reference ranges will be provided.

The interpretation of the ECGs as determined by a qualified physician (investigator or qualified designee) will be summarized at scheduled time points.

Descriptive statistics of temperature, oxygen saturation, pulse/heart rate, respiratory rate, and blood pressure (systolic and diastolic) values and changes from baseline will be summarized at each scheduled time point. The percentage of participants with values beyond clinically important limits will be summarized.

### TABLES

TABLE 9
International Myeloma Working Group Response Criteria.

| Response | Response Criteria |
|---|---|
| sCR | CR as defined below, plus |
| | Normal FLC ratio, and |
| | Absence of clonal PCs by IHC (κ/λ ratio ≤4:1 or ≥1:2 for κ and λ |
| | participants, respectively, after counting ≥100 PCs) or negative 2-4 color flow |
| | cytometry |
| CR^{a} | Negative immunofixation of serum and urine, and |
| | Disappearance of any soft-tissue plasmacytomas, and |
| | <5% PCs in bone marrow |
| | No evidence of initial M-protein isotype(s) on immunofixation of the |
| | serum and urine^{b} |
| VGPR^{a} | Serum and urine M-component detectable by immunofixation but not |
| | on electrophoresis, or |
| | ≥90% reduction in serum M-component plus urine M-component < |
| | 100 mg/24 hours |
| | In addition to the above criteria, if present at baseline >90% reduction |
| | in the SPD compared with baseline for soft-tissue plasmacytoma |
| PR | ≥50% reduction of serum M-protein and reduction in 24-hour urinary |
| | M-protein by ≥90% or to <200 mg/24 hours |
| | If serum and urine M-protein are not measurable, a decrease ≥50% in |
| | the difference between involved and uninvolved FLC levels is required in |
| | place of the M-protein criteria |
| | If serum and urine M-protein are not measurable, and serum free light |
| | assay is also not measurable, ≥50% reduction in bone marrow PCs is required |
| | in place of M-protein, provided baseline percentage was ≥30% |
| | In addition to the above criteria, if present at baseline, ≥50% reduction |
| | in the size (SPD) of soft-tissue plasmacytomas is also required |
| Minimal | ≥25% but ≤49% reduction of serum M-protein and reduction in 24- |
| response | hour urine M-protein by 50% to 89% |
| | In addition to the above criteria, if present at baseline, ≥50% reduction |
| | in the size (SPD) of soft-tissue plasmacytomas is also required |
| Stable | Not meeting criteria for sCR, CR, VGPR, PR, MR, or PD |
| disease | |
| PD^{c} | Any 1 or more of the following criteria: |
| | Increase of 25% from lowest response value in any of the following: |
| | Serum M-component (absolute increase must be ≥0.5 g/dL), and/or |
| | Urine M-component (absolute increase must be ≥200 mg/24 hours), and/or |
| | Only in participants without measurable serum and urine M-protein |
| | levels: the difference between involved and uninvolved FLC levels (absolute |
| | increase must be >10 mg/dL) |
| | Only in participants without measurable serum and urine M-protein |
| | levels and without measurable disease by FLC levels, bone marrow PC |
| | percentage (absolute increase must be ≥10%) |
| | Appearance of a new lesion(s), ≥50% increase from nadir in SPD of >1 |
| | lesion, or ≥50% increase in the longest diameter of a previous lesion >1 |
| | cm in short axis |
| | Definite development of new bone lesions or definite increase in the |
| | size of existing bone lesions |
| | ≥50% increase in circulating PCs (minimum of 200 cells/μL) if this is |
| | the only measure of disease |

NOTE:
All response categories (CR, sCR, VGPR, PR, MR, and PD) require 2 consecutive assessments made at any time before the institution of any new therapy; CR, sCR, VGPR, PR, MR, and stable disease categories also require no known evidence of progressive or new bone lesions if radiographic studies were performed. VGPR and CR categories require serum and urine studies regardless of whether disease at baseline was measurable on serum, urine, both, or neither. Bone marrow assessments need not be confirmed.
NOTE:
To qualify for VGPR or PR/minimal response, the sum of products of the perpendicular diameters of the existing extramedullary plasmacytomas must have decreased by >90% or ≥50%, respectively, and new plasmacytomas must not have developed. To qualify for disease progression, either the sum of products of the perpendicular diameters of the existing extramedullary plasmacytomas must have increased by ≥50%, or the longest diameter of previous lesion >1 cm in short axis must have increased ≥50%, or a new plasmacytoma must have developed. When not all existing extramedullary plasmacytomas are reported, but the sum of products of the perpendicular diameters of the reported plasmacytomas have increased by ≥50%, then the criterion for disease progression is met.
Radiographic studies are not required to satisfy these response requirements. For PD, serum M-component increases of ≥1 g/dL are sufficient to define relapse if lowest M-component is ≥5 g/dL.
^{a}Clarifications to the criteria for coding CR and VGPR in participants in whom the only measurable disease is by serum FLC levels: CR in such participants indicates a normal FLC ratio of 0.26 to 1.65 (or reference range in testing laboratory) in addition to CR criteria listed above. VGPR in such participants requires a ≥90% decrease in the difference between involved and uninvolved FLC levels.
^{b}In some cases, it is possible that the original M-protein light chain isotype is still detected on immunofixation but the accompanying heavy-chain component has disappeared; this would not be considered as a CR even though the heavy-chain component is not detectable, as it is possible that the clone evolved to one that secreted only light chains. Thus, if a participant has IgA lambda myeloma, then to qualify as CR there should be no IgA detectable on serum or urine immunofixation; if free lambda is detected without IgA, then it must be accompanied by a different heavy-chain isotype (IgG, IgM, etc).
^{c}Clarifications to the criteria for coding PD: bone marrow criteria for PD are to be used only in participants without measurable disease by M-protein and by FLC levels; “25% increase” refers to M-protein, and FLC, and does not refer to bone lesions, or soft-tissue plasmacytomas and the “lowest response value” does not need to be a confirmed value.
Source: Adapted from Durie 2015, Rajkumar 2011, and Kumar 2016.

TABLE 10A
Schedule of Activities for Study Procedures / Assessments - Cohort 1 in RRMM.

| | Assessments | | |
|---|---|---|---|
| | Screening | Apheresis | Conditioning Regimen |
| | Day | | |
| | | | −5*, −4, −3 |
| | | 7 to 14 days after | Window |
| | ≤28 d | enrollment | * for start D −7 to D −5 |
| Informed consent | X | | |
| Eligibility criteria | X | | |
| Eligibility for apheresis, | | X | X |
| conditioning regimen, | | | |
| and cilta-cel infusion | | | |
| Medical history | X | | |
| Cytogenetics | X | | |
| ECOG PS score | X | | X (Pre-1^{st} dose) |
| Weight | X | | X (Pre-1^{st} dose) |
| Vital signs including | X | X | X |
| oxygen saturation | | | |
| Temperature | X | | |
| Handwriting test | | | |
| Neurologic exam | | | |
| including ICE Tool | | | |
| Physical exam, height | X (complete | | Symptom-directed exam as clinically |
| | exam) | | indicated |
| 12-lead ECG | X | | |
| Echocardiogram or | X (≤8 wks | If clinically | If receiving bridging therapy, including |
| MUGA scan | prior | indicated | agents with known cardiac toxicity (per |
| | to apheresis) | | prescribing information), repeat |
| | | | assessment of cardiac function after |
| | | | completion of bridging therapy and |
| | | | prior to start of conditioning regimen; |
| | | | repeat assessment of cardiac function |
| | | | further, as clinically indicated |

Laboratory Assessments

| | | | |
|---|---|---|---|
| Hematology | X | Pre-apheresis | X (Pre-1^{st} dose) |
| | | (same day) | |
| Chemistry | X | X (≤72 h) | X (Pre-1^{st} dose) |
| Infectious disease | | X (≤8 wks pre- | As clinically indicated |
| testing | | apheresis) | |
| Serology | X | | As clinically indicated. For participants |
| | | | at risk of HBV reactivation, perform |
| | | | PCR testing |
| Coagulation | X | | As clinically indicated (including at |
| | | | CRS onset if applicable) |
| Serum or urine | X (serum) | X (≤72 h) | X (≤72 h pre-1^{st} dose) |
| pregnancy test | | | |
| (POCBP) | | | |

Disease Evaluations: Blood and 24-hour urine to be sent to the central laboratory (Section LII).
Local laboratory may be used in extenuating circumstances. Disease evaluations should continue
until confirmed PD, death, or start of a new antimyeloma treatment.

| | | |
|---|---|---|
| Serum β2- | X | X (pre-1^{st} dose [≤7 d]) |
| microglobulin | | |
| Quantitative | X | |
| Immunoglobulins^{b} | | |
| SPEP | X | |
| 24-hour UPEP | X^{c} | |
| Serum FLC and | X | |
| SIFE/UIFE | | |
| DSIFE | | To confirm a VGPR or better in |
| | | participants with IgG kappa myeloma |
| | | when daratumumab interference is |
| | | suspected based on SPEP and SIFE |
| | | results. |
| Skeletal Survey^{d} | X | |
| Assessment of STP | X | |
| Bone marrow aspirate | X | At time of suspected CR/sCR and PD |
| (if dry tap, BMB can be | | (prior to SST). At 6 months and 12 |
| used)^{e} | | months from suspected CR/sCR. |

PRO Assessments: Should be completed before any clinical tests, procedures, or other consultations

| | |
|---|---|
| PRO-CTCAE | |
| EORTC-Q168 | |
| PGI-S | X |

Ongoing Review

| | |
|---|---|
| aEs and concomitant | Continuous from the time of signing of ICF until Day 112 post cilta-cel |
| medications | infusion or 30 days after last dose of talquetamab. |
| SPM | Second primary malignancies should be collected and followed from the time |
| | of ICF signing to study completion |
| Survival status | Continuous from the time of signing of ICF to study completion |

^{a}Immediately before the start of infusion, at the end of infusion, and 0.5, 1, 2 hours after end of infusion. Monitor until normalized after a CRS event.
^{b}All participants will be evaluated for IgG, IgA, IgM. Testing for IgD and IgE will only be performed for participants with IgD and IgE-type myeloma.
^{c}UPEP sample collected as part of the standard of care and prior to the participant signing ICF may be used for analysis at the central laboratory.
^{d}If performed within 42 days before enrollment, results from screening may be used without these tests being repeated.
^{e}If bone marrow aspirate collected during treatment to evaluate MRD-negativity is deemed of low quality and/or yields no results, another fresh bone marrow aspirate may be requested.

TABLE 10B
Schedule of Activities for Study Procedures/Assessments - Cohort 1 in RRMM.

| | Assessments | | | | | | | |
|---|---|---|---|---|---|---|---|---|
| | Cilta-cel | Post infusion (any participant who received an infusion | | | | | Talquetamab | Follow- |
| | Infusion | of cilta-cel should continue all subsequent assessments) | | | | | Consolidation | Up |
| | Day | | | | | | | |
| | | | 7, 10, | | | | Refer to Tables | |
| | 1 | 3 | 14, 21 | 28 | 42 | 56 | Ca-Cb for SoA | |
| | Window | | | | | | | |
| | | | ±1 d | ±2 d | ±2 d | ±2 d | | |
| Informed consent | | | | | | | | |
| Eligibility criteria | | | | | | | | |
| Eligibility for | X (Table 14) | | | | | | | |
| apheresis, | | | | | | | | |
| conditioning | | | | | | | | |
| regimen, and | | | | | | | | |
| cilta-cel infusion | | | | | | | | |
| Medical history | | | | | | | | |
| Cytogenetics | | | | | | | | |
| ECOG PS score | X | | | | | X | | |
| Weight | X | | | | | | | |
| Vital signs | X a | | | | | | | |
| including oxygen | | | | | | | | |
| saturation | | | | | | | | |
| Temperature | | | | | | | | |
| Handwriting test | ≤24 h | X | X | X | X | X | | |
| | predose | | | | | | | |
| Neurologic exam | ≤24 h | As clinically indicated | | | | | | |
| including ICE | predose | | | | | | | |
| Tool | | | | | | | | |
| Physical exam, | Symptom directed exam as clinically indicated | | | | | | | |
| height | | | | | | | | |

| | |
|---|---|
| 12-lead ECG | As clinically indicated |

| | |
|---|---|
| Echocardiogram | If receiving bridging therapy, including agents with |
| or MUGA scan | known cardiac toxicity (per prescribing information), |

repeat assessment of cardiac function after completion
of bridging therapy and prior to start of conditioning
regimen; repeat assessment of cardiac function further,
as clinically indicated

Laboratory Assessments

| Hematology | X (predose) | X | X | X | X | X |
|---|---|---|---|---|---|---|
| Chemistry | X (predose) | X | X | X | X | X |
| Infectious disease | As clinically indicated | | | | | |
| testing | | | | | | |
| Serology | As clinically indicated. For participants at risk | | | | | |
| | of HBV reactivation, perform PCR testing | | | | | |
| Coagulation | As clinically indicated (including at CRS onset if | | | | | |
| | applicable) | | | | | |
| Serum or urine | As clinically indicated | | | | | |
| pregnancy test | | | | | | |
| (POCBP) | | | | | | |

Disease Evaluations: Blood and 24-hour urine to be sent to the central laboratory (Section LII).
Local laboratory may be used in extenuating circumstances. Disease evaluations should continue
until confirmed PD, death, or start of a new antimyeloma treatment.

| | | |
|---|---|---|
| Serum β2- | | |
| microglobulin | | |
| Quantitative | X | X |
| Immunoglobulins^{b} | | |
| SPEP | X | X |
| 24-hour UPEP | X | X |

| Serum FLC and | When CR is suspected or maintained; for participants with measurable disease |
|---|---|
| SIFE/UIFE | only by light chain criteria, perform serum FLC at every assessment when an |
| | SPEP is performed |
| DSIFE | To confirm a VGPR or better in participants with IgG kappa myeloma when |
| | daratumumab interference is suspected based on SPEP and SIFE results. |
| Skeletal Survey^{d} | As clinically indicated to document disease progression or response. |
| Assessment of | For participants with history of STP until confirmed CR, PD or start of SST For |
| STP | assessment by physical exam (if applicable), Q4W (±7 days); from C1D1 For |
| | assessment by radiology, on D 56 and then Q12W (±7 days) from C1D1 As |
| | clinically indicated for other participants |
| Bone marrow | At time of suspected CR/sCR and PD (prior to SST). At 6 months and 12 months |
| aspirate (if dry | from suspected CR/sCR. |
| tap, BMB can be | |
| used)^{e} | |

PRO Assessments: Should be completed before any clinical
tests, procedures, or other consultations

| | |
|---|---|
| PRO-CTCAE | |
| EORTC-Q168 | X |
| PGI-S | X |

Ongoing Review

| | |
|---|---|
| aEs and | Continuous from the time of signing of ICF until Day 112 post cilta-cel infusion |
| concomitant | or 30 days after last dose of talquetamab. |
| medications | |
| SPM | Second primary malignancies should be collected and followed from the time of |
| | ICF signing to study completion |
| Survival status | Continuous from the time of signing of ICF to study completion |

a. Immediately before the start of infusion, at the end of infusion, and 0.5, 1, 2 hours after end of infusion. Monitor until normalized after a CRS event.
^{b}All participants will be evaluated for IgG, IgA, IgM. Testing for IgD and IgE will only be performed for participants with IgD and IgE-type myeloma.
c. UPEP sample collected as part of the standard of care and prior to the participant signing ICF may be used for analysis at the central laboratory.
^{d}If performed within 42 days before enrollment, results from screening may be used without these tests being repeated.
^{e}If bone marrow aspirate collected during treatment to evaluate MRD-negativity is deemed of low quality and/or yields no results, another fresh bone marrow aspirate may be requested.

TABLE 11A
Schedule of Activities for Study Procedures/Assessments - Cohort 2 in NDMM.

| | Assessments | | | | | | |
|---|---|---|---|---|---|---|---|
| | Screening | Apheresis | DRd Induction Treatment (Cycles 1 to 4) (Tables 15A-15B) | | | | Conditioning Regimen |
| | Day | | | | | | |
| | | 7 to 14 days | | | | | |
| | 28 d | after enrollment | 1 | 8 | 15 | 22 | −5*, −4, −3 |
| | Window | | | | | | |
| | | | | | | | * for start |
| | | | ±1 d | ±1 d | ±1 d | ±1 d | D −7 to D −5 |
| Informed consent | X | | | | | | |
| Eligibility criteria | X | | | | | | |
| Eligibility for | | X | | | | | X |
| apheresis, | | | | | | | |
| conditioning | | | | | | | |
| regimen, and cilta- | | | | | | | |
| cel infusion | | | | | | | |
| Medical history | X | | | | | | |
| Cytogenetics | X | | | | | | |
| ECOG PS score | X | | C3D1 | | | | X (Pre-1^{st} dose) |
| Weight | X | | X | | | | X (Pre-1^{st} dose) |
| Vital signs | X | X | Q1W in C1 to 2, Q2Q | | | | X |
| including oxygen | | | thereafter | | | | |
| saturation | | | | | | | |
| Temperature | X | | | | | | |
| Handwriting test | | | | | | | |
| Neurologic exam | | | | | | | |
| including ICE Tool | | | | | | | |
| Physical exam, | X (complete | | | | | | Symptom- |
| height | exam) | | | | | | directed exam as |
| | | | | | | | clinically |
| | | | | | | | indicated |

| | | |
|---|---|---|
| 12-lead ECG | X | As clinically indicated |

| | | |
|---|---|---|
| Echocardiogram or | X (≤8 wks | As clinically indicated. Monitor participants for clinical signs or |
| MUGA scan | prior | symptoms of cardiac failure or cardiac ischemia. Evaluate |
| | to apheresis) | promptly if cardiac toxicity is suspected |

Laboratory Assessments

| Hematology | X | Pre- | Q1W in C1 to 2, Q2W | | X (Pre-1^{st} dose) |
|---|---|---|---|---|---|
| | | apheresis | thereafter | | |
| | | (same day) | | | |
| Chemistry | X | X (≤72 h) | Q2W C1 to C4 | | X (Pre-1^{st} dose) |
| Infectious disease | | X (≤8 wks | As clinically indicated | | |
| testing | | pre- | | | |
| | | apheresis) | | | |
| Serology | X | | C3D1 | As clinically indicated. For participants at | |
| | | | | risk of HBV reactivation, perform PCR | |
| | | | | testing | |
| Coagulation | X | | | | As clinically |
| | | | | | indicated |
| | | | | | (including at |
| | | | | | CRS onset if |
| | | | | | applicable) |
| Serum or urine | X (serum) | X (≤72 h) | X^{b} | | X (≤72 h pre-1^{st} |
| pregnancy test | | | | | dose) |
| (POCBP) | | | | | |

Disease Evaluations: Blood and 24-hour urine to be sent to the central laboratory (Section LII).
Local laboratory may be used in extenuating circumstances. Disease evaluations should continue
until confirmed PD, death, or start of a new antimyeloma treatment.

| Serum β2- | X | | | X (pre-1^{st} dose |
|---|---|---|---|---|
| microglobulin | | | | [≤7 d]) |
| Quantitative | X | X | Q4W (±7 days) | |
| Immunoglobulins^{c} | | | after 1^{st} dose | |
| SPEP | X | | | |
| 24-hour UPEP | X^{d} | X | | |

| | | | |
|---|---|---|---|
| Serum FLC and | X | X | When CR is |
| SIFE/UIFE | | | suspected or |
| | | | maintained; for |
| | | | participants with |
| | | | measurable disease |
| | | | only by light chain |
| | | | criteria, perform |
| | | | serum FLC at every |
| | | | assessment when an |
| | | | SPEP is performed. |

| DSIFE | | To confirm a VGPR or better | To confirm a |
|---|---|---|---|
| | | in participants with IgG | VGPR or better |
| | | kappa myeloma when | in participants |
| | | daratumumab interference is | with IgG kappa |
| | | suspected | myeloma when |
| | | based on SPEP and SIFE | daratumumab |
| | | results. | interference is |
| | | | suspected based |
| | | | on SPEP and |
| | | | SIFE results. |
| Skeletal Survey^{e} | X | As clinically indicated to | |
| | | document disease progression | |
| | | or response. | |
| Assessment of STP | X | For participants with a history | |
| | | of STP until confirmed CR, | |
| | | PD, or start of SST | |
| | | For assessment by physical | |
| | | exam (if applicable), Q4W | |
| | | (±7 days) from C1D1 | |
| | | For assessment by radiology, | |
| | | on D 56 and then Q12W (±7 | |
| | | days) from C1D1 | |
| | | As clinically indicated for | |
| | | other participants | |
| Bone marrow | X | At time of suspected CR/sCR | At time of |
| aspirate (if dry tap, | | and PD (prior to SST). At 6 | suspected |
| BMB can be used)^{f} | | months and 12 months from | CR/sCR and PD |
| | | suspected CR/sCR | (prior to SST). At |

6 months and 12
months from
suspected
CR/sCR.

PRO Assessments: Should be completed before any clinical
tests, procedures, or other consultations

| | |
|---|---|
| PRO-CTCAE | |
| EORTC-Q168 | |
| PGI-S | X |

Ongoing Review

| aEs and | Continuous from the time of signing of ICF until Day 112 post cilta-cel infusion |
|---|---|
| concomitant | or 30 days after last dose of talquetamab. |
| medications | |

| | |
|---|---|
| SPM | Second primary malignancies should be collected and followed from the time of |
| | ICF signing to study completion |
| Survival status | Continuous from the time of signing of ICF to study completion |

a. Immediately before the start of infusion, at the end of infusion, and 0.5, 1, 2 hours after end of infusion. Monitor until normalized after a CRS event.
^{b}Within 24 hrs prior to the first dose of DRd, every week for the first 4 weeks and then every 28 days for POCBP with regular menstrual cycles or every 14 days for POCBP with irregular menstrual cycles. Additional testing as clinically indicated and/or consistent with any country/territory-specific requirements per local prescribing information for lenalidomide.
^{c}All participants will be evaluated for IgG, IgA, IgM. Testing for IgD and IgE will only be performed for participants with IgD and IgE-type myeloma.
^{d}UPEP sample collected as part of the standard of care and prior to the participant signing ICF may be used for analysis at the central laboratory.
^{e}If performed within 42 days before enrollment, results from screening may be used without these tests being repeated.
^{f}If a bone marrow aspirate collected during treatment to evaluate MRD-negativity is deemed of low quality and/or yields no results, another fresh bone marrow aspirate may be requested.

TABLE 11B
Schedule of Activities for Study Procedures/Assessments - Cohort 2 in NDMM.

| Assessments | | | |
|---|---|---|---|
| Cilta-cel | Post infusion (any participant who received an infusion of | Talquetamab | Follow- |
| Infusion | cilta-cel should continue all subsequent assessments) | Consolidation | Up |

| | Day | | | | | | |
|---|---|---|---|---|---|---|---|
| | | | | | | | Refer to Tables |
| | 1 | 3 | 7, 10, 14, 21 | 28 | 42 | 56 | 5A-5B for SoA |
| | Window | | | | | | |
| | | | ±1 d | ±2 d | ±2 d | ±2 d | |
| Informed consent | | | | | | | |
| Eligibility criteria | | | | | | | |
| Eligibility for | X (Table 14) | | | | | | |
| apheresis, | | | | | | | |
| conditioning | | | | | | | |
| regimen, and | | | | | | | |
| cilta-cel infusion | | | | | | | |
| Medical history | | | | | | | |
| Cytogenetics | | | | | | | |
| ECOG PS score | X | | | | | X | |
| Weight | X | | | | | | |
| Vital signs | X^{a} | | | | | | |
| including oxygen | | | | | | | |
| saturation | | | | | | | |
| Temperature | | | | | | | |
| Handwriting test | ≤24 h | X | X | X | X | X | |
| | predose | | | | | | |
| Neurologic exam | ≤24 h | As clinically indicated | | | | | |
| including ICE | predose | | | | | | |
| Tool | | | | | | | |
| Physical exam, | Symptom directed exam as clinically indicated | | | | | | |
| height | | | | | | | |

| | |
|---|---|
| 12-lead ECG | As clinically indicated |
| Echocardiogram | As clinically indicated. Monitor participants for |
| or MUGA scan | clinical signs or symptoms of cardiac failure or |
| | cardiac ischemia. Evaluate promptly if cardiac |
| | toxicity is suspected |

Laboratory Assessments

| Hematology | X (predose) | X | X | X | X | X |
|---|---|---|---|---|---|---|
| Chemistry | X (predose) | X | X | X | X | X |
| Infectious disease | As clinically indicated | | | | | |
| testing | | | | | | |
| Serology | As clinically indicated. For participants at risk of HBV | | | | | |
| | reactivation, perform PCR testing | | | | | |
| Coagulation | As clinically indicated (including at | | | | | |
| | CRS onset if applicable) | | | | | |
| Serum or urine | As clinically indicated | | | | | |
| pregnancy test | | | | | | |
| (POCBP) | | | | | | |

Disease Evaluations: Blood and 24-hour urine to be sent to the central laboratory (Section LII).
Local laboratory may be used in extenuating circumstances. Disease evaluations should continue
until confirmed PD, death, or start of a new antimyeloma treatment.

| Serum β2- | | | |
|---|---|---|---|
| microglobulin | | | |
| Quantitative | | X | X |
| Immunoglobulins^{c} | | | |
| SPEP | | X | X |
| 24-hour UPEP | | X | X |
| Serum FLC and | When CR is suspected or maintained; for participants with measurable disease | | |
| SIFE/UIFE | only by light chain criteria, perform serum FLC at every assessment when an | | |
| | SPEP is performed | | |
| DSIFE | To confirm a VGPR or better in participants with IgG kappa myeloma when | | |
| | daratumumab interference is suspected based on SPEP and SIFE results. | | |
| Skeletal Survey^{e} | As clinically indicated to document disease progression or response. | | |
| Assessment of | For participants with history of STP until confirmed CR, PD or start of SST For | | |
| STP | assessment by physical exam (if applicable), Q4W (±7 days); from C1D1 For | | |
| | assessment by radiology, on D 56 and then Q12W (±7 days) from C1D1 As | | |
| | clinically indicated for other participants | | |
| Bone marrow | At time of suspected CR/sCR and PD (prior to SST). At 6 months and 12 months | | |
| aspirate (if dry | from suspected CR/sCR. | | |
| tap, BMB can be | | | |
| used)^{f} | | | |

PRO Assessments: Should be completed before any clinical
tests, procedures, or other consultations

| | |
|---|---|
| PRO-CTCAE | |
| EORTC-Q168 | X |
| PGI-S | X |

Ongoing Review

| | |
|---|---|
| aEs and | Continuous from the time of signing of ICF until Day 112 post cilta-cel infusion |
| concomitant | or 30 days after last dose of talquetamab. |
| medications | |
| SPM | Second primary malignancies should be collected and followed from the time of |
| | ICF signing to study completion |
| Survival status | Continuous from the time of signing of ICF to study completion |

^{a}Immediately before the start of infusion, at the end of infusion, and 0.5, 1, 2 hours after end of infusion. Monitor until normalized after a CRS event.
^{c}All participants will be evaluated for IgG, IgA, IgM. Testing for IgD and IgE will only be performed for participants with IgD and IgE-type myeloma.
d. UPEP sample collected as part of the standard of care and prior to the participant signing ICF may be used for analysis at the central laboratory.
^{e}If performed within 42 days before enrollment, results from screening may be used without these tests being repeated.
^{f}If a bone marrow aspirate collected during treatment to evaluate MRD-negativity is deemed of low quality and/or yields no results, another fresh bone marrow aspirate may be requested.

TABLE 12
Schedule of Activities for Study Procedures/Assessments During Talquetamab
Consolidation Treatment Phase- Cohort 1 in RRMM and Cohort 2 in NDMM.

| | Assessments | | | | | | | | | |
|---|---|---|---|---|---|---|---|---|---|---|
| | | Talquetamab Consolidation (28-day cycle) | | | | | | | | Follow- |
| | | Cycle 1 | | | | Cycles 2 to 6 | | Cycle 7+ | EOT | Up |
| | | Day | | | | | | | | |
| | | | | | | | | | 30 d after | |
| | | 1 | 4 | 8 | 15 | 1 | 15 | 1 | last dose | Q4W |
| | | Window | | | | | | | | |
| | Notes | | +2 d | +2 d | ±2 d | ±3 d | ±3 d | ±3 d | +7 d | ±7 d |
| ECOG | | X | | | | X | | X | X | |
| performance | | | | | | | | | | |
| status | | | | | | | | | | |
| Physical | Symptom- | As clinically indicated | | | | | | | | |
| examination | directed | | | | | | | | | |
| Neurologic exam, | | X | As clinically indicated | | | | | | | |
| including ICE | | | | | | | | | | |
| Tool | | | | | | | | | | |
| Weight | Use weight | X | | | | X | | X | | |
| | to dose per | | | | | | | | | |
| | IPPI | | | | | | | | | |
| Vital signs | | X | X | X | X | X | X | X | X | |
| including oxygen | | | | | | | | | | |
| saturation | | | | | | | | | | |
| Handwriting test | | X | | | | X | | X | X | X (monthly |
| | | | | | | | | | | for 2 years |
| | | | | | | | | | | then Q2 |
| | | | | | | | | | | months |
| | | | | | | | | | | until study |
| | | | | | | | | | | completion) |

| | |
|---|---|
| 12-lead ECG | As clinically indicated |

Laboratory Assessments

| Hematology | Must meet | X | X | X | X | X | X | X | X |
|---|---|---|---|---|---|---|---|---|---|
| | criteria | | | | | | | | |
| | within 72 h | | | | | | | | |
| | prior to | | | | | | | | |
| | C1D1. | | | | | | | | |
| Chemistry | | X | X | | X | X | | X | X |
| Serology | | PCR testing Q12W (±7 d) from C1D1, for up to 6 months after | | | | | | | |
| | | last dose of study treatment if history of HBV and HCV | | | | | | | |
| | | infection. | | | | | | | |
| Infectious disease | | As clinically indicated | | | | | | | |
| testing | | | | | | | | | |

| | | | |
|---|---|---|---|
| Coagulation | CRS or if otherwise indicated | | |
| Serum or urine | Within 24 h of C1D1, thereafter, on D 1 of | X | X (until |
| pregnancy test | each cycle (D 1 and D 15 for women with | | 100 days |
| (POCBP) | irregular menses); additional testing as | | after last |
| | clinically indicated | | dose) |

Disease Evaluations

| Quantitative | Collection | X | X | X | X (until PD |
|---|---|---|---|---|---|
| immunoglobulins^{a} | of all | | | | or start of |
| | samples | | | | SST) |
| SPEP | to be | X | X | X | X (until PD |
| | completed | | | | or start of |
| | predose on | | | | SST) |
| 24-hour UPEP | C1D1. | X | X | X | X (until PD |
| | | | | | or start of |
| | | | | | SST) |
| Serum FLC and | | If measurable disease only by light chain: D 1 of each cycle and, | | | |
| SIFE/UIFE | | if applicable, each pre-PD disease evaluation visit | | | |
| | | until PD or start of SST. | | | |
| | | If measurable disease by serum and/or urine M spike: at time of | | | |
| | | suspected CR or sCR. | | | |
| DSIFE | | To confirm a VGPR or better in participants with IgG kappa | | | |
| | | myeloma when daratumumab interference is suspected based on | | | |
| | | SPEP and SIFE results. | | | |
| Skeletal survey^{b} | | As clinically indicated to document response or disease | | | |
| | | progression or start of SST | | | |
| Assessment of | | For participants with a history of STP until development of | | | |
| STP | | confirmed CR, PD, or start of SST | | | |
| | | For assessment by physical examination (if | | | |
| | | applicable), Q4W (±7 d) from C1D1 | | | |
| | | For assessment by radiology, Q12W (±7 d) from C1D1 | | | |
| | | As clinically indicated for other participants | | | |
| Bone marrow | If dry tap, | At time of suspected CR/sCR and PD (prior to SST). At 6 months | | | |
| aspirate ^{c} | BMB can be | and 12 months from suspected CR/sCR | | | |
| | used | | | | |

PRO Assessments: Should be completed before any clinical
tests, procedures, or other consultations

| | | | | | | | | | |
|---|---|---|---|---|---|---|---|---|---|
| PRO-CTCAE | | X | X | X | X | X | X | X | X |
| | | | | | | | | | (Q16W + |
| | | | | | | | | | 14 d) |
| EORTC Q168 | | X | X | X | X | | X | X | |
| PGI-S | Continue to | X | | | X | | X | X | |
| | be collected | | | | | | | | |
| | even if | | | | | | | | |
| | subsequent | | | | | | | | |
| | antimyeloma | | | | | | | | |
| | therapy | | | | | | | | |
| | is begun | | | | | | | | |

Ongoing Review

| aEs and | Continuous from the time of signing of ICF until Day 112 post |
|---|---|
| concomitant | cilta-cel infusion or 30 days after last dose of talquetamab |
| therapy | |

| | |
|---|---|
| SPM | Second primary malignancies should be collected and followed from the time of |
| | ICF signing to study completion. |
| Survival status | Continuous from the time of signing of ICF to study completion |

^{a}All participants will be evaluated for IgG, IgA, IgM. Testing for IgD and IgE will only be performed for participants with IgD- and IgE-type myeloma.
^{b}If performed within 42 days before enrollment, results from screening may be used without these tests being repeated.
^{c} If a bone marrow aspirate collected during treatment to evaluate MRD-negativity is deemed of low quality and/or yields no results, another fresh bone marrow aspirate may be requested.

TABLE 13A
Schedule of Activities for Study Procedures / Assessments - Cohort 3 in RRMM.

| Assessment | | | |
|---|---|---|---|
| | Apheresis | | |
| | (before and | | |
| | after Tal | | Conditioning |
| Screening | bridging)^{a} | Tal Bridging | Regimen |
| Day | | | |
| | | | −5*, −4, −3 |
| | 7 to 14 days | | Window |
| | after | Refer to | * for start: |
| ≤28 d | enrollment | Tables 7A-7B | D −7 to D −5 |

| | | | |
|---|---|---|---|
| Informed consent | X | | |
| Eligibility criteria | X | | |
| Eligibility for | | X | X |
| apheresis, conditioning | | | |
| regimen, and cilta-cel | | | |
| infusion | | | |
| Medical history | X | | |
| Cytogenetics | X | | |
| ECOG PS score | X | | X (Pre-1^{st} dose) |
| Weight | X | | X (Pre-1^{st} dose) |
| Vital signs including | X | X | X |
| O2 saturation | | | |
| Temperature | X | | |
| Handwriting test | | | |
| Neurologic exam | | | |
| including ICE Tool | | | |
| Physical exam, height | X | | Symptom-directed |
| | (complete | | exam as clinically |
| | exam) | | indicated |

| | | | |
|---|---|---|---|
| 12-lead ECG | X | | |
| Echocardiogram or | X (≤8 wks prior | If clinically | If receiving bridging therapy, |
| MUGA scan | to apheresis) | indicated | including agents with known cardiac |
| | | | toxicity (per prescribing information), |
| | | | repeat assessment of cardiac function |
| | | | after completion of bridging therapy |
| | | | and prior to start of conditioning |
| | | | regimen; repeat assessment of cardiac |
| | | | function further, as clinically |
| | | | indicated |

Laboratory Assessments

| Hematology | X | Pre-apheresis | | X (Pre-1^{st} dose) |
|---|---|---|---|---|
| | | (same day) | | |
| Chemistry | X | X | | X (Pre-1^{st} dose) |
| | | [≤72 h] | | |
| Infectious disease | | X | As clinically indicated | |
| testing | | [≤8 wks prior | | |
| | | to apheresis] | | |
| Serology | X | | As clinically indicated. For | |
| | | | participants at risk of HBV | |
| | | | reactivation, perform PCR testing | |
| Coagulation | X | | | |
| Serum or urine | X | X | | X (≤72 h pre-1^{st} |
| pregnancy test | (serum) | (≤72 h) | | dose) |
| (POCBP) | | | | |

Disease Evaluations: Blood and 24-hour urine to be sent to the central laboratory. Local
laboratory may be used in extenuating circumstances. Disease
Evaluations should continue until confirmed PD, death or start of a new antimyeloma treatment.

| | | |
|---|---|---|
| Serum β2- | X | X (pre-1^{st} dose [≤7 d]) |
| microglobulin | | |
| Quantitative | X | |
| immunoglobulins^{d} | | |
| SPEP | X | |
| 24-hour UPEP | X^{e} | |
| Serum FLC and | X | |
| SIFE/UIFE | | |
| DSIFE | | To confirm a VGPR or better in |
| | | participants with IgG kappa myeloma |
| | | when daratumumab interference is |
| | | suspected based on SPEP and SIFE |
| | | results. |
| Skeletal survey^{f} | X | |
| Assessment of STP | X | |
| (Section 8.2.5) | | |

| | | |
|---|---|---|
| Bone marrow aspirate | X | At time of suspected CR/sCR and PD |
| (If dry tap, BMB can be | | (prior to SST). At 6 months and 12 |
| used)g | | months from suspected CR/sCR |

PRO ASSESSMENTS: Should be completed before any clinical tests, procedures, or consultations

| | | |
|---|---|---|
| PRO-CTCAE | | X |
| EORTC-Q168 | | X |
| PGI-S | X | X |

ONGOING REVIEW

| | |
|---|---|
| aEs and concomitant | Continuous from the time of signing of ICF until Day 112 |
| medications | post cilta-cel infusion or 30 days after last dose of |
| | talquetamab. |

| | |
|---|---|
| SPM | Second primary malignancies should be collected and followed from the |
| | time of ICF signing to study completion. |
| Survival status | Continuous from the time of signing of ICF to study completion. |

^{a}The first 5 enrolled participants will undergo a second apheresis after bridging therapy.
^{b}For participants who discontinue before Day 100, the Day 100 assessments should be performed prior to withdrawal if feasible. Participants who discontinue after Day 100 but before study completion should have urine and serum disease assessment performed prior to withdrawal, if feasible, at the time of discontinuation, unless performed within 14 days prior to discontinuation. Continue assessments monthly for 2 years then Q2 months thereafter until study completion, defined as 2 years after the last participant has received their dose of cilta-cel.
^{c}Immediately before the start of infusion, at the end of infusion, and 0.5, 1, 2 hours after end of infusion. Monitor until normalized after a CRS event.
^{d}All participants will be evaluated for IgG, IgA, IgM. Testing for IgD and IgE will only be performed for participants with IgD- and IgE-type myeloma.
^{e}UPEP sample collected as part of the standard of care and prior to the participant signing ICF may be used for analysis at the central laboratory.
^{f}If performed within 42 days before enrollment, results from screening may be used without these tests being repeated.
gIf a bone marrow aspirate collected during treatment to evaluate MRD-negativity is deemed of low quality and/or yields no results, another fresh bone marrow aspirate may be requested.

TABLE 13B
Schedule of Activities for Study Procedures/Assessments - Cohort 3 in RRMM.

| | Assessment | | | | | | | |
|---|---|---|---|---|---|---|---|---|
| | Cilta-cel | Post-infusion (any participant who received an infusion | | | | | | |
| | Infusion | of cilta-cel should continue all subsequent assessments)^{b} | | | | | | Follow-up |
| | Day | | | | | | | |
| | | 3, 7, 10, | | | | | | D 101 until study |
| | 1 | 14, 21 | 28 | 42 | 56 | 78 | 100^{b} | completion^{b} |
| | Window | | | | | | | |
| | | ±1 d | ±2 d | ±2 d | ±2 d | ±2 d | ±2 d | ±7 d |
| Informed consent | | | | | | | | |
| Eligibility criteria | | | | | | | | |
| Eligibility for | X | | | | | | | |
| apheresis, | (Table 14) | | | | | | | |
| conditioning | | | | | | | | |
| regimen, and | | | | | | | | |
| cilta-cel infusion | | | | | | | | |
| Medical history | | | | | | | | |
| Cytogenetics | | | | | | | | |
| (Table 28) | | | | | | | | |
| ECOG PS score | X | | | | X | | X | |
| Weight | X | | | | | | | |
| Vital signs | X^{c} | | | | | | | |
| including O2 | | | | | | | | |
| saturation | | | | | | | | |
| Temperature | | | | | | | | |
| Handwriting test | ≤24 h | X | X | X | X | X | X | X |
| | predose | | | | | | | |
| Neurologic exam | | As clinically indicated | | | | | | |
| including ICE | | | | | | | | |
| Tool | | | | | | | | |
| Physical exam, | Symptom-directed exam as clinically indicated | | | | | | | |
| height | | | | | | | | |
| 12-lead ECG | | | | | X | | | |

| | |
|---|---|
| Echocardiogram | If receiving bridging therapy, including agents with known cardiac toxicity (per |
| or MUGA scan | prescribing information), repeat assessment of cardiac function after completion |
| | of bridging therapy and prior to start of conditioning regimen; repeat assessment |
| | of cardiac function further, as clinically indicated |

Laboratory Assessments

| Hematology | X | X | X | X | X | X | X |
|---|---|---|---|---|---|---|---|
| | (predose) | | | | | | |
| Chemistry | X | X | X | X | X | X | X |
| | (predose) | | | | | | |
| Infectious disease | As clinically indicated | | | | | | |
| testing | | | | | | | |
| Serology | As clinically indicated. For participants at risk of | | | | | | |
| | HBV reactivation, perform PCR testing | | | | | | |
| Coagulation | As clinically indicated (including at | | | | | | |
| | CRS onset if applicable) | | | | | | |
| Serum or urine | As clinically indicated. | | | | | | |
| pregnancy test | | | | | | | |
| (POCBP) | | | | | | | |

Disease Evaluations: Blood and 24-hour urine to be sent to the central laboratory (Section LII).
Local laboratory may be used in extenuating circumstances. Disease Evaluations
should continue until confirmed PD, death or start of a new antimyeloma treatment.

| Serum β2- | | | | | | |
|---|---|---|---|---|---|---|
| microglobulin | | | | | | |
| Quantitative | | X | X | X | X | X |
| immunoglobulins^{d} | | | | | | |
| SPEP | | X | X | X | X | X |
| 24-hour UPEP | | X | X | X | X | X |
| Serum FLC and | When CR is suspected or maintained; for participants with measurable disease only | | | | | |
| SIFE/UIFE | by light chain criteria perform serum FLC at every assessment when an SPEP is | | | | | |
| | performed | | | | | |
| DSIFE | To confirm a VGPR or better in participants with IgG kappa myeloma when | | | | | |
| | daratumumab interference is suspected based on SPEP and SIFE results. | | | | | |
| Skeletal survey^{f} | As clinically indicated to document disease progression or response. | | | | | |
| Assessment of | For participants with history of STP until confirmed CR, PD, or start of SST For | | | | | |
| STP | assessment by physical exam (if applicable), Q4W (+7 days) from C1D1 For | | | | | |
| | assessment by radiology, on Day 56 and then Q12W (+7 days) from C1D1 As | | | | | |
| | clinically indicated for other participants | | | | | |
| Bone marrow | At time of suspected CR/sCR and PD (prior to SST). At 6 months and 12 months | | | | | |
| aspirate (If dry | from suspected CR/sCR | | | | | |
| tap, BMB can be | | | | | | |
| used)^{g} | | | | | | |

PRO ASSESSMENTS: Should be completed before any
clinical tests, procedures, or consultations

| | | | | | |
|---|---|---|---|---|---|
| PRO-CTCAE | | | | | X |
| EORTC-Q168 | X | | | | |
| PGI-S | X | X | X | X | |

ONGOING REVIEW

| | |
|---|---|
| aEs and | Continuous from the time of signing of ICF until Day 112 post cilta-cel infusion |
| concomitant | or 30 days after last dose of talquetamab. |
| medications | |
| SPM | Second primary malignancies should be collected and followed from the time of |
| | ICF signing to study completion. |
| Survival status | Continuous from the time of signing of ICF to study completion. |

a. The first 5 enrolled participants will undergo a second apheresis after bridging therapy.
^{b}For participants who discontinue before Day 100, the Day 100 assessments should be performed prior to withdrawal if feasible. Participants who discontinue after Day 100 but before study completion should have urine and serum disease assessment performed prior to withdrawal, if feasible, at the time of discontinuation, unless performed within 14 days prior to discontinuation. Continue assessments monthly for 2 years then Q2 months thereafter until study completion, defined as 2 years after the last participant has received their dose of cilta-cel.
c. Immediately before the start of infusion, at the end of infusion, and 0.5, 1, 2 hours after end of infusion. Monitor until normalized after a CRS event.
^{d}All participants will be evaluated for IgG, IgA, IgM. Testing for IgD and IgE will only be performed for participants with IgD- and IgE-type myeloma.
e. UPEP sample collected as part of the standard of care and prior to the participant signing ICF may be used for analysis at the central laboratory.
^{f}If performed within 42 days before enrollment, results from screening may be used without these tests being repeated.
^{g}If a bone marrow aspirate collected during treatment to evaluate MRD-negativity is deemed of low quality and/or yields no results, another fresh bone marrow aspirate may be requested.

TABLE 14A
Schedule of Activities for Study Procedures/Assessments During
Talquetamab Bridging Treatment Phase - Cohort 3 in RRMM.

| | Assessments | | | | | | |
|---|---|---|---|---|---|---|---|
| | | Treatment Phase (28-day cycle) | | | | | |
| | | Cycle 1 | | | | Cycles 2-4 | |
| | | Day | | | | | |
| | | 1 | 4 | 8 | 15 | 1 | 15 |
| | | Window | | | | | |
| | Notes | | +2 d | +2 d | ±2 d | ±3 d | ±3 d |
| ECOG | | X | | | | X | |
| performance | | | | | | | |
| status | | | | | | | |
| Physical | Symptom- | As clinically indicated | | | | | |
| examination | directed | | | | | | |
| Neurologic exam, | | X | As clinically indicated | | | | |
| including ICE | | | | | | | |
| Tool | | | | | | | |
| Weight | Use weight | X | | | | X | |
| | to dose per | | | | | | |
| | IPPI | | | | | | |
| Vital signs | | X | X | X | X | X | X |
| including oxygen | | | | | | | |
| saturation | | | | | | | |

| | |
|---|---|
| 12-lead ECG | As clinically indicated |

LABORATORY ASSESSMENTS

| Hematology | Must meet | X | X | X | X | X | X |
|---|---|---|---|---|---|---|---|
| | criteria | | | | | | |
| | within 72 h | | | | | | |
| | prior to | | | | | | |
| | C1D1. | | | | | | |
| Chemistry | | X | X | | X | X | |
| Infectious disease | | As clinically indicated | | | | | |
| testing | | | | | | | |

| | |
|---|---|
| Serology | PCR testing every 12 wks (±7 d) from C1D1, for up to 6 months |
| | after last dose of study treatment for participants with history of |
| | HBV & HCV infection. |
| Coagulation | CRS or if otherwise indicated |
| Serum or urine | Within 24 h of C1D1, thereafter, on D 1 of each cycle (D 1 and D 15 |
| pregnancy test | for women with irregular menses); additional testing as clinically |
| (POCBP) | indicated |

Disease Evaluations: Blood and 24-hour urine to be sent to the central laboratory (Section LII).
Local laboratory may be used in extenuating circumstances. dEs should continue until confirmed
PD, death, or start of a new antimyeloma treatment.

| Serum β2- | Collection | X | |
|---|---|---|---|
| microglobulin | of all | | |
| Quantitative | samples | X | X |
| immunoglobulins^{a} | to be | | |
| SPEP | completed | X | X |
| 24-hour UPEP | predose on | X | X |
| Serum FLC and | C1D1. | If measurable disease only by light chain: D 1 of each cycle and, if | |
| SIFE/UIFE | | applicable, each pre-PD disease evaluation visit until PD of start of | |
| | | SST. | |
| | | If measurable disease by serum and/or urine M spike: at time of | |
| | | suspected CR or sCR | |
| DSIFE | | To confirm a VGPR or better in participants with IgG kappa | |
| | | myeloma when daratumumab interference is | |
| | | suspected based on SPEP and SIFE results. | |
| Skeletal survey^{b} | | As clinically indicated to document disease progression or start of | |
| | | SST or response. | |
| Assessment of | | For participants with history of STP until confirmed CR, PD or | |
| STP | | start of SST | |
| | | For assessment by physical examination (if applicable), Q4W (±7 | |
| | | d) from C1D1 | |
| | | For assessment by radiology, Q12W (±7 d) from C1D1 | |
| | | As clinically indicated for other participants | |
| Bone marrow | If dry tap, | Collect samples at suspected CR/sCR and then 6 months and 12 | |
| aspirate^{c} | BMB can be | months after. | |
| | used. | | |

PRO Assessments: Should be completed before any
clinical tests, procedures, or other consultations

| | | | | | | |
|---|---|---|---|---|---|---|
| PRO-CTCAE | | X | X | X | X | X |
| EORTC-Q168 | | X | X | X | X | |
| PGI-S | Continue to | X | | | X | |
| | be collected | | | | | |
| | even if | | | | | |
| | subsequent | | | | | |
| | antimyeloma | | | | | |
| | therapy | | | | | |
| | has begun. | | | | | |

Ongoing Review

| aEs and | Continuous from the time of signing of ICF until Day 112 post cilta-cel infusion |
|---|---|
| concomitant | or 30 days after last dose of talquetamab. |
| therapy | |

| | |
|---|---|
| SPM | Second primary malignancies should be collected and followed from the time of |
| | ICF signing to study completion |
| Survival status | Continuous from the time of signing of ICF to study completion |

^{a}All participants will be evaluated for IgG, IgA, IgM. Testing for IgD and IgE will only be performed for participants with IgD- and IgE-type myeloma.
^{b}If performed within 42 days before enrollment, results from screening may be used without these tests being repeated.
^{c}If a bone marrow aspirate collected during treatment to evaluate MRD-negativity is deemed of low quality and/or yields no results, another fresh bone marrow aspirate may be requested.

TABLE 15A
Schedule of Activities for Pharmacokinetic, Immunogenicity,
and Biomarker Assessments (Cohorts 1 and 2)^{a}.

| | Assessment | | | |
|---|---|---|---|---|
| | | | DRd | |
| | | | Induction | Conditioning |
| | | | Treatment | Regimen |
| | | | Day | |
| | | | | −5*, −4, −3 |
| | | | | Window |
| | | | | * for start: |
| | Screening | Apheresis | 1 | D −7 to D −5^{c} |
| PK CAR | | | | |
| transgene levels | | | | |
| (blood) | | | | |
| Cilta-cel ADA | | | | |
| (serum) | | | | |
| Cilta-cel/ | X | | C1D1 only^{b} | D −5 only |
| talquetamab | | | | |
| immunopheno- | | | | |
| typing (blood) | | | | |
| Cytokines (blood) | X | | Collect additional pharmacodynamic | |
| | | | cytokine samples, if feasible, when any | |
| | | | of the following are observed or | |
| | | | reported: (1) suspected sARRs reaction | |
| | | | Grade ≥2; (2) CRS event Grade ≥2 (at | |
| | | | onset of the CRS event, and 24 h and | |
| | | | 72 h after); (3) as indicated based on | |
| | | | emerging data. | |
| RCL (blood) | X | | | |
| Immune and omic | | X^{d} | | |
| profiling (PBMC)^{f} | | | | |

^{a}All samples predose (on dosing days).
^{b}For Cohort 2 only.
^{c}If using a window, collection should be taken prior to the first dose of conditioning regimen.
^{d}If a repeat apheresis is to be performed, another apheresis sample should be collected for PD assessments at the time of apheresis.
^{e}Predose sample window −4 to 0 hour of cilta-cel administration for pharmacokinetic and immunogenicity sampling.
^{f}PBMC collection taken at the time of apheresis.

TABLE 15B
Schedule of Activities for Pharmacokinetic, Immunogenicity,
and Biomarker Assessments (Cohorts 1 and 2)^{a}.

| | Assessment | | | | | | | | | | |
|---|---|---|---|---|---|---|---|---|---|---|---|
| | | | | | | | | | Talqueta | | |
| | Cilta-cel | Infusion Consolidation Post-Infusion | | | | | | | mab | Follow-Up | |
| | Day | | | | | | | | | | |
| | | | | | | | | | | | EOT/ |
| | 1 | 7 | 10 | 14 | 21 | 28 | 42 | 56 | | Q4W | PD |
| | Window | | | | | | | | | | |
| | | ±1 d | ±1 d | ±1 d | ±1 d | ±2 d | ±2 d | ±2 d | | | |
| PK CAR | X^{c} | X | | X | | X | | X | Table 10 | X | X |
| transgene | | | | | | | | | | | |
| levels (blood) | | | | | | | | | | | |
| Cilta-cel ADA | X^{c} | | | X | | X | | X | Table 10 | X | X |
| (serum) | | | | | | | | | | | |
| Cilta-cel/ | X | X | X | X | X | X | X | X | Table 10 | | X |
| talquetamab | | | | | | | | | | | |
| immunopheno- | | | | | | | | | | | |
| typing (blood) | | | | | | | | | | | |
| Cytokines | Collect additional pharmacodynamic cytokine samples, if feasible, | | | | | | | | | | |
| (blood) | when any of the following are observed or reported: (1) suspected | | | | | | | | | | |
| | sARRs reaction Grade ≥2; (2) CRS event Grade ≥2 (at onset of the CRS | | | | | | | | | | |
| | event, and 24 h and 72 h after); (3) as indicated based on emerging | | | | | | | | | | |
| | data | | | | | | | | | | |
| RCL (blood) | At approximately 3 months (D 84), 6 months (D 168), and 12 months (D 364) (all with | | | | | | | | | | |
| | window of ±1 month) after cilta-cel infusion. Additional samples may be collected if | | | | | | | | | | |
| | triggered by events that may be relevant, but not limited to, RCL per clinical | | | | | | | | | | |
| | assessment. | | | | | | | | | | |
| Immune and | | | | | | | | | | | |
| omic profiling | | | | | | | | | | | |
| (PBMC)^{f} | | | | | | | | | | | |

^{a}All samples predose (on dosing days).
b. For Cohort 2 only.
^{c}If using a window, collection should be taken prior to the first dose of conditioning regimen.
d. If a repeat apheresis is to be performed, another apheresis sample should be collected for PD assessments at the time of apheresis.
e. Predose sample window −4 to 0 hour of cilta-cel administration for pharmacokinetic and immunogenicity sampling.
^{f}PBMC collection taken at the time of apheresis.

TABLE 16A
Schedule of Activities for Pharmacokinetic and Biomarker Assessments (Cohort 3)^{a}.

| | | 1^{st} | Tal Bridging | 2^{nd} | Conditioning |
|---|---|---|---|---|---|
| Assessment | Screening | Apheresis | Regimen | Apheresis^{b} | Regimen |
| Day | | | | | −5*, −4, −3 |
| Window | | | | | * for start: |
| | | | | | D −7 to D −5 |
| PK CAR | | | | | |
| transgene | | | | | |
| levels (blood) | | | | | |
| ADA (serum) | | | | | |
| Cilta-cel/ | X | | Table 10 | | |
| talquetamab | | | | | |
| immunophenotyping | | | | | |
| (blood) | | | | | |
| Cytokines | X | | Collect additional pharmacodynamic cytokine | | |
| (blood) | | | samples, if feasible when any of the following are | | |
| | | | observed or reported: (1) suspected sARRs | | |
| | | | reaction Grade 22; (2) CRS event Grade 22 (at | | |
| | | | onset of the CRS event, and 24 h and 72 h after); | | |
| | | | (3) as indicated based on emerging data. | | |
| RCL (blood) | X | | | | |
| Immune and | | X^{c} | | X^{c} | |
| omic profiling | | | | | |
| (PBMC)^{e} | | | | | |

^{a}All samples predose (on dosing days).
^{b}The first 5 participants will also undergo a second apheresis after bridging.
^{c}If a repeat apheresis is to be performed, another apheresis sample should be collected for pharmacodynamic assessments at the time of apheresis.
d. Predose sample window −4 to 0 hour of cilta-cel administration for pharmacokinetic and immunogenicity sampling.
^{e}PBMC collection taken at the time of apheresis.

TABLE 16B
Schedule of Activities for Pharmacokinetic and Biomarker Assessments (Cohort 3)^{a}.

| | Assessment | | | | | | | | | | | |
|---|---|---|---|---|---|---|---|---|---|---|---|---|
| | Cilta-cel | | | | | | | | | | | |
| | Infusion | Post-infusion | | | | | | | | | Follow-up | |
| | Day | | | | | | | | | | | |
| | | | | | | | | | | | Q4W | |
| | 1 | 7 | 10 | 14 | 21 | 28 | 42 | 56 | 78 | 100 | post-D 100 | Post-PD |
| | Window | | | | | | | | | | | |
| | | ±1 d | ±1 d | ±1 d | ±1 d | ±2 d | ±2 d | ±2 d | ±2 d | ±2 d | | |
| PK CAR | X^{d} | X | | X | | X | | X | | X | X | X |
| transgene | | | | | | | | | | | | |
| levels (blood) | | | | | | | | | | | | |
| ADA (serum) | X^{d} | | | X | | X | | X | | X | X | X |
| Cilta-cel/ | X | X | X | X | | X | | X | | X | | X |
| talquetamab | | | | | | | | | | | | |
| immunopheno | | | | | | | | | | | | |
| typing (blood) | | | | | | | | | | | | |
| Cytokines | Collect additional pharmacodynamic cytokine samples, if feasible | | | | | | | | | | | |
| (blood) | when any of the following are observed or reported: (1) suspected | | | | | | | | | | | |
| | sARRs reaction Grade 22; (2) CRS event Grade 22 (at onset of the | | | | | | | | | | | |
| | CRS event, and 24 h and 72 h after); (3) as indicated based on | | | | | | | | | | | |
| | emerging data. | | | | | | | | | | | |
| RCL (blood) | At approximately 3 months (D 84), 6 months (D 168), and 12 months (D 364) (all with | | | | | | | | | | | |
| | window of ±1 month) after cilta-cel infusion. Additional samples may be collected if | | | | | | | | | | | |
| | triggered by events that may be relevant, but not limited to RCL, per clinical | | | | | | | | | | | |
| | assessment. | | | | | | | | | | | |
| Immune and | | | | | | | | | | | | |
| omic profiling | | | | | | | | | | | | |
| (PBMC)^{e} | | | | | | | | | | | | |

^{a}All samples predose (on dosing days).
b. The first 5 participants will also undergo a second apheresis after bridging.
c. If a repeat apheresis is to be performed, another apheresis sample should be collected for pharmacodynamic assessments at the time of apheresis.
^{d}Predose sample window −4 to 0 hour of cilta-cel administration for pharmacokinetic and immunogenicity sampling.
^{e}PBMC collection taken at the time of apheresis.

TABLE 17
Schedule of Activities for Talquetamab Pharmacokinetic,
Immunogenicity, and Biomarker Assessment During Talquetamab
Bridging and Consolidation^{a} (Cohorts 1, 2, and 3).

| | Assessment | | | | | | | |
|---|---|---|---|---|---|---|---|---|
| | | | | | Cycle | Cycle | Cycle | Cycle |
| | Cycle 1 | | | | 2 | 3 | 7^{b} | 12^{b} |
| | Day | | | | | | | |
| | 1 | 4 | 8 | 15 | 1 | 1 | 1 | 1 |
| | Window | | | | | | | |
| | | ±2 d | ±2 d | ±2 d | ±3 d | ±3 d | ±3 d | ±3 d |
| Cilta-cel/ | X | | | X | X | X | X | X |
| talquetamab | | | | | | | | |
| phenotyping | | | | | | | | |
| (blood) | | | | | | | | |
| Talquetamab | X | X | X | X | X | X | X | X |
| PK (serum)^{d} | | | | | | | | |
| Talquetamab | X | | | | X | X | X | X |
| ADA (serum)^{d} | | | | | | | | |
| Cytokines^{c} | X^{b} | | | X^{b} | | | | |
| (blood) | | | | | | | | |

^{a}All samples predose.
^{b}For Cohorts 1 and 2 only.
^{c}Collect additional pharmacodynamic cytokine samples, if feasible, when any of the following are observed or reported: (1) suspected sARRs reaction Grade 22; (2) CRS event Grade 22 (at onset of the CRS event, and 24 h and 72 h after); (3) as indicated based on emerging data.
^{d}Predose sample window −4 to 0 hour of talquetamab administration for pharmacokinetic and immunogenicity sampling.

TABLE 18
Inclusion Criteria.

| | |
|---|---|
| 1. | ≥18 years of age (or the legal age of majority in the jurisdiction in which the study is taking |
| | place, whichever is greater) at the time of informed consent. |
| 2. | Documented diagnosis of MM as defined by the criteria below: |

| | |
|---|---|
| a. | MM diagnosis according to the IMWG diagnostic criteria. |
| b. | Measurable disease at screening as assessed by central laboratory, defined by any of the |

following:

| | |
|---|---|
| i. | Serum M-protein level ≥0.5 g/dL; or |
| ii. | Urine M-protein level ≥200 mg/24 hours; or |
| iii. | Serum immunoglobulin FLC ≥10 mg/dL and abnormal serum immunoglobulin |

kappa lambda FLC ratio

| | |
|---|---|
| | NOTE: In exceptional circumstances and after discussion with and written approval by the |
| | sponsor, the local laboratory results may be used to determine initial eligibility. |
| 3. | Have high-risk MM defined as one or more of the following: |

| | |
|---|---|
| a. | At least one cytogenetic abnormality [t(4; 14); t(14; 16) and/or del17p], or |
| b. | Baseline ISS Stage III, or |
| c. | At least one baseline extramedullary plasmacytoma |

| | |
|---|---|
| 4. | Cohorts 1 and 3: Received at least 3 prior lines of antimyeloma therapy including a PI, an |
| | iMiD, and an anti-CD38 mAb. Participant must have undergone ≥1 complete cycle of treatment |
| | for each regimen, unless PD was the best response to the regimen. Refer to Appendix 8 for the |
| | definition of a line of therapy. |
| | Cohort 2: Be newly diagnosed MM and not considered a candidate for high-dose chemotherapy |
| | with ASCT due to: |

| a. | Ineligible due to age ≥65 years; or |
|---|---|
| b. | Ineligible due to presence of comorbid condition(s) likely to have a negative impact on |
| tolerability of high-dose chemotherapy with ASCT; or | |
| c. | Deferral of high-dose chemotherapy with ASCT as initial treatment |

| | |
|---|---|
| 5. | Cohorts 1 and 3: Documented evidence of PD or failure to achieve a response to the last line of |
| | therapy based on investigator's determination of response by the IMWG response criteria. |
| | Relapsed or refractory disease as defined below: |

| a. | Relapsed disease is defined as an initial response to prior treatment, followed by |
|---|---|
| confirmed PD by IMWG response criteria >60 days after cessation of treatment. | |
| b. | Refractory disease is defined as failure to achieve a response or confirmed PD by |

IMWG response criteria during previous treatment or ≤60 days after cessation of treatment

| | Cohort 2: Not applicable | |
|---|---|---|
| 6. | ECOG performance status score of 0 or 1. | |
| 7. | Adequate organ function defined as follows: | |
| | Hematology | |
| | Hemoglobin | ≥8.0 g/dL (>5 mmol/L) (without prior RBC transfusion within 7 days |
| | | before the laboratory test; recombinant human erythropoietin use is |
| | | permitted) |
| | Platelets | ≥50 × 10⁹/L (must be without transfusion support in the 7 days prior |
| | | to the laboratory test) |
| | Lymphocyte count | ≥0.3 × 10⁹/L |
| | Absolute Neutrophil | ≥0.75 × 10⁹/L (prior growth factor support is permitted but must be |
| | Count (ANC) | without support in the 7 days for G-CSF or GM-CSF and for 14 |
| | | days for pegylated G-CSF prior to the laboratory test) |
| | Chemistry | |
| | AST and ALT | 2.5 × ULN |
| | Creatinine clearance | ≥40 mL/min based upon Modified Diet in Renal Disease formula |
| | eGFR | calculation or a 24-hour urine collection. |
| | Total bilirubin | 2.0 × ULN; except in participants with congenital nonhemolytic |
| | | bilirubinemia, such as Gilbert syndrome (in which case direct |
| | | bilirubin 1.5 × ULN is required) |
| | Serum calcium corrected | 14 mg/dL (3.5 mmol/L) or free ionized calcium 6.5 mg/dL (1.6 |
| | for albumin | mmol/L) |

| | |
|---|---|
| 8. | POCBP must have a negative pregnancy test using a highly sensitive β-hCG serum pregnancy |
| | test at screening. |
| 9 | A participant must be: |

Not of childbearing potential, or
Of childbearing potential and

| | |
|---|---|
| 1) | Practicing true abstinence; or |
| 2) | Practicing at least 2 reliable methods of contraception simultaneously, including |

1 highly effective method of contraception and 1 other effective method of
contraception (details in Appendix 4). Contraception must begin from the time of
signing the ICF or for at least 4 weeks prior to dosing of any study treatment, continue
during study treatment, including during dose interruptions, until at least 6 months
following the last dose of talquetamab or 1 year after receiving cilta-cel infusion
(whichever is the latest).

| | |
|---|---|
| | Reliable contraception is indicated even where there has been a history of infertility, unless due |
| | to hysterectomy or bilateral oophorectomy. |
| | NOTE: If a participant becomes of childbearing potential after the start of the study, they must |
| | comply with point (2) as described above. |
| | NOTE: An interaction between hormonal contraception and talquetamab has not been formally |
| | studied. Therefore, it is unknown whether talquetamab may reduce the efficacy of the |
| | contraception method. If a participant is receiving talquetamab and is using hormonal |
| | contraceptives, an additional barrier method must be used. |
| | NOTE: Sexual abstinence is considered a highly effective method only if defined as refraining |
| | from sexual intercourse where pregnancy is possible during the entire period of risk associated |
| | with the study treatment. The reliability of sexual abstinence needs to be evaluated in relation to |
| | the duration of the study and the preferred and usual lifestyle of the participant. |
| 10. | A participant using oral contraceptives must use an additional contraceptive method. |
| 11. | A participant must agree not to be pregnant, breastfeeding, or planning to become pregnant |
| | while enrolled in this study or within 6 months following the last dose of talquetamab or 1 year |
| | after receiving cilta-cel infusion (whichever is the latest). |
| 12. | A participant must agree not to donate gametes (ova, oocytes, sperm) or freeze for future use for |
| | the purposes of assisted reproduction during the study and for 6 months after receiving the last |
| | dose of talquetamab or 1 year after receiving cilta-cel infusion (whichever is the latest). |
| | Participants should consider preservation of gametes prior to study treatment as anticancer |
| | treatments may impair fertility. |
| | NOTE: If the participant's partner is a person of childbearing potential, the participant must use |
| | condoms (with or without spermicide) and the partner of the participant must also be practicing |
| | a highly effective method of contraception. A participant who is vasectomized must still use a |
| | condom (with or without spermicide), but the partner is not required to use contraception. |
| 13. | A participant must agree not to plan to conceive a child while enrolled in this study or within 6 |
| | months after the last dose of talquetamab or 1 year after receiving cilta-cel infusion (whichever |
| | is the latest). |
| 14. | Must sign an ICF (or their legally acceptable representative must sign in accordance with local |
| | legislation) indicating that the participant understands the purpose of and procedures required |
| | for the study and is willing to participate in the study. |
| 15. | Willing and able to adhere to the lifestyle restrictions specified in this protocol. |

TABLE 19
Exclusion Criteria.

| | |
|---|---|
| 1. | Cohorts 1 and 3: Prior treatment with CAR-T therapy directed at any target or any prior |
| | BCMA-directed therapy/prior GPRC5D-directed therapy. |
| | Cohort 2: Received any prior therapy for MM or smoldering myeloma other than a short |
| | course of corticosteroids (not to exceed 40 mg of dexamethasone or equivalent per day for a |
| | maximum of 4 days, total of 160 mg dexamethasone or equivalent). |
| 2. | Prior antimyeloma therapy as follows, prior to apheresis: |

| | |
|---|---|
| a. | Radiation therapy for treatment of plasmacytoma within 14 days of enrollment |
| | (palliative radiation for pain control secondary to lytic lesion is allowed within 14 days |
| | of enrollment). If the radiation portal covered <5% of the bone marrow reserve, the |
| | participant is eligible irrespective of the end date of radiation therapy |

Cohorts 1 and 3:

| | |
|---|---|
| a. | Targeted therapy, epigenetic therapy, or treatment with an investigational drug, |

investigational intervention (including investigational vaccines) or used an invasive
investigational medical device within 14 days or 25 half-lives, whichever is less

| | |
|---|---|
| b. | mAb therapy within 21 days |
| c. | Cytotoxic therapy within 14 days |
| d. | PI therapy within 14 days iMiD therapy within 7 days |

| | |
|---|---|
| 3. | Cohorts 1 and 3: Received either of the following: |

| a. | An allogenic stem cell transplant within 6 months before apheresis/first dose of study |
|---|---|
| drug. Participants who received an allogeneic transplant must be off all | |
| immunosuppressive medications for 6 weeks before the start of study treatment | |
| administration without signs of graft-versus-host disease. | |
| b. | An autologous stem cell transplant <12 weeks before apheresis/first dose of study |

treatment

| | |
|---|---|
| | Cohort 2: Not applicable |
| 4. | Cohorts 1 and 3: Received a cumulative dose of corticosteroids equivalent to 270 mg of |
| | prednisone within the 7 days prior to apheresis/first dose of study drug. |
| 5. | Live, attenuated vaccine within 4 weeks of enrollment. Non-live and non-replicating vaccines |
| | approved or authorized for emergency use (eg, COVID-19) by local health authorities are |
| | allowed. |
| 6. | Cohort 2: Received a strong CYP450 inducer within 5 half-lives prior to DRd induction |
| | therapy. |
| 7. | Toxicity from previous anticancer therapy not resolved to baseline levels or to Grade 1 or less |
| | except for alopecia or peripheral neuropathy. |
| 8. | Presence of any of the following: |

| a. | Any ongoing myelodysplastic syndrome or B-cell malignancy (other than MM). |
|---|---|
| b. | Any history of malignancy, other than MM, that is considered at high risk of |
| recurrence requiring systemic therapy. | |
| c. | Any active malignancy (ie, progressing or requiring treatment change in the last 24 |

months) other than MM. The only allowed exceptions are malignancies treated within the
last 24 months that are considered cured:

| i. | Non-muscle invasive bladder cancer (solitary Ta-PUNLMP or low-grade, <3 |
|---|---|
| cm, no CIS) | |
| ii. | Non-melanoma skin cancers treated with curative therapy or localized |
| melanoma treated with curative surgical resection alone | |
| iii. | Non-invasive cervical cancer |
| iv. | Breast cancer: adequately treated lobular carcinoma in situ or ductal |
| carcinoma in situ, or history of localized breast cancer (anti-hormonal therapy is | |
| permitted) | |
| v. | Localized prostate cancer (M0, N0) with a Gleason Score ≤7a, treated locally |
| only (RP/RT/focal treatment) | |
| vi. | Other malignancy that is considered cured with minimal risk of recurrence in |

consultation with the sponsor.

| | |
|---|---|
| | NOTE: In the event of any questions, consult with the sponsor prior to enrolling a participant. |
| 9. | Presence of the following cardiac conditions: |

| a. | NYHA Stage III or IV congestive heart failure |
|---|---|
| b. | Myocardial infarction or coronary artery bypass graft ≤6 months prior to enrollment |
| c. | History of clinically significant ventricular arrhythmia or unexplained syncope, not |
| believed to be vasovagal in nature or due to dehydration | |
| d. | History of severe non-ischemic cardiomyopathy |
| e. | Impaired cardiac function (LVEF <45%) as assessed by echocardiogram or MUGA |

scan (performed ≤8 weeks prior to apheresis)

| | |
|---|---|
| 10. | Known active or prior CNS involvement or exhibits clinical signs of meningeal involvement |
| | of MM. If either is suspected, negative whole brain MRI and lumbar cytology are required. |
| 11. | Stroke, transient ischemic attack, or seizure within 6 months of signing ICF. |
| 12. | Plasma cell leukemia at the time of screening (≥5% circulating PCs in peripheral blood |
| | smear), Waldenstrom's macroglobulinemia, POEMS syndrome (polyneuropathy, |
| | organomegaly, endocrinopathy, monoclonal protein, and skin changes), or primary AL |
| | amyloidosis. |
| 13. | Serious underlying medical conditions, such as: |

| a. | Evidence of active systemic viral or bacterial infection, requiring systemic |
|---|---|
| antimicrobial therapy, or uncontrolled systemic fungal infection | |
| b. | Active autoimmune disease or a history of autoimmune disease within 3 years |
| c. | Any history of Parkinson's disease or other neurodegenerative disorder |
| d. | Overt clinical evidence of dementia or altered mental status |

| | |
|---|---|
| 14. | Seropositive for HIV. |
| 15. | Any condition for which in the opinion of the investigator, participation would not be in the |
| | best interest of the participant (eg, compromise the well-being) or that could prevent, limit, or |
| | confound the protocol-specified assessments. |
| 16. | HBV infection. |
| 17. | Active HCV infection. |
| 18. | Require continuous supplemental oxygen. |
| 19. | Known life-threatening allergies, hypersensitivity, or intolerance to cyclophosphamide, |
| | fludarabine, cilta-cel, or talquetamab excipients, including DMSO (refer to Investigator's |
| | Brochures). |
| 20. | Major surgery (eg, requiring general anesthesia) within 2 weeks of screening, or will not have |
| | fully recovered from surgery, or has major surgery planned during the time the participant is |
| | expected to be treated in the study. |

TABLE 20A
Dose Schedule and Pretreatment- Medications - (Cohorts 1 and 2: Talquetamab
up to 12 cycles; Cohort 3: Talquetamab for 2 to 4 cycles).

| | Treatment Phase | | | |
|---|---|---|---|---|
| | (28-day cycle) | | | |
| | Cycle | | | |
| | 1 | | | |
| | Day | | | |
| Notes | 1 | 4 | 8 | 15 |

REQUIRED PRETREATMENT MEDICATIONS

| | | | | | |
|---|---|---|---|---|---|
| Dexamethasone | Oral/IV: Administer 1-3 h | X | X | X | X |
| 20 mg or equivalent | (±15 mins) prior to talquetamab | | | | |
| Diphenhydramine 25 to 50 mg | SC | X | X | X | X |
| or equivalent | Prior to all step-up doses and | | | | |
| Acetaminophen | first treatment dose in C1 | X | X | X | X |
| 650 to 1,000 mg | | | | | |

OPTIONAL PRETREATMENT MEDICATION: Additional pretreatment medications such as H2
antagonists or antiemetics may be used at investigator discretion.
STUDY TREATMENT: If occur, CRS (fever, hypoxia, and hypotension) and ICANS must fully
resolve before the next administration of talquetamab.

| | | | | | |
|---|---|---|---|---|---|
| Talquetamab SC SU1 (0.01 | | X | | | |
| mg/kg) | | | | | |
| Talquetamab SC SU2 (0.06 | Administer ≥2 d after SU1, | | X | | |
| mg/kg) | between Days 3-6 | | | | |
| Talquetamab SC SU3 (0.4 | Administer ≥2 d after SU2, | | | X | |
| mg/kg) | between Days 5-10 | | | | |
| Talquetamab SC Treatment | Administer ≥2 d after SU3, | | | | X |
| Dose (0.8 mg/kg) | between Days 7-15 | | | | |
| | C2-C6 must be administered | | | | |
| | 14 d (±3 d) after prior dose | | | | |
| | From C5, if confirmed VGPR | | | | |
| | or better, schedule can change to | | | | |
| | Q4W dosing (D 1 only) per | | | | |
| | investigator discretion | | | | |
| | Q4W from C7 for all | | | | |
| | participants | | | | |

TABLE 20B
Dose Schedule and Pretreatment- Medications - (Cohorts 1 and 2: Talquetamab
up to 12 cycles; Cohort 3: Talquetamab for 2 to 4 cycles).

| | Treatment Phase (28-day cycle) | |
|---|---|---|
| | Cycle | |
| | 2-6 | 7+ |
| | Day | |
| Notes | 1, 15 | 1 |

REQUIRED PRETREATMENT MEDICATIONS

| | | |
|---|---|---|
| Dexamethasone | Oral/IV: Administer 1-3 h (±15 | From Cycle 2 onwards premedications |
| 20 mg or equivalent | mins) prior to talquetamab SC | should not be administered, unless |
| Diphenhydramine | Prior to all step-up doses and | approved by the sponsor. If premedications |
| 25 to 50 mg or | first treatment dose in C1 | are required after Cycle 2, they can be |
| equivalent | | taken at home, following the specified |
| Acetaminophen | | timeframe^{a}. A participant who experiences |
| 650 to 1,000 mg | | Grade ≥2 CRS/sARR related to |
| | | talquetamab must receive pretreatment |
| | | medications for at least the subsequent |
| | | dose of talquetamab to which the event |
| | | was related. Administration of |
| | | premedications beyond this should be |
| | | discussed with the sponsor. The event- |
| | | driven premedications are not required if |
| | | already being administered as planned |
| | | premedications as described in this dose |
| | | schedule table. |

OPTIONAL PRETREATMENT MEDICATION: Additional pretreatment medications such as H2
antagonists or antiemetics may be used at investigator discretion.
STUDY TREATMENT: If occur, CRS (fever, hypoxia, and hypotension) and ICANS must fully
resolve before the next administration of talquetamab.

| | | | |
|---|---|---|---|
| Talquetamab SC | | | |
| SU1 (0.01 mg/kg) | | | |
| Talquetamab SC | Administer ≥2 d after SU1, | | |
| SU2 (0.06 mg/kg) | between Days 3-6 | | |
| Talquetamab SC | Administer ≥2 d after SU2, | | |
| SU3 (0.4 mg/kg) | between Days 5-10 | | |
| Talquetamab SC | Administer ≥2 d after SU3, | X | X |
| Treatment Dose (0.8 | between Days 7-15 | | |
| mg/kg) | C2-C6 must be administered | | |
| | 14 d (±3 d) after prior dose | | |
| | From C5, if confirmed VGPR or | | |
| | better, schedule can change to | | |
| | Q4W dosing (D 1 only) per | | |
| | investigator discretion | | |
| | Q4W from C7 for all | | |
| | participants | | |

TABLE 21
Cilta-cel Administration and Pre-cilta-cel-infusion Medication.
Cilta-cel pre-infusion Medication

| | |
|---|---|
| Medication/ | Antihistamine: Diphenhydramine (25-50 mg IV or PO) or equivalent: |
| Dose | PO - administer 1 hour (±15 minutes) prior to cilta-cel infusion or IV- start |
| Administration | infusion 30 minutes (±15 minutes) prior to cilta-cel infusion |
| | Antipyretic: Acetaminophen (650 mg to 1,000 mg) or equivalent: PO or IV - |
| | 30 minutes (±15 minutes) prior to cilta-cel infusion |

Cilta-cel administration

| | |
|---|---|
| Dose | Cilta-cel will be administered in one infusion. The target dose was 0.75 × 10⁶ |
| | CAR-positive viable T cells/kg (range: 0.5-1.0 × 10⁶ CAR-positive viable T |
| | cells/kg). The maximum total dose of cells administered to any participant was |
| | 1.0 × 10⁸ CAR-positive viable T cells. Product was manufactured based on weight at |
| | apheresis. A repeat infusion was not available because cilta-cel is manufactured |
| | and provided in a single-dose unit. |
| | If after apheresis and CAR-T cell preparation the quantity of cilta-cel manufactured |
| | was not sufficient for dosing at the lower end of the dosing range, dosing for that |
| | participant could proceed, provided that a measurable quantity of cilta-cel CAR- |
| | positive viable T cells that pass quality testing were generated and product was |
| | released per company exceptional release procedures criteria. If the quantity of |
| | cilta-cel manufactured exceeded the upper end of the dosing range, it was evaluated |
| | similarly per company exceptional release criteria or similar process. |
| Route/Regimen | Cilta-cel IV infusion was administered under the supervision of qualified site |
| | staff. Refer to the Investigational Product Preparation Instructions (IPPI) for cilta- |
| | cel infusion instructions. |
| Dosing | The actual dose for study treatment administration was based on the participant's |
| Instructions | weight (kg) at apheresis. |
| Schedule of | One IV infusion |
| Administration | |
| Hospitalization | Dependent on the participant's status, medical history, concurrent comorbidities, |
| Requirements | adequate social support (full-time company of a competent adult) or potential risk |
| | factors for developing CAR-T toxicities, including CRS and neurotoxicity, it was at |
| | the investigator's discretion, participant's willingness, and sponsor approval whether |
| | the participant: |
| | Was admitted for inpatient monitoring from the day of infusion (Day 1) |
| | through Day 14 post cilta-cel infusion (with potential discharge on Day |
| | 10, if there were no CRS, neurotoxicity or other clinically significant |
| | events), |
| | OR |
| | received cilta-cel infusion as an outpatient in close proximity (within 30 |
| | min) to the hospital, was monitored for outpatient follow-up and then |
| | admitted for the required inpatient monitoring from Day 5 to Day 14 after |
| | cilta-cel infusion (with potential discharge on Day 10, if there were no |
| | CRS, neurotoxicity or other clinically significant adverse events). |
| | This evaluation occurred at the time of apheresis, prior to administration of the |
| | conditioning regimen, and again prior to cilta-cel infusion, and in consultation |
| | with approval from the sponsor to determine whether outpatient administration |
| | and follow-up after cilta-cel infusion was suitable for a given participant and site. |
| | The participant was required to be clinically evaluated after cilta-cel infusion for |
| | at least 6 hours before being discharged from the outpatient facility. |
| | Subject to institutional guidance, local regulations, investigator discretion and |
| | sponsor approval. |
| | For countries or specific hospitals that require hospitalization for all participants |
| | treated with cellular therapy, the more stringent requirements for hospitalization |
| | will prevail. |
| | Participants will be asked to remain within a 1-hour travel time of the hospital |
| | and in the company of a competent adult at all times for 1 additional week after |
| | hospital discharge, or until study Day 21, whichever is sooner. |
| | At the first sign of CRS (such as fever), participants should be immediately |
| | hospitalized for evaluation. |
| | Hospitalization is required for Grade 2, 3, or 4 CAR-T cell-related neurotoxicity |
| | (eg, ICANS) temporally associated to CRS. |
| | Hospitalization for neurotoxicity that is not temporally associated with CRS, or |
| | any other neurologic aEs, is at the discretion of the investigator. |
| Vital Sign and | Monitor vital signs as indicated in the SoA. |
| Clinical Safety | |
| Monitoring | |

TABLE 22A
Dosing Schedule, Pretreatment, and Post-treatment Medications - DRd Induction (Cohort 2).

| | Treatment Phase | | | |
|---|---|---|---|---|
| | (28-day cycle) | | | |
| | Cycle | | | |
| | 1-2 | | | |
| | Day | | | |
| | 1 | 8 | 15 | 22 |
| | Window | | | |
| Notes | ±1 d^{a} | ±1 d | ±1 d | ±1 d |

REQUIRED PRETREATMENT MEDICATIONS: From Cycle 2 onward, all daratumumab oral
pretreatment medications may be administered at home, provided they are taken within the
timeframes specified below.

| Dexamethasone 40 mg | Oral/IV: Administer 1-3 hours | See Study Treatment section | | | |
|---|---|---|---|---|---|
| | (±15 min) prior to | below: dexamethasone is both a | | | |
| | administration of daratumumab | study treatment and a | | | |
| | SC. | daratumumab premedication | | | |
| Diphenhydramine 25 to 50 mg | Oral/IV: Administer 1 hour | X | X | X | X |
| or equivalent | (±15 min) prior to (first) study | | | | |
| Acetaminophen 650 to 1,000 mg | drug in Cycle 1. Beginning in | X | X | X | X |
| or equivalent | Cycle 2, administer ~1-3 hours | | | | |
| | (±15 min) prior to | | | | |
| | administration of the first study | | | | |
| | drug. | | | | |

OPTIONAL PRETREATMENT MEDICATIONS: Additional pretreatment medications such as
H2-antagonists or antiemetics may be used per investigator discretion.

| | | |
|---|---|---|
| Montelukast 10 mg | ~1-3 hours (±15 min) prior to | Per investigator discretion prior |
| | administration of daratumumab | to administration of |
| | SC. | daratumumab SC |

STUDY TREATMENT

| Daratumumab SC | There must be ≥5 days between | X | X | X | X |
|---|---|---|---|---|---|
| 1800 mg | doses of daratumumab SC. | | | | |
| Lenalidomide 25 mg | Oral administration | Day 1 to 21 of each cycle | | | |
| | Lenalidomide dose should be | | | | |
| | adjusted based on CrCl (see | | | | |
| | Section 6.2.3.4). | | | | |
| Dexamethasone 40 mg | Oral/IV: Dispense on Day 1 | Weekly (Day 1, 8, 15, and 22) | | | |
| | for self-administration. | | | | |
| | This dose of dexamethasone | | | | |
| | also serves as a pretreatment | | | | |
| | medication to be administered 1 | | | | |
| | to 3 hours (±15 min) prior to | | | | |
| | daratumumab SC (see Required | | | | |
| | Pretreatment Medications | | | | |
| | section above). | | | | |
| | For participants >75 years of | | | | |
| | age or with BMI <18.5 kg/m2, | | | | |
| | dexamethasone may be | | | | |
| | administered at a dose of 20 mg. | | | | |

POST-TREATMENT MEDICATIONS: All participants will be observed for ≥6 hours after the
end of the daratumumab SC injection on Cycle 1 Day 1 and, if deemed necessary by the
investigator, after subsequent injections.

| Diphenhydramine | For participants with a higher | Per investigator discretion to |
|---|---|---|
| 25 to 50 mg or equivalent | risk of respiratory complications | reduce the risk of delayed |
| | (eg, participants with mild | sARRs |
| Montelukast 10 mg | asthma or participants with | Per investigator discretion to |
| | COPD who have an FEV1 <80% | reduce the risk of delayed |
| | at screening or developed | sARRs |
| Short-acting β2 adrenergic | FEV1 <80% during the study | Per investigator discretion to |
| receptor agonist, eg, salbutamol | without any medical history). | reduce the risk of delayed |
| (albuterol) | Participants at risk for | sARRs |
| | respiratory complications may | |
| | be hospitalized for monitoring | |
| | for up to 2 nights after | |
| | daratumumab administration (if | |
| | hospitalization is for observation | |
| | only, without a significant | |
| | medical event, the | |
| | hospitalization should not be | |
| | reported as an SAE). | |
| | If participants are not | |
| | hospitalized, a follow-up | |
| | telephone call should be made to | |
| | monitor their condition within | |
| | 48 hours after 4 daratumumab | |
| | administrations.^{b} | |
| Control medications for lung | Inhaled corticosteroids ± long- | Per investigator discretion to |
| disease | acting β2 adrenergic receptor | reduce the risk of delayed |
| | agonists for participants with | sARRs |
| | asthma; | |
| | Long-acting bronchodilators, eg, | |
| | tiotropium or salmeterol ± | |
| | inhaled corticosteroids for | |
| | participants with COPD | |

^{a}Treatment with DRd should begin within 7 days after apheresis.
^{b}Investigators may prescribe bronchodilators, antihistamines, and corticosteroids if necessary to provide adequate supportive care in the event bronchospasm occurs after a participant is released from the hospital/clinic. If a participant at risk for respiratory complications experiences no major sARRs then these post-administration medications may be waived after 4 doses at the investigator's discretion.

TABLE 22B
Dosing Schedule, Pretreatment, and Post-treatment Medications - DRd Induction (Cohort 2).

| | Treatment Phase | | | |
|---|---|---|---|---|
| | (28-day cycle) | | | |
| | Cycle | | | |
| | 3-4 | | | |
| | Day | | | |
| | 1 | 8 | 15 | 22 |
| | Window | | | |
| Notes | ±1 d^{a} | ±1 d | ±1 d | ±1 d |

REQUIRED PRETREATMENT MEDICATIONS: From Cycle 2 onward, all daratumumab oral
pretreatment medications may be administered at home, provided they are taken within the
timeframes specified below.

| Dexamethasone 40 mg | Oral/IV: Administer 1-3 hours | See Study Treatment section | | | |
|---|---|---|---|---|---|
| | (±15 min) prior to | below: dexamethasone is both a | | | |
| | administration of daratumumab | study treatment and a | | | |
| | SC. | daratumumab premedication | | | |
| Diphenhydramine 25 to 50 mg | Oral/IV: Administer 1 hour | X | X | X | X |
| or equivalent | (±15 min) prior to (first) study | | | | |
| Acetaminophen 650 to 1,000 mg | drug in Cycle 1. Beginning in | X | X | X | X |
| or equivalent | Cycle 2, administer ~1-3 hours | | | | |
| | (±15 min) prior to | | | | |
| | administration of the first study | | | | |
| | drug. | | | | |

OPTIONAL PRETREATMENT MEDICATIONS: Additional pretreatment medications such as
H2-antagonists or antiemetics may be used per investigator discretion.

| | | |
|---|---|---|
| Montelukast 10 mg | ~1-3 hours (±15 min) prior to | Per investigator discretion prior |
| | administration of daratumumab | to administration of |
| | SC. | daratumumab SC |

STUDY TREATMENT

| | | | |
|---|---|---|---|
| Daratumumab SC | There must be ≥5 days between | X | X |
| 1800 mg | doses of daratumumab SC. | | |

| | | |
|---|---|---|
| Lenalidomide 25 mg | Oral administration | Day 1 to 21 of each cycle |
| | Lenalidomide dose should be | |
| | adjusted based on CrCl (see | |
| | Section 6.2.3.4). | |
| Dexamethasone 40 mg | Oral/IV: Dispense on Day 1 | Weekly (Day 1, 8, 15, and 22) |
| | for self-administration. | |
| | This dose of dexamethasone | |
| | also serves as a pretreatment | |
| | medication to be administered 1 | |
| | to 3 hours (±15 min) prior to | |
| | daratumumab SC (see Required | |
| | Pretreatment Medications | |
| | section above). | |
| | For participants >75 years of | |
| | age or with BMI <18.5 kg/m2, | |
| | dexamethasone may be | |
| | administered at a dose of 20 mg. | |

POST-TREATMENT MEDICATIONS: All participants will be observed for ≥6 hours after the
end of the daratumumab SC injection on Cycle 1 Day 1 and, if deemed necessary by the
investigator, after subsequent injections.

| | | |
|---|---|---|
| Diphenhydramine | For participants with a higher | Per investigator discretion to |
| 25 to 50 mg or equivalent | risk of respiratory complications | reduce the risk of delayed |
| | (eg, participants with mild | sARRs |
| Montelukast 10 mg | asthma or participants with | Per investigator discretion to |
| | COPD who have an FEV1 <80% | reduce the risk of delayed |
| | at screening or developed | sARRs |
| Short-acting β2 adrenergic | FEV1 <80% during the study | Per investigator discretion to |
| receptor agonist, eg, salbutamol | without any medical history). | reduce the risk of delayed |
| (albuterol) | Participants at risk for | sARRs |
| | respiratory complications may | |
| | be hospitalized for monitoring | |
| | for up to 2 nights after | |
| | daratumumab administration (if | |
| | hospitalization is for observation | |
| | only, without a significant | |
| | medical event, the | |
| | hospitalization should not be | |
| | reported as an SAE). | |
| | If participants are not | |
| | hospitalized, a follow-up | |
| | telephone call should be made to | |
| | monitor their condition within | |
| | 48 hours after 4 daratumumab | |
| | administrations.^{b} | |
| Control medications for lung | Inhaled corticosteroids ± long- | Per investigator discretion to |
| disease | acting β2 | reduce the risk of delayed |
| | adrenergic receptor agonists for | sARRs |
| | participants with asthma; | |
| | Long-acting bronchodilators, eg, | |
| | tiotropium or salmeterol ± | |
| | inhaled corticosteroids for | |
| | participants with COPD | |

^{a}Treatment with DRd should begin within 7 days after apheresis.
^{b}Investigators may prescribe bronchodilators, antihistamines, and corticosteroids if necessary to provide adequate supportive care in the event bronchospasm occurs after a participant is released from the hospital/clinic. If a participant at risk for respiratory complications experiences no major sARRs then these post-administration medications may be waived after 4 doses at the investigator's discretion.

TABLE 23
Study Treatment Administration Instruction - DRd.

| Study Drug | Daratumumab SC | Lenalidomide | Dexamethasone |
|---|---|---|---|
| Dosing | The volume of 1800 mg for SC | Lenalidomide will be | Dexamethasone may |
| Instructions | injection will be approximately | supplied as 5 mg, 10 | be given orally or IV. |
| | 15 mL and administration | mg, 15 mg, and 25 mg | |
| | should occur over | capsules | |
| | approximately 3 to 5 minutes | | |
| | SC injections will be prepared | Lenalidomide will be | Dexamethasone tablets |
| | as described in the IPPI or | self-administered | are to be taken with or |
| | equivalent documentation. | orally. | immediately after a |
| | The anatomical area of | Lenalidomide should | meal or snack, |
| | administration must be | be taken as a single | preferably in the |
| | recorded in the eCRF, and the | dose at the same time | morning. |
| | area observed for injection-site | daily. | |
| | reaction(s). Refer to the | Lenalidomide can be | |
| | prescribing information for | taken with or without | |
| | further details. | food. | |
| | | Breaking or dividing | |
| | | lenalidomide capsules | |
| | | is strongly | |
| | | discouraged. | |
| | | Dose may need to be | |
| | | adjusted for | |
| | | participants with | |
| | | reduced CrCl. | |
| Safety | Cycle 1 Day 1: Participants | Not applicable | Not applicable |
| Monitoring | will be observed for ≥6 hours | | |
| Requirement | after the end of the | | |
| | daratumumab SC injection on | | |
| | Cycle 1 Day 1 and, if deemed | | |
| | necessary by the investigator, | | |
| | after subsequent injections. | | |
| | Participants at risk for | | |
| | respiratory complications: see | | |
| | Table 7a-7b (post-treatment | | |
| | medications) | | |

| | |
|---|---|
| Vital Signs | On dosing days, vital signs including oxygen saturation should be performed as |
| (temperature, | follows: |
| pulse/heart rate, | Daratumumab SC: Perform before start of administration and at the end of the |
| respiratory rate, | injection. |
| blood pressure, | Monitor until resolution of sARR. |
| and oxygen | Any additional vital signs assessments supporting the start and end dates of an AE |
| saturation) | (eg, fever or hypotension) should be reported in the eCRF. |

TABLE 24
Dose Skips.

| | Dosing | Skip Dose if Dosing | Resume |
|---|---|---|---|
| Cycles | Frequency | Interrupted: | Dosing |
| Cycle 1 to 6 (participants with confirmed | Q2W | >7 days from | Next planned |
| VGPR or better may move to Q4W after 4 | | planned dose date^{a} | Q2W dosing |
| treatment cycles) | | | day |
| Cycle 7+ (all participants may move to Q4W | Q4W | Not applicable^{b} | Next planned |
| after 6 treatment cycles) | | | Q4W dosing |
| | | | day |

^{a}If a dose is delayed for <7 days, then it should be given and not skipped. The subsequent dose would be scheduled a minimum of 7 days later, and to resume the next cycle as scheduled. The next cycle can be delayed if treatment criteria are not yet met.
^{b}Day 1 of a cycle cannot be skipped. If talquetamab cannot be given, delay cycle and adjust Day 1 of subsequent cycles accordingly to maintain a 28-day cycle.

TABLE 25
Recommendations for Restarting Therapy After Dose Delay.

| Delayed | Duration Since | |
|---|---|---|
| Dose | Last Dose | Action |
| Step-up Dose 2 | 7 days or less | Continue talquetamab at Step-up Dose 2 (0.06 mg/kg). |
| 0.06 mg/kg | More than 7 days | Restart talquetamab at Step-up Dose 1 (0.01 mg/kg), then |
| | | increase the dose to Step-up Dose 2 (0.06 mg/kg). |
| Step-up Dose 3 | 7 days or less | Continue talquetamab at Step-up Dose 3 (0.4 mg/kg). |
| 0.4 mg/kg | 8 to 28 days | Restart talquetamab at Step-up Dose 2 (0.06 mg/kg), then |
| | | increase to Step-up Dose 3 (0.4 mg/kg). |
| | More than 28 days | Must be discussed with and approved by the sponsor. |
| | | Restart talquetamab at Step-up Dose 1 (0.01 mg/kg), |
| | | followed by Step-up Dose 2 (0.06 mg/kg), then increase to |
| | | Step-up Dose 3 (0.4 mg/kg). |
| Initial Q2W | 35 days or less | Continue talquetamab at Initial Q2W Treatment |
| Treatment | | Dose (0.8 mg/kg). |
| Dose | 36 to 56 days | Must be discussed with and approved by the sponsor. |
| 0.8 mg/kg | | Restart talquetamab at Step-up Dose 2 (0.06 mg/kg), |
| | | increase the dose to Step-up Dose 3 (0.4 mg/kg) for one |
| | | dose, and then to Q2W Treatment Dose (0.8 mg/kg). |
| | More than 56 days | Must be discussed with and approved by the sponsor. |
| | | Restart talquetamab at Step-up Dose 1 (0.01 mg/kg), |
| | | followed by |
| | | Step-up Dose 2 (0.06 mg/kg) then increase the dose to Step- |
| | | up |
| | | Dose 3 (0.4 mg/kg), and then to Q2W Treatment Dose |
| | | (0.8 mg/kg). |
| Subsequent | 42 days or less | Continue talquetamab at Q2W Treatment Dose (0.8 mg/kg). |
| Q2W | | |
| Treatment | 43 to 79 days | Must be discussed with and approved by the sponsor. |
| Dose | | Restart talquetamab at Step-up Dose 2 (0.06 mg/kg), |
| 0.8 mg/kg | | increase the dose to Step-up Dose 3 (0.4 mg/kg) for one |
| | | dose, and then to Q2W Treatment Dose (0.8 mg/kg). |
| | More than 70 days | Must be discussed with and approved by the sponsor. |
| | | Restart talquetamab at Step-up Dose 1 (0.01 mg/kg), Step- |
| | | up Dose 2 (0.06 mg/kg), increase to Step-up Dose 3 (0.4 |
| | | mg/kg), and then to Q2W Treatment Dose (0.8 mg/kg). |
| Q4W | 42 days or less | Continue talquetamab at Q4W Treatment Dose (0.8 mg/kg). |
| Treatment | | |
| Dose | 43 to 70 days | Must be discussed with and approved by the sponsor. |
| 0.8 mg/kg | | Restart talquetamab at Step-up Dose 2 (0.06 mg/kg), |
| | | increase to Step-up Dose 3 (0.4 mg/kg), and then to |
| | | Q4W Treatment Dose (0.8 mg/kg). |
| | More than 70 days | Must be discussed with and approved by the sponsor. |
| | | Restart talquetamab at Step-up Dose 1 (0.01 mg/kg), Step- |
| | | up Dose 2 (0.06 mg/kg), increase to Step-up Dose 3 (0.4 |
| | | mg/kg), and then to Q4W Treatment Dose (0.8 mg/kg). |

TABLE 26
Dosing Interruption for Hematologic Adverse Events.

| | |
|---|---|
| Hematologic AE | Talquetamab |

Neutropenia^{a}

| | |
|---|---|
| ANC <0.5 × 10⁹/L | Interrupt until ANC is ≥1 × 10⁹/L |
| Febrile neutropenia (fever ≥38.5° C. and | Monitor CBC at least weekly. |
| ANC <1 × 10⁹/L) | |
| ANC <1 × 10⁹/L and infection of any grade | |

Thrombocytopenia

| | |
|---|---|
| Grade 3: | Interrupt until platelet count is ≥50 × 10⁹/L |
| Platelet count 25-49 × 10⁹/L or evidence of | Monitor CBC at least weekly. |
| bleeding with platelet count ≥25 to 50 × 10₉/L | |
| Grade 4: | |
| Platelet count <25 × 10⁹/L | |

Anemia

| | |
|---|---|
| Grade 4 | Interrupt |
| | Monitor CBC at least weekly. |
| Return to Grade ≤3 | Resume. |

^{a}The investigator should consider the use of growth factors.

TABLE 27
Dosing Interruption for Nonhematologic Adverse Events.

| Non-hematologic Toxicity | Talquetamab |
|---|---|
| First sign of CRS | X |
| First sign of ICANS | X |
| Grade ≥3 TEAEs (except disease-related pain) or Grade ≥3 | X |
| laboratory toxicity that are clinically significant (eg, | |
| requiring intervention or associated with an AE) | |
| HBV reactivation | X |
| Any grade infection prior to/during step-up dosing and | If deemed related |
| throughout subsequent treatment doses. | to talquetamab |

TABLE 28
Daratumumab Dosing Interruption for Non-hematologic Adverse Events.

| Non-hematologic Toxicity | Daratumumab SC |
|---|---|
| Grade ≥3 TEAEs (except disease-related pain) or Grade ≥3 laboratory | X |
| toxicity that are clinically significant (eg, requiring intervention or | |
| associated with an AE)^{a} | |
| HBV reactivation | X |
| Any grade infection prior to/during step-up dosing and throughout | Investigator discretion |
| subsequent treatment doses | |

^{a}Isolated laboratory hypogammaglobulinemia does not require dose interruption of any drug.

TABLE 29
Retreatment Criteria Before the Start of Each DRd Induction Cycle.

| Laboratory Parameter | Requirements Before Each Study Agent Administration |
|---|---|
| Absolute neutrophil count | ≥1.0 10⁹/L |
| Platelet count | ≥50 × 10⁹/L |
| Hemoglobin | ≥7.5 g/dL (≥4.96 mmol/L) |

TABLE 30
Dose Reduction Guidance for Lenalidomid Adverse Events.

Lenalidomide Dose

| | | |
|---|---|---|
| Starting dose | 25 | mg, Days 1-21 |
| 1st dose reduction | 15 | mg, Days 1-21 |
| 2nd dose reduction | 10 | mg, Days 1-21 |
| 3rd dose reduction | 5 | mg, Days 1-21 |

| | |
|---|---|
| 4th dose reduction | Discontinue lenalidomide permanently. |

TABLE 31
Dose Reduction Guidance for Dexamethasone Adverse Events.

| | Dexamethasone Dose (DRd) |
|---|---|
| Starting dose | 40 mg |
| 1st dose reduction | 20 mg |
| 2nd dose reduction | 10 mg |
| 3rd dose reduction | Discontinue dexamethasone. |

TABLE 32
Daratumumab SC Dose Skips.

| Study | | Dosing | Skip Dose if Dosing | |
|---|---|---|---|---|
| Drug(s) | Cycles | Frequency | Interrupted: | Resume Dosing |
| Daratumumab | Cycle 1-2 | Weekly | >3 days from | Next planned weekly |
| SC | | | planned dose date | dosing day |
| | Cycle 3-4 | Q2W | >7 days from | Next planned Q2W |
| | | | planned dose date | dosing day |

TABLE 33
Daratumumab Dosing Interruption
for Hematologic Adverse Events.

| | |
|---|---|
| Hematologic AE | Daratumumab |

Neutropenia^{a}

| | |
|---|---|
| ANC <0.5 × 10⁹/L | Interrupt until ANC |
| Febrile neutropenia (fever ≥38.5° C. | is ≥1 × 10⁹/L |
| and ANC <1 × 10⁹/L) | Monitor CBC weekly. |
| ANC <1 × 10⁹/L and infection of | |
| any grade | |

Thrombocytopenia

| | |
|---|---|
| Grade 3: | Interrupt until platelet count |
| Platelet count 25-49 × 10⁹/L or evidence | is ≥50 × 10⁹/L |
| of bleeding with platelet count ≥25 to | |
| 50 × 10⁹/L | |
| Grade 4: | Monitor CBC weekly. |
| Platelet count <25 × 10⁹/L | |

Anemia

| | |
|---|---|
| Grade 4 | Interrupt |
| | Monitor CBC at least weekly. |
| Return to Grade ≤3 | Resume. |

^{a}The investigator should consider the use of growth factors.

TABLE 34
Lenalidomide Dose Modifications Due to Renal Impairment.

| | | Lenalidomide Dose | |
|---|---|---|---|
| | | Days 1-21 of 28-day | |
| Category | Renal Function^{a} | Cycle | |
| Moderate renal | CrCl 30 to 60 mL/min | 10 | mg^{b} |
| impairment | | | |
| Severe renal | CrCl <30 mL/min (not | 15 | mg every 48 hours |
| impairment | requiring dialysis) | | |
| End-stage renal | CrCl <30 | 5 | mg^{c} |
| disease | | | |

^{a}Estimated by CrCl as calculated by the Cockcroft-Gault.
^{b}Consider escalating dose to 15 mg daily after 2 cycles if well tolerated.
^{c}On dialysis days, administer dose after dialysis.

TABLE 35
Bone Marrow Testing.

| Timepoint | Notes | Local Testing | Central Testing |
|---|---|---|---|
| Screening | Disease | Evaluate BMPC % | FISH analysis^{a} MRD |
| | characterization | Evaluate clonality of | BMA (if dry tap, BMB |
| | (morphology and either | BMPCs by flow | can be used) |
| | IHC or flow cytometry | cytometry or IHC/IF | Immuno-phenotyping |
| | performed locally): | FISH analysis | |
| | standard of care testing | | |
| | within 42 days of | | |
| | apheresis can be used; | | |
| | fresh aspirate samples | | |
| | need to be obtained and | | |
| | sent to central | | |
| | laboratory for MRD | | |
| | and molecular | | |
| | assessments. | | |
| At time of suspected | If these timepoints | Evaluate BMPC % to | MRD |
| CR/sCR and PD (prior | occur within 1 month | confirm CR | |
| to SST). At 6 months | of another bone marrow | Evaluate clonality of | |
| and 12 months from | aspiration, a repeat | BMPCs by flow | |
| suspected CR/.sCR | bone marrow aspiration | cytometry or IHC/IF to | |
| | will not be requested. | confirm sCR | |
| Disease progression, if | IHC/IF (both require | Evaluate BMPC % | BMA (if dry tap, BMB |
| feasible | kappa/lambda ratio | Evaluate clonality of | can be used) |
| | from analysis of >100 | BMPCs by flow | Immuno-phenotyping |
| | PCs) or 2- to 4-color | cytometry or IHC/IF | |
| | flow cytometry are | | |
| | acceptable methods to | | |
| | evaluate PC clonality. | | |

^{a}Local FISH testing should be done for cytogenetics. If the local assessment fails and an archival FISH result is not available, a fresh aspirate sample needs to be obtained and sent to the central laboratory for FISH testing. Historical FISH result may be requested for some participants.

TABLE 36
Eligibility Related to Hepatitis B Test Results.

| | Hepatitis B Test Result | | | |
|---|---|---|---|---|
| Action | hBsAg | anti-HBS | anti-HBC | HBV-DNA^{a} |
| Exclude | Participants who are hBsAg positive or HBV-DNA positive are excluded | | | |
| | from the study regardless of the status of anti-HBs and anti-HBC. | | | |
| Include | Negative | Negative | Negative | Not required |
| | Negative | Positive | Positive | Negative |
| | Negative | Negative | Positive | Negative |
| | Negative | Positive | Negative | Negative^{b} |

^{a}In participants with negative hBsAg test, an HBV-DNA quantification test is required in the following participants to determine eligibility:
Participants who are anti-HBs positive and without history of vaccination.
Participants with positive anti-HBc and either positive or negative anti-HBS.
^{b}Participants with serologic findings suggestive of HBV vaccination (ie, anti-HBS positivity as the only serologic marker) and a known history of prior HBV vaccination do not need to be tested for HBV-DNA by PCR.

TABLE 37
Guidelines for the Management of CRS.

| CRS Grade^{a} | Tocilizumab^{b} | Corticosteroids^{f} |
|---|---|---|
| Grade 1 | Tocilizumab 8 mg/kg IV over 1 | May be considered |
| Temperature ≥38.0° C.^{c} | hour (not to exceed 800 mg) | |
| | may be considered | |
| Grade 2 | Administer tocilizumab 8 mg/kg | Consider methylprednisolone |
| Symptoms require and respond | IV over 1 hour (not to exceed | 1 mg/kg IV twice daily or |
| to moderate | 800 mg). | equivalent dexamethasone (eg, |
| intervention. | Repeat tocilizumab every 8 | 10 mg IV every 6 hours). |
| Temperature ≥38.0° C.^{c} with: | hours as needed if not | |
| Hypotension not requiring | responsive to IV fluids up to 1 | |
| vasopressors, and/or, | liter or increasing supplemental | |
| Hypoxia requiring oxygen via | oxygen. | |
| canulae or blow-by | If no improvement within 24 hours or rapid progression, repeat | |
| | tocilizumab and escalate dose and frequency of dexamethasone (20 | |
| | mg IV every 6 to 12 hours). | |
| | After 2 doses of tocilizumab, consider alternative anticytokine | |
| | agents.^{d} Do not exceed 3 doses of tocilizumab in 24 hours, or 4 | |
| | doses in total. | |
| Grade 3 | Per Grade 2 | Administer methylprednisolone |
| Symptoms require and respond | | 1 mg/kg IV twice daily or |
| to aggressive intervention. | | equivalent dexamethasone (eg, |
| | | 10 mg IV every 6 hours). |
| Temperature ≥38.0° C.^{c} with: | If no improvement within 24 hours or rapid progression, repeat | |
| Hypotension requiring 1 | tocilizumab and escalate dose and frequency of dexamethasone (20 | |
| vasopressor with or without | mg IV every 6 to 12 hours). | |
| vasopressin, | If no improvement within 24 hours or continued rapid progression, | |
| and/or, | switch to methylprednisolone 2 mg/kg IV every 12 hours. | |
| Hypoxia requiring oxygen via | After 2 doses of tocilizumab, consider alternative anticytokine | |
| high-flow nasal canulae, | agents.^{d} Do not exceed 3 doses of tocilizumab in 24 hours, or 4 | |
| facemask, nonrebreather mask, | doses in total. | |
| or Venturi mask | | |
| Grade 4 Life-threatening | Per Grade 2 | Administer dexamethasone 20 |
| symptoms. Requirements for | | mg IV every 6 hours. |

| | |
|---|---|
| ventilator support, CVVHD. | After 2 doses of tocilizumab, consider alternative anticytokine |
| Temperature ?38.0° C.^{c} with: | agents.^{d} Do not exceed 3 doses of tocilizumab in 24 hours, or 4 |
| Hypotension requiring multiple | doses in total. |
| vasopressors (excluding | If no improvement within 24 hours, consider methylprednisolone |
| vasopressin), | (1-2 g IV, repeat every 24 hours if needed; taper as clinically |
| and/or, | indicated) or other immunosuppressants (eg, other anti-T cell |
| Hypoxia requiring positive | therapies). |
| pressure (eg, CPAP, BiPAP, | |
| intubation, and mechanical | |
| ventilation) | |

^{a}Based on ASTCT consensus grading (Lee 2019).
^{b}Refer to tocilizumab prescribing information for details.
^{c}Attributed to CRS. Fever may not always be present concurrently with hypotension or hypoxia, as it may be masked by interventions such as antipyretics or anticytokine therapy (eg, tocilizumab or steroids). Absence of fever does not impact CRS management decision. In this case, CRS management is driven by hypotension and/or hypoxia and by the more severe symptom that is not attributable to any other cause.
^{d}Monoclonal antibodies targeting cytokines may be considered based on institutional practice for unresponsive CRS.
e. Low-flow nasal cannula is ≤6 L/min; high-flow nasal cannula is >6 L/min.
^{f}Continue corticosteroids use until the event is ≤Grade 1; taper steroids if total corticosteroid exposure is greater than 3 days.

TABLE 38
Guidelines for the Management of ICANS.

| ICANS Grade^{a} | Corticosteroids |
|---|---|
| Grade 1 | Consider dexamethasone^{c} 10 mg IV every 6 to 12 |
| ICE score 7-9^{b} | hours for 2 to 3 days |
| or depressed level of consciousness: awakens | Consider nonsedating, antiseizure medicines (eg, |
| spontaneously | levetiracetam) for seizure prophylaxis. |
| Grade 2 | Administer dexamethasone^{c} 10 mg IV every 6 |
| ICE score-3-6^{b} | hours for 2 to 3 days, or longer for persistent |
| or depressed level of consciousness: awakens to | symptoms. |
| voice | Consider steroid taper if total corticosteroid |
| | exposure is greater than 3 days. |
| | Consider nonsedating, antiseizure |
| Grade 3 | Administer dexamethasone^{c} 10 to 20 mg IV every |
| ICE score-0-2^{b} | 6 hours. |
| (If ICE score is 0, but participant is arousable | If no improvement after 48 hours or worsening of |
| (eg, awake with global aphasia) and able to | neurologic toxicity, escalate dexamethasone^{c} dose |
| perform assessment) | to at least 20 mg IV every 6 hours; taper within 7 |
| or depressed level of consciousness: awakens only | days, |
| to tactile stimulus, | or escalate to high-dose methylprednisolone |
| or seizures, either: | (1 g/day, repeat every 24 hours if needed; taper as |
| any clinical seizure, focal or generalized, that | clinically indicated). |
| resolves rapidly, or | Consider nonsedating, antiseizure medicines (eg, |
| non-convulsive seizures on EEG that resolve | levetiracetam) for seizure prophylaxis. |
| with intervention, | |
| or raised ICP: focal/local edema on | |
| neuroimaging.^{d} | |
| Grade 4 | Administer dexamethasone^{c} 10 to 20 mg IV every |
| ICE score-0^{b} (participant is unarousable and | 6 hours. |
| unable to perform ICE assessment), | If no improvement after 24 hours or worsening of |
| or depressed level of consciousness, either: | neurologic toxicity, escalate to high-dose |
| participant is unarousable or requires vigorous or | methylprednisolone (1 to 2 g/day, repeated every |
| repetitive tactile stimuli to arouse, or | 24 hours if needed; taper as clinically indicated). |
| stupor or coma, | Consider nonsedating, antiseizure medicines (eg, |
| or seizures, either: | levetiracetam) for seizure prophylaxis. |
| life-threatening prolonged seizure (>5 min), or | If raised ICP/cerebral edema is suspected, |
| repetitive clinical or electrical seizures without | consider hyperventilation and hyperosmolar |
| return to baseline in between, | therapy. Give high-dose methylprednisolone (1 to |
| or motor findings^{e}: | 2 g/day, repeat every 24 hours if needed; taper as |
| deep focal motor weakness such as hemiparesis | clinically indicated), and consider neurology |
| or paraparesis, | and/or neurosurgery consultation. |
| or raised ICP/cerebral edema, with | |
| signs/symptoms such as: | |
| diffuse cerebral edema on neuroimaging, or | |
| decerebrate or decorticate posturing, or | |
| cranial nerve VI palsy, or | |
| papilledema, or | |
| Cushing's triad. | |

^{a}Based on ASTCT consensus grading (Lee 2019).
^{b}If participant is arousable and able to perform ICE assessment, assess: Orientation (oriented to year, month, city, hospital = 4 points); Naming (name 3 objects, eg, point to clock, pen, button = 3 points); Following Commands (eg, “show me 2 fingers” or “close your eyes and stick out your tongue” = 1 point); Writing (ability to write a standard sentence = 1 point); and Attention (count backwards from 100 by 10 = 1 point). If participant is unarousable and unable to perform ICE assessment (Grade 4 ICANS) = 0 points.
^{c}All references to dexamethasone administration mean dexamethasone or equivalent.
^{d}Intracranial hemorrhage with or without associated edema is not considered a neurotoxicity feature and is excluded from ICANS grading. It may be graded according to NCI-CTCAE Version 5.0.
^{e}Tremors and myoclonus associated with immune effector cell therapies may be graded according to NCI-CTCAE Version 5.0, but they do not influence ICANS grading.

TABLE 39
Guidelines for the Management of sARRs.

| NCI-CTCAE | Presenting Symptoms | Recommended Treatment/Intervention |
|---|---|---|
| Grade 1 or | Mild or moderate | Start IV fluids; give diphenhydramine 50 mg (or |
| Grade 2 | reaction; requires | equivalent) IV and/or paracetamol (acetaminophen) 650 to |
| | therapy interruption but | 1,000 mg; consider corticosteroids and bronchodilator |
| | responds promptly to | therapy; monitor participant closely until recovery from |
| | symptomatic treatment | symptoms. |
| | | Consider hospitalization if Grade 2 laryngeal edema or |
| | | Grade 2 bronchospasm occurs. |
| Grade 3 | Severe, prolonged (ie, | Start IV saline infusion; recommend bronchodilators (if |
| | not rapidly responsive | indicated), epinephrine 0.2 to 1 mg of a 1:1,000 solution |
| | to symptomatic | for SC administration or 0.1 to 0.25 mg of a 1:10,000 |
| | medication; recurrence | solution injected slowly for IV administration, and/or |
| | of symptoms following | diphenhydramine 50 mg IV with methylprednisolone 100 |
| | initial improvement; | mg IV (or equivalent), as needed, and other drugs as |
| | hospitalization | appropriate. |
| | indicated for other | Investigators should follow their institutional guidelines for |
| | clinical sequelae [eg, | the treatment of anaphylaxis. |
| | renal impairment, | Monitor until medically stable, per the investigator's |
| | pulmonary infiltrates]) | medical judgment. |

| Grade 4 | Required hospitalization for subsequent administration(s) |
|---|---|
| Life-threatening; pressor or ventilator | of talquetamab after a Grade 3 sARR related to |
| support indicated | talquetamab. |
| | Participants who experience a Grade 4 sARR must |
| | discontinue talquetamab study treatment (see below). |
| Discontinuation of treatment | sARRs requiring discontinuation of study treatment |
| | (talquetamab SC) |
| General | Prophylactic medications (after initial event) |
| | In the case of late-occurring hypersensitivity symptoms |
| | (eg, appearance of a localized or generalized pruritis within |
| | 1 week after treatment), symptomatic treatment must be |
| | given (eg, PO antihistamine or corticosteroids), as |
| | appropriate. |

TABLE 40
Handwriting Adverse Event Toxicity Grading Criteria.

| Adverse Event Term | Grade 1 | Grade 2 |
|---|---|---|
| Micrographia: | Mildly smaller letters or reduced | Moderate to severely smaller |
| abnormally small or cramped | spacing (eg, <50% decrease | letters or reduced spacing |
| handwriting | from baseline) | (eg, >=50% decrease from baseline) |
| Dysgraphia: | Mildly slower writing, impaired | Moderate to severely slower |
| illegible writing or writing that | straightness of line, difficulty in | writing, impaired straightness of |
| takes an unusually long time or | completing task from baseline; | line, difficulty in completing |
| great effort | most words are legible | task from baseline; most words |
| | | are illegible |
| Agraphia: | Able to write part of a sentence | Able to write just 1 to 2 words, |
| pathologic loss of the ability to | (3 or more words); noted change | or unable to write any words; |
| write | from baseline | noted change from baseline |
| write | | |

TABLE 41A
Schedule of Activities for Pharmacokinetic, Immunogenicity,
and Biomarker Assessments (Cohorts 1 and 2).^{a}

| | | | DRd Induction | Conditioning |
|---|---|---|---|---|
| Assessment | Screening | Apheresis | Treatment | Regimen |
| Day | | | 1 | −5*, −4, −3 |
| Window | | | | * for start: D −7 |
| | | | | to D −5^{c} |
| PK CAR | | | | |
| transgene levels (blood) | | | | |
| Cilta-cel ADA (serum) | | | | |
| Cilta-cel/talquetamab | X | | C1D1 only^{b} | D −5 only |
| immunopheno-typing (blood) | | | | |
| Cytokines (blood) | X | | Collect additional pharmacodynamic | |
| | | | cytokine samples, if feasible, when any of | |
| | | | the following are observed or reported: (1) | |
| | | | suspected sARRs reaction Grade ≥2; (2) | |
| | | | CRS event Grade ≥2 (at onset of the CRS | |
| | | | event, and 24 h and 72 h after); (3) as | |
| | | | indicated based on emerging data. | |
| RCL (blood) | X | | At approximately 3 months (D 84), 6 months | |
| | | | (D 168), and 12 months (D 364) (all with | |
| | | | window of ±1 month) after cilta-cel | |
| | | | infusion. Additional samples may be | |
| | | | collected if triggered by events that may be | |
| | | | relevant, but not limited to, RCL per clinical | |
| | | | assessment. | |
| Immune and omic profiling | | X^{d} | | |
| (PBMC)^{f} | | | | |

^{a}All samples predose (on dosing days).
^{b}For Cohort 2 only.
^{c}If using a window, collection should be taken prior to the first dose of conditioning regimen.
^{d}If a repeat apheresis is to be performed, another apheresis sample should be collected for PD assessments at the time of apheresis.
e. Predose sample window −4 to 0 hour of cilta-cel administration for pharmacokinetic and immunogenicity sampling.
^{f}PBMC collection taken at the time of apheresis.

TABLE 41B
Schedule of Activities for Pharmacokinetic, Immunogenicity, and Biomarker Assessments (Cohorts 1 and 2).^{a}

| Assessment | | | |
|---|---|---|---|
| Cilta-cel | Infusion Consolidation Post-Infusion | Talquetamab | Follow-Up |

| Day | | | | | | | | | | |
|---|---|---|---|---|---|---|---|---|---|---|
| 1 | 1 | 7 | 10 | 14 | 21 | 28 | 42 | 56 | Q4W | FOT/PD |

Window

| | | ±1 d | ±1 d | ±1 d | ±1 d | ±2 d | ±2 d | ±2 d | | | |
|---|---|---|---|---|---|---|---|---|---|---|---|
| PK CAR | X^{e} | X | | X | | X | | X | See Table 8 | X | X |
| transgene | | | | | | | | | | | |
| levels | | | | | | | | | | | |
| (blood) | | | | | | | | | | | |
| Cilta-cel | X^{e} | | | X | | X | | X | See Table 8 | X | X |
| ADA | | | | | | | | | | | |
| (serum) | | | | | | | | | | | |
| Cilta-cel/ | X | X | X | X | X | X | X | X | See Table 8 | | X |
| talqueta | | | | | | | | | | | |
| mab | | | | | | | | | | | |
| immunopheno- | | | | | | | | | | | |
| typing | | | | | | | | | | | |
| (blood) | | | | | | | | | | | |

| | |
|---|---|
| Cytokines | Collect additional pharmacodynamic cytokine samples, if feasible, when any of |
| (blood) | the following are observed or reported: (1) suspected sARRs reaction Grade ≥2; |
| | (2) CRS event Grade ≥2 (at onset of the CRS event, and 24 h and 72 h after); (3) |
| | as indicated based on emerging data. |

| | |
|---|---|
| RCL (blood) | At approximately 3 months (D 84), 6 months (D 168), and 12 months (D 364) (all with |
| | window of ±1 month) after cilta-cel infusion. Additional samples may be collected if |
| | triggered by events that may be relevant, but not limited to, RCL per clinical assessment. |

^{a}All samples predose (on dosing days).
b. For Cohort 2 only.
c. If using a window, collection should be taken prior to the first dose of conditioning regimen.
d. If a repeat apheresis is to be performed, another apheresis sample should be collected for PD assessments at the time of apheresis.
^{e}Predose sample window −4 to 0 hour of cilta-cel administration for pharmacokinetic and immunogenicity sampling.
f. PBMC collection taken at the time of apheresis.

TABLE 42A
Schedule of Activities for Pharmacokinetic and Biomarker Assessments (Cohort 3).^{a}

| | | 1^{st} | Tal Bridging | 2^{nd} | Conditioning |
|---|---|---|---|---|---|
| Assessment | Screening | Apheresis | Regimen | Apheresis^{b} | Regimen |
| Day | | | | | −5*, −4, −3 |
| Window | | | | | * for start: D −7 to D −5 |
| PK CAR | | | | | |
| transgene levels | | | | | |
| (blood) | | | | | |
| ADA (serum) | | | | | |
| Cilta- | X | | Table 8 | | |
| cel/talquetamab | | | | | |
| typic (blood) | | | | | |
| Cytokines | X | | Collect additional pharmacodynamic cytokine samples, if | | |
| (blood) | | | feasible when any of the following are observed or | | |
| | | | reported: (1) suspected sARRs reaction Grade 22; (2) | | |
| | | | CRS event Grade 22 (at onset of the CRS event, and 24 h | | |
| | | | and 72 h after); (3) as indicated based on emerging data. | | |
| RCL (blood) | X | | | | |
| Immune and | | X^{c} | | X^{c} | |
| omic profiling | | | | | |
| (PBMC)^{c} | | | | | |

^{a}All samples predose (on dosing days).
^{b}The first 5 participants will also undergo a second apheresis after bridging.
^{c}If a repeat apheresis is to be performed, another apheresis sample should be collected for pharmacodynamic assessments at the time of apheresis.
d. Predose sample window −4 to 0 hour of cilta-cel administration for pharmacokinetic and immunogenicity sampling.
e. PBMC collection taken at the time of apheresis.

TABLE 42B
Schedule of Activities for Pharmacokinetic and Biomarker Assessments (Cohort 3).^{a}

| Assessment | | | | | | | | | | | |
|---|---|---|---|---|---|---|---|---|---|---|---|
| Cilta-cel | | | | | | | | | | | |
| Infusion | Post-Infusion | | | | | | | | | Follow-Up | |
| Day | | | | | | | | | | | |
| | | | | | | | | | | Q4W | |
| 1 | 7 | 10 | 14 | 21 | 28 | 42 | 56 | 78 | 100 | post-D 100 | Post-PD |

Window

| | | | | | | | | |
|---|---|---|---|---|---|---|---|---|
| ±1 d | ±1 d | ±1 d | ±1 d | ±2 d | ±2 d | ±2 d | ±2 d | ±2 d |

| | | | | | | | | | |
|---|---|---|---|---|---|---|---|---|---|
| PK CAR | X^{d} | X | | X | X | X | X | X | X |
| transgene levels | | | | | | | | | |
| (blood) | | | | | | | | | |
| ADA (serum) | X^{d} | | | X | X | X | X | X | X |
| Cilta-cel/ | X | X | X | X | X | X | X | | X |
| talquetamab | | | | | | | | | |
| immunopheno | | | | | | | | | |
| typing (blood) | | | | | | | | | |

| | |
|---|---|
| Cytokines | Collect additional pharmacodynamic cytokine samples, if feasible |
| (blood) | when any of the following are observed or reported: (1) |
| | suspected sARRs reaction Grade 22; (2) CRS event Grade 22 (at |
| | onset of the CRS event, and 24 h and 72 h after); (3) as |
| | indicated based on emerging data. |

| | |
|---|---|
| RCL (blood) | At approximately 3 months (D 84), 6 months (D 168), and 12 months (D 364) (all |
| | with window of ±1 month) after cilta-cel infusion. Additional samples may be |
| | collected if triggered by events that may be relevant, but not limited to RCL, per |
| | clinical assessment |

^{a}All samples predose (on dosing days).
b. The first 5 participants will also undergo a second apheresis after bridging.
c. If a repeat apheresis is to be performed, another apheresis sample should be collected for pharmacodynamic assessments at the time of apheresis.
^{d}Predose sample window −4 to 0 hour of cilta-cel administration for pharmacokinetic and immunogenicity sampling.
e. PBMC collection taken at the time of apheresis.

TABLE 43
Schedule of Activities for Talquetamab Pharmacokinetic,
Immunogenicity, and Biomarker Assessment During Talquetamab
Bridging and Consolidation^{a} (Cohorts 1, 2, and 3).

| | Assessment | | | | | | | |
|---|---|---|---|---|---|---|---|---|
| | | | | | Cycle | Cycle | Cycle | Cycle |
| | Cycle 1 | | | | 2 | 3 | 7b | 12b |
| | Day | | | | | | | |
| | 1 | 4 | 8 | 15 | 1 | 1 | 1 | 1 |
| | Window | | | | | | | |
| | | ±2 d | ±2 d | ±2 d | ±3 d | ±3 d | ±3 d | ±3 d |
| Cilta-cel/ | X | | | X | X | X | X | X |
| talquetamab | | | | | | | | |
| phenotyping | | | | | | | | |
| (blood) | | | | | | | | |
| Talquetamab PK | X | X | X | X | X | X | X | X |
| (serum)^{d} | | | | | | | | |
| Talquetamab | X | | | | X | X | X | X |
| ADA (serum)^{d} | | | | | | | | |
| Cytokines^{c} | X^{b} | | | X^{b} | | | | |
| (blood) | | | | | | | | |

^{a}All samples predose.
^{b}For Cohorts 1 and 2 only.
^{c}Collect additional pharmacodynamic cytokine samples, if feasible, when any of the following are observed or reported: (1) suspected sARRs reaction Grade 22; (2) CRS event Grade 22 (at onset of the CRS event, and 24 h and 72 h after); (3) as indicated based on emerging data.
^{d}Predose sample window −4 to 0 hour of talquetamab administration for pharmacokinetic and immunogenicity sampling.

TABLE 44
Analysis Sets.

| Analysis Sets | Description |
|---|---|
| Enrolled | All participants who sign the ICF |
| All-Treated | Participants who received at least one dose of any study treatment. This analysis set |
| | will be used for safety summaries. |
| Evaluable | For Cohort 1, participants who received cilta-cel at the target dose and at least one |
| | dose of talquetamab before disease progression. |
| | For Cohort 2, participants who received at least one dose of DRd induction therapy |
| | and cilta-cel at the targeted dose and at least 1 dose of talquetamab before disease |
| | progression. |
| | For Cohort 3, participants who received at least 2 cycles of talquetamab as bridging |
| | therapy and cilta-cel at the targeted dose before disease progression. |
| | This analysis set will be used for efficacy summaries. |
| PK Evaluable | For cilta-cel: all participants who received cilta-cel infusion and have at least 1 |
| | post-dose pharmacokinetic sample. |
| | For talquetamab: all participants who received at least one dose of talquetamab and |
| | have at least 1 post-dose pharmacokinetic sample. |
| Immunogenicity | For cilta-cel: all participants who received cilta-cel infusion and have at least 1 |
| Evaluable | post-dose immunogenicity sample. |
| | For talquetamab: all participants who received at least one dose of talquetamab and |
| | have at least 1 post-dose immunogenicity sample. |
| PRO Evaluable | All participants who received at least one dose of talquetamab and have at least 1 |
| | post-dose PRO-CTCAE, EORTC-Q168 and PGI-S assessment |

TABLE 45
Secondary Endpoint(s).

| Endpoint | Definition |
|---|---|
| Overall response | The proportion of participants who have a PR or better according to the |
| | IMWG response criteria. Response to treatment will be evaluated by |
| | validated computerized algorithm. |
| VPGR or better | The proportion of participants who achieve a VGPR or better response |
| (sCR + CR + VGPR) | according to the IMWG response criteria. |
| CR/sCR | The proportion of participants with best overall response of CR or sCR. |
| MRD-negativity | The proportion of participants who achieve MRD-negativity at a threshold of |
| | 10−5 at any timepoint after the date of the first study treatment and before |
| | disease progression or start of any subsequent antimyeloma therapy. |
| Sustained MRD- | The proportion of participants who sustained MRD-negative status, as |
| negativity (≥6 | determined by NGS with sensitivity of 10−5, for at least 6 months without |
| months) | examination showing MRD-positive or PD in between. |
| DOR | The time from the date of initial documentation of a response (PR or better) |
| | to the date of first documented evidence of PD, as defined in the IMWG |
| | response criteria or death due to any cause, whichever occurs first. Relapse |
| | from CR is not considered as disease progression. For participants who have |
| | not progressed or died, data will be censored at the last disease evaluation |
| | before the start of any subsequent antimyeloma therapy. |
| TTR | The time between date of the first study treatment and the first efficacy |
| | evaluation that the participant met all criteria for PR or better. |
| PFS | The time from the date of the first study treatment to the date of first |
| | documented disease progression, as defined in the IMWG response criteria, |
| | or death due to any cause, whichever occurs first. For participants who have |
| | not progressed and are alive, data will be censored at the last disease |
| | evaluation before the start of any subsequent antimyeloma therapy. |
| OS | Measured from the date of the first study treatment to the date of the |
| | participant's death. If the participant is alive or the vital status is unknown, |
| | then the participant's data will be censored at the date the participant was last |
| | known to be alive. |

### SEQUENCES

Ciltacabtagene autoleucel

SEQ ID NO: 1-Ciltacabtagene autoleucel CAR CD8α signal peptide,
CD8α SP amino acid sequence
MALPVTALLLPLALLLHAARP
SEQ ID NO: 2-Ciltacabtagene autoleucel CAR BCMA binding domain,
VHH1 amino acid sequence
QVKLEESGGGLVQAGRSLRLSCAASEHTFSSHVMGWFRQAPGKERESVAVIGWRDISTS
YADSVKGRFTISRDNAKKTLYLQMNSLKPEDTAVYYCAARRIDAADFDSWGQGTQVT
VSS
SEQ ID NO: 3-Ciltacabtagene autoleucel CAR BCMA binding domain,
G4S linker amino acid sequence
GGGGS
SEQ ID NO: 4-Ciltacabtagene autoleucel CAR BCMA binding domain,
VHH2 amino acid sequence
EVQLVESGGGLVQAGGSLRLSCAASGRTFTMGWFRQAPGKEREFVAAISLSPTLAYYAE
SVKGRFTISRDNAKNTVVLQMNSLKPEDTALYYCAADRKSVMSIRPDYWGQGTQVTVS
S
SEQ ID NO: 5-Ciltacabtagene autoleucel CAR CD8α hinge amino
acid sequence
TTTPAPRPPTPAPTIASQPLSLRPEACRPAAGGAVHTRGLDFACD
SEQ ID NO: 6-Ciltacabtagene autoleucel CAR CD8α transmembrane
amino acid sequence
IYIWAPLAGTCGVLLLSLVITLYC
SEQ ID NO: 7-Ciltacabtagene autoleucel CAR CD137 Cytoplasmic
amino acid sequence
KRGRKKLLYIFKQPFMRPVQTTQEEDGCSCRFPEEEEGGCEL
SEQ ID NO: 8-Ciltacabtagene autoleucel CAR CD3z Cytoplasmic
amino acid sequence
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 9-Ciltacabtagene autoleucel CAR CD8α signal peptide
CD8α SP nucleic acid sequence
ATGGCTCTGCCCGTCACCGCTCTGCTGCTGCCTCTGGCTCTGCTGCTGCACGCTGCTC
GCCCT
SEQ ID NO: 10-Ciltacabtagene autoleucel CAR BCMA binding domain,
VHH1 nucleic acid sequence
CAGGTCAAACTGGAAGAATCTGGCGGAGGCCTGGTGCAGGCAGGACGGAGCCTGCG
CCTGAGCTGCGCAGCATCCGAGCACACCTTCAGCTCCCACGTGATGGGCTGGTTTCG
GCAGGCCCCAGGCAAGGAGAGAGAGAGCGTGGCCGTGATCGGCTGGAGGGACATC
TCCACATCTTACGCCGATTCCGTGAAGGGCCGGTTCACCATCAGCCGGGACAACGCC
AAGAAGACACTGTATCTGCAGATGAACAGCCTGAAGCCCGAGGACACCGCCGTGTA
CTATTGCGCAGCAAGGAGAATCGACGCAGCAGACTTTGATTCCTGGGGCCAGGGCA
CCCAGGTGACAGTGTCTAGC
SEQ ID NO:111-Ciltacabtagene autoleucel CAR BCMA binding domain,
G4S linker nucleic acid sequence
GGAGGAGGAGGATCT
SEQ ID NO: 12-Ciltacabtagene autoleucel CAR BCMA binding domain,
VHH2 nucleic acid sequence
GAGGTGCAGCTGGTGGAGAGCGGAGGCGGCCTGGTGCAGGCCGGAGGCTCTCTGAG
GCTGAGCTGTGCAGCATCCGGAAGAACCTTCACAATGGGCTGGTTTAGGCAGGCAC
CAGGAAAGGAGAGGGAGTTCGTGGCAGCAATCAGCCTGTCCCCTACCCTGGCCTAC
TATGCCGAGAGCGTGAAGGGCAGGTTTACCATCTCCCGCGATAACGCCAAGAATAC
AGTGGTGCTGCAGATGAACTCCCTGAAACCTGAGGACACAGCCCTGTACTATTGTGC
CGCCGATCGGAAGAGCGTGATGAGCATTAGACCAGACTATTGGGGGCAGGGAACAC
AGGTGACCGTGAGCAGC
SEQ ID NO: 13-Ciltacabtagene autoleucel CAR CD8α hinge nucleic
acid sequence
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGAT
SEQ ID NO: 14-Ciltacabtagene autoleucel CAR CD8α transmembrane
nucleic acid sequence
ATCTACATCTGGGCGCCCTTGGCCGGGACTTGTGGGGTCCTTCTCCTGTCACTGGTTA
TCACCCTTTACTGC
SEQ ID NO: 15-Ciltacabtagene autoleucel CAR CD137 Cytoplasmic
nucleic acid sequence
AAACGGGGCAGAAAGAAACTCCTGTATATATTCAAACAACCATTTATGAGACCAGT
ACAAACTACTCAAGAGGAAGATGGCTGTAGCTGCCGATTTCCAGAAGAAGAAGAAG
GAGGATGTGAACTG
SEQ ID NO: 16-Ciltacabtagene autoleucel CAR CD3z Cytoplasmic
nucleic acid sequence
AGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCG
CTAA
SEQ ID NO: 17-Ciltacabtagene autoleucel CAR amino acid sequence
MALPVTALLLPLALLLHAARPQVKLEESGGGLVQAGRSLRLSCAASEHTFSSHVMGWF
RQAPGKERESVAVIGWRDISTSYADSVKGRFTISRDNAKKTLYLQMNSLKPEDTAVYYC
AARRIDAADFDSWGQGTQVTVSSGGGGSEVQLVESGGGLVQAGGSLRLSCAASGRTFT
MGWFRQAPGKEREFVAAISLSPTLAYYAESVKGRFTISRDNAKNTVVLQMNSLKPEDT
ALYYCAADRKSVMSIRPDYWGQGTQVTVSSTSTTTPAPRPPTPAPTIASQPLSLRPEACR
PAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLY
QGLSTATKDTYDALHMQALPPR
SEQ ID NO: 18-Ciltacabtagene autoleucel CAR BCMA binding domain,
VHH1 CDR1 amino acid sequence
SHVMG
SEQ ID NO: 19-Ciltacabtagene autoleucel CAR BCMA binding domain,
VHH1 CDR2 amino acid sequence
VIGWRDISTSYADSVKG
SEQ ID NO: 20-Ciltacabtagene autoleucel CAR BCMA binding domain,
VHH1 CDR3 amino acid sequence
ARRIDAADFDS
SEQ ID NO: 21-Ciltacabtagene autoleucel CAR BCMA binding domain,
VHH2 CDR1amino acid sequence
TFTMG
SEQ ID NO: 22-Ciltacabtagene autoleucel CAR BCMA binding domain,
VHH2 CDR2 amino acid sequence
AISLSPTLAYYAESVKG
SEQ ID NO: 23-Ciltacabtagene autoleucel CAR BCMA binding domain,
VHH2 CDR3 amino acid sequence
ADRKSVMSIRPDY
GPRC5DxCD3 bispecific antibody
SEQ ID NO: 101-GPRC5D binding domain heavy chain CDR1
GYTMN
SEQ ID NO: 102-GPRC5D binding domain heavy chain CDR2
LINPYNSDTNYAQKLQG
SEQ ID NO: 103-GPRC5D binding domain heavy chain CDR3
VALRVALDY
SEQ ID NO: 104-GPRC5D binding domain light chain CDR1
KASQNVATHVG
SEQ ID NO: 105-GPRC5D binding domain light chain CDR2
SASYRYS
SEQ ID NO: 106-GPRC5D binding domain light chain CDR3
QQYNRYPYT
SEQ ID NO: 107-CD3 binding domain heavy chain CDR1
TYAMN
SEQ ID NO: 108-CD3 binding domain heavy chain CDR2
RIRSKYNNYATYYAASVKG
SEQ ID NO: 109-CD3 binding domain heavy chain CDR3
HGNFGNSYVSWFAY
SEQ ID NO: 110-CD3 binding domain light chain CDR1
RSSTGAVTTSNYAN
SEQ ID NO: 111-CD3 binding domain light chain CDR2
GTNKRAP
SEQ ID NO: 112-CD3 binding domain light chain CDR3
ALWYSNLWV
SEQ ID NO: 113-GPRC5D binding domain heavy chain variable region
QVQLVQSGAEVKKPGASVKVSCKASGYSFTGYTMNWVRQAPGQGLEWMGLINPYNS
DTNYAQKLQGRVTMTTDTSTSTAYMELRSLRSDDTAVYYCARVALRVALDYWGQGTL
VTVSS
SEQ ID NO: 114-GPRC5D binding domain light chain variable region
DIQMTQSPSSLSASVGDRVTITCKASQNVATHVGWYQQKPGKAPKRLIYSASYRYSGVP
SRFSGSGSGTEFTLTISNLQPEDFATYYCQQYNRYPYTFGQGTKLEIK
SEQ ID NO: 115-CD3 binding domain heavy chain variable region
EVOLVESGGGLVQPGGSLRLSCAASGFTFNTYAMNWVRQAPGKGLEWVARIRSKYNN
YATYYAASVKGRFTISRDDSKNSLYLQMNSLKTEDTAVYYCARHGNFGNSYVSWFAY
WGQGTLVTVSS
SEQ ID NO: 116-CD3 binding domain light chain variable region
QTVVTQEPSLTVSPGGTVTLTCRSSTGAVTTSNYANWVQQKPGQAPRGLIGGTNKRAP
GTPARFSGSLLGGKAALTLSGVQPEDEAEYYCALWYSNLWVFGGGTKLTVLGQP
SEQ ID NO: 117-GPRC5D binding domain heavy chain sequence
QVQLVQSGAEVKKPGASVKVSCKASGYSFTGYTMNWVRQAPGQGLEWMGLINPYNS
DTNYAQKLQGRVTMTTDTSTSTAYMELRSLRSDDTAVYYCARVALRVALDYWGQGTL
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEAA
GGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 118-GPRC5D binding domain light chain sequence
DIQMTQSPSSLSASVGDRVTITCKASQNVATHVGWYQQKPGKAPKRLIYSASYRYSGVP
SRFSGSGSGTEFTLTISNLQPEDFATYYCQQYNRYPYTFGQGTKLEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 119-CD3 binding domain heavy chain sequence
EVOLVESGGGLVQPGGSLRLSCAASGFTFNTYAMNWVRQAPGKGLEWVARIRSKYNN
YATYYAASVKGRFTISRDDSKNSLYLQMNSLKTEDTAVYYCARHGNFGNSYVSWFAY
WGQGTLVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGPPCPP
CPAPEAAGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSF
LLYSKLTVDKSRWQEGNVFSCSVMHEALHNHYTQKSLSLSLGK
SEQ ID NO: 120-CD3 binding domain light chain sequence
QTVVTQEPSLTVSPGGTVTLTCRSSTGAVTTSNYANWVQQKPGQAPRGLIGGTNKRAP
GTPARFSGSLLGGKAALTLSGVQPEDEAEYYCALWYSNLWVFGGGTKLTVLGQPKAAP
SVTLFPPSSEELQANKATLVCLISDFYPGAVTVAWKADSSPVKAGVETTTPSKQSNNKY
AASSYLSLTPEQWKSHRSYSCQVTHEGSTVEKTVAPTECS
SEQ ID NO: 121-GPRC5D protein sequence
MYKDCIESTGDYFLLCDAEGPWGIILESLAILGIVVTILLLLAFLFLMRKIQDCSQWNVLP
TQLLFLLSVLGLFGLAFAFIIELNQQTAPVRYFLFGVLFALCESCLLAHASNLVKLVRGC
VSFSWTTILCIAIGCSLLQIIIATEYVTLIMTRGMMFVNMTPCQLNVDFVVLLVYVLFLM
ALTFFVSKATFCGPCENWKQHGRLIFITVLFSIIIWVVWISMLLRGNPQFQRQPQWDDPV
VCIALVTNAWVFLLLYIVPELCILYRSCRQECPLQGNACPVTAYQHSFQVENQELSRAR
DSDGAEEDVALTSYGTPIQPQTVDPTQECFIPQAKLSPQQDAGGV
SEQ ID NO: 122-human CD3 protein sequence
MQSGTHWRVLGLCLLSVGVWGQDGNEEMGGITQTPYKVSISGTTVILTCPQYPGSEILW
QHNDKNIGGDEDDKNIGSDEDHLSLKEFSELEQSGYYVCYPRGSKPEDANFYLYLRARV
CENCMEMDVMSVATIVIVDICITGGLLLLVYYWSKNRKAKAKPVTRGAGAGGRQRGQ
NKERPPPVPNPDYEPIRKGQRDLYSGLNQRRI
SEQ ID NO: 123-human CD3 protein extracellular domain sequence
DGNEEMGGITQTPYKVSISGTTVILTCPQYPGSEILWOHNDKNIGGDEDDKNIGSDEDHL
SLKEFSELEQSGYYVCYPRGSKPEDANFYLYLRARVCENCMEMD
SEQ ID NO: 124-GPRC5D heavy chain CDR1, Chothia numbering
GYSFTGY
SEQ ID NO: 125-GPRC5D heavy chain CDR2, Chothia numbering
NPYNSD
SEQ ID NO: 126-CD3 heavy chain CDR1, Chothia numbering
GFTFNTY
SEQ ID NO: 127-CD3 heavy chain CDR2, Chothia numbering
RSKYNNYA
SEQ ID NO: 128-GPRC5D heavy chain CDR1, AbM numbering
GYSFTGYTMN
SEQ ID NO: 129-GPRC5D heavy chain CDR2, AbM numbering
LINPYNSDTN
SEQ ID NO: 130-CD3 heavy chain CDR1, AbM numbering
GFTFNTYAMN
SEQ ID NO: 131-CD3 heavy chain CDR2, AbM numbering
RIRSKYNNYATY
SEQ ID NO: 132-GPRC5D heavy chain CDR1, IMGT numbering
GYSFTGYT
SEQ ID NO: 133-GPRC5D heavy chain CDR2, IMGT numbering
INPYNSDT
SEQ ID NO: 134-GPRC5D heavy chain CDR3, IMGT numbering
ARVALRVALDY
SEQ ID NO: 135-GPRC5D light chain CDR1, IMGT numbering
QNVATH
SEQ ID NO: 136-CD3 heavy chain CDR1, IMGT numbering
GFTFNTYA
SEQ ID NO: 137-CD3 heavy chain CDR2, IMGT numbering
IRSKYNNYAT
SEQ ID NO: 138-CD3 heavy chain CDR3, IMGT numbering
ARHGNFGNSYVSWFAY
SEQ ID NO: 139-CD3 light chain CDR1, IMGT numbering
TGAVTTSNY

1. A method of treating multiple myeloma in a subject in need thereof, the method comprising:
administering ciltacabtagene autoleucel to the subject at a dosage of 0.5-1.0×10⁶ CAR-positive viable T cells/kg, and
administering a GPRC5D×CD3 bispecific antibody to the subject;
wherein the administration of the GPRC5D×CD3 bispecific antibody occurs after the administration of ciltacabtagene autoleucel; and
wherein the subject has relapsed and/or refractory multiple myeloma, and received at least three prior lines of therapies, including a proteasomal inhibitor (PI), an immunomodulatory drug (IMiD), and an anti-CD38 antibody.

2. The method of claim 1, wherein the GPRC5D×CD3 bispecific antibody comprises a GPRC5D binding domain comprising a heavy chain complementarity determining region (CDR) 1 (HCDR1) of SEQ ID NO: 101, a HCDR2 of SEQ ID NO: 102, a HCDR3 of SEQ ID NO: 103, a light chain CDR1 (LCDR1) of SEQ ID NO: 104, a LCDR2 of SEQ ID NO: 105 and a LCDR3 of SEQ ID NO: 106, and a CD3 binding domain comprising a HCDR1 of SEQ ID NO: 107, a HCDR2 of SEQ ID NO: 108, a HCDR3 of SEQ ID NO: 109, a LCDR1 of SEQ ID NO: 110, a LCDR2 of SEQ ID NO: 111 and a LCDR3 of SEQ ID NO: 112.

3. The method of claim 1, wherein the GPRC5D×CD3 bispecific antibody comprises a GPRC5D binding domain comprising a heavy chain variable region (VH) comprising the amino acid sequence of SEQ ID NO: 113 and a light chain variable region (VL) comprising the amino acid sequence of SEQ ID NO: 114, and a CD3 binding domain comprising a VH comprising the amino acid sequence of SEQ ID NO: 115 and a VL comprising the amino acid sequence of SEQ ID NO: 116.

4. The method of claim 1, wherein the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

5. The method of claim 4, wherein the GPRC5D×CD3 bispecific antibody further comprises proline at position 228, alanine at position 234 and alanine at position 235 in both the HC1 and the HC2.

6. The method of claim 1, wherein the GPRC5D×CD3 bispecific antibody comprises a first heavy chain (HC1) comprising the amino acid sequence of SEQ ID NO: 117, a first light chain (LC1) comprising the amino acid sequence of SEQ ID NO: 118, a second heavy chain (HC2) comprising the amino acid sequence of SEQ ID NO: 119 and a second light chain (LC2) comprising the amino acid sequence of SEQ ID NO: 120.

7. The method of claim 1, wherein the GPRC5D×CD3 bispecific antibody is talquetamab.

8. The method of claim 1, wherein the administration of the GPRC5D×CD3 bispecific antibody is once every 2 weeks (Q2W) at a dosage of 0.8 mg/kg.

9. The method of claim 1, wherein the administration of the GPRC5D×CD3 bispecific antibody is weekly at a dosage of 0.4 mg/kg.

10. The method of claim 1, further comprising administering a conditioning regimen to the subject prior to administering ciltacabtagene autoleucel, wherein the conditioning regimen comprises one or more of cyclophosphamide and/or fludarabine.

11. The method of claim 10, wherein the conditioning regimen comprises cyclophosphamide at a dosage of 300 mg/m².

12. The method of claim 10, wherein the conditioning regimen comprises fludarabine at a dosage of 30 mg/m².

13. The method of claim 10, wherein the conditioning regimen comprises cyclophosphamide at a dosage of 300 mg/m² and fludarabine at a dosage of 30 mg/m².

14. The method of claim 10, wherein the conditioning regimen is administered to the subject daily, for up to 3 days.

15. The method of claim 10, wherein the ciltacabtagene autoleucel is administered to the subject 5 to 7 days after the start of the administration of the conditioning regimen.

16. The method of claim 1, wherein the ciltacabtagene autoleucel is administered to the subject at a dose of 0.75×10⁶ CAR-positive viable T cells/kg.

17. The method of claim 1, wherein the GPRC5D×CD3 GPRC5D×CD3-bispecific antibody is administered subcutaneously.

18. The method of claim 1, wherein the subject is administered up to 12 cycles of the GPRC5D×CD3 bispecific antibody.

19. The method of claim 1, wherein the subject is administered up to 12 cycles of the GPRC5D×CD3 bispecific antibody, and wherein the first cycle of the up to 12 cycles comprises three consecutive step-up doses of the GPRC5D×CD3 bispecific antibody of 0.01 mg/kg, 0.06 mg/kg, and 0.4 mg/kg, prior to receiving a dose of 0.8 mg/kg once every 2 weeks.

20. The method of claim 19, wherein the three consecutive step-up doses are administered 2, 3, 4, or 5 days apart.

21. The method of claim 19, wherein the subject is administered up to 12 cycles of the GPRC5D×CD3 bispecific antibody, and wherein the first cycle of the up to 12 cycles of the GPRC5D×CD3 bispecific antibody comprises:
the administration of a first step-up dose of 0.01 mg/kg of the GPRC5D×CD3 bispecific antibody,
the administration of a second step-up dose of 0.06 mg/kg of the GPRC5D×CD3 bispecific antibody 2, 3, 4, or 5 days after the administration of the first step-up dose,
the administration of a third step-up dose of 0.4 mg/kg of the GPRC5D×CD3 bispecific antibody 2, 3, or 4 days after the administration of the second step-up dose, and
the administration of 0.8 mg/kg of the GPRC5D×CD3 bispecific antibody 2, 3, 4, 5, 6, 7, 8, 9, or 10 days after the administration of the third step-up dose.

22. The method of claim 21, wherein the second through fourth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the fourth cycle comprises the administration of 0.8 mg/kg of the GPRC5D×CD3 bispecific antibody once every 4 weeks (Q4W).

23. The method of claim 21, wherein the second through fifth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the fifth cycle comprises the administration of 0.8 mg/kg of the GPRC5D×CD3 bispecific antibody once every 4 weeks (Q4W).

24. The method of claim 21, wherein the second through sixth cycles of the up to 12 cycles of the GPRC5D×CD3 bispecific antibody each comprise the administration of 0.8 mg/kg of the GPRC5D×CD3 bispecific antibody once every 2 weeks (Q2W),
and wherein each remaining cycle past the sixth cycle comprises the administration of 0.8 mg/kg of the GPRC5D×CD3 bispecific antibody once every 4 weeks (Q4W).

25. The method of claim 1, wherein the administration of the GPRC5D×CD3 bispecific antibody occurs no earlier than 56 days from the administration of ciltacabtagene autoleucel.

26. The method of claim 1, wherein the method achieves a partial response (PR), very good partial response (VGPR), complete response (CR) or stringent complete response (sCR) in the subject, according to IMWG criteria.

27.-29. (canceled)

30. The method of claim 1, wherein the method achieves MRD-negativity at a threshold of 10⁻⁵ before disease progression or start of a subsequent antimyeloma therapy.

31. The method of claim 1, wherein the method achieves sustained MRD-negative status, as determined by NGS with sensitivity of 105, for at least 6 months without examination showing MRD-positive or PD in between.

32. A method of treating multiple myeloma in a subject in need thereof, the method comprising:
administering ciltacabtagene autoleucel to the subject at a dosage of 0.5-1.0×10⁶ CAR-positive viable T cells/kg, and
administering a GPRC5D×CD3 bispecific antibody to the subject;
wherein the administration of the GPRC5D×CD3 bispecific antibody occurs after the administration of ciltacabtagene autoleucel, and
wherein the subject has newly diagnosed multiple myeloma and is transplant ineligible.

33.-63. (canceled)

64. A method of treating multiple myeloma in a subject in need thereof, the method comprising:
administering a GPRC5D×CD3 bispecific antibody to the subject, and
administering ciltacabtagene autoleucel to the subject at a dosage of 0.5-1.0×10⁶ CAR-positive viable T cells/kg;
wherein the administration of ciltacabtagene autoleucel occurs after the administration of the GPRC5D×CD3 bispecific antibody, and
wherein the subject has relapsed and/or refractory multiple myeloma, and received at least three prior lines of therapies, including a proteasomal inhibitor (PI), an immunomodulatory drug (IMiD), and an anti-CD38 antibody.

65.-94. (canceled)
